(19) United States
(12) Patent Application Publication
(10) Pub. No.: US 2026/0285831 A1
(43) Pub. Date: Sep. 24, 2026

(54) LACTAM DERIVATIVES FOR INHIBITING NLRP3 AND USES THEREOF

(71) Applicant: Ventus Therapeutics U.S., Inc., Waltham, MA (US)

(72) Inventors: Stéphane DORICH, Pointe-Claire (CA); Stéphane CIBLAT, Montréal (CA)

(21) Appl. No.: 19/677,250

(22) Filed: May 14, 2026

Related U.S. Application Data
(63) Continuation of application No. PCT/US2024/056195, filed on Nov. 15, 2024.
(60) Provisional application No. 63/599,319, filed on Nov. 15, 2023.

(51) Int. Cl. C07D 401/04; A61K 31/4439; A61K 31/4709; A61K 31/4725; A61K 31/497; A61K 31/501; A61K 31/506; C07D 403/04

(52) U.S. Cl. CPC C07D 401/04; A61K 31/4439; A61K 31/4709; A61K 31/4725; A61K 31/497; A61K 31/501; A61K 31/506; C07D 403/04

(57) ABSTRACT

The present disclosure relates to compounds of Formula (I):

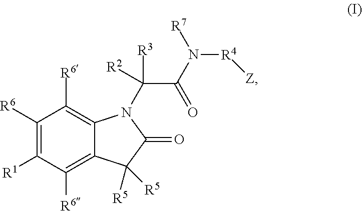

and pharmaceutically acceptable salts thereof, wherein R¹, R³, R⁴, R^{4a}, R^{4b}, Z, R⁵, R⁶, R^{6′}, and R^{6″}, are described herein, and R⁷ and R² are joined to form a 5- to 10-membered monocyclic or bicyclic heterocyclyl or 5- to 10-membered monocyclic or bicyclic heteroaryl, and methods of preparation, methods of treatment and prevention, and pharmaceutical compositions comprising same. The present disclosure further relates to the use of the compounds of Formula (I), and pharmaceutically acceptable salts thereof, in the treatment or prevention of NLRP3-related diseases and disorders.

### RELATED APPLICATIONS

This application is a continuation of International Application No. PCT/US2024/056195, filed on Nov. 15, 2024, which claims priority to, and the benefit of, U.S. Provisional Patent Application No. 63/599,319, filed on Nov. 15, 2023, the entire contents of each which are incorporated herein by reference.

### BACKGROUND

Innate immune responses are mediated by different types of receptors termed pattern-recognition receptors (PRRs). PRR⁵ recognize the presence of pathogen-associated molecular patterns (PAMPs) and damage-associated molecular patterns (DAMPs). Once engaged these receptors trigger the activation of downstream inflammatory pathways that will help resolve injury. However, in many instances this activation can be uncontrolled and leads to disease.

The inflammasomes represent a class of PRR⁵ that are crucial components of the innate immune response. Activation of the inflammasomes trigger a cascade of events that releases IL-10, IL-18, and promotes an inflammatory form of cell death called pyroptosis induced by the activation of Gasdermin.

Pyroptosis is a unique form of inflammatory cell death that leads to the release of not only cytokines but also other intracellular components that promote a broader immune response both of the innate and acquired immune system. Thus, inflammasome activation is a major regulatory of the inflammatory cascade.

NLRP3 is the most characterized inflammasome and has been shown to be critical in innate immunity and inflammatory responses. While several other NLR complexes, such as NLRC4, are activated under very specific circumstances, NLRP3 can be activated by numerous stimuli and should be seen as a sensor of intracellular homeostatic imbalance. Therefore, its precise functioning is essential. In addition to playing a role in host immune defense, dysregulation of NLRP3 has been linked to the pathogenesis of many inflammatory disorders. These include genetic diseases such as cryopyrin-associated periodic syndromes (CAPS) which is caused by gain-of-function mutations in the NLRP3 gene, as well as many prevalent neurologic and systemic diseases. Importantly, NLRP3 hyperactivation has been demonstrated pre-clinically to play a critical role in a plethora of inflammatory and degenerative diseases including, NASH, atherosclerosis and other cardiovascular diseases, Alzheimer's disease, Parkinson's disease, diabetes, gout, and numerous other autoinflammatory diseases. See, e.g., Li et al., European Journal of Pharmacology (2022) 928:175091; Nguyen et al., Journal of Parkinson's Disease (2022) 12:2117-2133; Su et al., Current Medicinal Chemistry (2021) 28:569-582; Zahid et al., Frontiers in Immunology (2019) 10:2538. Thus, there is an unmet need in the field to develop small molecules for modulating NLRP3 activity to treat various diseases and disorders.

### SUMMARY

Provided herein are NLRP3 inhibitors of Formula (I):

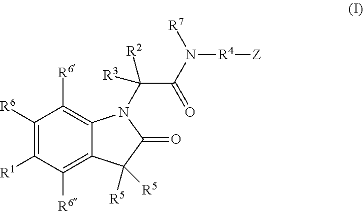

and pharmaceutically acceptable salts thereof, wherein R¹, R³, R⁴, R⁵, R⁶, R^{6′}, R^{6″}, and Z are described herein, and R⁷ and R² are joined to form a 5- to 10-membered monocyclic or bicyclic heterocyclyl or 5- to 10-membered monocyclic or bicyclic heteroaryl.

Further provided are methods of preparation, methods of treatment and prevention, and pharmaceutical compositions comprising same.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, separatable atropisomer (arising from hindered rotation resulting in axial chirality), or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972). Compounds described herein can additionally encompasses individual isomers substantially free of other isomers, and alternatively, as mixtures of various isomers.

Unless otherwise stated, compounds described herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C), 3-pentanyl (C₅), amyl (C₅), neopentyl (C₅), 3-methyl-2-butanyl (C₅), tertiary amyl (C₅), and n-hexyl (C₆).

“Haloalkyl” refers to a substituted alkyl group, as defined herein, wherein one or more of the hydrogen atoms are independently replaced by a halogen, e.g., fluoro, bromo, chloro, or iodo. In some embodiments, the haloalkyl group has 1 to 6 carbon atoms (“C₁₋₆ haloalkyl”). In some embodiments, the haloalkyl group has 1 to 5 carbon atoms (“C₁₋₅ haloalkyl”). In some embodiments, the haloalkyl group has 1 to 4 carbon atoms (“C₁₋₄ haloalkyl”). In some embodiments, the haloalkyl group has 1 to 3 carbon atoms (“C₁₋₃ haloalkyl”). In some embodiments, the haloalkyl group has 1 to 2 carbon atoms (“C₁₋₂ haloalkyl”).

In some embodiments, all of the haloalkyl hydrogen atoms are replaced with fluoro to provide a perfluoroalkyl group. In some embodiments, all of the haloalkyl hydrogen atoms are replaced with chloro to provide a “perchloroalkyl” group. Examples of haloalkyl groups include —CF₃, —CF₂CF₃, —CF₂CF₂CF₃, —CCl₃, —CFCl₂, —CF₂Cl, and the like.

“Cycloalkyl” refers to a radical of a non-aromatic, fully saturated or partially saturated, cyclic hydrocarbon group having from 3 to 6 ring carbon atoms (“C₃₋₆ cycloalkyl”) and zero heteroatoms in the non-aromatic ring system. In some embodiments, the cycloalkyl group has 3 carbon atoms (“C₃ cycloalkyl”). In some embodiments, the cycloalkyl group has 4 carbon atoms (“C₄ cycloalkyl”). In some embodiments, the cycloalkyl group has 5 carbon atoms (“C₅ cycloalkyl”). In some embodiments, the cycloalkyl group has 6 carbon atoms (“C₆ cycloalkyl”). Exemplary C₃₋₆ cycloalkyl groups include, without limitation, cyclopropyl (C₃), cyclopropenyl (C₃), cyclobutyl (C₄), cyclobutenyl (C₄), cyclopentyl (C), cyclopentenyl (C), cyclohexyl (C₆), cyclohexenyl (C₆), cyclohexadienyl (C₆), and the like.

“Heterocyclyl” refers to a radical of a 3- to 10-membered non-aromatic, fully saturated or partially saturated, monocyclic or bicyclic ring system having ring carbon atoms and one or more ring heteroatoms (e.g., 1, 2, 3, or 4 ring heteroatoms), wherein each ring heteroatom is independently selected from nitrogen, oxygen, and sulfur (“3- to 10-membered heterocyclyl”). In heterocyclyl groups that contain a nitrogen atom, the point of attachment can be on a carbon or nitrogen ring atom, as valency permits. Exemplary 3-membered heterocyclyl groups containing 1 ring heteroatom include, without limitation, aziridinyl, oxiranyl, and thiiranyl. Exemplary 4-membered heterocyclyl groups containing 1 ring heteroatom include, without limitation, azetidinyl, oxetanyl and thietanyl. Exemplary 5-membered heterocyclyl groups containing 1 ring heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 ring heteroatoms include, without limitation, dioxolanyl, oxathiolanyl and dithiolanyl. Exemplary 5-membered heterocyclyl groups containing 3 ring heteroatoms include, without limitation, triazolinyl, oxadiazolinyl, and thiadiazolinyl. Exemplary 6-membered heterocyclyl groups containing 1 ring heteroatom include, without limitation, piperidinyl, tetrahydropyranyl, dihydropyridinyl, and thianyl. Exemplary 6-membered heterocyclyl groups containing 2 ring heteroatoms include, without limitation, piperazinyl, morpholinyl, dithianyl, and dioxanyl. Exemplary 6-membered heterocyclyl groups containing 3 ring heteroatoms include, without limitation, triazinanyl. Exemplary 7-membered heterocyclyl groups containing 1 ring heteroatom include, without limitation, azepanyl, oxepanyl and thiepanyl. Exemplary 8-membered heterocyclyl groups containing 1 ring heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, and the like. For clarity, a substituted or unsubstituted pyrrolidine-2-one moiety, such as the divalent moiety depicted below, is considered a heterocyclyl group:

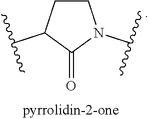 [SMILES: *C1CCN(*)C1=O]

As used herein, “aryl” refers to a radical of a monocyclic aromatic ring system having 6 ring carbon atoms and zero ring heteroatoms provided in the aromatic ring system (“C₆ aryl”, i.e., phenyl).

As used herein, the term “heteroaryl” refers to a 5-, 6-, or 7-membered monocyclic or 7-, 8-, 9-, 10-, 11-, or 12-membered bicyclic aromatic ring which consists of ring carbon atoms and one or more ring heteroatoms, e.g., 1 or 1-2 or 1-3 or 1-4 or 1-5 or 1-6 ring heteroatoms, or e.g., 1, 2, 3, 4, 5, or 6 ring heteroatoms, independently selected from the group consisting of nitrogen, oxygen, and sulfur. In heteroaryl groups that contain one or more ring nitrogen atoms, the point of attachment can be a carbon or nitrogen atom, as valency permits. The ring nitrogen atom may be substituted or unsubstituted, as valency and aromaticity permits. Exemplary 5-membered heteroaryl groups containing 1 ring heteroatom include, without limitation, pyrrolyl, furanyl and thiophenyl. Exemplary 5-membered heteroaryl groups containing 2 ring heteroatoms include, without limitation, imidazolyl, pyrazolyl, oxazolyl, isoxazolyl, thiazolyl, and isothiazolyl. Exemplary 5-membered heteroaryl groups containing 3 ring heteroatoms include, without limitation, triazolyl, oxadiazolyl, and thiadiazolyl. Exemplary 5-membered heteroaryl groups containing 4 ring heteroatoms include, without limitation, tetrazolyl. Exemplary 6-membered heteroaryl groups containing 1 ring heteroatom include, without limitation, pyridinyl. Exemplary 6-membered heteroaryl groups containing 2 ring heteroatoms include, without limitation, pyridazinyl, pyrimidinyl, and pyrazinyl. Exemplary 6-membered heteroaryl groups containing 3 or 4 ring heteroatoms include, without limitation, triazinyl and tetrazinyl, respectively. Exemplary 7-membered heteroaryl groups containing 1 ring heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. For clarity, substituted or unsubstituted pyridine-2(1H)-one, pyrazin-2(1H)-one, pyrimidine-4(3H)-one, and pyridazine-3(2H)one moieties, such as the divalent moieties depicted below, are considered heteroaryl groups:

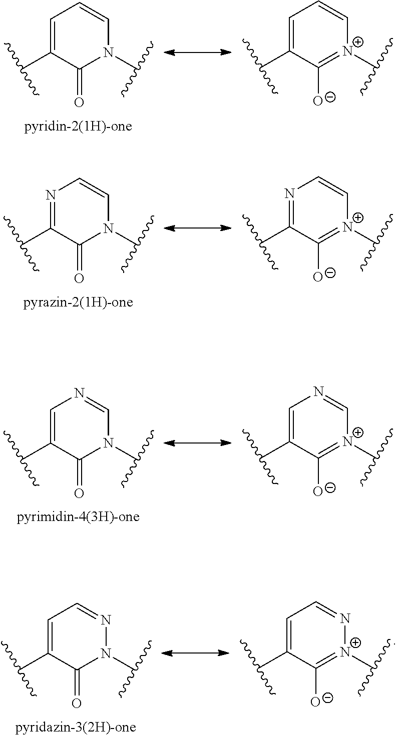

“Halo” or “halogen” refers to fluorine (fluoro, —F), chlorine (chloro, —Cl), bromine (bromo, —Br), or iodine (iodo, —I) radicals.

Affixing the suffix “-ene” to a group indicates the group is a divalent moiety, e.g., C₁-C₆ alkylene is the divalent moiety of branched or linear C₁-C₆ alkyl. By way of example, a C₁₋₃ alkylene, which may be linear or branched, include, but are not limited to, —CH₂—, —CH(CH₃)—, —C(CH₃)₂—, —CH₂CH₂—, —CH₂CH(CH₃)—, —CH₂C(CH₃)₂—, and —CH₂CH₂CH₂—.

The term “alkoxy” or “alkoxyl” includes an alkyl group covalently linked by a direct bond to an oxygen atom, wherein the radical (point of attachment) is on the oxygen atom. Examples of alkoxy groups include, but are not limited to, methoxy, ethoxy, isopropyloxy, propoxy, butoxy and pentoxy groups.

The term “haloalkoxy” or “haloalkoxyl” includes an haloalkyl group covalently linked by a direct bond to an oxygen atom, wherein the radical (point of attachment) is on the oxygen atom. Examples of alkoxy groups include, but are not limited to, halomethoxy, haloethoxy, haloisopropyloxy, halopropoxy, halobutoxy and halopentoxy groups.

Salts, pharmaceutically acceptable salts, and non-ionized compounds (e.g., non-ionized acids (also referred to as free acids) and/or free bases) of compounds of Formula (I) and intermediates thereof are contemplated herein.

“Salt” refers to any and all salts.

A “non-ionized” compound refers to a neutral form of a compound which is not a salt or pharmaceutically acceptable salt.

“Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Examples of pharmaceutically acceptable acid addition salts include, but are not limited to, salts formed from inorganic acids, such as hydrochloric acid, hydrobromic acid, phosphoric acid, sulfuric acid and perchloric acid salts, or salts formed from organic acids such as acetic acid, oxalic acid, maleic acid, tartaric acid, citric acid, succinic acid or malonic acid or by using other methods used in the art such as ion exchange.

A “leaving group” is an art-understood term referring to a molecular fragment that departs with a pair of electrons in heterolytic bond cleavage, wherein the molecular fragment is an anion or neutral molecule. See, for example, Smith, March Advanced Organic Chemistry 6th ed. (501-502). Exemplary leaving groups include, but are not limited to, halo (e.g., chloro, bromo, iodo) and sulfonyl substituted hydroxyl groups (e.g., —O-tosyl, —O-mesyl, and —O-besyl).

A “patient” or “subject” is used interchangeably herein, and refers to a mammal, e.g., a human, mouse, rat, guinea pig, dog, cat, horse, cow, pig, or non-human primate, such as a monkey, chimpanzee, baboon, or rhesus. In some embodiments, the patient or subject is human.

“Effective amount” refers to an amount of a compound, or a pharmaceutically acceptable salt thereof, sufficient to provide a benefit in the treatment of a disease or disorder in a subject. The effective amount of a compound, or a pharmaceutically acceptable salt thereof, may vary depending on such factors as, e.g., the desired biological endpoint, the pharmacokinetics of the compound, the disease being treated, the mode of administration, and the age, health, and condition of the subject.

“Disease” or “disorder” are used interchangeably herein.

“Treating,” “treat,” or “treatment” describes the management and care of a subject in need thereof, for the purpose of combating a disease or disorder in the subject, and includes the administration of a compound, or a pharmaceutically acceptable salt thereof, as described herein, to alleviate one or more symptoms or complications of a disease or disorder, or to eliminate the disease or disorder from the subject. The term “treat” can also include treatment of a cell in vitro or treatment of an animal model (in vivo).

As used herein, the term “preventing,” “prevent,” or “protecting against” describes the management and care of a subject in need thereof that may have or has a predisposition for the disease or disorder but has not yet experienced or displayed symptoms or complications of a disease or disorder, for the purpose of preventing the appearance of one or more symptoms or complications of a disease or disorder in the subject, and includes the administration of a compound, or a pharmaceutically acceptable salt thereof, as described herein.

“Inhibition,” “inhibiting,” “inhibit,” and “inhibitor,” and the like, refer to the ability of a compound, or a pharmaceutically acceptable salt thereof, to reduce, slow, halt or prevent activity of a particular biological process (e.g., NLRP3 activity) in a cell relative to vehicle.

The phrase “at least one” refers to one instance or more than one instance.

The articles “a” and “an” are used in this disclosure to refer to one or more than one (i.e., to at least one) of the grammatical object of the article.

The term “and/or” is used in this disclosure to mean either “and” or “or” unless indicated otherwise.

### DETAILED DESCRIPTION OF SOME EMBODIMENTS

### (i) Compounds

Provided herein are compounds of Formula (I):

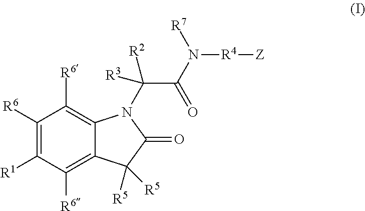

and pharmaceutically acceptable salts thereof,
wherein:
R¹ is halo, C₁-C₆ alkyl, C₁-C₆ haloalkyl, or C₃-C₆ cycloalkyl;
R⁷ and R² are joined to form a 5- to 10-membered monocyclic or bicyclic heterocyclyl or 5- to 10-membered monocyclic or bicyclic heteroaryl, wherein the heterocyclyl or heteroaryl are optionally substituted with one or more R^{7a}, wherein each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃-C₅ cycloalkyl, or 4 to 5-membered heterocyclyl, wherein the alkyl, haloalkyl, alkoxy, haloalkoxy, aryl, heteroaryl, cycloalkyl, and heterocyclyl are optionally substituted with one or more R^{7b}, further wherein R^{7b} is halo, —CN, —OH, C₁-C₆ alkyl, C₁-C₆ haloalkyl, C₁-C₆ alkoxy, or C₁-C₆ haloalkoxy;
R³ is absent, H, C₁-C₆ alkyl, or C₁-C₆ haloalkyl;
R⁴ is —C₁-C₆ alkylene-optionally substituted with one or more R^{4a}, wherein each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl, or two R^{4a} are joined to form a C₃-C₅ cycloalkyl or 4- to 6-membered heterocyclyl ring, wherein the alkyl, haloalkyl, alkoxy, haloalkoxy, cycloalkyl, and heterocyclyl are optionally substituted with one or more R^{4b}, further wherein R^{4b} is halo, —CN, —OH, C₁-C₆ alkyl, C₁-C₆ haloalkyl, C₁-C₆ alkoxy, or C₁-C₆ haloalkoxy;
Z is a —CO₂H or —CO₂PG, wherein PG is C₁-C₆ alkyl, C₆ aryl, or (C₆ aryl)(C₁-C₆ alkyl), further wherein alkyl and aryl are optionally substituted with one or more halo, C₁-C₆ alkyl, C₁-C₆ alkoxy, or C₁-C₆ haloalkoxy;
each R⁵ is independently halo, C₁-C₆ alkyl, or C₁-C₆ haloalkyl; and
each R⁶, R^{6′}, or R^{6″} is independently H, halo, —OH, C₁-C₆ alkoxy, C₁-C₆ haloalkoxy, C₁-C₆ alkyl, or C₁-C₆ haloalkyl.

In some embodiments, the compound is of Formula (I), or a pharmaceutically acceptable salt thereof, wherein:
R¹ is halo, C₁-C₆ haloalkyl, or C₃-C₄ cycloalkyl;
R⁷ and R² are joined to form a 5- to 10-membered monocyclic or bicyclic heteroaryl, wherein the heteroaryl is optionally substituted with one or more R^{7a}, wherein each R^{7a} is independently C₁-C₆ alkyl, C₁-C₆ haloalkyl, C₆ aryl, or C₃-C₅ cycloalkyl;
R³ is absent;
R⁴ is —C₁-C₆ alkylene-;
Z is a —CO₂H;
each R⁵ is independently C₁-C₆ alkyl; and
each R⁶, R^{6′}, or R^{6″} is independently H, halo, or —OH.

In some embodiments, the compound is of Formula (I), or a pharmaceutically acceptable salt thereof, wherein:
R¹ is halo, C₁-C₆ haloalkyl, or C₃-C₄ cycloalkyl;
R⁷ and R² are joined to form a 6-membered monocyclic heteroaryl, wherein the heteroaryl is optionally substituted with one or more R^{7a}, wherein each R^{7a} is independently C₁-C₆ alkyl, C₁-C₆ haloalkyl, C₆ aryl, or C₃-C₅ cycloalkyl;
R³ is absent;
R⁴ is —C₁-C₆ alkylene-;
Z is a —CO₂H;
each R⁵ is independently C₁-C₆ alkyl; and
each R⁶, R^{6′}, or R^{6″} is independently H, halo, or —OH.

In some embodiments, the compound is of Formula (I), or a pharmaceutically acceptable salt thereof, wherein:
R¹ is halo, C₁-C₆ haloalkyl, or C₃-C₄ cycloalkyl;
R⁷ and R² are joined to form a 5- to 10-membered monocyclic or bicyclic heterocyclyl, wherein the heterocyclyl is optionally substituted with one or more R^{7a}, wherein each R^{7a} is independently C₁-C₆ alkyl, C₁-C₆ haloalkyl, C₆ aryl, or C₃-C₅ cycloalkyl;
R³ is absent;
R⁴ is —C₁-C₆ alkylene-;
Z is a —CO₂H;
each R⁵ is independently C₁-C₆ alkyl; and
each R⁶, R^{6′}, or R^{6″} is independently H, halo, or —OH.

In some embodiments, the compound is of Formula (I), or a pharmaceutically acceptable salt thereof, wherein:
R¹ is halo, C₁-C₆ haloalkyl, or C₃-C₄ cycloalkyl;
R⁷ and R² are joined to form a 5-membered monocyclic heterocyclyl, wherein the heterocyclyl is optionally substituted with one or more R^{7a}, wherein each R^{7a} is independently C₁-C₆ alkyl, C₁-C₆ haloalkyl, C₆ aryl, or C₃-C₅ cycloalkyl;
R³ is absent;
R⁴ is —C₁-C₆ alkylene-;
Z is a —CO₂H;
each R⁵ is independently C₁-C₆ alkyl; and
each R⁶, R^{6′}, or R^{6″} is independently H, halo, or —OH.

It is understood that, as valency permits, R³ being absent and R³ being hydrogen may be used interchangeably (e.g., a carbon attached to R³ allows the presence of a hydrogen). For the avoidance of doubt, if valency permits a hydrogen at the R³ position, defining R³ as being absent is interchangeable with R³ being hydrogen.

It is further understood that, for a compound of Formula (I), R¹, R², R³, R⁴, R^{4a}, R^{4b}, R⁵, R⁶, R^{6′}, R^{6″}, R¹, R^{7a}, R^{7b}, and Z can each be, where applicable, selected from the groups described herein, and any group described herein for any of R, R², R³, R⁴, R^{4a}, R^{4b}, R⁵, R⁶, R¹, R^{6″}, R^{6″}, R^{7a}, R^{7b}, and Z can be combined, where applicable, with any group described herein for one or more of the remainder of R¹, R, R³, R⁴, R^{4a}, R^{4b}, R⁵, R⁶, R^{6′}, R^{6″}, R⁷, R^{7a}, R^{7b}, and Z.

Additional embodiments are further described below and herein.

(a) R¹, R², R³, R⁵, R⁶, R^{6′}, R^{6″} and R⁷

As generally described herein, R¹ is halo, C₁-C₆ alkyl, C₁-C₆ haloalkyl, or C₃-C₆ cycloalkyl.

In some embodiments, R¹ is halo. In some embodiments, R¹ is F, Br, Cl, or I. In some embodiments, R¹ is F, Br, or Cl. In some embodiments, R¹ is F. In some embodiments, R¹ is Br. In some embodiments, R¹ is Cl. In some embodiments, R¹ is I.

In some embodiments, R¹ is C₁-C₆ alkyl C₁-C₆ haloalkyl, or C₃-C₆ cycloalkyl.

In some embodiments, R¹ is C₁-C₆ alkyl.

In some embodiments, R¹ is methyl. In some embodiments, R¹ is ethyl. In some embodiments, R¹ is propyl. In some embodiments, R¹ is butyl. In some embodiments, R¹ is pentyl. In some embodiments, R¹ is hexyl. In some embodiments, R¹ is isopropyl. In some embodiments, R¹ is isobutyl. In some embodiments, R¹ is isopentyl. In some embodiments, R¹ is isohexyl. In some embodiments, R¹ is secbutyl. In some embodiments, R¹ is secpentyl. In some embodiments, R¹ is sechexyl. In some embodiments, R¹ is tertbutyl.

In some embodiments, R¹ is C₁-C₆ haloalkyl.

In some embodiments, R¹ is halomethyl. In some embodiments, R¹ is haloethyl. In some embodiments, R¹ is halopropyl. In some embodiments, R¹ is halobutyl. In some embodiments, R¹ is halopentyl. In some embodiments, R¹ is halohexyl.

In some embodiments, R¹ is C₃-C₆ cycloalkyl.

In some embodiments, R¹ is C₃-C₄ cycloalkyl.

In some embodiments, R¹ is C₃ cycloalkyl. In some embodiments, R¹ is C₄ cycloalkyl. In some embodiments, R¹ is C₅ cycloalkyl. In some embodiments, R¹ is C₆ cycloalkyl.

In some embodiments, R¹ is halo, C₁-C₆ haloalkyl, or C₃-C₆ cycloalkyl.

In some embodiments, R¹ is Br, Cl, CF₃, or cyclopropyl.

In some embodiments, R¹ is CF₃ or cyclopropyl.

As generally described herein, R³ is absent, H, C₁-C₆ alkyl, or C₁-C₆ haloalkyl.

In some embodiments, R³ is absent.

In some embodiments, R³ is H or C₁-C₆ alkyl.

In some embodiments, R³ is H.

In some embodiments, R³ is C₁-C₆ alkyl.

In some embodiments, R³ is methyl. In some embodiments, R³ is ethyl. In some embodiments, R³ is propyl. In some embodiments, R³ is butyl. In some embodiments, R³ is pentyl. In some embodiments, R³ is hexyl. In some embodiments, R³ is isopropyl. In some embodiments, R³ is isobutyl. In some embodiments, R³ is isopentyl. In some embodiments, R³ is isohexyl. In some embodiments, R³ is secbutyl. In some embodiments, R³ is secpentyl. In some embodiments, R³ is sechexyl. In some embodiments, R³ is tertbutyl.

In some embodiments, R³ is C₁-C₆ haloalkyl.

In some embodiments, R³ is halomethyl. In some embodiments, R³ is haloethyl. In some embodiments, R³ is halopropyl. In some embodiments, R³ is halobutyl. In some embodiments, R³ is halopentyl. In some embodiments, R³ is halohexyl.

As generally described herein, each R⁵ is independently halo, C₁-C₆ alkyl, or C₁-C₆ haloalkyl.

In some embodiments, each instance of R⁵ is the same group.

In some embodiments, each instance of R⁵ is halo. For example, in some embodiments, each instance of R⁵ is fluoro.

In some embodiments, each R⁵ is independently halo. In some embodiments, each R⁵ is independently F, Br, Cl, or I. In some embodiments, each R⁵ is independently F, Br, or Cl. In some embodiments, each R⁵ is independently F. In some embodiments, each R⁵ is independently Br. In some embodiments, each R⁵ is independently Cl. In some embodiments, each R⁵ is independently I.

In some embodiments, each instance of R⁵ is the same optionally substituted C₁-C₆ alkyl group, e.g., —CH₃. In some embodiments, each instance of R⁵ is different.

In some embodiments, each instance of R⁵ is independently C₁-C₆ alkyl.

In some embodiments, at least one instance of R⁵ is methyl (—CH₃). In some embodiments, at least one instance of R is ethyl. In some embodiments, at least one instance of R is propyl. In some embodiments, at least one instance of R⁵ is butyl. In some embodiments, at least one instance of R is pentyl. In some embodiments, at least one instance of R⁵ is hexyl. In some embodiments, at least one instance of R⁵ is isopropyl. In some embodiments, at least one instance of R is isobutyl. In some embodiments, at least one instance of R is isopentyl. In some embodiments, at least one instance of R⁵ is isohexyl. In some embodiments, at least one instance of R⁵ is secbutyl. In some embodiments, at least one instance of R is secpentyl. In some embodiments, at least one instance of R is sechexyl. In some embodiments, at least one instance of R⁵ is tertbutyl.

In some embodiments, each R⁵ is independently methyl (—CH₃). In some embodiments, each R⁵ is independently ethyl. In some embodiments, each R⁵ is independently propyl. In some embodiments, each R⁵ is independently butyl. In some embodiments, each R⁵ is independently pentyl. In some embodiments, each R⁵ is independently hexyl. In some embodiments, each R⁵ is independently isopropyl. In some embodiments, each R⁵ is independently isobutyl. In some embodiments, each R⁵ is independently isopentyl. In some embodiments, each R⁵ is independently isohexyl. In some embodiments, each R⁵ is independently secbutyl. In some embodiments, each R⁵ is independently secpentyl. In some embodiments, each R⁵ is independently sechexyl. In some embodiments, each R⁵ is independently tertbutyl.

In some embodiments, each R⁵ is independently C₁-C₆ haloalkyl.

In some embodiments, each R⁵ is independently halomethyl. In some embodiments, each R⁵ is independently haloethyl. In some embodiments, each R⁵ is independently halopropyl. In some embodiments, each R⁵ is independently halobutyl. In some embodiments, each R⁵ is independently halopentyl. In some embodiments, each R⁵ is independently halohexyl.

As generally described herein, each R⁶, R^{6′}, or R^{6″} is independently H, halo, —OH, C₁-C₆ alkoxy, C₁-C₆ haloalkoxy, C₁-C₆ alkyl, or C₁-C₆ haloalkyl.

As generally described herein, R⁶ is H, halo, —OH, C₁-C₆ alkoxy, C₁-C₆ haloalkoxy, C₁-C₆ alkyl, or C₁-C₆ haloalkyl.

In some embodiments, R⁶ is H.

In some embodiments, R⁶ is halo, —OH, C₁-C₆ alkyl, or C₁-C₆ haloalkyl.

In some embodiments, R⁶ is C₁-C₆ alkoxy or C₁-C₆ haloalkoxy.

In some embodiments, R⁶ is halo or —OH.

In some embodiments, R⁶ is halo.

In some embodiments, R⁶ is F, Cl, Br, or I. In some embodiments, R⁶ is F, Cl, or Br.

In some embodiments, R⁶ is F. In some embodiments, R⁶ is Cl. In some embodiments, R⁶ is Br. In some embodiments, R⁶ is I.

In some embodiments, R⁶ is —OH.

In some embodiments, R⁶ is C₁-C₆ alkoxy.

In some embodiments, R⁶ is C₁-C₆ haloalkoxy.

In some embodiments, R⁶ is C₁-C₆ alkyl.

In some embodiments, R⁶ is methyl. In some embodiments, R⁶ is ethyl. In some embodiments, R⁶ is propyl. In some embodiments, R⁶ is butyl. In some embodiments, R⁶ is pentyl. In some embodiments, R⁶ is hexyl. In some embodiments, R⁶ is isopropyl. In some embodiments, R⁶ is isobutyl. In some embodiments, R⁶ is isopentyl. In some embodiments, R⁶ is isohexyl. In some embodiments, R⁶ is secbutyl. In some embodiments, R⁶ is secpentyl. In some embodiments, R⁶ is sechexyl. In some embodiments, R⁶ is tertbutyl.

In some embodiments, R⁶ is C₁-C₆ haloalkyl.

In some embodiments, R⁶ is halomethyl. In some embodiments, R⁶ is haloethyl. In some embodiments, R⁶ is halopropyl. In some embodiments, R⁶ is halobutyl. In some embodiments, R⁶ is halopentyl. In some embodiments, R⁶ is halohexyl.

As generally described herein, R^{6′} is H, halo, —OH, C₁-C₆ alkoxy, C₁-C₆ haloalkoxy, C₁-C₆ alkyl, or C₁-C₆ haloalkyl.

In some embodiments, R^{6′} is H.

In some embodiments, R^{6′} is halo, —OH, C₁-C₆ alkyl, or C₁-C₆ haloalkyl.

In some embodiments, R^{6′} is C₁-C₆ alkoxy or C₁-C₆ haloalkoxy.

In some embodiments, R^{6′} is halo or —OH.

In some embodiments, R^{6′} is halo.

In some embodiments, R^{6′} is F, Cl, Br, or I. In some embodiments, R⁶ is F, Cl, or Br.

In some embodiments, R^{6′} is F. In some embodiments, R^{6′} is Cl. In some embodiments, R^{6′} is Br.

In some embodiments, R^{6′} is I.

In some embodiments, R^{6′} is —OH.

In some embodiments, R^{6′} is C₁-C₆ alkoxy.

In some embodiments, R^{6′} is C₁-C₆ haloalkoxy.

In some embodiments, R^{6′} is C₁-C₆ alkyl.

In some embodiments, R^{6′} is methyl. In some embodiments, R^{6′} is ethyl. In some embodiments, R^{6′} is propyl. In some embodiments, R^{6′} is butyl. In some embodiments, R^{6′} is pentyl. In some embodiments, R^{6′} is hexyl. In some embodiments, R^{6′} is isopropyl. In some embodiments, R^{6′} is isobutyl.

In some embodiments, R^{6′} is isopentyl. In some embodiments, R^{6′} is isohexyl. In some embodiments, R^{6′} is secbutyl. In some embodiments, R^{6′} is secpentyl. In some embodiments, R^{6′} is sechexyl. In some embodiments, R^{6′} is tertbutyl.

In some embodiments, R^{6′} is C₁-C₆ haloalkyl.

In some embodiments, R^{6′} is halomethyl. In some embodiments, R^{6′} is haloethyl. In some embodiments, R^{6′} is halopropyl. In some embodiments, R^{6′} is halobutyl. In some embodiments, R^{6′} is halopentyl. In some embodiments, R^{6′} is halohexyl.

In some embodiments, R⁶ is F or OH.

As generally described herein, R^{6″} is H, halo, —OH, C₁-C₆ alkoxy, C₁-C₆ haloalkoxy, C₁-C₆ alkyl, or C₁-C₆ haloalkyl.

In some embodiments, R⁶ is H.

In some embodiments, R^{6″} is halo, —OH, C₁-C₆ alkyl, or C₁-C₆ haloalkyl.

In some embodiments, R^{6″} is C₁-C₆ alkoxy or C₁-C₆ haloalkoxy.

In some embodiments, R^{6″} is halo or —OH.

In some embodiments, R^{6″} is halo.

In some embodiments, R^{6″} is F, Cl, Br, or I. In some embodiments, R⁶ is F, Cl, or Br.

In some embodiments, R^{6″} is F. In some embodiments, R^{6″} is Cl. In some embodiments, R^{6″} is Br.

In some embodiments, R^{6″} is I.

In some embodiments, R^{6″} is —OH.

In some embodiments, R^{6″} is C₁-C₆ alkoxy.

In some embodiments, R^{6″} is C₁-C₆ haloalkoxy.

In some embodiments, R^{6″} is C₁-C₆ alkyl.

In some embodiments, R^{6″} is methyl. In some embodiments, R^{6″} is ethyl. In some embodiments, R^{6″} is propyl. In some embodiments, R^{6″} is butyl. In some embodiments, R^{6″} is pentyl. In some embodiments, R^{6″} is hexyl. In some embodiments, R^{6″} is isopropyl. In some embodiments, R^{6″} is isobutyl. In some embodiments, R^{6″} is isopentyl. In some embodiments, R^{6″} is isohexyl. In some embodiments, R^{6″} is secbutyl. In some embodiments, R^{6″} is secpentyl. In some embodiments, R^{6″} is sechexyl. In some embodiments, R^{6″} is tertbutyl.

In some embodiments, R^{6″} is C₁-C₆ haloalkyl.

In some embodiments, R^{6″} is halomethyl. In some embodiments, R⁶ is haloethyl. In some embodiments, R^{6″} is halopropyl. In some embodiments, R^{6″} is halobutyl. In some embodiments, R^{6″} is halopentyl. In some embodiments, R^{6″} is halohexyl.

In some embodiments, R⁶ is H or F.

In some embodiments, R^{6′} is halo and R^{6″} is H. In some embodiments, R^{6′} is F and R⁶ is H.

In some embodiments, R^{6′} is H and R^{6″} is halo. In some embodiments, R^{6′} is H and R^{6″} is F.

In some embodiments, each of R^{6′} and R^{6″} is independently halo. In some embodiments, each of R^{6′} and R^{6″} is F.

As generally described herein, R⁷ and R² are joined to form a 5- to 10-membered monocyclic or bicyclic heterocyclyl or 5- to 10-membered monocyclic or bicyclic heteroaryl, wherein the alkyl, haloalkyl, alkoxy, haloalkoxy, heterocyclyl or heteroaryl are optionally substituted with one or more R^{7a} wherein each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃-C₅ cycloalkyl, or 4- to 5-membered heterocyclyl, wherein the alkyl, haloalkyl, alkoxy, haloalkoxy, aryl, heteroaryl, cycloalkyl, and heterocyclyl are optionally substituted with one or more Re, further wherein Re is halo, —CN, —OH, C₁-C₆ alkyl, C₁-C₆ haloalkyl, C₁-C₆ alkoxy, or C₁-C₆ haloalkoxy.

In some embodiments, R⁷ and R² are joined to form a 5- to 10-membered monocyclic or bicyclic heterocyclyl.

In some embodiments, R⁷ and R² are joined to form a 5- to 10-membered monocyclic or bicyclic heterocyclyl optionally substituted with one or more R^{7a}.

In some embodiments, R⁷ and R² are joined to form a 5- to 10-membered monocyclic or bicyclic heterocyclyl substituted with one or more R^{7a}.

In some embodiments, R⁷ and R² are joined to form a 5- to 10-membered monocyclic heterocyclyl.

In some embodiments, R⁷ and R² are joined to form a 5- to 10-membered monocyclic heterocyclyl optionally substituted with one or more R^{7a}.

In some embodiments, R⁷ and R² are joined to form a 5- to 10-membered monocyclic heterocyclyl substituted with one or more R^{7a}.

In some embodiments, R⁷ and R² are joined to form a 5-membered monocyclic heterocyclyl.

In some embodiments, R⁷ and R² are joined to form a 5-membered monocyclic heterocyclyl optionally substituted with one or more R^{7a}.

In some embodiments, R⁷ and R² are joined to form a 5-membered monocyclic heterocyclyl substituted with one or more R^{7a}.

In some embodiments, R⁷ and R² are joined to form a 5- to 10-membered bicyclic heterocyclyl.

In some embodiments, R⁷ and R² are joined to form a 5- to 10-membered bicyclic heterocyclyl optionally substituted with one or more R^{7a}.

In some embodiments, R⁷ and R² are joined to form a 5- to 10-membered bicyclic heterocyclyl substituted with one or more R^{7a}.

In some embodiments, R⁷ and R² are joined to form a 5- to 10-membered monocyclic or bicyclic heteroaryl.

In some embodiments, R⁷ and R² are joined to form a 5- to 10-membered monocyclic or bicyclic heteroaryl optionally substituted with one or more R^{7a}.

In some embodiments, R⁷ and R² are joined to form a 5- to 10-membered monocyclic or bicyclic heteroaryl substituted with one or more R^{7a}.

In some embodiments, R⁷ and R² are joined to form a 5- to 10-membered monocyclic heteroaryl.

In some embodiments, R⁷ and R² are joined to form a 5- to 10-membered monocyclic heteroaryl optionally substituted with one or more R^{7a}.

In some embodiments, R⁷ and R² are joined to form a 5- to 10-membered monocyclic heteroaryl substituted with one or more R^{7a}.

In some embodiments, R⁷ and R² are joined to form a 5- to 10-membered bicyclic heteroaryl.

In some embodiments, R⁷ and R² are joined to form a 5- to 10-membered bicyclic heteroaryl optionally substituted with one or more R^{7a}.

In some embodiments, R⁷ and R² are joined to form a 5- to 10-membered bicyclic heteroaryl substituted with one or more R^{7a}.

In some embodiments, R⁷ and R² are joined to form a 6-membered monocyclic heteroaryl.

In some embodiments, R⁷ and R² are joined to form a 6-membered monocyclic heteroaryl optionally substituted with one or more R^{7a}.

In some embodiments, R⁷ and R² are joined to form a 6-membered monocyclic heteroaryl substituted with one or more R^{7a}.

In some embodiments, R⁷ and R² are joined to form a 10-membered bicyclic heteroaryl.

In some embodiments, R⁷ and R² are joined to form a 10-membered bicyclic heteroaryl optionally substituted with one or more R^{7a}.

In some embodiments, R⁶ and R² are joined to form a 10-membered bicyclic heteroaryl substituted with one or more R^{7a}.

In some embodiments, the 6-membered monocyclic heteroaryl comprises one N atom.

In some embodiments, the 6-membered monocyclic heteroaryl comprises two N atoms.

In some embodiments, the 10-membered bicyclic heteroaryl comprises one N atom.

As generally described herein, each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃-C₅ cycloalkyl, or 4 to 5-membered heterocyclyl, wherein the alkyl, haloalkyl, alkoxy, haloalkoxy, aryl, heteroaryl, cycloalkyl, and heterocyclyl are optionally substituted with one or more R^{7b}.

In some embodiments, each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃_C₅ cycloalkyl, or 4- to 5-membered heterocyclyl, wherein the alkyl, haloalkyl, alkoxy, haloalkoxy, aryl, heteroaryl, cycloalkyl, and heterocyclyl are substituted with one or more R^{7b}.

In some embodiments, each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃_C₅ cycloalkyl, or 4- to 5-membered heterocyclyl.

In some embodiments, at least one R^{7a} is independently halo.

In some embodiments, at least one R^{7a} is independently —CN.

In some embodiments, at least one R^{7a} is independently —OH.

In some embodiments, at least one R^{7a} is independently C₁-C₆ alkyl optionally substituted with one or more R^{7b}.

In some embodiments, at least one R^{7a} is independently C₁-C₆ alkyl.

In some embodiments, R^{7a} is methyl. In some embodiments, R^{7a} is ethyl. In some embodiments, R^{7a} is propyl. In some embodiments, R^{7a} is butyl. In some embodiments, R^{7a} is pentyl. In some embodiments, R^{7a} is hexyl. In some embodiments, R^{7a} is isopropyl. In some embodiments, R^{7a} is isobutyl.

In some embodiments, R^{7a} is isopentyl. In some embodiments, R^{7a} is isohexyl. In some embodiments, R^{7a} is secbutyl. In some embodiments, R^{7a} is secpentyl. In some embodiments, R^{7a} is sechexyl. In some embodiments, R^{7a} is tertbutyl.

In some embodiments, at least one R^{7a} is independently C₁-C₆ haloalkyl optionally substituted with one or more R^{7b}.

In some embodiments, at least one R^{7a} is independently C₁-C₆ haloalkyl.

In some embodiments, R^{7a} is halomethyl. In some embodiments, R^{7a} is haloethyl. In some embodiments, R^{7a} is halopropyl. In some embodiments, R^{7a} is halobutyl. In some embodiments, R^{7a} is halopentyl. In some embodiments, R^{7a} is halohexyl.

In some embodiments, at least one R^{7a} is independently C₁-C₆ alkoxy optionally substituted with one or more Rh.

In some embodiments, at least one R^{7a} is independently C₁-C₆ alkoxy.

In some embodiments, at least one R^{7a} is independently C₁-C₆ haloalkoxy optionally substituted with one or more Rib.

In some embodiments, at least one R^{7a} is independently C₁-C₆ haloalkoxy.

In some embodiments, at least one R^{7a} is independently C₆ aryl or 5- to 6-membered heteroaryl, wherein the aryl and heteroaryl are optionally substituted with one or more R^{7b}.

In some embodiments, at least one R^{7a} is independently C₆ aryl.

In some embodiments, at least one R^{7a} is independently C₆ aryl optionally substituted with one or more Rib.

In some embodiments, at least one R^{7a} is independently C₆ aryl substituted with one or more R^{7b}.

In some embodiments, at least one R^{7a} is independently 5- to 6-membered heteroaryl.

In some embodiments, at least one R^{7a} is independently 5- to 6-membered heteroaryl optionally substituted with one or more R^{7b}.

In some embodiments, at least one R^{7a} is independently 5- to 6-membered heteroaryl substituted with one or more R^{7b}.

In some embodiments, at least one R^{7a} is independently C₃-C₅ cycloalkyl or 4- to 5-membered heterocyclyl, wherein the cycloalkyl and heterocyclyl are optionally substituted with one or more R^{7b}.

In some embodiments, at least one R^{7a} is independently C₃-C₅ cycloalkyl.

In some embodiments, at least one R^{7a} is independently C₃-C₅ cycloalkyl optionally substituted with one or more R^{7b}.

In some embodiments, at least one R^{7a} is independently C₃-C₅ cycloalkyl substituted with one or more Rib.

In some embodiments, at least one R^{7a} is independently cyclopropyl.

In some embodiments, at least one R^{7a} is independently cyclopropyl optionally substituted with one or more Rh.

In some embodiments, at least one R^{7a} is independently cyclopropyl substituted with one or more Rib.

In some embodiments, at least one R^{7a} is independently 4- to 5-membered heterocyclyl.

In some embodiments, at least one R^{7a} is independently 4- to 5-membered heterocyclyl optionally substituted with one or more Rh.

In some embodiments, at least one R^{7a} is independently 4- to 5-membered heterocyclyl substituted with one or more R^{7b}.

In some embodiments, at least one R^{7a} is independently C₁-C₆ alkyl, C₁-C₆ haloalkyl, C₆ aryl, or C₃-C₅ cycloalkyl.

In some embodiments, at least one R^{7a} is independently methyl, —CF₃, C₆ aryl, or cyclopropyl.

As generally described herein, R^{7b} is halo, —CN, —OH, C₁-C₆ alkyl, C₁-C₆ haloalkyl, C₁-C₆ alkoxy, or C₁-C₆ haloalkoxy.

In some embodiments, R^{7b} is halo.

In some embodiments, R^{7b} is —CN.

In some embodiments, R^{7b} is —OH.

In some embodiments, R^{7b} is C₁-C₆ alkyl.

In some embodiments, R^{7b} is C₁-C₆ haloalkyl.

In some embodiments, R^{7b} is C₁-C₆ alkoxy.

In some embodiments, R^{7b} is C₁-C₆ haloalkoxy.

In some embodiments, R⁷ and R² are joined to form a 5- to 10-membered monocyclic or bicyclic heteroaryl optionally substituted with one R^{1a}, wherein R^{1a} is C₁-C₆ alkyl, C₁-C₆ haloalkyl, C₆ aryl, or C₃-C₅ cycloalkyl.

In some embodiments, R⁷ and R² are joined to form a 6-membered monocyclic heteroaryl optionally substituted with one R^{7a}, wherein R^{7a} is C₁-C₆ alkyl, C₁-C₆ haloalkyl, C₆ aryl, or C₃-C₈ cycloalkyl.

In some embodiments, R⁷ and R² are joined to form a 6-membered monocyclic heteroaryl optionally substituted with one R^{7a}, wherein R^{7a} is methyl, —CF₃, C₆ aryl, or cyclopropyl.

In some embodiments, R⁷ and R² are joined to form a 10-membered bicyclic heteroaryl optionally substituted with one R^{7a}, wherein R^{7a} is C₁-C₆ alkyl, C₁-C₆ haloalkyl, C₆ aryl, or C₃-C₅ cycloalkyl.

### (b) R⁴ and Z

As generally described herein, R⁴ is —C₁-C₆ alkylene- optionally substituted with one or more R^{4a} wherein each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl, or two R^{4a} are joined to form a C₃-C₈ cycloalkyl or 4- to 6-membered heterocyclyl ring, wherein the alkyl, haloalkyl, alkoxy, haloalkoxy, cycloalkyl, and heterocyclyl are optionally substituted with one or more R^{4b}, further wherein R^{4b} is halo, —CN, —OH, C₁-C₆ alkyl, C₁-C₆ haloalkyl, C₁-C₆ alkoxy, or C₁-C₆ haloalkoxy.

In some embodiments, R⁴ is unsubstituted or independently substituted with 1 or 2 R^{4a}

In some embodiments, R⁴ is unsubstituted.

In some embodiments, R⁴ is independently substituted with 1 or 2 R^{4a}

In some embodiments, R⁴ is independently substituted with 1 R^{4a}

In some embodiments, R⁴ is independently substituted with 2 R^{4a}

In some embodiments, R⁴ is —C₁-C₆ alkylene- optionally substituted with one or more R^{4a}, wherein each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl, wherein the alkyl, haloalkyl, alkoxy, haloalkoxy, cycloalkyl, and heterocyclyl are optionally substituted with one or more R^{4b}, further wherein R^{4b} is halo, —CN, —OH, C₁-C₆ alkyl, C₁-C₆ haloalkyl, C₁-C₆ alkoxy, or C₁-C₆ haloalkoxy.

In some embodiments, R⁴ is —C₁-C₆ alkylene- optionally substituted with one or more R^{4a}, wherein each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl.

In some embodiments, R⁴ is —C₁-C₆ alkylene- optionally substituted with one or more R^{4a}, wherein two R^{4a} are joined to form a C₃-C₅ cycloalkyl or 4- to 6-membered heterocyclyl ring, wherein the cycloalkyl and heterocyclyl are optionally substituted with one or more R^{4b}, further wherein R^{4b} is halo, —CN, —OH, C₁-C₆ alkyl, C₁-C₆ haloalkyl, C₁-C₆ alkoxy, or C₁-C₆ haloalkoxy.

In some embodiments, R⁴ is —C₁-C₆ alkylene- optionally substituted with one or more R^{4a}, wherein two R^{4a} are joined to form a C₃-C₅ cycloalkyl or 4- to 6-membered heterocyclyl ring.

In some embodiments, R⁴ is —C₁-C₆ alkylene-.

In some embodiments, R⁴ is —C₁ alkylene-. In some embodiments, R⁴ is —C₂ alkylene-. In some embodiments, R⁴ is —C₃ alkylene-. In some embodiments, R⁴ is —C₄ alkylene-. In some embodiments, R⁴ is —C₅ alkylene-. In some embodiments, R⁴ is —C₆ alkylene-.

In some embodiments, R⁴ is —C₁ alkylene- optionally substituted with one or more R^{4a}. In some embodiments, R⁴ is —C₂ alkylene- optionally substituted with one or more R^{4a}. In some embodiments, R⁴ is —C₃ alkylene- optionally substituted with one or more R^{4a}. In some embodiments, R⁴ is —C₄ alkylene-optionally substituted with one or more R^{4a}. In some embodiments, R⁴ is —C₅ alkylene- optionally substituted with one or more R^{4a}. In some embodiments, R⁴ is —C₆ alkylene- optionally substituted with one or more R^{4a}

As generally described herein, each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl, or two R^{4a} are joined to form a C₃-C₅ cycloalkyl or 4- to 6-membered heterocyclyl ring, wherein the alkyl, haloalkyl, alkoxy, haloalkoxy, cycloalkyl, and heterocyclyl are optionally substituted with one or more R^{4b}.

In some embodiments, each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl ring, wherein the alkyl, haloalkyl, alkoxy, haloalkoxy, cycloalkyl, and heterocyclyl are optionally substituted with one or more R^{4b}.

In some embodiments, two R^{4a} are joined to form a C₃-C₅ cycloalkyl or 4- to 6-membered heterocyclyl ring, wherein the cycloalkyl and heterocyclyl are optionally substituted with one or more R^{4b}.

In some embodiments, at least one R^{4a} is independently halo.

In some embodiments, at least one R^{4a} is independently —CN.

In some embodiments, at least one R^{4a} is independently —OH.

In some embodiments, at least one R^{4a} is independently C₁-C₆ alkyl optionally substituted with one or more R^{4b}. In some embodiments, at least one R^{4a} is independently C₁-C₃ alkyl. In some embodiments, at least one R^{4a} is methyl (—CH₃).

In some embodiments, at least one R^{4a} is independently C₁-C₆ alkyl.

In some embodiments, at least one R^{4a} is independently C₁-C₆ haloalkyl optionally substituted with one or more R^{4b}.

In some embodiments, at least one R^{4a} is independently C₁-C₆ haloalkyl.

In some embodiments, at least one R^{4a} is independently C₁-C₆ alkoxy optionally substituted with one or more R^{4b}.

In some embodiments, at least one R^{4a} is independently C₁-C₆ alkoxy.

In some embodiments, at least one R^{4a} is independently C₁-C₆ haloalkoxy optionally substituted with one or more R^{4b}.

In some embodiments, at least one R^{4a} is independently C₁-C₆ haloalkoxy.

In some embodiments, at least one R^{4a} is independently C₃₋₄ cycloalkyl or 4- to 5-membered heterocyclyl, wherein the cycloalkyl and heterocyclyl are optionally substituted with one or more R^{4b}.

In some embodiments, at least one R^{4a} is independently C₃₋₄ cycloalkyl.

In some embodiments, at least one R^{4a} is independently C₃₋₄ cycloalkyl optionally substituted with one or more R^{4b}.

In some embodiments, at least one R^{4a} is independently C₃₋₄ cycloalkyl substituted with one or more R^{4b}.

In some embodiments, at least one R^{4a} is independently 4- to 5-membered heterocyclyl.

In some embodiments, at least one R^{4a} is independently 4- to 5-membered heterocyclyl optionally substituted with one or more R^{4b}.

In some embodiments, at least one R^{4a} is independently 4- to 5-membered heterocyclyl substituted with one or more R^{4b}.

In some embodiments, two R^{4a} are joined to form a C₃-C₅ cycloalkyl.

In some embodiments, two R^{4a} are joined to form a C₃-C₅ cycloalkyl optionally substituted with one or more R^{4b}.

In some embodiments, two R^{4a} are joined to form a C₃-C₅ cycloalkyl substituted with one or more R^{4b}.

In some embodiments, two R^{4a} are joined to form a C₅ cycloalkyl.

In some embodiments, two R^{4a} are joined to form a C₅ cycloalkyl optionally substituted with one or more R^{4b}.

In some embodiments, two R^{4a} are joined to form a C₅ cycloalkyl substituted with one or more R^{4b}.

In some embodiments, two R^{4a} are joined to form a 4- to 6-membered heterocyclyl ring.

In some embodiments, two R^{4a} are joined to form a 4- to 6-membered heterocyclyl ring optionally substituted with one or more R^{4b}.

In some embodiments, two R^{4a} are joined to form a 4- to 6-membered heterocyclyl ring substituted with one or more R^{4b}.

As generally described herein, R^{4b} is halo, —CN, —OH, C₁-C₆ alkyl, C₁-C₆ haloalkyl, C₁-C₆ alkoxy, or C₁-C₆ haloalkoxy.

In some embodiments, at least one R^{4b} is halo.

In some embodiments, at least one R^{4b} is —CN.

In some embodiments, at least one R^{4b} is —OH.

In some embodiments, at least one R^{4b} is C₁-C₆ alkyl.

In some embodiments, at least one R^{4b} is C₁-C₆ haloalkyl.

In some embodiments, at least one R^{4b} is C₁-C₆ alkoxy.

In some embodiments, at least one R^{4b} is C₁-C₆ haloalkoxy.

As generally described herein, Z is a —CO₂H or —CO₂PG, wherein PG is C₁-C₆ alkyl, C₆ aryl, or (C₆ aryl)(C₁-C₆ alkyl), further wherein alkyl and aryl are optionally substituted with one or more halo, C₁-C₆ alkyl, C₁-C₆ alkoxy, or C₁-C₆ haloalkoxy.

In some embodiments, Z is —CO₂H.

In some embodiments, Z is —CO₂PG.

As generally described herein, PG is C₁-C₆ alkyl, C₆ aryl, or (C₆ aryl)(C₁-C₆ alkyl), wherein alkyl and aryl are optionally substituted with one or more halo, C₁-C₆ alkyl, C₁-C₆ alkoxy, or C₁-C₆ haloalkoxy.

In some embodiments, PG is C₁-C₆ alkyl, C₆ aryl, or (C₆ aryl)(C₁-C₆ alkyl).

In some embodiments, PG is C₁-C₆ alkyl, C₆ aryl, or (C₆ aryl)(C₁-C₆ alkyl), wherein alkyl and aryl are substituted with one or more halo, C₁-C₆ alkyl, C₁-C₆ alkoxy, or C₁-C₆ haloalkoxy.

In some embodiments, PG is C₁-C₆ alkyl optionally substituted with one or more halo, C₁-C₆ alkyl, C₁-C₆ alkoxy, or C₁-C₆ haloalkoxy.

In some embodiments, PG is C₁-C₆ alkyl.

In some embodiments, PG is C₁-C₆ alkyl substituted with one or more halo, C₁-C₆ alkyl, C₁-C₆ alkoxy, or C₁-C₆ haloalkoxy.

In some embodiments, PG is C₆ aryl optionally substituted with one or more halo, C₁-C₆ alkyl, C₁-C₆ alkoxy, or C₁-C₆ haloalkoxy.

In some embodiments, PG is C₆ aryl.

In some embodiments, PG is C₆ aryl substituted with one or more halo, C₁-C₆ alkyl, C₁-C₆ alkoxy, or C₁-C₆ haloalkoxy.

In some embodiments, PG is (C₆ aryl)(C₁-C₆ alkyl), wherein alkyl and aryl are optionally substituted with one or more halo, C₁-C₆ alkyl, C₁-C₆ alkoxy, or C₁-C₆ haloalkoxy.

In some embodiments, PG is (C₆ aryl)(C₁-C₆ alkyl).

In some embodiments, PG is (C₆ aryl)(C₁-C₆ alkyl), wherein alkyl and aryl are substituted with one or more halo, C₁-C₆ alkyl, C₁-C₆ alkoxy, or C₁-C₆ haloalkoxy.

### (c) Additional Embodiments

In some embodiments of Formula (I), wherein Z is —CO₂H, the compound is of Formula (I-A):

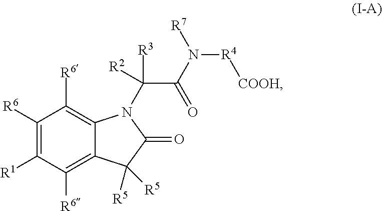

or a pharmaceutically acceptable salt thereof.

In some embodiments of Formula (I-A), or a pharmaceutically acceptable salt thereof, R¹ is halo; R³ is absent; R⁷ and R² are joined to form a 5- to 10-membered monocyclic or bicyclic heteroaryl or 5- to 10-membered monocyclic or bicyclic heterocyclyl; R⁴ is —C₁-C₆ alkylene-; each R⁵ is independently C₁-C₆ alkyl; R⁶ is H; R^{6′} is halo or OH; and R^{6″} is H.

In some embodiments of Formula (I-A), or a pharmaceutically acceptable salt thereof, R¹ is halo; R³ is absent; R⁷ and R² are joined to form a 5- to 10-membered monocyclic or bicyclic heteroaryl; R⁴ is —C₁-C₆ alkylene-; each R⁵ is independently C₁-C₆ alkyl; R⁶ is H; R^{6′} is H or halo or OH; and R^{6″} is H. In some embodiments, R¹ is Br or Cl; R³ is absent; R⁷ and R² are joined to form a 6-membered monocyclic heteroaryl; R⁴ is —C₃ alkylene-; each R⁵ is independently methyl; R⁶ is H; R^{6′} is H or F or OH; and R^{6″} is H. In some embodiments, R¹ is Br; R³ is absent; R⁷ and R² are joined to form a 6-membered monocyclic heteroaryl; R⁴ is —C₃ alkylene-; each R⁵ is independently methyl; R⁶ is H; R^{6′} is H or F; and R^{6″} is H. In some embodiments, R¹ is Cl; R³ is absent; R⁷ and R² are joined to form a 6-membered monocyclic heteroaryl; R⁴ is —C₃ alkylene-; each R⁵ is independently methyl; R⁶ is H; R^{6′} is H or OH; and R^{6″} is H.

In some embodiments of Formula (I-A), or a pharmaceutically acceptable salt thereof, R¹ is C₃-C₄ cycloalkyl; R³ is absent; R⁷ and R² are joined to form a 5- to 10-membered monocyclic or bicyclic heteroaryl optionally substituted with one or more C₁-C₆ alkyl, C₁-C₆ haloalkyl, C₆ aryl, or C₃-C₈ cycloalkyl; R⁴ is —C₁-C₆ alkylene-; each R⁵ is independently C₁-C₆ alkyl; R⁶ is H; R^{6′} is H or halo; and R^{6″} is H or halo. In some embodiments, R¹ is C₃-C₄ cycloalkyl; R³ is absent; R⁷ and R² are joined to form a 6-membered monocyclic heteroaryl; R⁴ is —C₃ alkylene-; each R⁵ is independently C₁-C₆ alkyl; R⁶ is H; R^{6′} is H or halo; and R^{6″} is H. In some embodiments, R¹ is C₃-C₄ cycloalkyl; R³ is absent; R⁷ and R² are joined to form a 6-membered monocyclic heteroaryl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R¹ and R² are joined to form a 6-membered monocyclic heteroaryl; R⁴ is —C₃ alkylene-; each R⁵ is independently methyl; R⁶ is H; R^{6′} is H or F; and R^{6″} is H. In some embodiments, R¹ is cyclopropyl; R³ is absent; R⁷ and R² are joined to form a 6-membered monocyclic heteroaryl; R⁴ is —C₃ alkylene-; each R⁵ is independently methyl; R⁶ is H; R^{6′} is H or F; and R^{6″} is F.

In some embodiments of Formula (I-A), or a pharmaceutically acceptable salt thereof, R¹ is C₃-C₄ cycloalkyl; R³ is absent; R⁷ and R² are joined to form a 6-membered monocyclic heteroaryl optionally substituted with one or more C₁-C₆ alkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with methyl; R⁴ is —C₃ alkylene-; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 6-membered monocyclic heteroaryl optionally substituted with one or more C₁-C₆ haloalkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with —CF₃; R⁴ is —C₃ alkylene-; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 6-membered monocyclic heteroaryl optionally substituted with one or more C₆ aryl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with phenyl; R⁴ is —C₃ alkylene-; each R is independently methyl; R⁶ is H; R^{6′} is H or F; and R^{6″} is F. In some embodiments, R¹ is C₃-C₄ cycloalkyl; R³ is absent; R⁷ and R² are joined to form a 6-membered monocyclic heteroaryl optionally substituted with one or more C₃-C₅ cycloalkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with cyclopropyl; R⁴ is —C₃ alkylene-; each R⁵ is independently methyl; R⁶ is H; R^{6′} is H or F; and R^{6″} is F.

In some embodiments of Formula (I-A), or a pharmaceutically acceptable salt thereof, R¹ is C₃-C₄ cycloalkyl; R³ is absent; R⁷ and R² are joined to form a 10-membered bicyclic heteroaryl; R⁴ is —C₁-C₆ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 10-membered bicyclic heteroaryl; R⁴ is —C₃ alkylene-; each R⁵ is independently methyl; R⁶ is H; R^{6′} is H or F; and R^{6″} is F.

In some embodiments of Formula (I-A), or a pharmaceutically acceptable salt thereof, R¹ is halo; R³ is absent; R⁷ and R² are joined to form a 5- to 10-membered heterocyclyl; R⁴ is —C₁-C₆ alkylene-; each R⁵ is independently C₁-C₆ alkyl; R⁶ is H; R^{6′} is H or halo or OH; and R^{6″} is H. In some embodiments, R¹ is Br or Cl; R³ is absent; R⁷ and R² are joined to form a 5-membered heterocyclyl; R⁴ is —C₃ alkylene-; each R⁵ is independently methyl; R⁶ is H; R^{6′} is H or F or OH; and R^{6″} is H. In some embodiments, R¹ is Br; R³ is absent; R⁷ and R² are joined to form a 5-membered heterocyclyl; R⁴ is —C₃ alkylene-; each R⁵ is independently methyl; R⁶ is H; R^{6′} is H or F; and R^{6″} is H. In some embodiments, R¹ is Cl; R³ is absent; R⁷ and R² are joined to form a 5-membered heterocyclyl; R⁴ is —C₃ alkylene-; each R⁵ is independently methyl; R⁶ is H; R^{6′} is H or OH; and R^{6″} is H.

In some embodiments of Formula (I-A), or a pharmaceutically acceptable salt thereof, R¹ is C₃-C₄ cycloalkyl; R³ is absent; R⁷ and R² are joined to form a 5- to 10-membered monocyclic or bicyclic heterocyclyl optionally substituted with one or more C₁-C₆ alkyl, C₁-C₆ haloalkyl, C₆ aryl, or C₃-C₅ cycloalkyl; R⁴ is —C₁-C₆ alkylene-; each R⁵ is independently C₁-C₆ alkyl; R⁶ is H; R^{6′} is H or halo; and R^{6″} is H or halo. In some embodiments, R¹ is C₃-C₄ cycloalkyl; R³ is absent; R⁷ and R² are joined to form a 5-membered monocyclic heterocyclyl; R⁴ is —C₃ alkylene-; each R⁵ is independently C₁-C₆ alkyl; R⁶ is H; R^{6′} is H or halo; and R^{6″} is H. In some embodiments, R¹ is C₃-C₄ cycloalkyl; R³ is absent; R¹ and W are joined to form a 5-membered monocyclic heterocyclyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl; R⁴ is —C₃ alkylene-; each R⁵ is independently methyl; R⁶ is H; R^{6′} is H or F; and R^{6″} is H. In some embodiments, R¹ is cyclopropyl; R³ is absent; R⁷ and R² are joined to form a 5-membered monocyclic heterocyclyl; R⁴ is —C₃ alkylene-; each R⁵ is independently methyl; R⁶ is H; R^{6′} is H or F; and R^{6″} is F.

In some embodiments of Formula (I-A), or a pharmaceutically acceptable salt thereof, R¹ is C₃-C₄ cycloalkyl; R³ is absent; R⁷ and R² are joined to form a 5-membered monocyclic heterocyclyl optionally substituted with one or more C₁-C₆ alkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with methyl; R⁴ is —C₃ alkylene-; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 5-membered monocyclic heterocyclyl optionally substituted with one or more C₁-C₆ haloalkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with —CF₃; R⁴ is —C₃ alkylene-; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 5-membered monocyclic heterocyclyl optionally substituted with one or more C₆ aryl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with phenyl; R⁴ is —C₃ alkylene-; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 5-membered monocyclic heterocyclyl optionally substituted with one or more C₃-C₅ cycloalkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with cyclopropyl; R⁴ is —C₃ alkylene-; each R⁵ is independently methyl; R⁶ is H; R^{6′} is H or F; and R^{6″} is F.

In some embodiments of Formula (I-A), wherein R⁴ is —C₃ alkylene-, the compound is of Formula (I-A′):

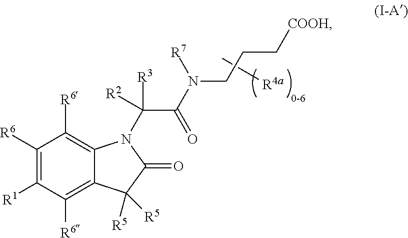

or a pharmaceutically acceptable salt thereof, comprising 0 R^{4a} groups (whereby R^{4a} is absent), or 1, 2, 3, 4, 5, or 6 independent R^{4a} groups.

In some embodiments of Formula (I-A′), or a pharmaceutically acceptable salt thereof, R¹ is halo; R³ is absent; R⁷ and R² are joined to form a 5- to 10-membered monocyclic heteroaryl or 5- to 10-membered monocyclic heterocyclyl; R^{4a} is absent; each R⁵ is independently C₁-C₆ alkyl; R⁶ is H; R¹ is H, halo, or —OH; and R^{6″} is H.

In some embodiments of Formula (I-A′), or a pharmaceutically acceptable salt thereof, R¹ is halo; R³ is absent; R⁷ and R² are joined to form a 5- to 10-membered monocyclic heteroaryl; R^{4a} is absent; each R⁵ is independently C₁-C₆ alkyl; R⁶ is H; R^{6′} is H, halo, or —OH; and R^{6″} is H. In some embodiments, R¹ is Br or Cl; R³ is absent; R⁷ and R² are joined to form a 6-membered monocyclic heteroaryl; R^{4a} is absent; each R⁵ is independently methyl; R⁶ is H; R^{6′} is H, F, or —OH; and R^{6″} is H. In some embodiments, R¹ is Br; R³ is absent; R⁷ and R² are joined to form a 6-membered monocyclic heteroaryl; R^{4a} is absent; each R⁵ is independently methyl; R⁶ is H; R^{6′} is H or F; and R^{6″} is H. In some embodiments, R¹ is Cl; R³ is absent; R⁷ and R² are joined to form a 6-membered monocyclic heteroaryl; R^{4a} is absent; each R⁵ is independently methyl; R⁶ is H; R^{6′} is H or —OH; and R^{6″} is H.

In some embodiments of Formula (I-A′), or a pharmaceutically acceptable salt thereof, R¹ is C₃-C₄ cycloalkyl; R³ is absent; R⁷ and R² are joined to form a 5- to 10-membered monocyclic heteroaryl optionally substituted with one or more C₁-C₆ alkyl, C₁-C₆ haloalkyl, C₆ aryl, or C₃-C₅ cycloalkyl; R^{4a} is absent; each R⁵ is independently C₁-C₆ alkyl; R⁶ is H; R^{6′} is H or halo; and R^{6″} is H or halo. In some embodiments, R¹ is C₃-C₄ cycloalkyl; R³ is absent; R⁷ and R² are joined to form a 6-membered monocyclic heteroaryl; R^{4a} is absent; each R⁵ is independently C₁-C₆ alkyl; R⁶ is H; R^{6′} is H or halo; and R^{6″} is H. In some embodiments, R¹ is C₃-C₄ cycloalkyl; R³ is absent; R⁷ and R² are joined to form a 6-membered monocyclic heteroaryl; R^{4a} is absent; each R⁵ is independently C₁-C₆ alkyl; R⁶ is H; R^{6′} is H or halo; and R is halo. In some embodiments, R¹ is cyclopropyl; R³ is absent; R⁷ and R² are joined to form a 6-membered monocyclic heteroaryl; R^{4a} is absent; each R⁵ is independently methyl; R⁶ is H; R^{6′} is H or F; and R^{6″} is H. In some embodiments, R¹ is cyclopropyl; R³ is absent; R⁷ and R² are joined to form a 6-membered monocyclic heteroaryl; R^{4a} is absent; each R⁵ is independently methyl; R⁶ is H; R^{6′} is H or F; and R^{6″} is F.

In some embodiments of Formula (I-A′), or a pharmaceutically acceptable salt thereof, R¹ is C₃-C₄ cycloalkyl; R³ is absent; R⁷ and R² are joined to form a 6-membered monocyclic heteroaryl optionally substituted with one or more C₁-C₆ alkyl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with methyl; R^{4a} is absent; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 6-membered monocyclic heteroaryl optionally substituted with one or more C₁-C₆ haloalkyl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with —CF₃; R^{4a} is absent; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 6-membered monocyclic heteroaryl optionally substituted with one or more C₆ aryl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 6-membered monocyclic heteroaryl substituted with phenyl; R^{4a} is absent; each R⁵ is independently methyl; R⁶ is H; R^{6′} is H or F; and R is F. In some embodiments, R¹ is C₃-C₄ cycloalkyl; R³ is absent; R⁷ and W are joined to form a 6-membered monocyclic heteroaryl optionally substituted with one or more C₃-C₅ cycloalkyl; R^{4a} is absent; each R⁵ is independently C₁-C₆ alkyl; R⁶ is H; R^{6′} is H or halo; and R is halo. In some embodiments, R¹ is cyclopropyl; R³ is absent; R⁷ and R² are joined to form a 6-membered monocyclic heteroaryl substituted with cyclopropyl; R^{4a} is absent; each R⁵ is independently methyl; R⁶ is H; R^{6′} is H or F; and R^{6″} is F.

In some embodiments of Formula (I-A′), or a pharmaceutically acceptable salt thereof, R¹ is C₃-C₄ cycloalkyl; R³ is absent; R⁷ and R² are joined to form a 10-membered bicyclic heteroaryl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 10-membered bicyclic heteroaryl; R^{4a} is absent; each R⁵ is independently methyl; R⁶ is H; R^{6′} is H or F; and R^{6″} is F.

In some embodiments of Formula (I-A′), or a pharmaceutically acceptable salt thereof, R¹ is halo; R³ is absent; R⁷ and R² are joined to form a 5- to 10-membered heterocyclyl; R^{4a} is absent; each R⁵ is independently C₁-C₆ alkyl; R⁶ is H; R^{6′} is H, halo, or —OH; and R^{6″} is H. In some embodiments, R¹ is Br or Cl; R³ is absent; R⁷ and R² are joined to form a 5-membered heterocyclyl; R^{4a} is absent; each R⁵ is independently methyl; R⁶ is H; R^{6′} is H, F, or —OH; and R^{6″} is H. In some embodiments, R¹ is Br; R³ is absent; R⁷ and R² are joined to form a 5-membered heterocyclyl; R^{4a} is absent; each R⁵ is independently methyl; R⁶ is H; R^{6′} is H or F; and R^{6″} is H. In some embodiments, R¹ is Cl; R³ is absent; R⁷ and R² are joined to form a 5-membered heterocyclyl; R^{4a} is absent; each R⁵ is independently methyl; R⁶ is H; R¹ is H or —OH; and R^{6″} is H.

In some embodiments of Formula (I-A′), or a pharmaceutically acceptable salt thereof, R¹ is C₃-C₄ cycloalkyl; R³ is absent; R⁷ and R² are joined to form a 5- to 10-membered heterocyclyl optionally substituted with one or more C₁-C₆ alkyl, C₁-C₆ haloalkyl, C₆ aryl, or C₃-C₅ cycloalkyl; R^{4a} is absent; each R⁵ is independently C₁-C₆ alkyl; R⁶ is H; R^{6′} is H or halo; and R^{6″} is H or halo. In some embodiments, R¹ is C₃-C₄ cycloalkyl; R³ is absent; R⁷ and R² are joined to form a 5-membered heterocyclyl; R^{4a} is absent; each R⁵ is independently C₁-C₆ alkyl; R⁶ is H; R^{6′} is H or halo; and R^{6″} is H. In some embodiments, R¹ is C₃-C₄ cycloalkyl; R³ is absent; R⁷ and R² are joined to form a 5-membered heterocyclyl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered heterocyclyl; R^{4a} is absent; each R⁵ is independently methyl; R⁶ is H; R^{6′} is H or F; and R^{6″} is H. In some embodiments, R¹ is cyclopropyl; R³ is absent; R⁷ and R² are joined to form a 5-membered heterocyclyl; R^{4a} is absent; each R⁵ is independently methyl; R⁶ is H; R^{6′} is H or F; and R^{6″} is F.

In some embodiments of Formula (I-A′), or a pharmaceutically acceptable salt thereof, R¹ is C₃-C₄ cycloalkyl; R³ is absent; R⁷ and R² are joined to form a 5-membered monocyclic heterocyclyl optionally substituted with one or more C₁-C₆ alkyl; R^{4a} is absent; each R⁵ is independently C₁-C₆ alkyl; R⁶ is H; R^{6′} is H or halo; and R is halo. In some embodiments, R¹ is cyclopropyl; R³ is absent; R⁷ and R² are joined to form a 5-membered monocyclic heterocyclyl substituted with methyl; R^{4a} is absent; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 5-membered monocyclic heterocyclyl optionally substituted with one or more C₁-C₆ haloalkyl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with —CF₃; R^{4a} is absent; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 5-membered monocyclic heterocyclyl optionally substituted with one or more C₆ aryl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with phenyl; R^{4a} is absent; each R⁵ is independently methyl; R⁶ is H; R^{6′} is H or F; and R^{6″} is F. In some embodiments, R¹ is C₃-C₄ cycloalkyl; R³ is absent; R⁷ and R² are joined to form a 5-membered monocyclic heterocyclyl optionally substituted with one or more C₃-C₅ cycloalkyl; R^{4a} is absent; each R⁵ is independently C₁-C₆ alkyl; R⁶ is H; R^{6′} is H or halo; and R^{6″} is halo. In some embodiments, R¹ is cyclopropyl; R³ is absent; R⁷ and R² are joined to form a 5-membered monocyclic heterocyclyl substituted with cyclopropyl; R^{4a} is absent; each R⁵ is independently methyl; R⁶ is H; R^{6′} is H or F; and R^{6″} is F.

In some embodiments of Formula (I-A), wherein R³ is absent and R² and R are joined to form a 5- to 10-membered monocyclic or bicyclic heteroaryl ring, the compound is of Formula (I-B), (I-C), (I-D), (I-E), (I-F), or (I-G):

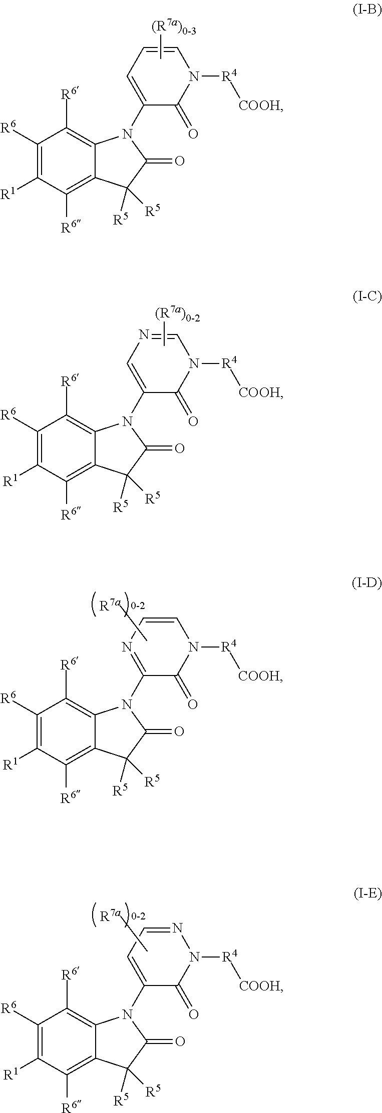

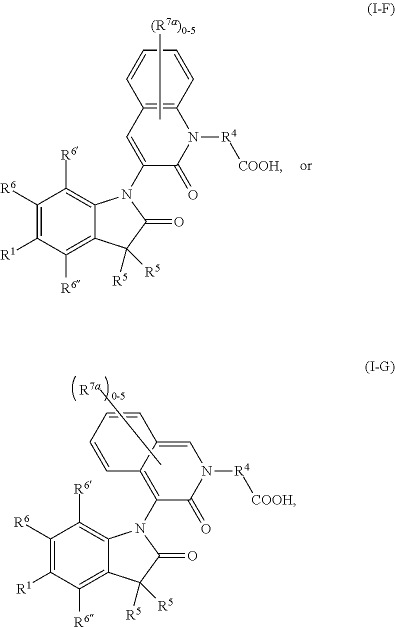

or a pharmaceutically acceptable salt thereof, comprising 0 R^{7a} groups (whereby R^{7a} is absent), or 1, 2, 3, 4, or 5 independent R^{7a} groups.

In some embodiments of Formula (I-B), (I-C), (I-D), (I-E), (I-F), or (I-G), or a pharmaceutically acceptable salt thereof, R¹ is halo; R^{7a} is absent; R⁴ is —C₁-C₆ alkylene-; each R⁵ is independently C₁-C₆ alkyl; R⁶ is H; R^{6′} is H, halo, or —OH; and R^{6″} is H. In some embodiments, R¹ is Br or Cl; R^{7a} is absent; R⁴ is —C₃ alkylene-; each R⁵ is independently methyl; R⁶ is H; R^{6′} is H, F, or —OH; and R^{6″} is H. In some embodiments, R¹ is Br; R^{7a} is absent; R⁴ is —C₃ alkylene-; each R⁵ is independently methyl; R⁶ is H; R^{6′} is H or F; and R^{6″} is H. In some embodiments, R¹ is Cl; R^{7a} is absent; R⁴ is —C₃ alkylene-; each R⁵ is independently methyl; R⁶ is H; R^{6′} is H or —OH; and R^{6″} is H.

In some embodiments of Formula (I-B), (I-C), (I-D), (I-E), (I-F), or (I-G), or a pharmaceutically acceptable salt thereof, R¹ is C₃-C₄ cycloalkyl; R^{7a} is C₁-C₆ alkyl, C₁-C₆ haloalkyl, C₆ aryl, or C₃-C₅ cycloalkyl; R⁴ is —C₁-C₆ alkylene-; each R⁵ is independently C₁-C₆ alkyl; R⁶ is H; R^{6′} is H or halo; and R^{6″} is H or halo. In some embodiments, R¹ is C₃-C₄ cycloalkyl; R^{7a} is absent; R⁴ is —C₃ alkylene-; each R⁵ is independently C₁-C₆ alkyl; R⁶ is H; R^{6′} is H or halo; and R^{6″} is H. In some embodiments, R¹ is C₃-C₄ cycloalkyl; R^{7a} is absent; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; R^{7a} is absent; R⁴ is —C₃ alkylene-; each R is independently methyl; R⁶ is H; R^{6′} is H or F; and R^{6″} is H. In some embodiments, R¹ is cyclopropyl; R^{7a} is absent; R⁴ is —C₃ alkylene-; each R⁵ is independently methyl; R⁶ is H; R^{6′} is H or F; and R^{6″} is F.

In some embodiments of Formula (I-B), (I-C), (I-D), (I-E), (I-F), or (I-G), or a pharmaceutically acceptable salt thereof, R¹ is C₃-C₄ cycloalkyl; one R^{7a} is present, which is C₁-C₆ alkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; one R^{7a} is present, which is methyl; R⁴ is —C₃ alkylene-; each R⁵ is independently methyl; Ru is H; R^{6′} is H or F; and R^{6″} is F. In some embodiments, R¹ is C₃-C₄ cycloalkyl; one R^{7a} is present, which is C₁-C₆ haloalkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; one R^{7a} is present, which is —CF₃; R⁴ is —C₃ alkylene-; each R⁵ is independently methyl; R⁶ is H; R^{6′} is H or F; and R^{6″} is F. In some embodiments, R¹ is C₃-C₄ cycloalkyl; one R^{7a} is present, which is C₆ aryl; R⁴ is —C₃ alkylene-; each R⁵ is independently C₁-C₆ alkyl; R⁶ is H; R^{6′} is H or halo; and R^{7a} is halo. In some embodiments, R¹ is cyclopropyl; one R^{7a} is present, which is phenyl; R⁴ is —C₃ alkylene-; each R⁵ is independently methyl; R⁶ is H; R^{6′} is H or F; and R^{6″} is F. In some embodiments, R¹ is C₃-C₄ cycloalkyl; one R^{7a} is present, which is C₃-C₅ cycloalkyl; R⁴ is —C₃ alkylene-; each R⁵ is independently C₁-C₆ alkyl; R⁶ is H; R^{6′} is H or halo; and R^{6″} is halo. In some embodiments, R¹ is cyclopropyl; one R^{a} is present, which is cyclopropyl; R⁴ is —C₃ alkylene-; each R⁵ is independently methyl; R⁶ is H; R^{6′} is H or F; and R^{6″} is F.

In some embodiments, the compound of Formula (I) is a compound of Formula (I-C), (I-D), or (I-E), or a pharmaceutically acceptable salt thereof.

In some embodiments of Formula (I-B), (I-C), (I-D), (I-E), (I-F), or (I-G), wherein R⁴ is —C₃ alkylene-, the compound is of Formula (I-B′), (I-C′), (I-D′), (I-E′), (I-F′), or (I-G′):

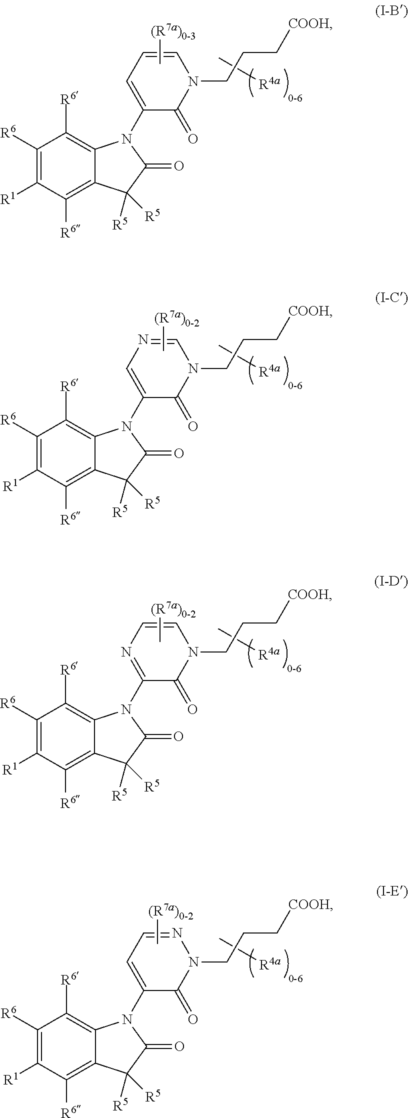

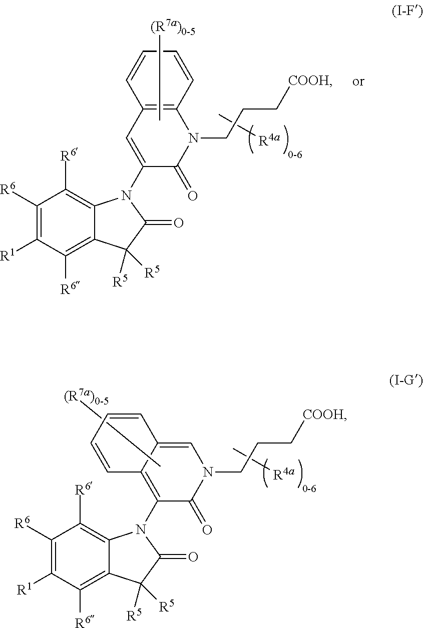

or a pharmaceutically acceptable salt thereof, comprising 0 R^{7a} groups (whereby R^{7a} is absent), or 1, 2, 3, 4, or 5 independent R^{7a} groups, and further comprising 0 R^{4a} groups (whereby R^{4a} is absent), or 1, 2, 3, 4, 5, or 6 independent R^{4a} groups.

In some embodiments of Formula (I-B′), (I-C′), (I-D′), (I-E′), (I-F′), or (I-G′), or a pharmaceutically acceptable salt thereof, R¹ is halo; R^{7a} is absent; R^{4a} is absent; each R⁵ is independently C₁-C₆ alkyl; R⁶ is H; R¹ is H, halo, or —OH; and R^{6″} is H. In some embodiments, R¹ is Br or Cl; R^{7a} is absent; R^{4a} is absent; each R⁵ is independently methyl; R⁶ is H; R^{6′} is H, F, or —OH; and R⁶ is H. In some embodiments, R¹ is Br; R^{7a} is absent; R^{4a} is absent; each R⁵ is independently methyl; R⁶ is H; R^{6′} is H or F; and R⁶ is H. In some embodiments, R¹ is Cl; R^{7a} is absent; R^{4a} is absent; each R⁵ is independently methyl; R⁶ is H; R⁶ is H or —OH; and R^{6″} is H.

In some embodiments of Formula (I-B′), (I-C′), (I-D′), (I-E′), (I-F′), or (I-G′), or a pharmaceutically acceptable salt thereof, R¹ is C₃-C₄ cycloalkyl; R^{7a} is C₁-C₆ alkyl, C₁-C₆ haloalkyl, C₆ aryl, or C₃-C₅ cycloalkyl; R^{4a} is absent; each R⁵ is independently C₁-C₆ alkyl; R⁶ is H; R^{6′} is H or halo; and R^{6″} is H or halo. In some embodiments, R¹ is C₃-C₄ cycloalkyl; R^{7a} is absent; R^{4a} is absent; each R⁵ is independently C₁-C₆ alkyl; R⁶ is H; R^{6′} is H or halo; and R^{6″} is H. In some embodiments, R¹ is C₃-C₄ cycloalkyl; R^{7a} is absent; R^{4a} is absent; each R⁵ is independently C₁-C₆ alkyl; R⁶ is H; R^{6′} is H or halo; and R^{6″} is halo. In some embodiments, R¹ is cyclopropyl; R^{7a} is absent; R^{4a} is absent; each R⁵ is independently methyl; R⁶ is H; R^{6′} is H or F; and R^{6″} is H. In some embodiments, R¹ is cyclopropyl; R^{7a} is absent; R^{4a} is absent; each R⁵ is independently methyl; R⁶ is H; R^{6′} is H or F; and R^{6″} is F.

In some embodiments of Formula (I-B′), (I-C′), (I-D′), (I-E′), (I-F′), or (I-G′), or a pharmaceutically acceptable salt thereof, R¹ is C₃-C₄ cycloalkyl; one R^{7a} is present, which is C₁-C₆ alkyl; R^{4a} is absent; each R⁵ is independently C₁-C₆ alkyl; R⁶ is H; R^{6′} is H or halo; and R^{6″} is halo. In some embodiments, R¹ is cyclopropyl; one R^{a} is present, which is methyl; R^{4a} is absent; each R⁵ is independently methyl; R⁶ is H; R^{6′} is H or F; and R^{6″} is F. In some embodiments, R¹ is C₃-C₄ cycloalkyl; one R^{a} is present, which is C₁-C₆ haloalkyl; R^{4a} is absent; each R⁵ is independently C₁-C₆ alkyl; R⁶ is H; R^{6′} is H or halo; and R^{6″} is halo. In some embodiments, R¹ is cyclopropyl; one R^{7a} is present, which is —CF₃; R^{4a} is absent; each R⁵ is independently methyl; R⁶ is H; R^{6′} is H or F; and R is F. In some embodiments, R¹ is C₃-C₄ cycloalkyl; one R^{7a} is present, which is C₆ aryl; R^{4a} is absent; each R⁵ is independently C₁-C₆ alkyl; R⁶ is H; R^{6′} is H or halo; and R^{6″} is halo. In some embodiments, R¹ is cyclopropyl; one R^{a} is present, which is phenyl; R^{4a} is absent; each R⁵ is independently methyl; R⁶ is H; R^{6′} is H or F; and R^{6″} is F. In some embodiments, R¹ is C₃-C₄ cycloalkyl; one R^{7a} is present, which is C₃-C₅ cycloalkyl; R^{4a} is absent; each R⁵ is independently C₁-C₆ alkyl; R⁶ is H; R^{6′} is H or halo; and R^{6″} is halo. In some embodiments, R¹ is cyclopropyl; one R^{7a} is present, which is cyclopropyl; R^{4a} is absent; each R⁵ is independently methyl; R⁶ is H; R^{6′} is H or F; and R^{6″} is F.

In some embodiments, the compound of Formula (I) is a compound of Formula (I-C′), (I-D′), or (I-E′), or a pharmaceutically acceptable salt thereof.

In some embodiments Formula (I-B), (I-C), (I-D), (I-E), (I-F), or (I-G), wherein R⁶ is hydrogen, the compound is of Formula (I-B-a), (I-C-a), (I-D-a), (I-E-a), (I-F-a), or (I-G-a):

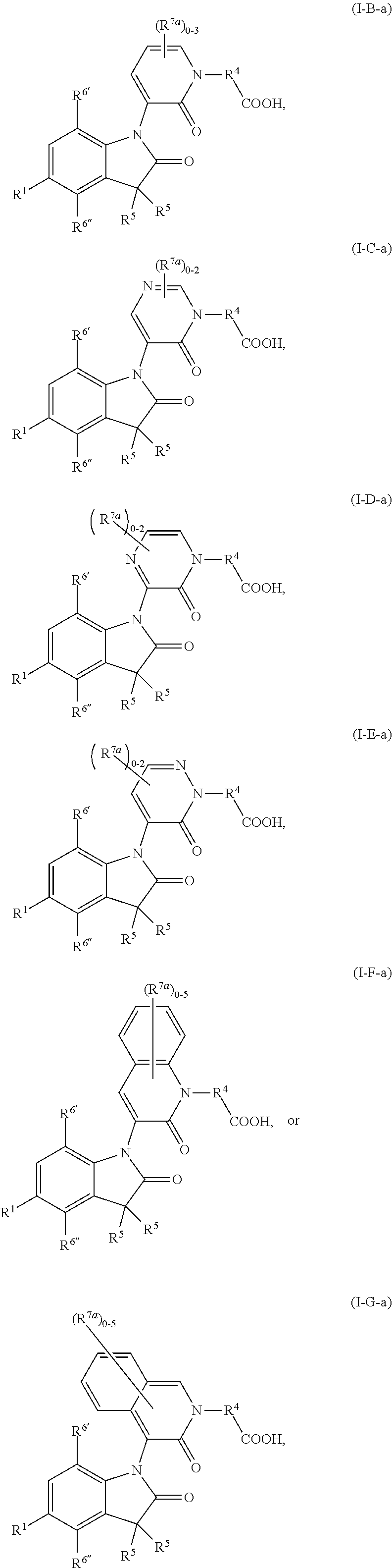

or a pharmaceutically acceptable salt thereof, comprising 0 R^{7a} groups (whereby R^{7a} is absent), or 1, 2, 3, 4, or 5 independent R^{7a} groups.

In some embodiments of Formula (I-B-a), (I-C-a), (I-D-a), (I-E-a), (I-F-a), or (I-G-a), or a pharmaceutically acceptable salt thereof, R¹ is halo; R^{7a} is absent; R⁴ is —C₁-C₆ alkylene-; each R⁵ is independently C₁-C₆ alkyl; R¹ is H, halo, or —OH; and R^{6″} is H. In some embodiments, R¹ is Br or Cl; R⁷ is absent; R⁴ is —C₃ alkylene-; each R⁵ is independently methyl; R^{6′} is H, F, or —OH; and R^{6″} is H. In some embodiments, R¹ is Br; R^{7a} is absent; R⁴ is —C₃ alkylene-; each R⁵ is independently methyl; R^{6′} is H or F; and R is H. In some embodiments, R¹ is Cl; R^{7a} is absent; R⁴ is —C₃ alkylene-; each R⁵ is independently methyl; R⁶ is H or —OH; and R^{6″} is H.

In some embodiments of Formula (I-B-a), (I-C-a), (I-D-a), (I-E-a), (I-F-a), or (I-G-a), or a pharmaceutically acceptable salt thereof, R¹ is C₃-C₄ cycloalkyl; R^{7a} is C₁-C₆ alkyl, C₁-C₆ haloalkyl, C₆ aryl, or C₃-C₅ cycloalkyl; R⁴ is —C₁-C₆ alkylene-; each R⁵ is independently C₁-C₆ alkyl; R^{6′} is H or halo; and R^{6″} is H or halo. In some embodiments, R¹ is C₃-C₄ cycloalkyl; R^{7a} is absent; R⁴ is —C₃ alkylene-; each R⁵ is independently C₁-C₆ alkyl; R^{6′} is H or halo; and R^{6″} is H. In some embodiments, R¹ is C₃-C₄ cycloalkyl; R^{7a} is absent; R⁴ is —C₃ alkylene-; each R⁵ is independently C₁-C₆ alkyl; R^{6′} is H or halo; and R^{6″} is halo. In some embodiments, R¹ is cyclopropyl; R^{7a} is absent; R⁴ is —C₃ alkylene-; each R⁵ is independently methyl; R^{6′} is H or F; and R^{6″} is H. In some embodiments, R¹ is cyclopropyl; R^{7a} is absent; R⁴ is —C₃ alkylene-; each R⁵ is independently methyl; R^{6′} is H or F; and R^{6″} is F.

In some embodiments of Formula (I-B-a), (I-C-a), (I-D-a), (I-E-a), (I-F-a), or (I-G-a), or a pharmaceutically acceptable salt thereof, R¹ is C₃-C₄ cycloalkyl; one R^{7a} is present, which is C₁-C₆ alkyl; R⁴ is —C₃ alkylene-; each R⁵ is independently C₁-C₆ alkyl; RE is H or halo; and R^{6″} is halo. In some embodiments, R¹ is cyclopropyl; one R^{7a} is present, which is methyl; R⁴ is —C₃ alkylene-; each R⁵ is independently methyl; R^{6′} is H or F; and R^{6″} is F. In some embodiments, R¹ is C₃-C₄ cycloalkyl; one R^{7a} is present, which is C₁-C₆ haloalkyl; R⁴ is —C₃ alkylene-; each R⁵ is independently C₁-C₆ alkyl; R^{6′} is H or halo; and R^{6″} is halo. In some embodiments, R¹ is cyclopropyl; one R^{7a} is present, which is —CF₃; R⁴ is —C₃ alkylene-; each R⁵ is independently methyl; R^{6′} is H or F; and R^{6″} is F. In some embodiments, R¹ is C₃-C₄ cycloalkyl; one R^{7a} is present, which is C₆ aryl; R⁴ is —C₃ alkylene-; each R⁵ is independently C₁-C₆ alkyl; R^{6′} is H or halo; and R^{6″} is halo. In some embodiments, R¹ is cyclopropyl; one R^{7a} is present, which is phenyl; R⁴ is —C₃ alkylene-; each R⁵ is independently methyl; R^{6′} is H or F; and R^{6″} is F. In some embodiments, R¹ is C₃-C₄ cycloalkyl; one R^{7a} is present, which is C₃-C₅ cycloalkyl; R⁴ is —C₃ alkylene-; each R⁵ is independently C₁-C₆ alkyl; R^{6′} is H or halo; and R^{6″} is halo. In some embodiments, R¹ is cyclopropyl; one R^{7a} is present, which is cyclopropyl; R⁴ is —C₃ alkylene-; each R⁵ is independently methyl; R^{6′} is H or F; and R^{6″} is F.

In some embodiments, the compound of Formula (I) is a compound of Formula (I-C-a), (I-D-a), or (I-E-a), or a pharmaceutically acceptable salt thereof.

In some embodiments of Formula (I-B), (I-C), (I-D), (I-E), (I-F), or (I-G), wherein R⁶ is hydrogen and R⁴ is —C₃ alkylene-, the compound is of Formula (I-B′-a), (I-C′-a), (I-D′-a), (I-E′-a), (I-F′-a), or (I-G′-a):

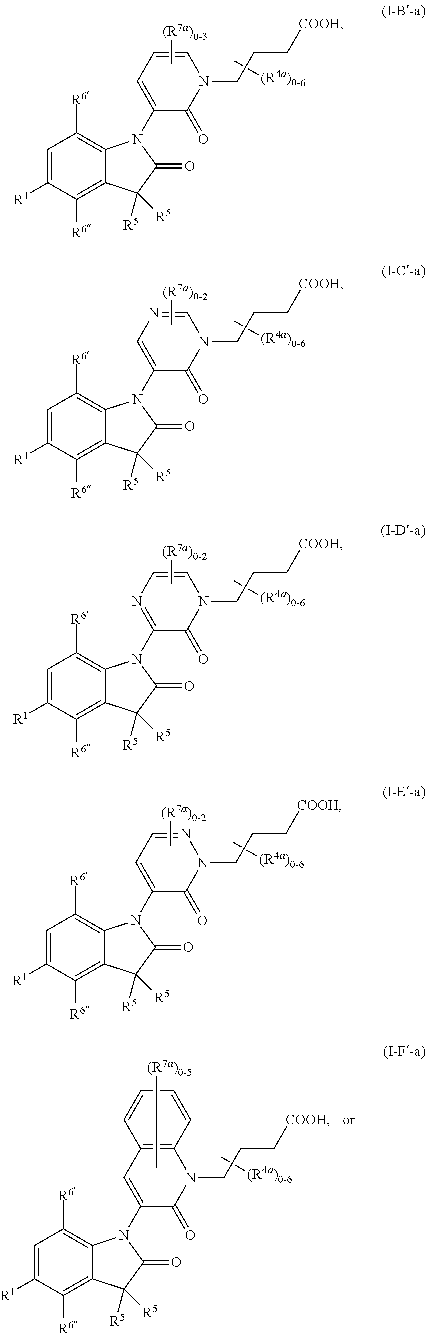

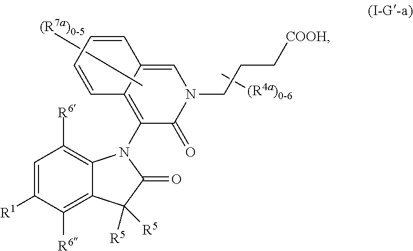

or a pharmaceutically acceptable salt thereof, comprising 0 R^{7a} groups (whereby R^{7a} is absent), or 1, 2, 3, 4, or 5 independent R^{7a} groups, and further comprising 0 R^{4a} groups (whereby R^{4a} is absent), or 1, 2, 3, 4, 5, or 6 independent R^{4a} groups.

In some embodiments of Formula (I-B′-a), (I-C′-a), (I-D′-a), (I-E′-a), (I-F′-a), or (I-G′-a), or a pharmaceutically acceptable salt thereof, R¹ is halo; R^{7a} is absent; R^{4a} is absent; each R⁵ is independently C₁-C₆ alkyl; R¹ is H, halo, or —OH; and R^{6″} is H. In some embodiments, R¹ is Br or Cl; R^{7a} is absent; R^{4a} is absent; each R⁵ is independently methyl; R^{6′} is H, F, or —OH; and R^{6″} is H. In some embodiments, R¹ is Br; R^{7a} is absent; R^{4a} is absent; each R⁵ is independently methyl; R^{6′} is H or F; and R^{6″} is H. In some embodiments, R¹ is Cl; R^{7a} is absent; R^{4a} is absent; each R⁵ is independently methyl; R^{6′} is H or —OH; and R^{6″} is H.

In some embodiments of Formula (I-B′-a), (I-C′-a), (I-D′-a), (I-E′-a), (I-F′-a), or (I-G′-a), or a pharmaceutically acceptable salt thereof, R¹ is C₃-C₄ cycloalkyl; R^{7a} is C₁-C₆ alkyl, C₁-C₆ haloalkyl, C₆ aryl, or C₃-C₅ cycloalkyl; R^{4a} is absent; each R⁵ is independently C₁-C₆ alkyl; R^{6′} is H or halo; and R^{6″} is H or halo. In some embodiments, R¹ is C₃-C₄ cycloalkyl; R^{7a} is absent; R^{4a} is absent; each R⁵ is independently C₁-C₆ alkyl; R^{6′} is H or halo; and R^{6″} is H. In some embodiments, R¹ is C₃-C₄ cycloalkyl; R^{7a} is absent; R^{4a} is absent; each R⁵ is independently C₁-C₆ alkyl; R^{6′} is H or halo; and R^{6″} is halo. In some embodiments, R¹ is cyclopropyl; R^{7a} is absent; R^{4a} is absent; each R⁵ is independently methyl; R^{6′} is H or F; and R^{6″} is H. In some embodiments, R¹ is cyclopropyl; R^{7a} is absent; R^{4a} is absent; each R⁵ is independently methyl; R^{6′} is H or F; and R^{6″} is F.

In some embodiments of Formula (I-B′-a), (I-C′-a), (I-D′-a), (I-E′-a), (I-F′-a), or (I-G′-a), or a pharmaceutically acceptable salt thereof, R¹ is C₃-C₄ cycloalkyl; 1 R^{7a} is present, which is C₁-C₆ alkyl; R^{4a} is absent; each R⁵ is independently C₁-C₆ alkyl; R^{6′} is H or halo; and R^{6″} is halo. In some embodiments, R¹ is cyclopropyl; one R^{a} is present, which is methyl; R^{4a} is absent; each R⁵ is independently methyl; R^{6′} is H or F; and R^{6″} is F. In some embodiments, R¹ is C₃-C₄ cycloalkyl; one R^{7a} is present, which is R^{7a} is C₁-C₆ haloalkyl; R^{4a} is absent; each R⁵ is independently C₁-C₆ alkyl; R^{6′} is H or halo; and R^{6″} is halo. In some embodiments, R¹ is cyclopropyl; one R^{7a} is present, which is —CF₃; R^{4a} is absent; each R⁵ is independently methyl; R^{6′} is H or F; and R^{6″} is F. In some embodiments, R¹ is C₃-C₄ cycloalkyl; one R^{7a} is present, which is C₆ aryl; R^{4a} is absent; each R⁵ is independently C₁-C₆ alkyl; R^{6′} is H or halo; and R^{6″} is halo. In some embodiments, R¹ is cyclopropyl; one R^{7a} is present, which is phenyl; R^{4a} is absent; each R is independently methyl; R^{6′} is H or F; and R^{6″} is F. In some embodiments, R¹ is C₃-C₄ cycloalkyl; one R^{7a} is present, which is C₃-C₅ cycloalkyl; R^{4a} is absent; each R⁵ is independently C₁-C₆ alkyl; R^{6′} is H or halo; and R^{6″} is halo. In some embodiments, R¹ is cyclopropyl; one R^{7a} is present, which is cyclopropyl; R^{4a} is absent; each R⁵ is independently methyl; R^{6′} is H or F; and R^{6″} is F.

In some embodiments, the compound of Formula (I) is a compound of Formula (I-C′-a), (I-D′-a), or (I-E′-a), or a pharmaceutically acceptable salt thereof.

In some embodiments of Formula (I-A), wherein R³ is absent (or R³ is hydrogen) and R² and R are joined to form a 5-membered monocyclic heterocyclyl ring, the compound is of Formula (I-J):

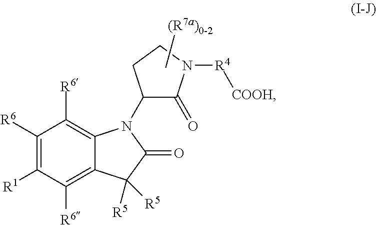

or a pharmaceutically acceptable salt thereof, comprising 0 R^{7a} groups (whereby R^{7a} is absent), or 1 or 2 independent R^{7a} groups.

In some embodiments of Formula (I-J), or a pharmaceutically acceptable salt thereof, R¹ is C₁-C₆ haloalkyl or C₃-C₅ cycloalkyl; R^{7a} is absent; R⁴ is —C₁-C₆ alkylene-; each R⁵ is independently C₁-C₆ alkyl; Ru is H; R^{6′} is halo or H; and R^{6″} is halo or H.

In some embodiments of Formula (I-J), or a pharmaceutically acceptable salt thereof, R¹ is C₁-C₆ haloalkyl; R^{7a} is absent; R⁴ is —C₁-C₆ alkylene-; each R⁵ is independently C₁-C₆ alkyl; R⁶ is H; R^{6′} is halo or H; and R^{6″} is halo or H. In some embodiments, R¹ is CF₃; R^{7a} is absent; R⁴ is —C₃ alkylene-; each R⁵ is independently methyl; R⁶ is H; R^{6′} is F or H; and R^{6″} is F or H. In some embodiments, R¹ is CF₃; R^{7a} is absent; R⁴ is —C₃ alkylene-; each R⁵ is independently methyl; R⁶ is H; R^{6′} is F or H; and R^{6″} is F. In some embodiments, R¹ is CF₃; R^{7a} is absent; R⁴ is —C₃ alkylene-; each R⁵ is independently methyl; R⁶ is H; R^{6′} is F or H; and R^{6″} is H.

In some embodiments of Formula (I-J), or a pharmaceutically acceptable salt thereof, R¹ is C₁-C₆ haloalkyl; R^{7a} is absent; R⁴ is —C₁-C₆ alkylene-; each R⁵ is independently C₁-C₆ alkyl; R⁶ is H; R^{6′} is halo; and R^{6″} is halo or H. In some embodiments, R¹ is CF₃; R^{7a} is absent; R⁴ is —C₃ alkylene-; each R⁵ is independently methyl; R⁶ is H; R^{6′} is F; and R^{6″} is F or H. In some embodiments, R¹ is CF₃; R^{7a} is absent; R⁴ is —C₃ alkylene-; each R⁵ is independently methyl; R⁶ is H; R^{6′} is F; and R^{6″} is F. In some embodiments, R¹ is CF₃; R^{7a} is absent; R⁴ is —C₃ alkylene-; each R⁵ is independently methyl; R⁶ is H; R^{6′} is F; and R^{6″} is H.

In some embodiments of Formula (I-J), or a pharmaceutically acceptable salt thereof, R¹ is C₁-C₆ haloalkyl; R^{7a} is absent; R⁴ is —C₁-C₆ alkylene-; each R⁵ is independently C₁-C₆ alkyl; R⁶ is H; R^{6′} is H; and R^{6″} is halo or H. In some embodiments, R¹ is CF₃; R^{7a} is absent; R⁴ is —C₃ alkylene-; each R⁵ is independently methyl; R⁶ is H; R^{6′} is H; and R^{6″} is F or H. In some embodiments, R¹ is CF₃; R^{7a} is absent; R⁴ is —C₃ alkylene-; each R⁵ is independently methyl; R⁶ is H; R^{6′} is H; and R^{6″} is F. In some embodiments, R¹ is CF₃; R^{7a} is absent; R⁴ is —C₃ alkylene-; each R⁵ is independently methyl; R⁶ is H; R^{6′} is H; and R^{6″} is H.

In some embodiments of Formula (I-J), or a pharmaceutically acceptable salt thereof, R¹ is C₃-C₅ cycloalkyl; R^{7a} is absent; R⁴ is —C₁-C₆ alkylene-; each R⁵ is independently C₁-C₆ alkyl; R⁶ is H; R¹ is halo or H; and R^{6″} is halo or H. In some embodiments, R¹ is cyclopropyl; R^{7a} is absent; R⁴ is —C₃ alkylene-; each R⁵ is independently methyl; R⁶ is H; R^{6′} is F or H; and R^{6″} is F or H. In some embodiments, R¹ is cyclopropyl; R^{7a} is absent; R⁴ is —C₃ alkylene-; each R⁵ is independently methyl; R⁶ is H; R^{6′} is F or H; and R^{6″} is F. In some embodiments, R¹ is cyclopropyl; R^{7a} is absent; R⁴ is —C₃ alkylene-; each R⁵ is independently methyl; R⁶ is H; R^{6′} is F or H; and R^{6″} is H.

In some embodiments of Formula (I-J), or a pharmaceutically acceptable salt thereof, R¹ is C₃-C₅ cycloalkyl; R^{7a} is absent; R⁴ is —C₁-C₆ alkylene-; each R⁵ is independently C₁-C₆ alkyl; R⁶ is H; R^{6′} is halo; and R^{6″} is halo or H. In some embodiments, R¹ is cyclopropyl; R^{7a} is absent; R⁴ is —C₃ alkylene-; each R⁵ is independently methyl; R⁶ is H; R^{6′} is F; and R^{6″} is F or H. In some embodiments, R¹ is cyclopropyl; R^{7a} is absent; R⁴ is —C₃ alkylene-; each R⁵ is independently methyl; R⁶ is H; R^{6′} is F; and R^{6″} is F. In some embodiments, R¹ is cyclopropyl; R^{7a} is absent; R⁴ is —C₃ alkylene-; each R⁵ is independently methyl; R⁶ is H; R^{6′} is F; and R^{6″} is H.

In some embodiments of Formula (I-J), or a pharmaceutically acceptable salt thereof, R¹ is C₃-C₅ cycloalkyl; R^{7a} is absent; R⁴ is —C₁-C₆ alkylene-; each R⁵ is independently C₁-C₆ alkyl; R⁶ is H; R^{6′} is H; and R^{6″} is halo or H. In some embodiments, R¹ is cyclopropyl; R^{7a} is absent; R⁴ is —C₃ alkylene-; each R⁵ is independently methyl; R⁶ is H; R^{6′} is H; and R^{6″} is F or H. In some embodiments, R¹ is cyclopropyl; R^{7a} is absent; R⁴ is —C₃ alkylene-; each R⁵ is independently methyl; R⁶ is H; R^{6′} is H; and R^{6″} is F. In some embodiments, R¹ is cyclopropyl; R^{7a} is absent; R⁴ is —C₃ alkylene-; each R⁵ is independently methyl; R⁶ is H; R^{6′} is H; and R^{6″} is H.

In some embodiments of Formula (I-J), wherein R⁴ is a —C₃ alkylene-, the compound is of Formula (I-J′):

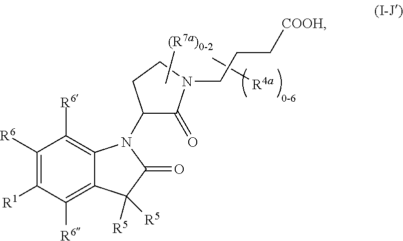

or a pharmaceutically acceptable salt thereof, comprising 0 R^{7a} groups (whereby R^{7a} is absent), or 1 or 2 independent R^{7a} groups, and further comprising 0 R^{4a} groups (whereby R^{4a} is absent), or 1, 2, 3, 4, 5, or 6 independent R^{4a} groups.

In some embodiments of Formula (I-J′), or a pharmaceutically acceptable salt thereof, R¹ is C₁-C₆ haloalkyl or C₃-C₅ cycloalkyl; R^{7a} is absent; each R⁵ is independently C₁-C₆ alkyl; R⁶ is H; R^{6′} is halo or H; and R^{6″} is halo or H.

In some embodiments of Formula (I-J′), or a pharmaceutically acceptable salt thereof, R¹ is C₁-C₆ haloalkyl; R^{7a} is absent; each R⁵ is independently C₁-C₆ alkyl; R⁶ is H; R^{6′} is halo or H; and R^{6″} is halo or H. In some embodiments, R¹ is —CF₃; R^{7a} is absent; each R⁵ is independently methyl; R⁶ is H; R^{6′} is F or H; and R^{6″} is F or H. In some embodiments, R¹ is —CF₃; R^{7a} is absent; each R⁵ is independently methyl; R⁶ is H; R^{6′} is F or H; and R^{6″} is F. In some embodiments, R¹ is —CF₃; R^{7a} is absent; each R⁵ is independently methyl; R⁶ is H; R^{6′} is F or H; and R^{6″} is H.

In some embodiments of Formula (I-J′), or a pharmaceutically acceptable salt thereof, R¹ is C₁-C₆ haloalkyl; R^{7a} is absent; each R⁵ is independently C₁-C₆ alkyl; R⁶ is H; R^{6′} is halo; and R^{6″} is halo or H. In some embodiments, R¹ is —CF₃; R^{7a} is absent; each R⁵ is independently methyl; R⁶ is H; R^{6′} is F; and R^{6″} is F or H. In some embodiments, R¹ is —CF₃; R^{7a} is absent; each R⁵ is independently methyl; R⁶ is H; R^{6′} is F; and R^{6″} is F. In some embodiments, R¹ is —CF₃; R^{7a} is absent; each R⁵ is independently methyl; R⁶ is H; R^{6′} is F; and R^{6″} is H.

In some embodiments of Formula (I-J′), or a pharmaceutically acceptable salt thereof, R¹ is C₁-C₆ haloalkyl; R^{7a} is absent; each R⁵ is independently C₁-C₆ alkyl; R⁶ is H; R^{6′} is H; and R^{6″} is halo or H. In some embodiments, R¹ is —CF₃; R^{7a} is absent; each R⁵ is independently methyl; R⁶ is H; R^{6′} is H; and R is F or H. In some embodiments, R¹ is —CF₃; R^{7a} is absent; each R⁵ is independently methyl; R⁶ is H; R^{6′} is H; and R^{6″} is F. In some embodiments, R¹ is —CF₃; R^{7a} is absent; each R⁵ is independently methyl; R⁶ is H; R^{6′} is H; and R^{6″} is H.

In some embodiments of Formula (I-J′), or a pharmaceutically acceptable salt thereof, R¹ is C₃-C₅ cycloalkyl; R^{7a} is absent; each R⁵ is independently C₁-C₆ alkyl; R⁶ is H; R^{6′} is halo or H; and R^{6″} is halo or H. In some embodiments, R¹ is cyclopropyl; R^{7a} is absent; each R⁵ is independently methyl; R⁶ is H; R^{6′} is F or H; and R^{6″} is F or H. In some embodiments, R¹ is cyclopropyl; R^{7a} is absent; each R⁵ is independently methyl; R⁶ is H; R^{6′} is F or H; and R^{6″} is F. In some embodiments, R¹ is cyclopropyl; R^{7a} is absent; each R⁵ is independently methyl; R⁶ is H; R^{6′} is F or H; and R^{6″} is H. In some embodiments, R¹ is C₃-C₅ cycloalkyl; R^{7a} is absent; each R⁵ is independently C₁-C₆ alkyl; R⁶ is H; R^{6′} is halo; and R^{6″} is halo or H. In some embodiments, R¹ is cyclopropyl; R^{7a} is absent; each R⁵ is independently methyl; R⁶ is H; R^{6′} is F; and R^{6″} is F or H. In some embodiments, R¹ is cyclopropyl; R^{7a} is absent; each R⁵ is independently methyl; R⁶ is H; R^{6′} is F; and R^{6″} is F. In some embodiments, R¹ is cyclopropyl; R^{7a} is absent; each R⁵ is independently methyl; R⁶ is H; R^{6′} is F; and R^{6″} is H.

In some embodiments of Formula (I-J′), or a pharmaceutically acceptable salt thereof, R¹ is C₃-C₅ cycloalkyl; R^{7a} is absent; each R⁵ is independently C₁-C₆ alkyl; R⁶ is H; R^{6′} is H; and R^{6″} is halo or H. In some embodiments, R¹ is cyclopropyl; R^{7a} is absent; each R⁵ is independently methyl; R⁶ is H; R^{6′} is H; and R^{6″} is F or H. In some embodiments, R¹ is cyclopropyl; R^{7a} is absent; each R⁵ is independently methyl; R⁶ is H; R^{6′} is H; and R^{6′} is F. In some embodiments, R¹ is cyclopropyl; R^{7a} is absent; each R⁵ is independently methyl; R⁶ is H; R^{6′} is H; and R^{6″} is H.

In some embodiments of Formula (I-J), wherein R⁶ is hydrogen, the compound is of Formula (I-J-a).

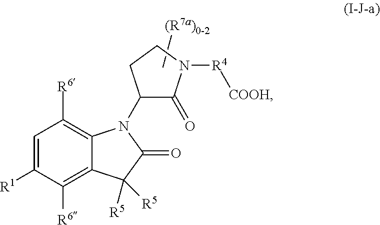

or a pharmaceutically acceptable salt thereof, comprising 0 R^{7a} groups (whereby R^{7a} is absent), or 1 or 2 independent R^{7a} groups.

In some embodiments of Formula (I-J-a), or a pharmaceutically acceptable salt thereof, R¹ is C₁-C₆ haloalkyl or C₃-C₅ cycloalkyl; R^{7a} is absent; R⁴ is —C₁-C₆ alkylene-; each R⁵ is independently C₁-C₆ alkyl; R^{6′} is halo or H; and R^{6″} is halo or H.

In some embodiments of Formula (I-J-a), or a pharmaceutically acceptable salt thereof, R¹ is C₁-C₆ haloalkyl; R^{7a} is absent; R⁴ is —C₁-C₆ alkylene-; each R⁵ is independently C₁-C₆ alkyl; R^{6′} is halo or H; and R^{6″} is halo or H. In some embodiments, R¹ is —CF₃; R^{7a} is absent; R⁴ is —C₃ alkylene-; each R⁵ is independently methyl; R^{6′} is F or H; and R^{6″} is F or H. In some embodiments, R¹ is —CF₃; R^{7a} is absent; R⁴ is —C₃ alkylene-; each R⁵ is independently methyl; R^{6′} is F or H; and R^{6″} is F. In some embodiments, R¹ is —CF₃; R^{7a} is absent; R⁴ is —C₃ alkylene-; each R⁵ is independently methyl; R^{6′} is F or H; and R⁶ is H.

In some embodiments of Formula (I-J-a), or a pharmaceutically acceptable salt thereof, R¹ is C₁-C₆ haloalkyl; R^{7a} is absent; R⁴ is —C₁-C₆ alkylene-; each R⁵ is independently C₁-C₆ alkyl; R^{6′} is halo; and R^{6″} is halo or H. In some embodiments, R¹ is —CF₃; R^{7a} is absent; R⁴ is —C₃ alkylene-; each R⁵ is independently methyl; R^{6′} is F; and R^{6″} is F or H. In some embodiments, R¹ is —CF₃; R^{7a} is absent; R⁴ is —C₃ alkylene-; each R⁵ is independently methyl; R^{6′} is F; and R^{6″} is F. In some embodiments, R¹ is —CF₃; R^{7a} is absent; R⁴ is —C₃ alkylene-; each R⁵ is independently methyl; R^{6′} is F; and R^{6″} is H.

In some embodiments of Formula (I-J-a), or a pharmaceutically acceptable salt thereof, R¹ is C₁-C₆ haloalkyl; R^{7a} is absent; R⁴ is —C₁-C₆ alkylene-; each R⁵ is independently C₁-C₆ alkyl; R^{6′} is H; and R^{6″} is halo or H. In some embodiments, R¹ is —CF₃; R^{7a} is absent; R⁴ is —C₃ alkylene-; each R⁵ is independently methyl; R^{6′} is H; and R^{6″} is F or H. In some embodiments, R¹ is —CF₃; R^{7a} is absent; R⁴ is —C₃ alkylene-; each R⁵ is independently methyl; R^{6′} is H; and R^{6″} is F. In some embodiments, R¹ is —CF₃; R^{7a} is absent; R⁴ is —C₃ alkylene-; each R⁵ is independently methyl; R^{6′} is H; and R^{6″} is H.

In some embodiments of Formula (I-J-a), or a pharmaceutically acceptable salt thereof, R¹ is C₃-C₅ cycloalkyl; R^{7a} is absent; R⁴ is —C₁-C₆ alkylene-; each R⁵ is independently C₁-C₆ alkyl; R^{6′} is halo or H; and R^{6″} is halo or H. In some embodiments, R¹ is cyclopropyl; R^{7a} is absent; R⁴ is —C₃ alkylene-; each R⁵ is independently methyl; R^{6′} is F or H; and R^{6″} is F or H. In some embodiments, R¹ is cyclopropyl; R^{7a} is absent; R⁴ is —C₃ alkylene-; each R⁵ is independently methyl; R^{6′} is F or H; and R^{6″} is F. In some embodiments, R¹ is cyclopropyl; R^{7a} is absent; R⁴ is —C₃ alkylene-; each R⁵ is independently methyl; R^{6′} is F or H; and R^{6″} is H.

In some embodiments of Formula (I-J-a), or a pharmaceutically acceptable salt thereof, R¹ is C₃-C₅ cycloalkyl; R^{7a} is absent; R⁴ is —C₁-C₆ alkylene-; each R⁵ is independently C₁-C₆ alkyl; R^{6′} is halo; and R^{6″} is halo or H. In some embodiments, R¹ is cyclopropyl; R^{7a} is absent; R⁴ is —C₃ alkylene-; each R⁵ is independently methyl; R^{6′} is F; and R^{6″} is F or H. In some embodiments, R¹ is cyclopropyl; R^{7a} is absent; R⁴ is —C₃ alkylene-; each R⁵ is independently methyl; R^{6′} is F; and R^{6″} is F. In some embodiments, R¹ is cyclopropyl; R^{7a} is absent; R⁴ is —C₃ alkylene-; each R⁵ is independently methyl; R^{6′} is F; and R^{6″} is H.

In some embodiments of Formula (I-J-a), or a pharmaceutically acceptable salt thereof, R¹ is C₃-C₅ cycloalkyl; R^{7a} is absent; R⁴ is —C₁-C₆ alkylene-; each R⁵ is independently C₁-C₆ alkyl; R^{6′} is H; and R^{6″} is halo or H. In some embodiments, R¹ is cyclopropyl; R^{7a} is absent; R⁴ is —C₃ alkylene-; each R⁵ is independently methyl; R^{6′} is H; and R^{6″} is F or H. In some embodiments, R¹ is cyclopropyl; R^{7a} is absent; R⁴ is —C₃ alkylene-; each R⁵ is independently methyl; R^{6′} is H; and R^{6″} is F. In some embodiments, R¹ is cyclopropyl; R^{7a} is absent; R⁴ is —C₃ alkylene-; each R⁵ is independently methyl; R^{6′} is H; and R^{6″} is H.

In some embodiments of Formula (I-J-a), wherein R⁶ is hydrogen and R⁴ is —C₃ alkylene-, the compound is of Formula (I-J′-a):

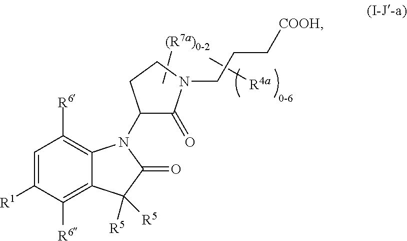

or a pharmaceutically acceptable salt thereof, comprising 0 R^{7a} groups (whereby R^{7a} is absent), or 1 or 2 independent R^{7a} groups, and further comprising 0 R^{4a} groups (whereby R^{4a} is absent), or 1, 2, 3, 4, 5, or 6 independent R^{4a} groups.

In some embodiments of Formula (I-J′-a), or a pharmaceutically acceptable salt thereof, R¹ is C₁-C₆ haloalkyl or C₃-C₅ cycloalkyl; R^{7a} is absent; each R⁵ is independently C₁-C₆ alkyl; R^{6′} is halo or H; and R^{6″} is halo or H.

In some embodiments of Formula (I-J′-a), or a pharmaceutically acceptable salt thereof, R¹ is C₁-C₆ haloalkyl; R^{7a} is absent; each R⁵ is independently C₁-C₆ alkyl; R^{6′} is halo or H; and R^{6″} is halo or H. In some embodiments, R¹ is CF₃; R^{7a} is absent; each R⁵ is independently methyl; R^{6′} is F or H; and R^{6″} is F or H. In some embodiments, R¹ is CF₃; R^{7a} is absent; each R⁵ is independently methyl; R^{6′} is F or H; and R^{6″} is F. In some embodiments, R¹ is CF₃; R^{7a} is absent; each R⁵ is independently methyl; R^{6′} is F or H; and R^{6″} is H.

In some embodiments of Formula (I-J′-a), or a pharmaceutically acceptable salt thereof, R¹ is C₁-C₆ haloalkyl; R^{7a} is absent; each R⁵ is independently C₁-C₆ alkyl; R^{6′} is halo; and R^{6″} is halo or H. In some embodiments, R¹ is CF₃; R^{7a} is absent; each R⁵ is independently methyl; R^{6′} is F; and R^{6″} is F or H. In some embodiments, R¹ is CF₃; R^{7a} is absent; each R⁵ is independently methyl; R^{6′} is F; and R^{6″} is F. In some embodiments, R¹ is CF₃; R^{7a} is absent; each R⁵ is independently methyl; R^{6′} is F; and R^{6″} is H.

In some embodiments of Formula (I-J′-a), or a pharmaceutically acceptable salt thereof, R¹ is C₁-C₆ haloalkyl; R^{7a} is absent; each R⁵ is independently C₁-C₆ alkyl; R^{6′} is H; and R^{6″} is halo or H. In some embodiments, R¹ is CF₃; R^{7a} is absent; each R⁵ is independently methyl; R^{6′} is H; and R^{6″} is F or H. In some embodiments, R¹ is CF₃; R^{7a} is absent; each R⁵ is independently methyl; R⁶ is H; and R⁶ is F. In some embodiments, R¹ is CF₃; R^{7a} is absent; each R⁵ is independently methyl; R^{6′} is H; and R^{6″} is H.

In some embodiments of Formula (I-J′-a), or a pharmaceutically acceptable salt thereof, R¹ is C₃-C₅ cycloalkyl; R^{7a} is absent; each R⁵ is independently C₁-C₆ alkyl; R^{6′} is halo or H; and R^{6″} is halo or H. In some embodiments, R¹ is cyclopropyl; R^{7a} is absent; each R⁵ is independently methyl; R^{6′} is F or H; and R^{6″} is F or H. In some embodiments, R¹ is cyclopropyl; R^{7a} is absent; each R⁵ is independently methyl; R^{6′} is F or H; and R^{6″} is F. In some embodiments, R¹ is cyclopropyl; R^{7a} is absent; each R⁵ is independently methyl; R^{6′} is F or H; and R^{6″} is H.

In some embodiments of Formula (I-J′-a), or a pharmaceutically acceptable salt thereof, R¹ is C₃-C₅ cycloalkyl; R^{7a} is absent; each R⁵ is independently C₁-C₆ alkyl; R^{6′} is halo; and R^{6″} is halo or H. In some embodiments, R¹ is cyclopropyl; R^{7a} is absent; each R⁵ is independently methyl; R^{6′} is F; and R^{6″} is F or H. In some embodiments, R¹ is cyclopropyl; R^{7a} is absent; each R⁵ is independently methyl; R^{6′} is F; and R^{6″} is F. In some embodiments, R¹ is cyclopropyl; R^{7a} is absent; each R⁵ is independently methyl; R^{6′} is F; and R^{6″} is H.

In some embodiments of Formula (I-J′-a), or a pharmaceutically acceptable salt thereof, R¹ is C₃-C₅ cycloalkyl; R^{7a} is absent; each R⁵ is independently C₁-C₆ alkyl; R^{6′} is H; and R^{6″} is halo or H. In some embodiments, R¹ is cyclopropyl; R^{7a} is absent; each R⁵ is independently methyl; R^{6′} is H; and R^{6″} is F or H. In some embodiments, R¹ is cyclopropyl; R^{7a} is absent; each R⁵ is independently methyl; R¹ is H; and R^{6″} is F. In some embodiments, R¹ is cyclopropyl; R^{7a} is absent; each R⁵ is independently methyl; R^{6′} is H; and R^{6″} is H.

In some embodiments of Formula (I-J′-a), the compound has the following (R) stereochemistry to provide a compound of Formula I-J′-a-R

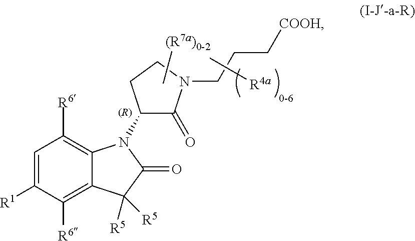

or a pharmaceutically acceptable salt thereof, comprising 0 R^{7a} groups (whereby R^{7a} is absent), or 1 or 2 independent R^{7a} groups, and further comprising 0 R^{4a} groups (whereby R^{4a} is absent), or 1, 2, 3, 4, 5, or 6 independent R^{4a} groups.

In some embodiments of Formula (I-J′-a-R), or a pharmaceutically acceptable salt thereof, R¹ is C₁-C₆ haloalkyl or C₃-C₅ cycloalkyl; R^{7a} is absent; each R⁵ is independently C₁-C₆ alkyl; R^{6′} is halo or H; and R^{6″} is halo or H.

In some embodiments of Formula (I-J′-a-R), or a pharmaceutically acceptable salt thereof, R¹ is C₁-C₆ haloalkyl; R^{7a} is absent; each R⁵ is independently C₁-C₆ alkyl; R^{6′} is halo or H; and R^{6″} is halo or H.

In some embodiments, R¹ is —CF₃; R^{a} is absent; each R⁵ is independently methyl; R^{6′} is F or H; and R⁶ is F or H. In some embodiments, R¹ is —CF₃; R^{7a} is absent; each R⁵ is independently methyl; R¹ is F or H; and R^{6″} is F. In some embodiments, R¹ is —CF₃; R^{7a} is absent; each R⁵ is independently methyl; R^{6′} is F or H; and R^{6″} is H.

In some embodiments of Formula (I-J′-a-R), or a pharmaceutically acceptable salt thereof, R¹ is C₁-C₆ haloalkyl; R^{7a} is absent; each R⁵ is independently C₁-C₆ alkyl; R^{6′} is halo; and R^{6″} is halo or H. In some embodiments, R¹ is —CF₃; R^{7a} is absent; each R⁵ is independently methyl; R^{6′} is F; and R^{6″} is F or H.

In some embodiments, R¹ is —CF₃; R^{7a} is absent; each R⁵ is independently methyl; R^{6′} is F; and R^{6″} is F. In some embodiments, R¹ is —CF₃; R^{7a} is absent; each R⁵ is independently methyl; R^{6′} is F; and R^{6″} is H.

In some embodiments of Formula (I-J′-a-R), or a pharmaceutically acceptable salt thereof, R¹ is C₁-C₆ haloalkyl; R^{7a} is absent; each R⁵ is independently C₁-C₆ alkyl; R^{6′} is H; and R^{6″} is halo or H. In some embodiments, R¹ is —CF₃; R^{7a} is absent; each R⁵ is independently methyl; R^{6′} is H; and R^{6″} is F or H.

In some embodiments, R¹ is —CF₃; R^{7a} is absent; each R⁵ is independently methyl; R⁶ is H; and R^{6″} is F. In some embodiments, R¹ is —CF₃; R^{7a} is absent; each R⁵ is independently methyl; R^{6′} is H; and R^{6″} is H.

In some embodiments of Formula (I-J′-a-R), or a pharmaceutically acceptable salt thereof, R¹ is C₃-C₅ cycloalkyl; R^{7a} is absent; each R⁵ is independently C₁-C₆ alkyl; R^{6′} is halo or H; and R^{6″} is halo or H. In some embodiments, R¹ is cyclopropyl; R^{7a} is absent; each R⁵ is independently methyl; R^{6′} is F or H; and R^{6″} is F or H. In some embodiments, R¹ is cyclopropyl; R^{7a} is absent; each R⁵ is independently methyl; R^{6′} is F or H; and R^{6″} is F. In some embodiments, R¹ is cyclopropyl; R^{7a} is absent; each R⁵ is independently methyl; R^{6′} is F or H; and R^{6″} is H.

In some embodiments of Formula (I-J′-a-R), or a pharmaceutically acceptable salt thereof, R¹ is C₃-C₅ cycloalkyl; R^{7a} is absent; each R⁵ is independently C₁-C₆ alkyl; R^{6′} is halo; and R^{6″} is halo or H. In some embodiments, R¹ is cyclopropyl; R^{7a} is absent; each R⁵ is independently methyl; R^{6′} is F; and R^{6″} is F or H. In some embodiments, R¹ is cyclopropyl; R^{7a} is absent; each R⁵ is independently methyl; R^{6′} is F; and R^{6″} is F. In some embodiments, R¹ is cyclopropyl; R^{7a} is absent; each R⁵ is independently methyl; R^{6′} is F; and R^{6″} is H.

In some embodiments of Formula (I-J′-a-R), or a pharmaceutically acceptable salt thereof, R¹ is C₃-C₅ cycloalkyl; R^{7a} is absent; each R⁵ is independently C₁-C₆ alkyl; R^{6′} is H; and R^{6″} is halo or H. In some embodiments, R¹ is cyclopropyl; R^{7a} is absent; each R⁵ is independently methyl; R^{6′} is H; and R^{6″} is F or H. In some embodiments, R¹ is cyclopropyl; R^{7a} is absent; each R⁵ is independently methyl; R^{6′} is H; and R^{6″} is F. In some embodiments, R¹ is cyclopropyl; R^{7a} is absent; each R⁵ is independently methyl; R^{6′} is H; and R^{6″} is H.

In some embodiments of Formula (I-J′-a), the compound has the following (S) stereochemistry to provide a compound of Formula (I-J′-a-S):

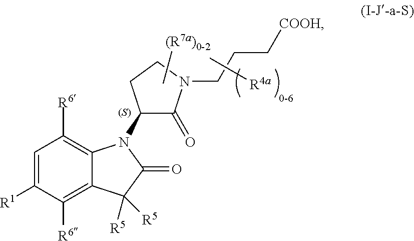

or a pharmaceutically acceptable salt thereof, comprising 0 R^{7a} groups (whereby R^{7a} is absent), or 1 or 2 independent R^{7a} groups, and further comprising 0 R^{4a} groups (whereby R^{4a} is absent), or 1, 2, 3, 4, 5, or 6 independent R^{4a} groups.

In some embodiments of Formula (I-J′-a-S), or a pharmaceutically acceptable salt thereof, R¹ is C₁-C₆ haloalkyl or C₃-C₅ cycloalkyl; R^{7a} is absent; each R⁵ is independently C₁-C₆ alkyl; R^{6′} is halo or H; and R^{6″} is halo or H.

In some embodiments, R¹ is C₁-C₆ haloalkyl; R^{7a} is absent; each R⁵ is independently C₁-C₆ alkyl; R^{6′} is halo or H; and R^{6″} is halo or H. In some embodiments, R¹ is —CF₃; R^{7a} is absent; each R⁵ is independently methyl; R¹ is F or H; and R^{6″} is F or H. In some embodiments, R¹ is —CF₃; R^{7a} is absent; each R⁵ is independently methyl; R^{6′} is F or H; and R^{6″} is F. In some embodiments, R¹ is —CF₃; R^{7a} is absent; each R⁵ is independently methyl; R^{6′} is F or H; and R^{6″} is H.

In some embodiments of Formula (I-J′-a-S), or a pharmaceutically acceptable salt thereof, R¹ is C₁-C₆ haloalkyl; R^{7a} is absent; each R⁵ is independently C₁-C₆ alkyl; R^{6′} is halo; and R^{6″} is halo or H. In some embodiments, R¹ is —CF₃; R^{7a} is absent; each R⁵ is independently methyl; R^{6′} is F; and R^{6″} is F or H.

In some embodiments, R¹ is —CF₃; R^{7a} is absent; each R⁵ is independently methyl; R^{6′} is F; and R^{6″} is F. In some embodiments, R¹ is —CF₃; R^{7a} is absent; each R⁵ is independently methyl; R^{6′} is F; and R^{6″} is H.

In some embodiments of Formula (I-J′-a-S), or a pharmaceutically acceptable salt thereof, R¹ is C₁-C₆ haloalkyl; R^{7a} is absent; each R⁵ is independently C₁-C₆ alkyl; R^{6′} is H; and R^{6″} is halo or H. In some embodiments, R¹ is —CF₃; R^{7a} is absent; each R⁵ is independently methyl; R^{6′} is H; and R^{6″} is F or H.

In some embodiments, R¹ is —CF₃; R^{7a} is absent; each R⁵ is independently methyl; R⁶ is H; and R^{6″} is F. In some embodiments, R¹ is —CF₃; R^{7a} is absent; each R⁵ is independently methyl; R^{6′} is H; and R^{6″} is H.

In some embodiments of Formula (I-J′-a-S), or a pharmaceutically acceptable salt thereof, R¹ is C₃-C₅ cycloalkyl; R^{7a} is absent; each R⁵ is independently C₁-C₆ alkyl; R^{6′} is halo or H; and R^{6″} is halo or H. In some embodiments, R¹ is cyclopropyl; R^{7a} is absent; each R⁵ is independently methyl; R^{6′} is F or H; and R^{6″} is F or H. In some embodiments, R¹ is cyclopropyl; R^{7a} is absent; each R⁵ is independently methyl; R^{6′} is F or H; and R^{6″} is F. In some embodiments, R¹ is cyclopropyl; R^{7a} is absent; each R⁵ is independently methyl; R^{6′} is F or H; and R^{6″} is H.

In some embodiments of Formula (I-J′-a-S), or a pharmaceutically acceptable salt thereof, R¹ is C₃-C₅ cycloalkyl; R^{7a} is absent; each R⁵ is independently C₁-C₆ alkyl; R^{6′} is halo; and R^{6″} is halo or H. In some embodiments, R¹ is cyclopropyl; R^{7a} is absent; each R⁵ is independently methyl; R^{6′} is F; and R^{6″} is F or H. In some embodiments, R¹ is cyclopropyl; R^{7a} is absent; each R⁵ is independently methyl; R^{6′} is F; and R^{6″} is F. In some embodiments, R¹ is cyclopropyl; R^{7a} is absent; each R⁵ is independently methyl; R^{6′} is F; and R^{6″} is H.

In some embodiments of Formula (I-J′-a-S), or a pharmaceutically acceptable salt thereof, R¹ is C₃-C₅ cycloalkyl; R^{7a} is absent; each R⁵ is independently C₁-C₆ alkyl; R^{6′} is H; and R^{6″} is halo or H. In some embodiments, R¹ is cyclopropyl; R^{7a} is absent; each R⁵ is independently methyl; R^{6′} is H; and R⁶ is F or H. In some embodiments, R¹ is cyclopropyl; R^{7a} is absent; each R⁵ is independently methyl; R^{6′} is H; and R^{6″} is F. In some embodiments, R¹ is cyclopropyl; R^{7a} is absent; each R⁵ is independently methyl; R¹ is H; and R^{6″} is H.

In some embodiments, the compound of Formula (I) is a compound of Formula (I-J′-a-R), or a pharmaceutically acceptable salt thereof.

In some embodiments, the compound of Formula (I) is selected from any one of the compounds of Tables 1 and 2A-2B, or a pharmaceutically acceptable salt thereof. The below Tables 1 and 2A-2B also provide the location of the compound in the Examples (Ex) by Example Number (Ex) or as provided in Table B (TA) of the Examples. The Asterix (*) next to the Compound Number (#) signifies that stereochemistry has been arbitrarily or rationally assigned.

In some embodiments, the compound of Formula (I) is a non-ionized compound (e.g., free acid) selected from any one of the compounds of Tables 1 or 2A-2B.

In some embodiments, the compound of Formula (I) is a salt (e.g., pharmaceutically acceptable salt) selected from any one of the compounds of Tables 1 or Tables 2A-2B.

TABLE 1
Compounds of Formula I

| Ex | # | Compound Structure/Name |
|---|---|---|
| 1 | 1 | 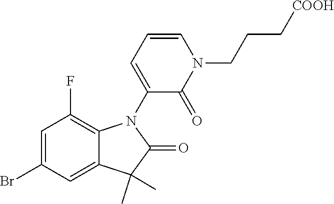 [SMILES: CC1(C)C(=O)N(c2cccn(CCCC(=O)O)c2=O)c2c(F)cc(Br)cc21] |
| | | 4-(3-(5-bromo-7-fluoro-3,3-dimethyl-2-oxoindolin-1-yl)-2-oxopyridin-1(2H)- |
| | | yl)butanoic acid |
| 1 | 1-OtBu | 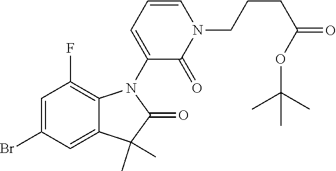 [SMILES: CC(C)(C)OC(=O)CCCn1cccc(N2C(=O)C(C)(C)c3cc(Br)cc(F)c32)c1=O] |
| | | tert-butyl 4-(3-(5-bromo-7-fluoro-3,3-dimethyl-2-oxoindolin-1-yl)-2- |
| | | oxopyridin-1(2H)-yl)butanoate |
| TA | 2 | 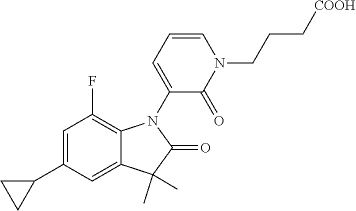 [SMILES: CC1(C)C(=O)N(c2cccn(CCCC(=O)O)c2=O)c2c(F)cc(C3CC3)cc21] |
| | | 4-(3-(5-cyclopropyl-7-fluoro-3,3-dimethyl-2-oxoindolin-1-yl)-2-oxopyridin- |
| | | 1(2H)-yl)butanoic acid |
| TA | 3 | 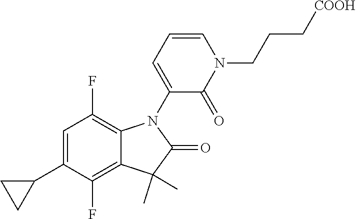 [SMILES: CC1(C)C(=O)N(c2cccn(CCCC(=O)O)c2=O)c2c(F)cc(C3CC3)c(F)c21] |
| | | 4-(3-(5-cyclopropyl-4,7-difluoro-3,3-dimethyl-2-oxoindolin-1-yl)-2- |
| | | oxopyridin-1(2H)-yl)butanoic acid |
| 2 | 4 | 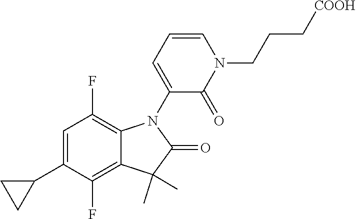 [SMILES: CC1(C)C(=O)N(c2cccn(CCCC(=O)O)c2=O)c2c(F)cc(C3CC3)c(F)c21] |
| | | 4-(3-(5-cyclopropyl-4,7-difluoro-3,3-dimethyl-2-oxoindolin-1-yl)-4-methyl-2- |
| | | oxopyridin-1(2H)-yl)butanoic acid |
| 2 | 4A* | 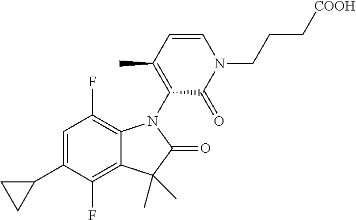 |
| | | (Sa)-4-(3-(5-cyclopropyl-4,7-difluoro-3,3-dimethyl-2-oxoindolin-1-yl)-4- |
| | | methyl-2-oxopyridin-1(2H)-yl)butanoic acid |
| 2 | 4B* | 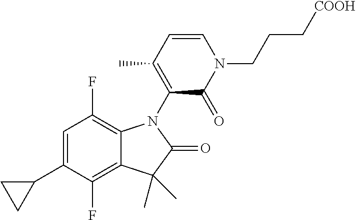 |
| | | (Ra)-4-(3-(5-cyclopropyl-4,7-difluoro-3,3-dimethyl-2-oxoindolin-1-yl)-4- |
| | | methyl-2-oxopyridin-1(2H)-yl)butanoic acid |
| 2 | 4-OMe | 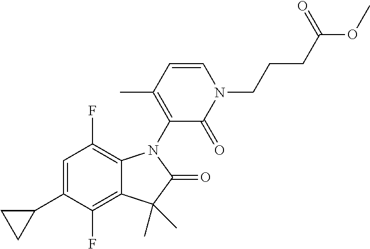 [SMILES: COC(=O)CCCn1ccc(C)c(N2C(=O)C(C)(C)c3c(F)c(C4CC4)cc(F)c32)c1=O] |
| | | methyl 4-(3-(5-cyclopropyl-4,7-difluoro-3,3-dimethyl-2-oxoindolin-1-yl)-4- |
| | | methyl-2-oxopyridin-1(2H)-yl)butanoate |
| 3 | 5 | 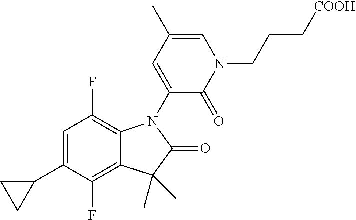 [SMILES: Cc1cc(N2C(=O)C(C)(C)c3c(F)c(C4CC4)cc(F)c32)c(=O)n(CCCC(=O)O)c1] |
| | | 4-(3-(5-cyclopropyl-4,7-difluoro-3,3-dimethyl-2-oxoindolin-1-yl)-5-methyl-2- |
| | | oxopyridin-1(2H)-yl)butanoic acid |
| 3 | 5-OMe | 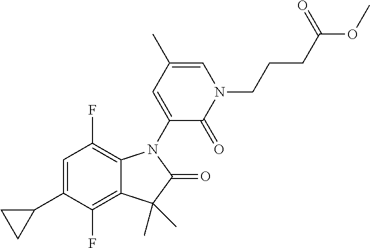 [SMILES: COC(=O)CCCn1cc(C)cc(N2C(=O)C(C)(C)c3c(F)c(C4CC4)cc(F)c32)c1=O] |
| | | methyl 4-(3-(5-cyclopropyl-4,7-difluoro-3,3-dimethyl-2-oxoindolin-1-yl)-5- |
| | | methyl-2-oxopyridin-1(2H)-yl)butanoate |
| 4 | 6 | 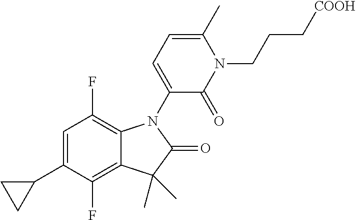 [SMILES: Cc1ccc(N2C(=O)C(C)(C)c3c(F)c(C4CC4)cc(F)c32)c(=O)n1CCCC(=O)O] |
| | | 4-(3-(5-cyclopropyl-4,7-difluoro-3,3-dimethyl-2-oxoindolin-1-yl)-6-methyl-2- |
| | | oxopyridin-1(2H)-yl)butanoic acid |
| 4 | 6-OMe | 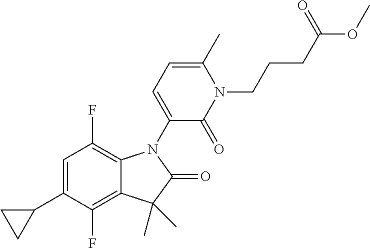 [SMILES: COC(=O)CCCn1c(C)ccc(N2C(=O)C(C)(C)c3c(F)c(C4CC4)cc(F)c32)c1=O] |
| | | methyl 4-(3-(5-cyclopropyl-4,7-difluoro-3,3-dimethyl-2-oxoindolin-1-yl)-6- |
| | | methyl-2-oxopyridin-1(2H)-yl)butanoate |
| 5 | 7 | 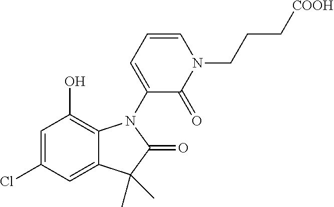 [SMILES: CC1(C)C(=O)N(c2cccn(CCCC(=O)O)c2=O)c2c(O)cc(Cl)cc21] |
| | | 4-(3-(5-chloro-7-hydroxy-3,3-dimethyl-2-oxoindolin-1-yl)-2-oxopyridin-1(2H)- |
| | | yl)butanoic acid |
| 5 | 7-OtBu | 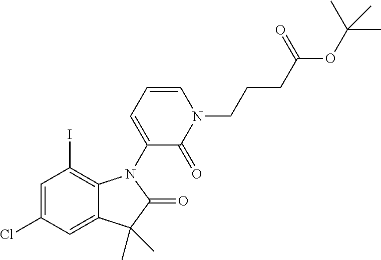 [SMILES: CC(C)(C)OC(=O)CCCn1cccc(N2C(=O)C(C)(C)c3cc(Cl)cc(I)c32)c1=O] |
| | | tert-butyl 4-(3-(5-chloro-7-iodo-3,3-dimethyl-2-oxoindolin-1-yl)-2-oxopyridin- |
| | | 1(2H)-yl)butanoate |
| 6 | 8 | 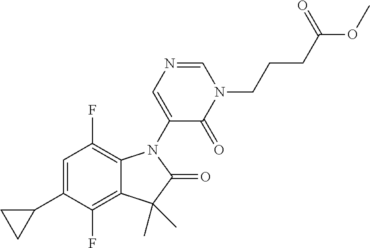 [SMILES: COC(=O)CCCn1cncc(N2C(=O)C(C)(C)c3c(F)c(C4CC4)cc(F)c32)c1=O] |
| | | 4-(5-(5-cyclopropyl-4,7-difluoro-3,3-dimethyl-2-oxoindolin-1-yl)-6- |
| | | oxopyrimidin-1(6H)-yl)butanoic acid |
| 6 | 8-OMe | 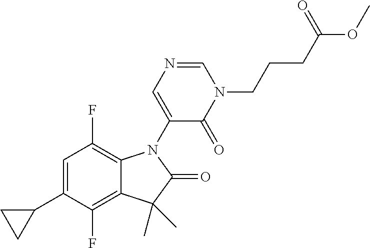 [SMILES: COC(=O)CCCn1cncc(N2C(=O)C(C)(C)c3c(F)c(C4CC4)cc(F)c32)c1=O] |
| | | methyl 4-(5-(5-cyclopropyl-4,7-difluoro-3,3-dimethyl-2-oxoindolin-1-yl)-6- |
| | | oxopyrimidin-1(6H)-yl)butanoate |
| 7 | 9 | 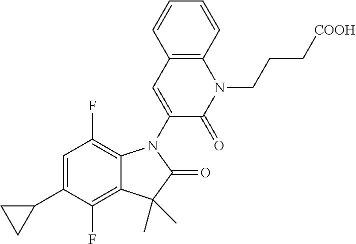 [SMILES: CC1(C)C(=O)N(c2cc3ccccc3n(CCCC(=O)O)c2=O)c2c(F)cc(C3CC3)c(F)c21] |
| | | 4-(3-(5-cyclopropyl-4,7-difluoro-3,3-dimethyl-2-oxoindolin-1-yl)-2- |
| | | oxoquinolin-1(2H)-yl)butanoic acid |
| 7 | 9-OMe | 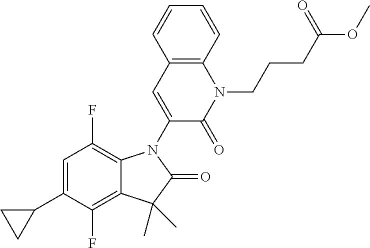 [SMILES: COC(=O)CCCn1c(=O)c(N2C(=O)C(C)(C)c3c(F)c(C4CC4)cc(F)c32)cc2ccccc21] |
| | | methyl 4-(3-(5-cyclopropyl-4,7-difluoro-3,3-dimethyl-2-oxoindolin-1-yl)-2- |
| | | oxoquinolin-1(2H)-yl)butanoate |
| 8 | 10 | 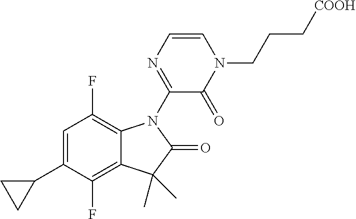 [SMILES: CC1(C)C(=O)N(c2nccn(CCCC(=O)O)c2=O)c2c(F)cc(C3CC3)c(F)c21] |
| | | 4-(3-(5-cyclopropyl-4,7-difluoro-3,3-dimethyl-2-oxoindolin-1-yl)-2- |
| | | oxopyrazin-1(2H)-yl)butanoic acid |
| 8 | 10-OtBu | 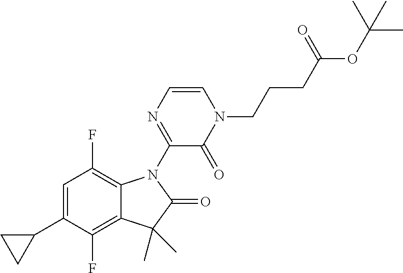 [SMILES: CC(C)(C)OC(=O)CCCn1ccnc(N2C(=O)C(C)(C)c3c(F)c(C4CC4)cc(F)c32)c1=O] |
| | | tert-butyl 4-(3-(5-cyclopropyl-4,7-difluoro-3,3-dimethyl-2-oxoindolin-1-yl)-2- |
| | | oxopyrazin-1(2H)-yl)butanoate |
| 9 | 11 | 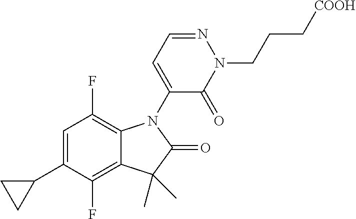 [SMILES: CC1(C)C(=O)N(c2ccnn(CCCC(=O)O)c2=O)c2c(F)cc(C3CC3)c(F)c21] |
| | | 4-(5-(5-cyclopropyl-4,7-difluoro-3,3-dimethyl-2-oxoindolin-1-yl)-6- |
| | | oxopyridazin-1(6H)-yl)butanoic acid |
| 9 | 11-OtBu | 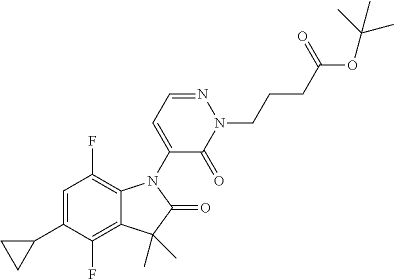 [SMILES: CC(C)(C)OC(=O)CCCn1nccc(N2C(=O)C(C)(C)c3c(F)c(C4CC4)cc(F)c32)c1=O] |
| | | tert-butyl 4-(5-(5-cyclopropyl-4,7-difluoro-3,3-dimethyl-2-oxoindolin-1-yl)-6- |
| | | oxopyridazin-1(6H)-yl)butanoate |
| TA | 12 | 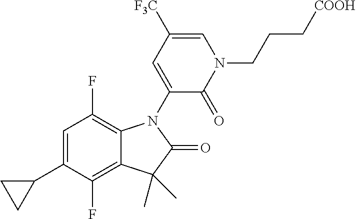 [SMILES: CC1(C)C(=O)N(c2cc(C(F)(F)F)cn(CCCC(=O)O)c2=O)c2c(F)cc(C3CC3)c(F)c21] |
| | | 4-(3-(5-cyclopropyl-4,7-difluoro-3,3-dimethyl-2-oxoindolin-1-yl)-2-oxo-5- |
| | | (trifluoromethyl)pyridin-1(2H)-yl)butanoic acid |
| TA | 13 | 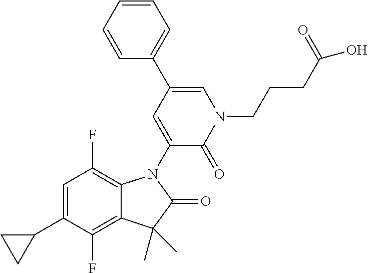 [SMILES: CC1(C)C(=O)N(c2cc(-c3ccccc3)cn(CCCC(=O)O)c2=O)c2c(F)cc(C3CC3)c(F)c21] |
| | | 4-(3-(5-cyclopropyl-4,7-difluoro-3,3-dimethyl-2-oxoindolin-1-yl)-2-oxo-5- |
| | | phenylpyridin-1(2H)-yl)butanoic acid |
| TA | 14 | 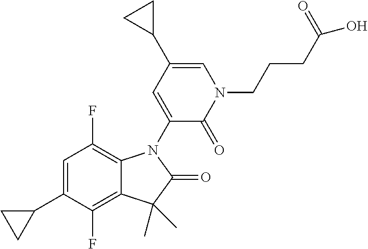 [SMILES: CC1(C)C(=O)N(c2cc(C3CC3)cn(CCCC(=O)O)c2=O)c2c(F)cc(C3CC3)c(F)c21] |
| | | 4-(5-cyclopropyl-3-(5-cyclopropyl-4,7-difluoro-3,3-dimethyl-2-oxoindolin-1- |
| | | yl)-2-oxopyridin-1(2H)-yl)butanoic acid |
| TA | 15 | 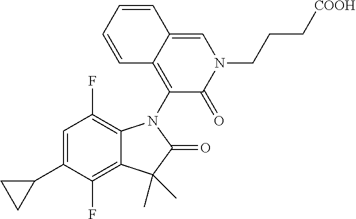 [SMILES: CC1(C)C(=O)N(c2c(=O)n(CCCC(=O)O)cc3ccccc23)c2c(F)cc(C3CC3)c(F)c21] |
| | | 4-(4-(5-cyclopropyl-4,7-difluoro-3,3-dimethyl-2-oxoindolin-1-yl)-3- |
| | | oxoisoquinolin-2(3H)-yl)butanoic acid |
| TA | 17 | 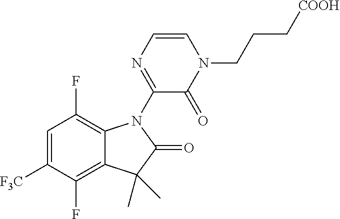 [SMILES: CC1(C)C(=O)N(c2nccn(CCCC(=O)O)c2=O)c2c(F)cc(C(F)(F)F)c(F)c21] |
| | | 4-(3-(4,7-difluoro-3,3-dimethyl-2-oxo-5-(trifluoromethyl)indolin-1-yl)-2- |
| | | oxopyrazin-1(2H)-yl)butanoic acid |
| 11 | 20 | 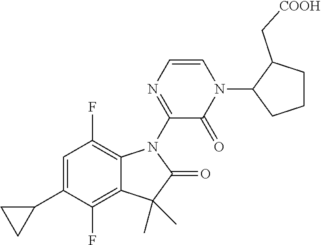 [SMILES: CC1(C)C(=O)N(c2nccn(C3CCCC3CC(=O)O)c2=O)c2c(F)cc(C3CC3)c(F)c21] |
| | | 2-(2-(3-(5-cyclopropyl-4,7-difluoro-3,3-dimethyl-2-oxoindolin-1-yl)-2- |
| | | oxopyrazin-1(2H)-yl)cyclopentyl)acetic acid |
| 11 | 20A* | 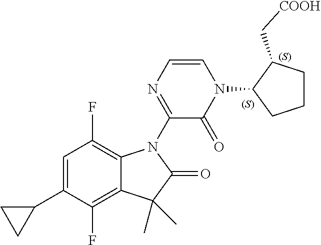 |
| | | 2-((1S,2S)-2-(3-(5-cyclopropyl-4,7-difluoro-3,3-dimethyl-2-oxoindolin-1-yl)-2- |
| | | oxopyrazin-1(2H)-yl)cyclopentyl)acetic acid |
| 11 | 20B* | 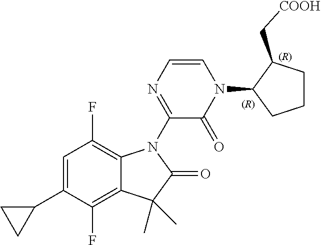 |
| | | 2-((1R,2R)-2-(3-(5-cyclopropyl-4,7-difluoro-3,3-dimethyl-2-oxoindolin-1-yl)- |
| | | 2-oxopyrazin-1(2H)-yl)cyclopentyl)acetic acid |
| 11 | 20C* | 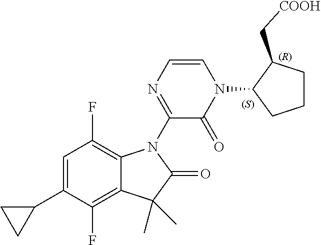 |
| | | 2-((1R,2S)-2-(3-(5-cyclopropyl-4,7-difluoro-3,3-dimethyl-2-oxoindolin-1-yl)-2- |
| | | oxopyrazin-1(2H)-yl)cyclopentyl)acetic acid |
| 11 | 20D* | 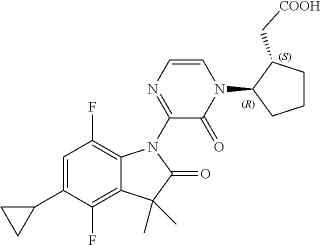 |
| | | 2-((1S,2R)-2-(3-(5-cyclopropyl-4,7-difluoro-3,3-dimethyl-2-oxoindolin-1-yl)-2- |
| | | oxopyrazin-1(2H)-yl)cyclopentyl)acetic acid |
| 11 | 20A*-OEt | 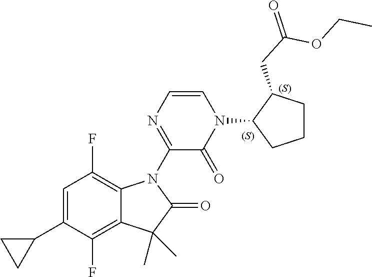 |
| | | ethyl 2-((1S,2S)-2-(3-(5-cyclopropyl-4,7-difluoro-3,3-dimethyl-2-oxoindolin-1- |
| | | yl)-2-oxopyrazin-1(2H)-yl)cyclopentyl)acetate |
| 11 | 20B*-OEt | 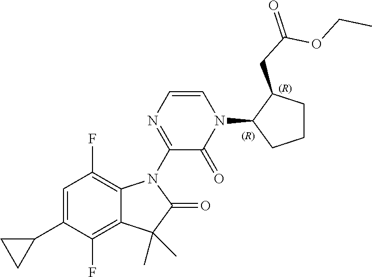 |
| | | ethyl 2-((1R,2R)-2-(3-(5-cyclopropyl-4,7-difluoro-3,3-dimethyl-2-oxoindolin- |
| | | 1-yl)-2-oxopyrazin-1(2H)-yl)cyclopentyl)acetate |
| 11 | 20A*-OtBu | 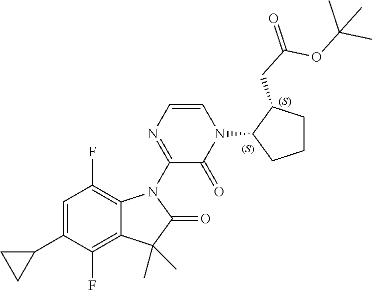 |
| | | tert-butyl 2-((1S,2S)-2-(3-(5-cyclopropyl-4,7-difluoro-3,3-dimethyl-2- |
| | | oxoindolin-1-yl)-2-oxopyrazin-1(2H)-yl)cyclopentyl)acetate |
| 11 | 20B*-OtBu | 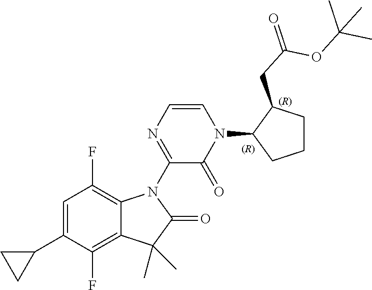 |
| | | tert-butyl 2-((1R,2R)-2-(3-(5-cyclopropyl-4,7-difluoro-3,3-dimethyl-2- |
| | | oxoindolin-1-yl)-2-oxopyrazin-1(2H)-yl)cyclopentyl)acetate |
| 11 | 20C*-OtBu | 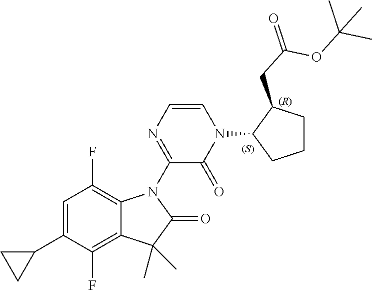 |
| | | tert-butyl 2-((1R,2S)-2-(3-(5-cyclopropyl-4,7-difluoro-3,3-dimethyl-2- |
| | | oxoindolin-1-yl)-2-oxopyrazin-1(2H)-yl)cyclopentyl)acetate |
| 11 | 20D*-OtBu | 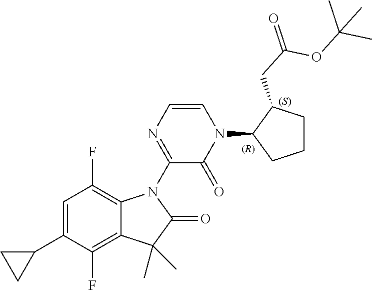 |
| | | tert-butyl 2-((1S,2R)-2-(3-(5-cyclopropyl-4,7-difluoro-3,3-dimethyl-2- |
| | | oxoindolin-1-yl)-2-oxopyrazin-1(2H)-yl)cyclopentyl)acetate |
| TA | 21 | 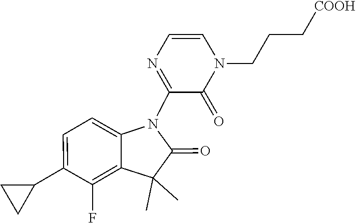 [SMILES: CC1(C)C(=O)N(c2nccn(CCCC(=O)O)c2=O)c2ccc(C3CC3)c(F)c21] |
| | | 4-(3-(5-cyclopropyl-4-fluoro-3,3-dimethyl-2-oxoindolin-1-yl)-2-oxopyrazin- |
| | | 1(2H)-yl)butanoic acid |

TABLE 2A
Compounds of Formula (I)

| Ex | # | Compound Structure/Name |
|---|---|---|
| 10 | 16 | 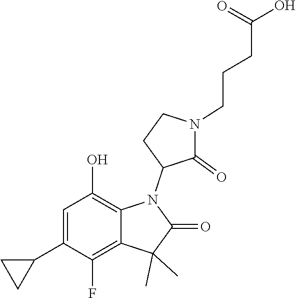 [SMILES: CC1(C)C(=O)N(C2CCN(CCCC(=O)O)C2=O)c2c(O)cc(C3CC3)c(F)c21] |
| | | 4-(3-(5-cyclopropyl-4,7-difluoro-3,3-dimethyl-2-oxoindolin-1-yl)-2- |
| | | oxopyrrolidin-1-yl)butanoic acid |
| 10 | 16-OtBu | 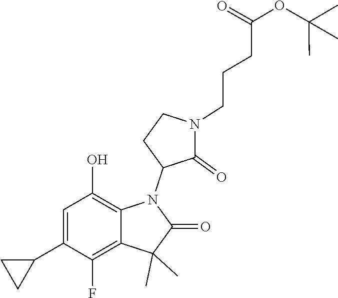 [SMILES: CC(C)(C)OC(=O)CCCN1CCC(N2C(=O)C(C)(C)c3c(F)c(C4CC4)cc(O)c32)C1=O] |
| | | tert-butyl 4-(3-(5-cyclopropyl-4,7-difluoro-3,3-dimethyl-2-oxoindolin-1-yl)- |
| | | 2-oxopyrrolidin-1-yl)butanoate |
| 10 | 16A | 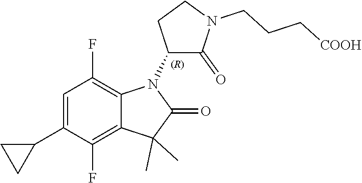 |
| | | (R)-4-(3-(5-cyclopropyl-4,7-difluoro-3,3-dimethyl-2-oxoindolin-1-yl)-2- |
| | | oxopyrrolidin-1-yl)butanoic acid |
| 10 | 16B | 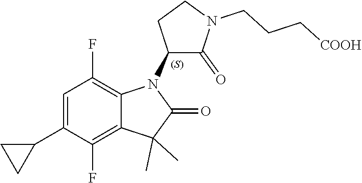 |
| | | (S)-4-(3-(5-cyclopropyl-4,7-difluoro-3,3-dimethyl-2-oxoindolin-1-yl)-2- |
| | | oxopyrrolidin-1-yl)butanoic acid |
| TA | 18 | 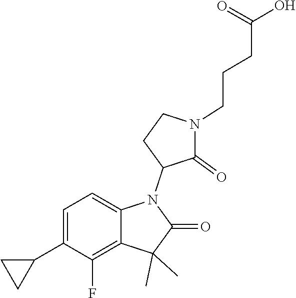 [SMILES: CC1(C)C(=O)N(C2CCN(CCCC(=O)O)C2=O)c2ccc(C3CC3)c(F)c21] |
| | | 4-(3-(5-cyclopropyl-4-fluoro-3,3-dimethyl-2-oxoindolin-1-yl)-2- |
| | | oxopyrrolidin-1-yl)butanoic acid |
| TA | 18A* | 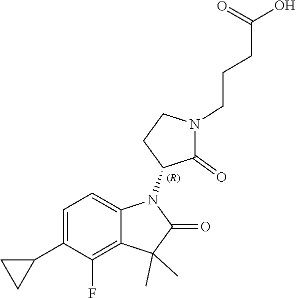 |
| | | (R)-4-(3-(5-cyclopropyl-4-fluoro-3,3-dimethyl-2-oxoindolin-1-yl)-2- |
| | | oxopyrrolidin-1-yl)butanoic acid |
| TA | 18B* | 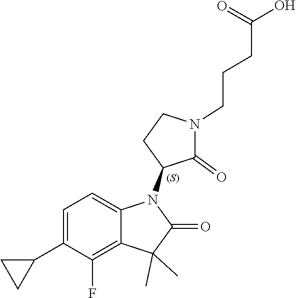 |
| | | (S)-4-(3-(5-cyclopropyl-4-fluoro-3,3-dimethyl-2-oxoindolin-1-yl)-2- |
| | | oxopyrrolidin-1-yl)butanoic acid |
| 12 | 19 | 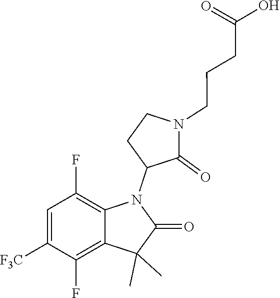 [SMILES: CC1(C)C(=O)N(C2CCN(CCCC(=O)O)C2=O)c2c(F)cc(C(F)(F)F)c(F)c21] |
| | | 4-(3-(4,7-difluoro-3,3-dimethyl-2-oxo-5-(trifluoromethyl)indolin-1-yl)-2- |
| | | oxopyrrolidin-1-yl)butanoic acid |
| 12 | 19A* | 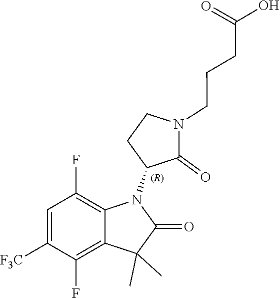 |
| | | (R)-4-(3-(4,7-difluoro-3,3-dimethyl-2-oxo-5-(trifluoromethyl)indolin-1-yl)-2- |
| | | oxopyrrolidin-1-yl)butanoic acid |
| 12 | 19B* | 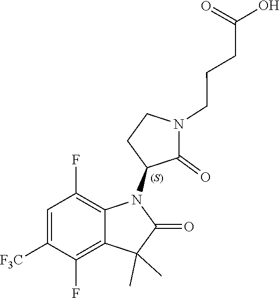 |
| | | (S)-4-(3-(4,7-difluoro-3,3-dimethyl-2-oxo-5-(trifluoromethyl)indolin-1-yl)-2- |
| | | oxopyrrolidin-1-yl)butanoic acid |
| 12 | 19A*-OtBu | 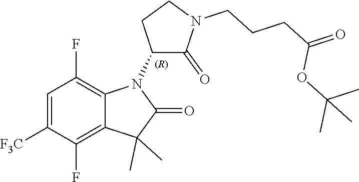 |
| | | tert-butyl (R)-4-(3-(4,7-difluoro-3,3-dimethyl-2-oxo-5- |
| | | (trifluoromethyl)indolin-1-yl)-2-oxopyrrolidin-1-yl)butanoate |
| 12 | 19B*-OtBu | 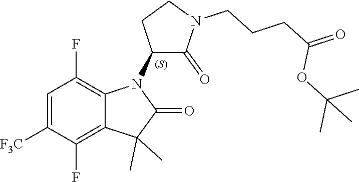 |
| | | tert-butyl (S)-4-(3-(4,7-difluoro-3,3-dimethyl-2-oxo-5- |
| | | (trifluoromethyl)indolin-1-yl)-2-oxopyrrolidin-1-yl)butanoate |

TABLE 2B
Compounds of Formula (I)

| Ex | # | Compound Structure/Name |
|---|---|---|
| 13 | 22 | 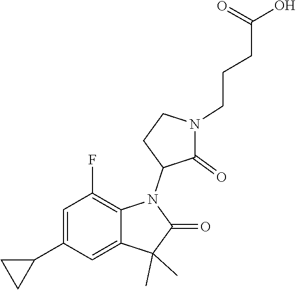 [SMILES: CC1(C)C(=O)N(C2CCN(CCCC(=O)O)C2=O)c2c(F)cc(C3CC3)cc21] |
| | | 4-(3-(5-cyclopropyl-7-fluoro-3,3-dimethyl-2-oxoindolin-1-yl)-2- |
| | | oxopyrrolidin-1-yl)butanoic acid |
| 13 | 22-OtBu | 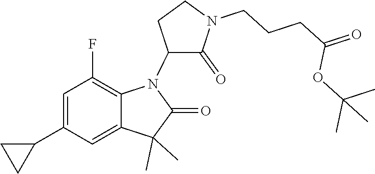 [SMILES: CC(C)(C)OC(=O)CCCN1CCC(N2C(=O)C(C)(C)c3cc(C4CC4)cc(F)c32)C1=O] |
| | | tert-butyl 4-(3-(5-cyclopropyl-7-fluoro-3,3-dimethyl-2-oxoindolin-1-yl)-2- |
| | | oxopyrrolidin-1-yl)butanoate |
| 13 | 22A* | 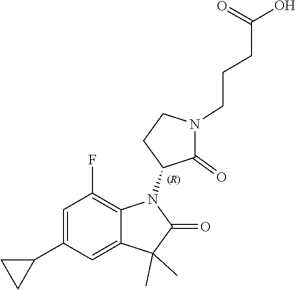 |
| | | (R)-4-(3-(5-cyclopropyl-7-fluoro-3,3-dimethyl-2-oxoindolin-1-yl)-2- |
| | | oxopyrrolidin-1-yl)butanoic acid |
| 13 | 22B* | 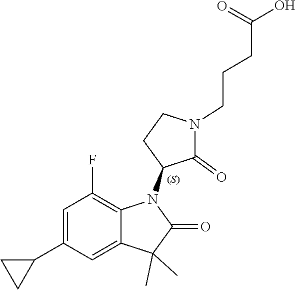 |
| | | (S)-4-(3-(5-cyclopropyl-7-fluoro-3,3-dimethyl-2-oxoindolin-1-yl)-2- |
| | | oxopyrrolidin-1-yl)butanoic acid |
| TA | 23 | 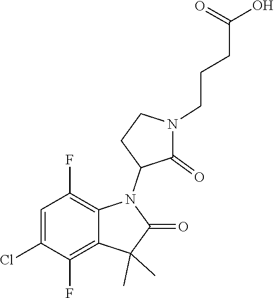 [SMILES: CC1(C)C(=O)N(C2CCN(CCCC(=O)O)C2=O)c2c(F)cc(Cl)c(F)c21] |
| | | 4-(3-(5-chloro-4,7-difluoro-3,3-dimethyl-2-oxoindolin-1-yl)-2-oxopyrrolidin- |
| | | 1-yl)butanoic acid |
| TA | 23A* | 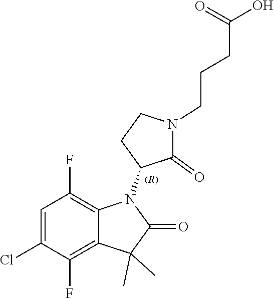 |
| | | (R)-4-(3-(5-chloro-4,7-difluoro-3,3-dimethyl-2-oxoindolin-1-yl)-2- |
| | | oxopyrrolidin-1-yl)butanoic acid |
| TA | 23B* | 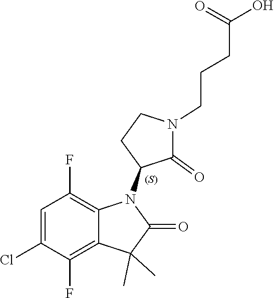 |
| | | (S)-4-(3-(5-chloro-4,7-difluoro-3,3-dimethyl-2-oxoindolin-1-yl)-2- |
| | | oxopyrrolidin-1-yl)butanoic acid |
| 14 | 24 | 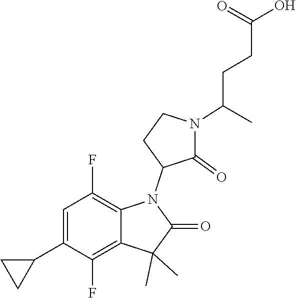 [SMILES: CC(CCC(=O)O)N1CCC(N2C(=O)C(C)(C)c3c(F)c(C4CC4)cc(F)c32)C1=O] |
| | | 4-(3-(5-cyclopropyl-4,7-difluoro-3,3-dimethyl-2-oxoindolin-1-yl)-2- |
| | | oxopyrrolidin-1-yl)pentanoic acid |
| 14 | 24-OEt | 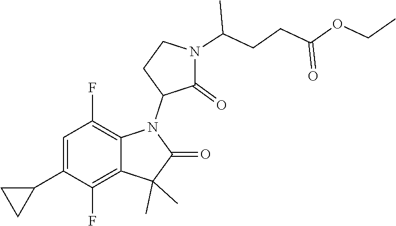 [SMILES: CCOC(=O)CCC(C)N1CCC(N2C(=O)C(C)(C)c3c(F)c(C4CC4)cc(F)c32)C1=O] |
| | | ethyl 4-(3-(5-cyclopropyl-4,7-difluoro-3,3-dimethyl-2-oxoindolin-1-yl)-2- |
| | | oxopyrrolidin-1-yl)pentanoate |
| 14 | 24A* | 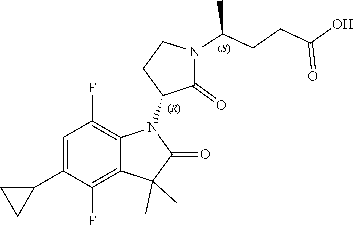 |
| | | (S)-4-((R)-3-(5-cyclopropyl-4,7-difluoro-3,3-dimethyl-2-oxoindolin-1-yl)-2- |
| | | oxopyrrolidin-1-yl)pentanoic acid |
| 14 | 24B* | 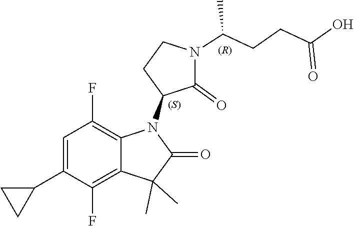 |
| | | (R)-4-((S)-3-(5-cyclopropyl-4,7-difluoro-3,3-dimethyl-2-oxoindolin-1-yl)-2- |
| | | oxopyrrolidin-1-yl)pentanoic acid |
| 14 | 24C* | 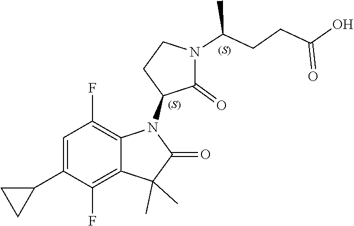 |
| | | (S)-4-((S)-3-(5-cyclopropyl-4,7-difluoro-3,3-dimethyl-2-oxoindolin-1-yl)-2- |
| | | oxopyrrolidin-1-yl)pentanoic acid |
| 14 | 24D* | 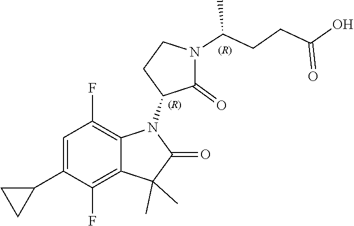 |
| | | (R)-4-((R)-3-(5-cyclopropyl-4,7-difluoro-3,3-dimethyl-2-oxoindolin-1-yl)-2- |
| | | oxopyrrolidin-1-yl)pentanoic acid |
| TA | 25 | 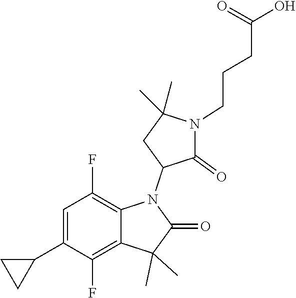 [SMILES: CC1(C)C(=O)N(C2CC(C)(C)N(CCCC(=O)O)C2=O)c2c(F)cc(C3CC3)c(F)c21] |
| | | 4-(4-(5-cyclopropyl-4,7-difluoro-3,3-dimethyl-2-oxoindolin-1-yl)-2,2- |
| | | dimethyl-5-oxopyrrolidin-1-yl)butanoic acid |
| TA | 25A* | 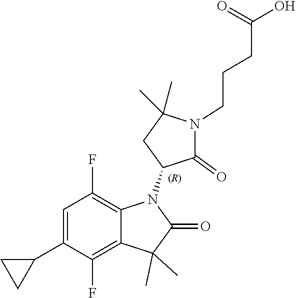 |
| | | (R)-4-(4-(5-cyclopropyl-4,7-difluoro-3,3-dimethyl-2-oxoindolin-1-yl)-2,2- |
| | | dimethyl-5-oxopyrrolidin-1-yl)butanoic acid |
| TA | 25B* | 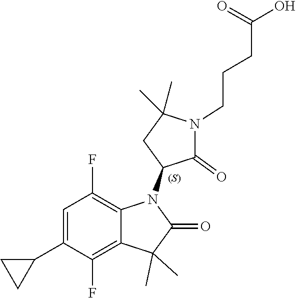 |
| | | (S)-4-(4-(5-cyclopropyl-4,7-difluoro-3,3-dimethyl-2-oxoindolin-1-yl)-2,2- |
| | | dimethyl-5-oxopyrrolidin-1-yl)butanoic acid |
| 15 | 26 | 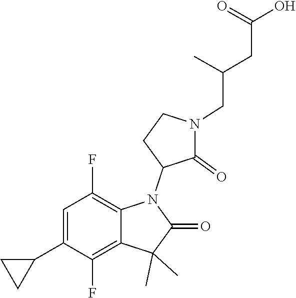 [SMILES: CC(CC(=O)O)CN1CCC(N2C(=O)C(C)(C)c3c(F)c(C4CC4)cc(F)c32)C1=O] |
| | | 4-(3-(5-cyclopropyl-4,7-difluoro-3,3-dimethyl-2-oxoindolin-1-yl)-2- |
| | | oxopyrrolidin-1-yl)-3-methylbutanoic acid |
| 15 | 26-OEt | 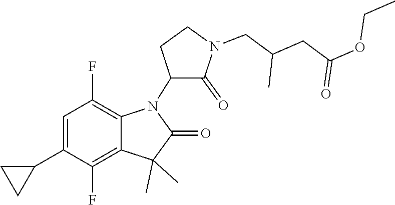 [SMILES: CCOC(=O)CC(C)CN1CCC(N2C(=O)C(C)(C)c3c(F)c(C4CC4)cc(F)c32)C1=O] |
| | | ethyl 4-(3-(5-cyclopropyl-4,7-difluoro-3,3-dimethyl-2-oxoindolin-1-yl)-2- |
| | | oxopyrrolidin-1-yl)-3-methylbutanoate |
| 15 | 26A* | 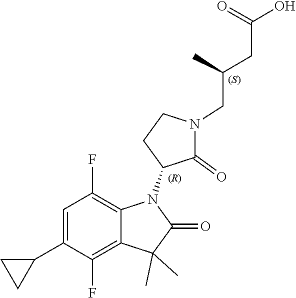 |
| | | (S)-4-((R)-3-(5-cyclopropyl-4,7-difluoro-3,3-dimethyl-2-oxoindolin-1-yl)-2- |
| | | oxopyrrolidin-1-yl)-3-methylbutanoic acid |
| 15 | 26B* | 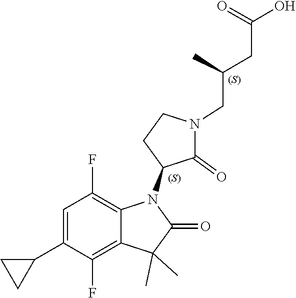 |
| | | (S)-4-((S)-3-(5-cyclopropyl-4,7-difluoro-3,3-dimethyl-2-oxoindolin-1-yl)-2- |
| | | oxopyrrolidin-1-yl)-3-methylbutanoic acid |
| 15 | 26C* | 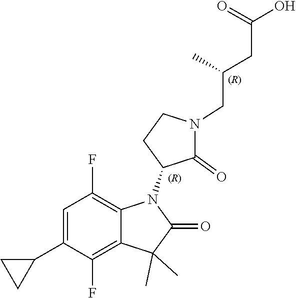 |
| | | (R)-4-((R)-3-(5-cyclopropyl-4,7-difluoro-3,3-dimethyl-2-oxoindolin-1-yl)-2- |
| | | oxopyrrolidin-1-yl)-3-methylbutanoic acid |
| 15 | 26D* | 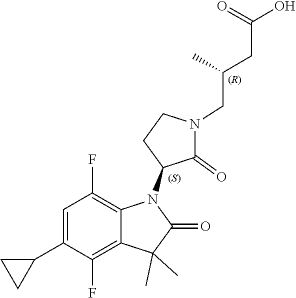 |
| | | (R)-4-((S)-3-(5-cyclopropyl-4,7-difluoro-3,3-dimethyl-2-oxoindolin-1-yl)-2- |
| | | oxopyrrolidin-1-yl)-3-methylbutanoic acid |

### (ii) Pharmaceutical Compositions

Pharmaceutical compositions comprising a compound of Formula (I), or a pharmaceutically acceptable salt thereof, and one or more pharmaceutically acceptable carriers, are further contemplated herein.

For example, in some aspects, provided is a pharmaceutical composition comprising a compound of Formula (I), or a pharmaceutically acceptable salt thereof, and one or more pharmaceutically acceptable carriers.

Exemplary pharmaceutical acceptable carriers may include diluents, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine.

Administration to the subject can be accomplished via any mode of administration, for example, by oral administration, topical administration, or by injection. Depending on the intended mode of administration, the pharmaceutical composition comprising the compound of Formula (I), or a pharmaceutically acceptable salt thereof, can be in solid, semi-solid or liquid dosage form.

A compound of Formula (I), or a pharmaceutically acceptable salt thereof, may be administered alone in the pharmaceutical composition as the sole therapeutic agent, or may be administered in combination with another therapeutic agent. Combination treatment may be achieved by way of co-administration (e.g., the two agents being administered at the same time) or sequential administration (e.g., one agent being administered first, then the other). In the case of co-administration, the compound of Formula (I), or a pharmaceutically acceptable salt thereof, may be administered in the same pharmaceutical composition as the other therapeutic agent, or may be administered in a separate pharmaceutical composition. The choice of the other therapeutic agent will depend upon the diagnosis of the attending physicians and their judgment of the condition of the subject and the appropriate treatment protocol.

(iii) Methods of Treatment

Compounds of Formula (I), and pharmaceutically acceptable salts thereof, have been found useful as inhibitors of NLRP3 activity.

In some aspects, provided is a method of treating or preventing a disease or disorder in a subject in need thereof, comprising administering to the subject a compound of Formula (I), or a pharmaceutically acceptable salt thereof, or a pharmaceutical composition comprising same. In some aspects, provided is a method of treating or preventing a disease or disorder in a subject in need thereof, comprising administering to the subject an effective amount of a compound of Formula (I), or a pharmaceutically acceptable salt thereof, or a pharmaceutical composition comprising same. In some aspects, provided is a method of treating or preventing a disease or disorder in a subject in need thereof, comprising administering to the subject a compound of Formula (I), or a pharmaceutically acceptable salt thereof, or a pharmaceutical composition comprising same. In some embodiments, the disease or disorder is associated with aberrant NLRP3 activity, and the method comprises inhibiting the aberrant NLRP3 activity such that the subject is treated.

In some embodiments, the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), an NLRP3-related disease in a subject that has been determined to carry a germline or somatic non-silent mutation in NLRP3, or obesity.

In some embodiments, the disease or disorder is a disease or disorder of central nervous system and/or peripheral nervous system (“PNS”), such as dementia, Alzheimer's disease (“AD”) epilepsy, traumatic brain injury (“TBI”), multiple sclerosis (“MS”), a developmental disturbance, acute disseminated encephalopathy, transverse myelitis, Parkinson's disease (“PD”), amyotrophic lateral sclerosis (“ALS”), Huntington's disease (“HD”), spinal cord injury, or obesity related to neuroinflammation.

In some embodiments, the disease or disorder is a primary neurological disease of the muscle, such as a dystrophy or spinal muscular atrophy.

In some embodiments, the disease or disorder is an inflammatory disorder, such as gout or anemia of inflammation.

In some embodiments, the disease or disorder is an autoimmune disease, such as ulcerative colitis.

In some embodiments, the disease or disorder is cancer, such as skin cancer or colon cancer.

In some embodiments, the disease or disorder is an infection, such as a neuro-infection.

In some embodiments, the disease or disorder is a metabolic disease, such as diabetes, e.g., type 2 diabetes.

In some embodiments, the disease or disorder is obesity. In some embodiments, the obesity is related to neuroinflammation, e.g., hypothalamic inflammation and/or gliosis. In some embodiments, the obesity is related to a metabolic disorder, e.g., diabetes.

In some embodiments, the disease or disorder is a cardiovascular disease, such as stroke, atherosclerosis or atherosclerotic cardiovascular disease (ASCVD).

In some embodiments, the disease or disorder is a respiratory disease, such as asthma (e.g., steroid-resistant asthma, severe steroid-resistant asthma) or chronic obstructive pulmonary disease (“COPD”).

In some embodiments, the disease or disorder is a kidney disease, such as acute kidney disease, a chronic kidney disease, or a rare kidney disease. In some embodiments, the chronic kidney disease is chronic kidney failure.

In some embodiments, the disease or disorder is a liver disease, such as nonalcoholic fatty liver disease (NAFLD) or non-alcoholic steatohepatitis (NASH, also known as MASH or metabolic dysfunction-associated steatohepatitis).

In some embodiments, the disease or disorder is an ocular disease, such as optic neuritis or macular degeneration.

In some embodiments, the disease or disorder is a skin disease, such as psoriasis, hidradenitis suppurativa (HS), or atopic dermatitis.

In some embodiments, the disease or disorder is a lymphatic disease.

In some embodiments, the disease or disorder is a rheumatic disease, such as osteoarthritis, dermatomyositis, Still's disease, or juvenile idiopathic arthritis.

In some embodiments, the disease or disorder is a psychological disease, such as a neuropsychiatric condition, including depression, major depressive disorder, or refractory depression.

In some embodiments, the disease or disorder is a graft versus host disease.

In some embodiments, the disease or disorder is pain (including disorders related to pain management), such as headache pain, pain management addiction, osteoarthritis pain, or allodynia.

In some embodiments, the NLRP3-related disease in a subject that has been determined to carry a germline or somatic non-silent mutation in NLRP3 is cryopyrin-associated autoinflammatory syndrome.

In some embodiments, the cryopyrin-associated autoinflammatory syndrome is familial cold autoinflammatory syndrome, Muckle-Wells syndrome, or neonatal onset multisystem inflammatory disease (NOMID).

In some embodiments, the disease or disorder is dementia, Alzheimer's disease (“AD”), epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), spinal muscular atrophy, Huntington's disease (“HD”), a spinal cord injury, a dystrophy, a neuro-infection, pain (e.g., headache pain, osteoarthritis pain, or allodynia, a pain management addiction), a neuropsychiatric condition (e.g. depression, major depressive disorder, refractory depression), neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, or hidradenitis suppurativa.

In other aspects, provided is a method of modulating (e.g., inhibiting) NLRP3 activity (e.g., in vitro or in vivo in a cell, or in a subject) in a cell, comprising contacting the cell with or administering to the subject a compound of Formula (I), or a pharmaceutically acceptable salt thereof. In some embodiments, the compound or a pharmaceutically acceptable salt thereof is administered to the cell or subject in an effective amount.

### (iv) Methods of Preparation

Compounds of Formula (I), intermediates thereof, and salts of any of the foregoing, may be synthesized following General Method Scheme A, as provided below, comprising coupling of a substituted indolinone (i), or salt thereof, with a lactam of formula (ii), or salt thereof, wherein R⁷ and R² are joined to form a 5- to 10-membered monocyclic or bicyclic heterocyclyl or 5- to 10-membered monocyclic or bicyclic heteroaryl, X is a leaving group, and Z is CO₂PG, to provide a compound of Formula (I), or salt thereof, wherein Z is CO₂PG, followed by optional deprotection to provide a compound of Formula (I), or salt thereof, wherein Z is CO₂H. The Examples further describe non-limiting examples of this and other general syntheses.

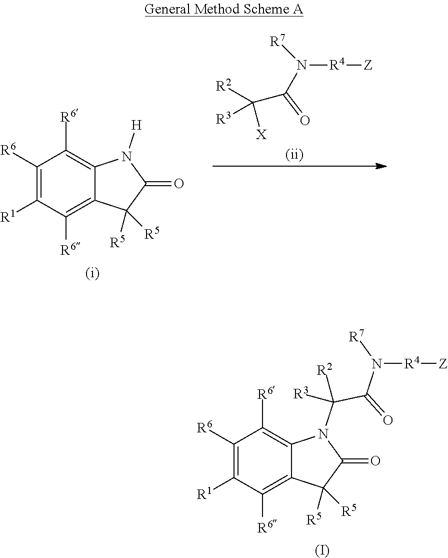

### (v) Biological Assays

Compounds designed, selected and/or optimized by methods described above, once produced, can be characterized using a variety of assays known to those skilled in the art to determine whether the compounds have biological activity. For example, the compounds can be characterized by conventional assays, including but not limited to those assays described below, to determine whether they have a predicted activity, binding activity and/or binding specificity. Effectiveness of compounds of the disclosure can be determined by industry-accepted assays/disease models according to standard practices of elucidating the same as described in the art and are found in the current general knowledge.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, binding assays, cellular assays (cell lines, primary cells and whole blood), in vitro cell viability assays, as well as assays for determining NLRP3 inhibitory activity, unbound clearance, and solubility.

In some embodiments, the compounds of the instant disclosure may be tested for their human-NLRP3 inhibitory activity using known procedures, such as the methodology reported in Coll et al. Nat Med. (2015) 21(3):248-255. See also the Examples, Human whole blood (hWB) NLRP3 Assay description.

In some embodiments, the compounds of the instant disclosure may be tested for unbound clearance (Clu) following known procedures, such as described in Miller et al., J. Med. Chem. (2020) 63:12156-12170. For example, unbound clearance (Clu) may be calculated by dividing total clearance (‘CL’ in mL/min/kg) as measured in blood or plasma by the unbound fraction in plasma (fu).

In some embodiments, the solubility of compounds of the instant disclosure may be determined following known procedures, such as described in Alsenz and Kansy, Advanced Drug Delivery Reviews (2007) 59:546-567, and Wang et al. J Mass Spectrom. (2000) 35:71-76. For example, the kinetic solubility in physiologically relevant media may be measured using serial dilution and two-hour incubation period, followed by filtration, and reported in mM by LC-MS/MS. Thermodynamic solubility in physiologically relevant media may be measured by LC-MS/MS, after a twenty-four-hour incubation, followed by filtration, and reported in mg/mL.

### Additional Embodiments

Embodiments 1-53 are contemplated herein.

Embodiment 1. A compound of Formula (I):

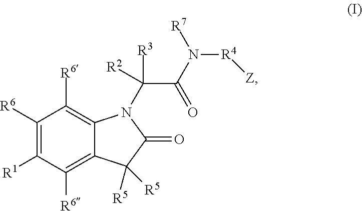

or a pharmaceutically acceptable salt thereof, wherein:
R¹ is halo, C₁-C₆ alkyl, C₁-C₆ haloalkyl, or C₃-C₆ cycloalkyl;
R⁷ and R² are joined to form a 5- to 10-membered monocyclic or bicyclic heterocyclyl or 5- to 10-membered monocyclic or bicyclic heteroaryl, wherein the heterocyclyl or heteroaryl are optionally substituted with one or more R^{7a}, wherein each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃-C₅ cycloalkyl, or 4 to 5-membered heterocyclyl, wherein the alkyl, haloalkyl, alkoxy, haloalkoxy, aryl, heteroaryl, cycloalkyl, and heterocyclyl are optionally substituted with one or more R^{7b}, further wherein R^{7b} is halo, —CN, —OH, C₁-C₆ alkyl, C₁-C₆ haloalkyl, C₁-C₆ alkoxy, or C₁-C₆ haloalkoxy;
R³ is absent, H, C₁-C₆ alkyl, or C₁-C₆ haloalkyl;
R⁴ is —C₁-C₆ alkylene- optionally substituted with one or more R^{4a}, wherein each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl, or two R^{4a} are joined to form a C₃-C₅ cycloalkyl or 4-6 membered heterocyclyl ring, wherein the alkyl, haloalkyl, alkoxy, haloalkoxy, cycloalkyl, and heterocyclyl are optionally substituted with one or more R^{4b}, further wherein R^{4b} is halo, —CN, —OH, C₁-C₆ alkyl, C₁-C₆ haloalkyl, C₁-C₆ alkoxy, or C₁-C₆ haloalkoxy;
Z is a —CO₂H or —CO₂PG, wherein PG is C₁-C₆ alkyl, C₆ aryl, or (C₆ aryl)(C₁-C₆ alkyl), further wherein alkyl and aryl are optionally substituted with one or more halo, C₁-C₆ alkyl, C₁-C₆ alkoxy, or C₁-C₆ haloalkoxy;
each R⁵ is independently halo, C₁-C₆ alkyl, or C₁-C₆ haloalkyl; and
each R⁶, R^{6′}, or R^{6″} is independently H, halo, —OH, C₁-C₆ alkoxy, C₁-C₆ haloalkoxy, C₁-C₆ alkyl, or C₁-C₆ haloalkyl.

Embodiment 2. The compound of Embodiment 1, or a pharmaceutically acceptable salt thereof, wherein:
R¹ is halo, C₁-C₆ haloalkyl, or C₃-C₄ cycloalkyl;
R⁷ and R² are joined to form a 5- to 10-membered monocyclic or bicyclic heteroaryl, wherein the heteroaryl is optionally substituted with one or more R^{7a}, wherein each R^{7a} is independently C₁-C₆ alkyl, C₁-C₆ haloalkyl, C₆ aryl, or C₃-C₅ cycloalkyl;
R³ is absent;
R⁴ is —C₁-C₆ alkylene-;
Z is a —CO₂H;
each R⁵ is independently C₁-C₆ alkyl; and
each R⁶, R^{6′}, or R^{6″} is independently H, halo, or —OH.

Embodiment 3. The compound of any one of the preceding Embodiments, or a pharmaceutically acceptable salt thereof, wherein R¹ is Br, Cl, CF₃, or cyclopropyl.

Embodiment 4. The compound of any one of the preceding Embodiments, or a pharmaceutically acceptable salt thereof, wherein R³ is absent.

Embodiment 5. The compound of any one of the preceding Embodiments, or a pharmaceutically acceptable salt thereof, wherein each instance of R⁵ is the same group.

Embodiment 6. The compound of any one of the preceding Embodiments, or a pharmaceutically acceptable salt thereof, wherein each R⁵ is independently methyl (—CH₃).

Embodiment 7. The compound of any one of the preceding Embodiments, or a pharmaceutically acceptable salt thereof, wherein R⁶ is H.

Embodiment 8. The compound of any one of the preceding Embodiments, or a pharmaceutically acceptable salt thereof, wherein R^{6′} is F or —OH.

Embodiment 9. The compound of any one of the preceding Embodiments, or a pharmaceutically acceptable salt thereof, wherein R^{6″} is H or F.

Embodiment 10. The compound of any one of the preceding Embodiments, or a pharmaceutically acceptable salt thereof, wherein R⁷ and R² are joined to form a 6-membered monocyclic heteroaryl optionally substituted with one or more R^{7a}.

Embodiment 11. The compound of any one of Embodiments 1-9, or a pharmaceutically acceptable salt thereof, wherein R⁷ and R² are joined to form a 10-membered bicyclic heteroaryl.

Embodiment 12. The compound of any one of the preceding Embodiments, or a pharmaceutically acceptable salt thereof, wherein R^{7a} is methyl, CF₃, C₆ aryl, or cyclopropyl.

Embodiment 13. The compound of any one of the preceding Embodiments, or a pharmaceutically acceptable salt thereof, wherein R⁴ is —C₃ alkylene-.

Embodiment 14. The compound of any one of the preceding Embodiments, or a pharmaceutically acceptable salt thereof, wherein Z is CO₂H.

Embodiment 15. The compound of any one of the preceding Embodiments, wherein the compound is of Formula (I-A):

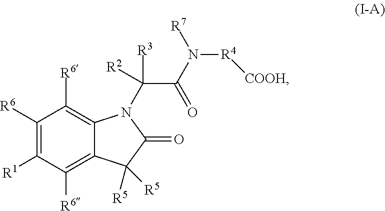

or a pharmaceutically acceptable salt thereof.

Embodiment 16. The compound of any one of the preceding Embodiments, wherein the compound is of Formula (I-A-a):

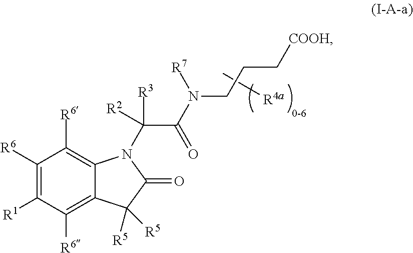

or a pharmaceutically acceptable salt thereof.

Embodiment 17. The compound of any one of the preceding Embodiments, wherein the compound is of Formula (I-B), (I-C), (I-D), (I-E), (I-F), or (I-G):

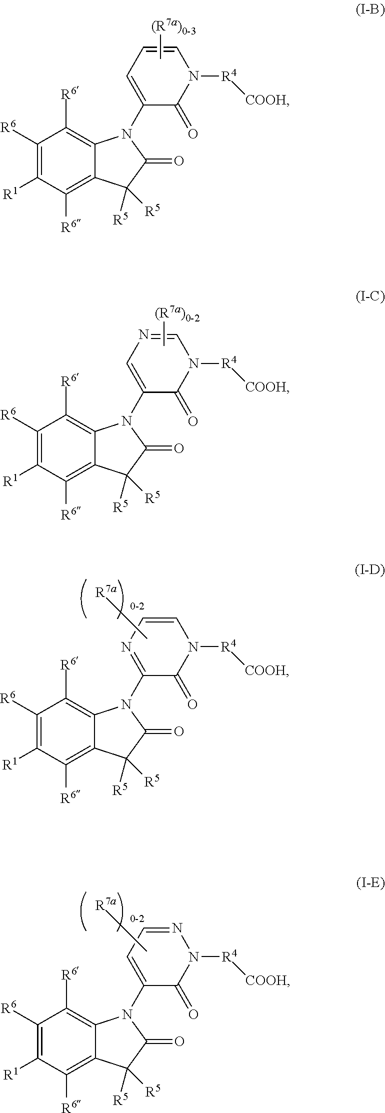

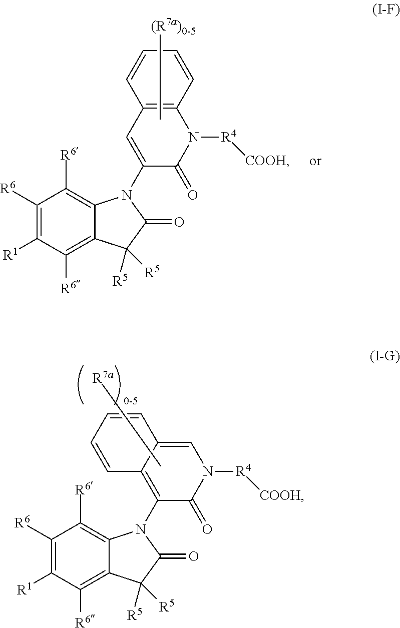

or a pharmaceutically acceptable salt thereof.

Embodiment 18. The compound of any one of the preceding Embodiments, wherein the compound is of Formula (I-B′), (I-C′), (I-D′), (I-E′), (I-F′), or (I-G′):

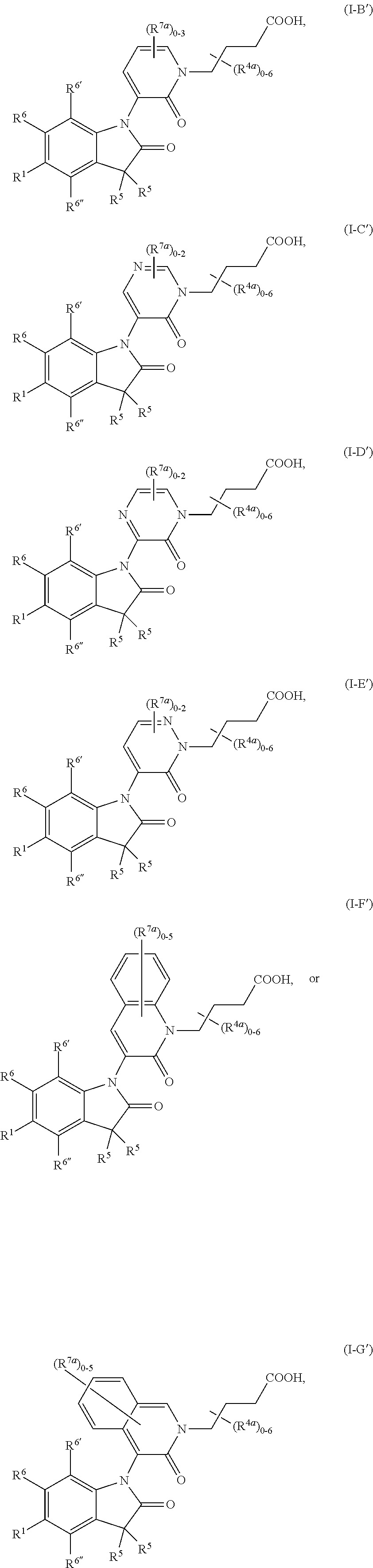

or a pharmaceutically acceptable salt thereof.

Embodiment 19. The compound of any one of the preceding Embodiments, wherein the compound is of Formula (I-B-a), (I-C-a), (I-D-a), (I-E-a), (I-F-a), or (I-G-a):

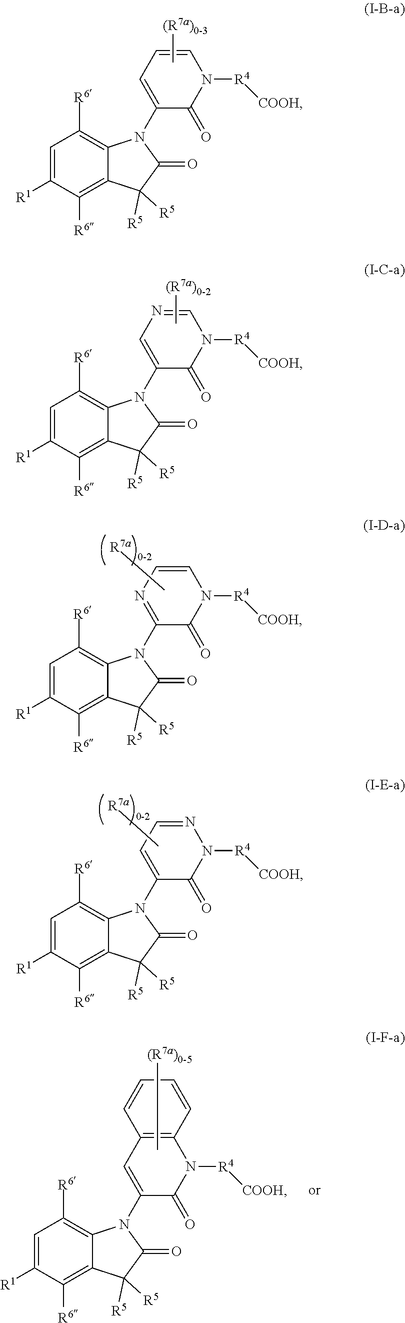

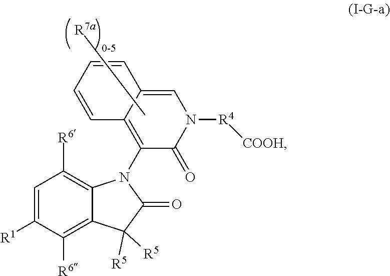

or a pharmaceutically acceptable salt thereof.

Embodiment 20. The compound of any one of the preceding Embodiments, wherein the compound is of Formula (I-B′-a), (I-C′-a), (I-D′-a), (I-E′-a), (I-F′-a), or (I-G′-a):

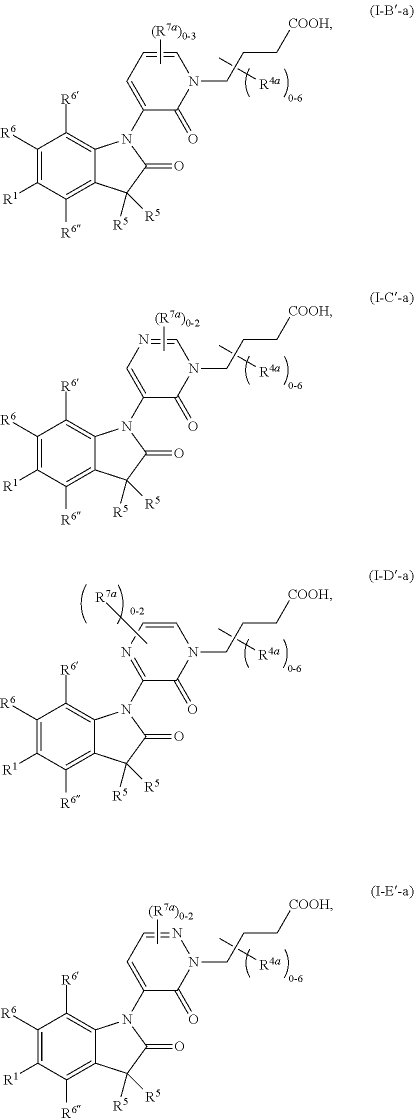

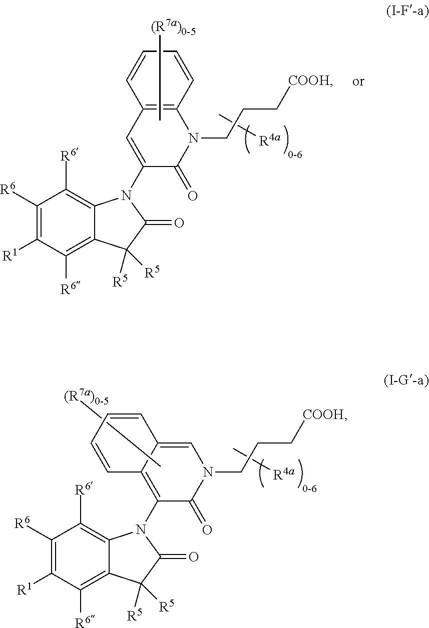

or a pharmaceutically acceptable salt thereof.

Embodiment 21. The compound of any one of the preceding Embodiments, wherein the compound is of Formula (I-J):

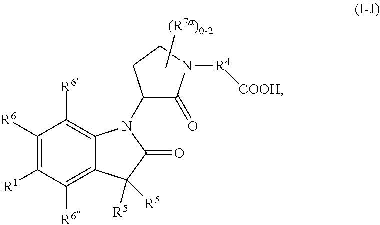

or a pharmaceutically acceptable salt thereof.

Embodiment 22. The compound of any one of the preceding Embodiments, wherein the compound is of Formula (I-J′)

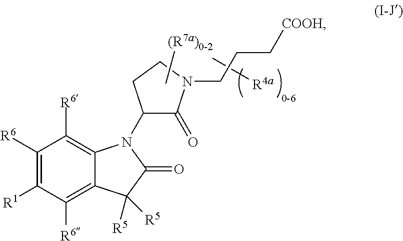

or a pharmaceutically acceptable salt thereof.

Embodiment 23. The compound of any one of the preceding Embodiments, wherein the compound is of Formula (I-J-a)

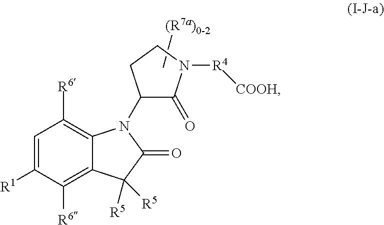

or a pharmaceutically acceptable salt thereof.

Embodiment 24. The compound of any one of the preceding Embodiments, wherein the compound is of Formula (I-J′-a)

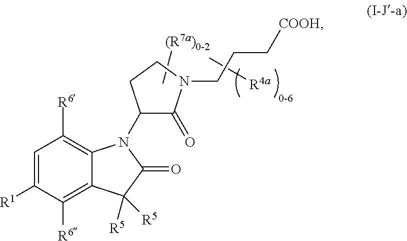

or a pharmaceutically acceptable salt thereof.

Embodiment 25. The compound of any one of Embodiments 1-24, wherein the compound is selected from the compounds described in Table 1 or Table 2A, or a pharmaceutically acceptable salt thereof.

Embodiment 26. A process for preparing a compound, or a pharmaceutically acceptable salt thereof, of Formula (I) of any one of the preceding Embodiments, or an intermediate thereof, wherein the compound or intermediate is synthesized according to General Method Scheme A.

Embodiment 27. A pharmaceutical composition comprising the compound of any one of Embodiments 1-25 or Embodiments 1A-6A, or a pharmaceutically acceptable salt thereof, and one or more pharmaceutically acceptable carriers.

Embodiment 28. A method of modulating NLRP3, the method comprising administering to the subject a compound of any one of Embodiments 1-25 or Embodiments 1A-6A, or a pharmaceutically acceptable salt thereof, or a pharmaceutical composition of Embodiment 27.

Embodiment 29. A method of treating or preventing a disease or disorder, the method comprising administering to the subject a compound of any one of Embodiments 1-25 or Embodiments 1A-6A, or a pharmaceutically acceptable salt thereof, or a pharmaceutical composition of Embodiment 27.

Embodiment 30. The compound of any one of Embodiments 1-25 or Embodiments 1A-6A, or a pharmaceutically acceptable salt thereof, or a pharmaceutical composition of Embodiment 27, for use in treating or preventing a disease or disorder.

Embodiment 31. Use of the compound of any one of Embodiments 1-25 or Embodiments 1A-6A, or a pharmaceutically acceptable salt thereof, in the manufacture of a medicament, for the treatment or prevention of a disease or disorder.

Embodiment 32. Use of the compound of any one of Embodiments 1-25 or Embodiments 1A-6A, or a pharmaceutically acceptable salt thereof, for the treatment or prevention of a disease or disorder.

Embodiment 33. The method, compound, or use of any one of Embodiments 29-32, wherein the disease or disorder is an NLRP3-related disease or disorder.

Embodiment 34. The method, compound, or use of any one of Embodiments 28-29, wherein the subject is a human.

Embodiment 35. The method, compound, or use of any one of Embodiments 29-34, wherein the disease or disorder is a disease or disorder of the central nervous system (CNS), a disease or disorder of the peripheral nervous system (PNS), a primary neurological disease of the muscles, an inflammatory disorder, an autoimmune disorder, cancer, an infection, a metabolic disease, a cardiovascular disease, a respiratory disease, a kidney disease, a liver disease, an ocular disease, a skin disease, a lymphatic disease, a rheumatic disease, a psychological disease, graft versus host disease, pain (including disorders related to pain management), or an NLRP3-related disease in a subject that has been determined to carry a germline or somatic non-silent mutation in NLRP3.

Embodiment 36. The method, compound, or use of any one of Embodiments 29-35, wherein the disease or disorder of the central nervous system is dementia, Alzheimer's disease (“AD”) epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), Huntington's disease (“HD”), or spinal cord injury.

Embodiment 37. The method, compound, or use of any one of Embodiments 29-35, wherein the primary neurological disease of the muscle is dystrophies or spinal muscular atrophy.

Embodiment 38. The method, compound, or use of any one of Embodiments 29-35, wherein the inflammatory disorder is gout or anemia of inflammation.

Embodiment 39. The method, compound, or use of any one of Embodiments 29-35, wherein the autoimmune disease is ulcerative colitis.

Embodiment 40. The method, compound, or use of any one of Embodiments 29-35, wherein the cancer is skin cancer or colon cancer.

Embodiment 41. The method, compound, or use of any one of Embodiments 29-35, wherein the infection is a neuro-infection.

Embodiment 42. The method, compound, or use of any one of Embodiments 29-35, wherein the metabolic disease is diabetes.

Embodiment 43. The method, compound, or use of any one of Embodiments 29-35, wherein the cardiovascular disease is stroke.

Embodiment 44. The method, compound, or use of any one of Embodiments 29-35, wherein the respiratory disease is asthma or chronic obstructive pulmonary disease.

Embodiment 45. The method, compound, or use of any one of Embodiments 29-35, wherein the kidney disease is acute kidney disease, a chronic kidney disease, or a rare kidney disease.

Embodiment 46. The method, compound, or use of any one of Embodiments 29-35, wherein the liver disease is nonalcoholic fatty liver disease (NAFLD) or non-alcoholic steatohepatitis (NASH).

Embodiment 47. The method, compound, or use of any one of Embodiments 29-35, wherein the ocular disease is optic neuritis or macular degeneration.

Embodiment 48. The method, compound, or use of any one of Embodiments 29-35, wherein the skin disease is psoriasis, hidradenitis suppurativa (HS), or atopic dermatitis.

Embodiment 49. The method, compound, or use of any one of Embodiments 29-35, wherein the rheumatic disease is osteoarthritis, dermatomyositis, Still's disease, orjuvenile idiopathic arthritis.

Embodiment 50. The method, compound, or use of any one of Embodiments 29-35, wherein the psychological disease is a neuropsychiatric condition selected from the group consisting of depression, major depressive disorder, and refractory depression.

Embodiment 51. The method, compound, or use of any one of Embodiments 29-35, wherein the pain is pain management addiction, osteoarthritis pain, or allodynia.

Embodiment 52. The method, compound, or use of any one of Embodiments 29-35, wherein the NLRP3-related disease in a subject that has been determined to carry a germline or somatic non-silent mutation in NLRP3 is cryopyrin-associated autoinflammatory syndrome.

Embodiment 53. The method, compound, or use of any one of Embodiments 29-34, wherein the disease or disorder is dementia, Alzheimer's disease (“AD”), epilepsy, traumatic brain injury (“TBI”), multiple sclerosis (“MS”), developmental disturbances, acute disseminated encephalopathy, transverse myelitis, Parkinson's disease (“PD”), amyotrophic lateral sclerosis (“ALS”), spinal muscular atrophy, Huntington's disease (“HD”), spinal cord injury, dystrophies, neuro-infections, pain management addiction, neuropsychiatric conditions (e.g. depression, major depressive disorder, refractory depression), neonatal onset multisystem inflammatory disease (“NOMID”), asthma, osteoarthritis, ulcerative colitis, gout, anemia of inflammation, Still's disease, chronic obstructive pulmonary disease (“COPD”), osteoarthritis pain, or hidradenitis suppurativa.

Additional Embodiments 1A-9A are also contemplated herein.

Embodiment 1A. The compound of Embodiment 24, wherein the compound is of Formula (I-J′-a-R).

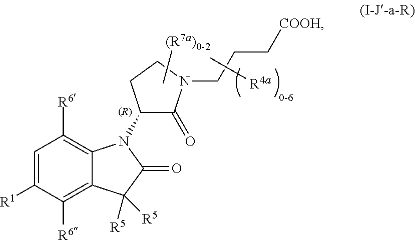

or a pharmaceutically acceptable salt thereof.

Embodiment 2A. The compound of any one of Embodiments 16, 18, 20, 22, 24, or 1A, or a pharmaceutically acceptable salt thereof, wherein R^{4a} is absent.

Embodiment 3A. The compound of any one of Embodiments 16, 18, 20, 22, 24, or 1A-2A, or a pharmaceutically acceptable salt thereof, wherein R^{7a} is absent.

Embodiment 4A. The compound of any one of Embodiments 16, 18, 20, 22, 24, or 1A-3A, or a pharmaceutically acceptable salt thereof, wherein R¹ is —CF₃ or cyclopropyl.

Embodiment 5A. The compound of any one of Embodiments 16, 18, 20, 22, 24, or 1A-4A, or a or a pharmaceutically acceptable salt thereof, wherein R^{6′} is halo and R^{6″} is H, or each of R¹ and R^{6″} is independently halo.

Embodiment 6A. The compound of any one of the preceding Embodiments, wherein the compound is selected from the compounds described in Table 1 or Tables 2A-2B, or a pharmaceutically acceptable salt thereof.

Embodiment 7A. The method, compound, or use of any one of Embodiments 29-34 and Embodiments 1A-6A, wherein the disease or disorder is obesity.

Embodiment 8A. A process for preparing a compound of Formula (I), or a pharmaceutically acceptable salt thereof, the method comprising coupling a substituted indolinone (i), or salt thereof, with a lactam of formula (ii), or salt thereof:

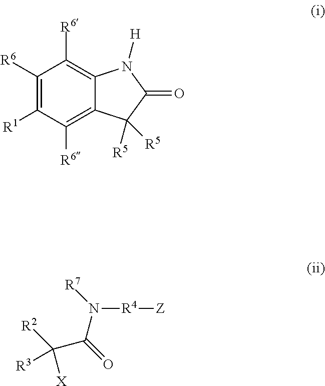

wherein R¹, R², R³, R⁴, R⁵, R⁶, R^{6′}, and R^{6″} are as defined in claim 1, X is a leaving group, and Z is —CO₂PG, wherein PG is as defined in claim 1, to provide a compound of Formula (I), wherein Z is —CO₂PG, or salt thereof.

Embodiment 9A. The process of Embodiment 8A, further comprising deprotecting the compound of Formula (I), or salt thereof, wherein Z is —CO₂PG, to provide a compound of Formula (I), or salt thereof, wherein Z is —CO₂H.

### Exemplification

In order that this disclosure may be more fully understood, the following Examples are set forth. It should be understood that these Examples are for illustrative purposes only and are not to be construed as limiting this disclosure in any manner.

### Analytical Methods

Nuclear magnetic resonance (NMR) spectra were recorded at 400 megahertz (MHz) as stated and at 300.3 K unless otherwise stated; the chemical shifts (6) are reported in parts per million (ppm). Spectra were recorded using a Bruker Avance 400 instrument with 8, 16 or 32 scans. Exemplary NMR solvents include deuterated dimethylsulfoxide (DMSO-d₆), deuterated methanol (CD₃OD), and deuterated chloroform (CDCl₃). Other abbreviations: s=singlet; d=doublet; t=triplet; m=multiplet; br=broad.

Liquid Chromatography—Mass Spectrometry (LCMS) and spectra were recorded using a Shimadzu LCMS-2020. Injection volumes were 0.7-8.0 μl and the flow rates were typically 0.8 or 1.2 ml/min. Detection methods were diode array (DAD) or evaporative light scattering (ELSD) as well as positive ion electrospray ionization (ESI). MS range was 100-1000 Da. Solvents were gradients of water (H₂O) and/or acetonitrile (MeCN) may contain a modifier (typically 0.01-0.04%) such as formic acid (FA), trifluoroacetic acid (TFA) or ammonium carbonate (NH₄HCO₃). ESI or ES=electrospray ionization; m/z=mass/charge; RT=retention time (minutes).

Gas Chromatography—Mass Spectrometry (GCMS) chromatograms and spectra were recorded using Agilent GCMS 8890-5977 and Detector Channel FID. GC Parameters: DB-5MS, 12 m×0.20 mm×0.33 um; Column Oven Temp: 50.0; Injection volume: 0.5 μL; Column Flow: 1.2 ml/min; Injection temperature: 300° C.; Injection Mode: Split; Split Ratio: 20:1; Detector temperature: 300° C.; Initial temperature: 50° C. for 1 min then 40° C./min to 300° C. for 1.75 min. Makeup Gas: He; Makeup Flow: 25.0 mL/min; H2; Flow: 30.0 mL/min; Air Flow: 400.0 mL/min; Final temperature: 300° C. The MS detector of acquisition mode: Start Time: 2.00 min; End Time: 9.00 min; Acquisition Mode: Scan;Interface Type: El Threshold: 150; Scan Speed: 1562; Start m/z: 50.00; End m/z: 550.00; MS Source: 230.00° C.; MS Quad: 150.00° C.; Solvent Cut Time: 2.00 min.

Purification/Separation Methods. The Synthetic methods describe purification and/or separation chromatographic methods which have been employed in the purification and/or isolation of the exemplified compounds. Rf=retention factor; RT=retention time (minutes); Prep-HPLC=Preparative High-performance liquid chromatography; Prep-SFC=Preparative Supercritical Fluid Chromatography; TLC=thin layer chromatography.

If a stereochemical position is arbitrarily or rationally assigned, an Asterix (*) is included as part of the compound number. Rational assignment signifies there is a correlation between the designated assignment, such as activity, and a known (e.g., absolute) assignment. “Rac” as a prefix to a compound number signifies a mixture of two or more stereoisomers in equal or unequal amounts. Future tense (“may be” prepared/synthesized) language signify examples not yet conducted.

### INTERMEDIATES

### Intermediate Example 1. 5-bromo-7-fluoro-3,3-dimethyl-1H-indol-2-one (Intermediate 1)

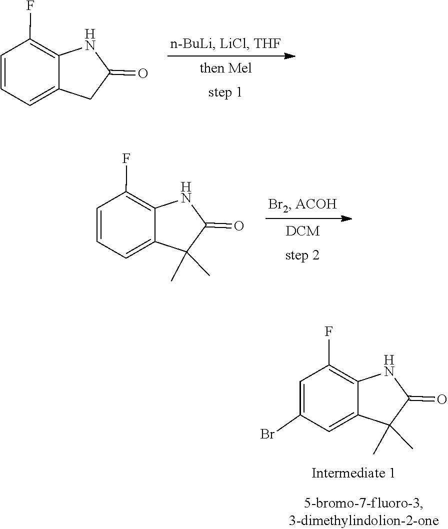

Step 1: To a solution of 7-fluoro-1,3-dihydroindol-2-one (2 g, 13.23 mmol, 1 equiv) and lithium chloride (1.40 g, 33.08 mmol, 2.5 equiv) in tetrahydrofuran (THF) (40 mL) at 0° C. was added dropwise added n-butyl lithium (n-BuLi) (2.5M in hexanes, 10.5 mL, 26.46 mmol, 2 equiv) in portions under nitrogen atmosphere. The resulting mixture was stirred for 30 min at 0° C. Then methyl iodide (Mel) (4.69 g, 33.08 mmol, 2.5 equiv) was added slowly and the mixture was stirred at 0° C. for 2 h, and then further stirred at ambient temperature for 16 h. The reaction was monitored by LCMS. The reaction was quenched with sat. NH₄Cl (40 mL). The aqueous layer was extracted with ethyl acetate (EtOAc) (4×50 mL). The combined organic layers were washed with brine (1×50 mL) and dried over anhydrous Na₂SO₄. After filtration, the filtrate was concentrated under reduced pressure. The residue was purified by reverse flash chromatography (C18 silica gel; mobile phase, acetonitrile in water, 10% to 50% gradient in 30 min; detector, UV 254 nm) to provide 7-fluoro-3,3-dimethyl-1H-indol-2-one (1.3 g, 54% yield).

LCMS: (ES, m/z): RT=0.73 min, m/z=180[M+H]⁺.

Step 2: To a solution of 7-fluoro-3,3-dimethyl-1H-indol-2-one (1 g, 5.58 mmol, 1 equiv) in acetic acid (AcOH) (4 mL) and dichloromethane (DCM) (40 mL), was added bromine (Br₂) (892 mg, 5.58 mmol, 1 equiv) was added by dropwise over 4 min at 0° C. The resulting mixture was stirred for additional 2 h at room temperature. The reaction was monitored by LCMS. The reaction was quenched with saturated NaHSO₃ aq (20 mL) at 0° C. The aqueous layer was extracted with ethyl acetate (EtOAc) (3×50 mL). The combined organic layers were washed with brine (1×50 mL), dried over anhydrous Na₂SO₄. After filtration, the filtrate was concentrated under reduced pressure. The residue was purified by silica gel column chromatography, eluting with petroleum ether/ethyl acetate (4:1), to afford 5-bromo-7-fluoro-3,3-dimethyl-1H-indol-2-one (Intermediate 1) (1.10 g, 73% yield). LCMS: (ES, m/z): RT=0.83 min, m/z=258[M+H]⁺.

### Intermediate Example 2. 5-cyclopropyl-7-fluoro-3,3-dimethylindolin-2-one (Intermediate 2)

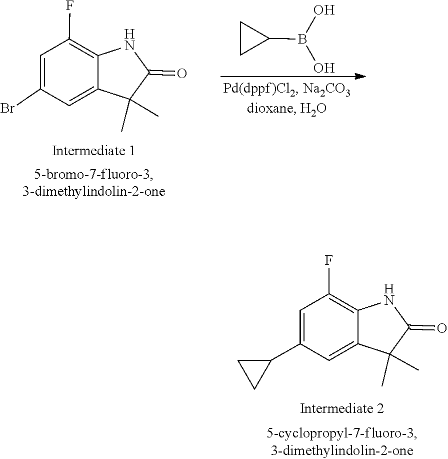 [SMILES: CC1(C)C(=O)Nc2c(F)cc(Br)cc21 ; OB(O)C1CC1 ; CC1(C)C(=O)Nc2c(F)cc(C3CC3)cc21]

To a solution of 5-bromo-7-fluoro-3,3-dimethyl-1H-indol-2-one (Intermediate 1) (170.0 mg, 0.66 mmol, 1 equiv) and cyclopropylboronic acid (170.3 mg, 1.98 mmol, 3 equiv) in dioxane (6 mL) and H₂O (1.20 mL) was added Na₂CO₃ (209.9 mg, 1.98 mmol, 3 equiv) and [1,1′-bis(diphenylphosphino)ferrocene]dichloropalladium(II) complex with dichloromethane (Pd(dppf)Cl₂-DCM) (107.8 mg, 0.13 mmol, 0.20 equiv). The resulting mixture was stirred overnight at 80° C. under a nitrogen atmosphere. The reaction was monitored by LCMS. The resulting mixture was diluted with water (60 mL), extracted with ethyl acetate (EtOAc) (3×50 mL). The combined organic layers were washed with brine (1×50 mL) and dried over anhydrous Na₂SO₄. After filtration, the filtrate was concentrated under reduced pressure. The residue was purified silica gel column chromatography, eluting with petroleum ether/ethyl acetate (5:1) to afford 5-cyclopropyl-7-fluoro-3,3-dimethylindolin-2-one (Intermediate 2) (70 mg, 48% yield). LCMS: (ES, m/z): RT=0.81 min, m/z=220[M+H]⁺.

### Intermediate Example 3. 5-cyclopropyl-4,7-difluoro-3,3-dimethylindolin-2-one (Intermediate 3) and 5-bromo-4,7-difluoro-3,3-dimethylindolin-2-one (Intermediate 5)

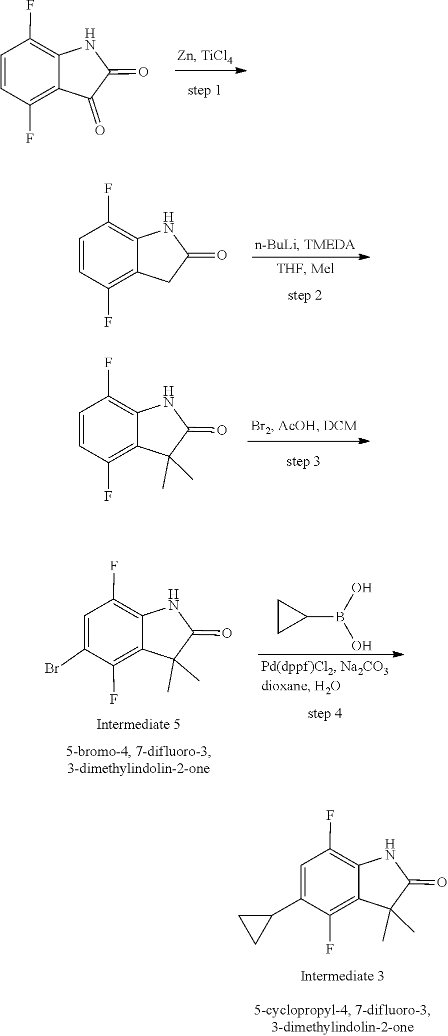

Step 1: To a stirred mixture of Zn powder (3.21 g, 49.1 mmol, 9 equiv) in tetrahydrofuran (THF) (100 mL) was added TiCl₄ (3.79 mL, 27.3 mmol, 5 equiv) in portions at 0° C. under nitrogen atmosphere.

The resulting mixture was stirred for 2 h at 70° C. under nitrogen atmosphere. To the above mixture was added 4,7-difluoro-1H-indole-2,3-dione (1 g, 5.46 mmol, 1 equiv) by dropwise at room temperature. The resulting mixture was stirred for additional 12 h at room temperature. The reaction was monitored by LCMS. The reaction was quenched with water (150 mL). The resulting mixture was extracted with CH₂Cl₂ (3×300 mL) and dried over anhydrous Na₂SO₄. After filtration, the filtrate was concentrated under reduced pressure. The residue was purified by silica gel column chromatography, eluting with petroleum ether/ethyl acetate (9:1), to afford 4,7-difluoro-1,3-dihydroindol-2-one (450 mg, 27% yield).

LCMS (LCMS, ESI): RT=0.72 min, m/z=168 [M−H]⁻.

Step 2: A solution of 4,7-difluoro-1,3-dihydroindol-2-one (450 mg, 0.25 mmol, 1 equiv) and tetramethylethylenedianine (TMEDA) (805 mg, 8.26 mmol, 2.5 equiv) in tetrahydrofuran (THF) (50 mL) was treated with n-butyl lithium (n-BuLi) (3.3 mL, 8.26 mmol, 2.5 equiv, 2.5M in THF) for 30 min at 0° C. under nitrogen atmosphere followed by the addition of methyl iodide (Mel) (785 mg, 6.52 mmol, 2 equiv) by dropwise at room temperature. The resulting mixture was stirred for 1 h at room temperature under nitrogen atmosphere. The reaction was monitored by LCMS. The reaction was quenched by the addition of water (200 mL) at room temperature. The resulting mixture was extracted with ethyl acetate (EtOAc) (3×150 mL) and dried over anhydrous Na₂SO₄. After filtration, the filtrate was concentrated under reduced pressure and the residue was purified by silica gel column chromatography, eluting with petroleum ether/ethyl acetate (6:1), to afford 4,7-difluoro-3,3-dimethyl-1H-indol-2-one (230 mg, 41% yield). LCMS (ESI): RT=0.92 min, m/z=196 [M−H]⁻.

Step 3: To a stirred mixture of 4,7-difluoro-3,3-dimethyl-1H-indol-2-one (230 mg, 1.31 mmol, 1 equiv) and acetic acid (AcOH) (2 mL) in dichloromethane (DCM) (10 mL) was added Br₂ (1.05 g, 6.59 mmol, 5 equiv, in 3 mL DCM) by dropwise at 0° C. The resulting mixture was stirred for 1 h at room temperature under nitrogen atmosphere. The reaction was monitored by LCMS, and upon completion, was quenched by the addition of sat. NaHCO₃ (aq.) (50 mL) at room temperature. The resulting mixture was extracted with ethyl acetate (EtOAc) (3×200 mL) and dried over anhydrous Na₂SO₄. After filtration, the filtrate was concentrated under reduced pressure to provide a residue, which was purified by silica gel column chromatography, eluting with petroleum ether/ethyl acetate (5:1), to afford 5-bromo-4,7-difluoro-3,3-dimethylindolin-2-one (Intermediate 5) (300 mg, 69% yield). LCMS (LCMS, ESI): RT=1.05 min, m/z=274 [M−H]⁻.

Step 4: Intermediate 5 (1 g, 3.62 mmol, 1 equiv), cyclopropylboronic acid (3.11 g, 36.2 mmol, 10 equiv), 1,1′-bis(diphenylphosphino)ferrocene]dichloropalladium (II) complex with dichloromethane (Pd(dppf)Cl₂—DCM) (0.53 g, 0.72 mmol, 0.20 equiv), Na₂CO₃ (1.15 g, 10.9 mmol, 3 equiv), dioxane (20 mL) and H₂O (4 mL) were stirred for 12 h at 100° C. under nitrogen atmosphere. The reaction was monitored by LCMS. The reaction was quenched by the addition of water (100 mL) at room temperature. The resulting mixture was extracted with ethyl acetate (EtOAc) (3×100 mL) and dried over anhydrous Na₂SO₄. After filtration, the filtrate was concentrated under reduced pressure. The residue was purified by silica gel column chromatography, eluting with petroleum ether/ethyl acetate (7:1), to afford 5-cyclopropyl-4,7-difluoro-3,3-dimethylindolin-2-one (Intermediate 3) (800 mg, 75% yield). LCMS (LCMS, ESI): RT=0.84 min, m/z=238.1 [M+H]⁺.

### Intermediate Example 4. 5-chloro-7-iodo-3,3-dimethylindolin-2-one (Intermediate 4) and 5-chloro-3,3-dimethylindolin-2-one (Intermediate 18)

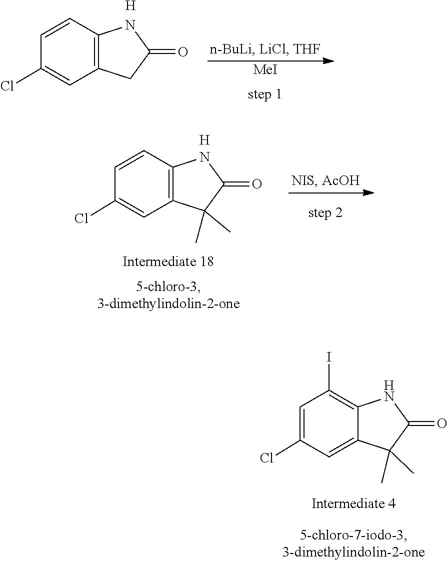

Step 1: Into a 250 mL 3-necked round-bottom flask was added 5-chloro-1,3-dihydroindol-2-one (5 g, 29.83 mmol, 1 equiv), LiCl (3.79 g, 89.5 mmol, 3 equiv) and tetrahydrofuran (THF) (100 mL) at room temperature, then n-butyl lithium (n-BuLi in 2.5 M in n-Hexanes, 29.8 mL, 74.6 mmol, 2.5 equiv) was added at −78° C. by dropwise. The resulting mixture was stirred for 30 min at 0° C. under nitrogen atmosphere. To the above mixture was added methyl iodide (Mel) (8.47 g, 59.7 mmol, 2 equiv) dropwise over 5 min at −78° C. The resulting mixture was stirred for 2 h at 0° C. under nitrogen atmosphere. The reaction was monitored by LCMS. The reaction was quenched by the addition of NH₄Cl (aq, 150 mL) at room temperature. The resulting mixture was extracted with ethyl acetate (EtOAc) (3×150 mL), and dried over anhydrous Na₂SO₄. After filtration, the filtrate was concentrated under reduced pressure. The residue was purified by silica gel column chromatography, eluting with petroleum ether/ethyl acetate (1:1), to afford 5-chloro-3,3-dimethylindolin-2-one (Intermediate 18) (3.80 g, 65% yield). LCMS: (ES, m/z): RT=0.788 min, m/z=195.6 [M+H]⁺.

Step 2: Into a 40 mL vial was added Intermediate 18 (2 g, 10.22 mmol, 1 equiv), N-iodosuccinimide (NIS) (13.8 g, 61.3 mmol, 6 equiv) and acetic acid (HOAc) (40 mL) at room temperature. The resulting mixture was stirred for overnight at 80° C. The reaction was monitored by LCMS. The reaction was quenched by the addition of NH₄Cl (aq, 100 mL) at room temperature. The resulting mixture was extracted with ethyl acetate (EtOAc) (3×100 mL), and the organic layer dried over anhydrous Na₂SO₄. After filtration, the filtrate was concentrated under reduced pressure. The residue was purified by reversed-phase flash chromatography (C18 silica gel; mobile phase, acetonitrile in water, 0% to 100% gradient in 30 min; detector, UV 254/220 nm) to provide 5-chloro-7-iodo-3,3-dimethylindolin-2-one (Intermediate 4) (2.4 g, 73% yield). LCMS: (ES, m/z): RT=0.854 min, m/z=321.9 [M+H]⁺.

### Intermediate Example 5. 5-bromo-3,3,7-trifluoroindolin-2-one (Intermediate 6)

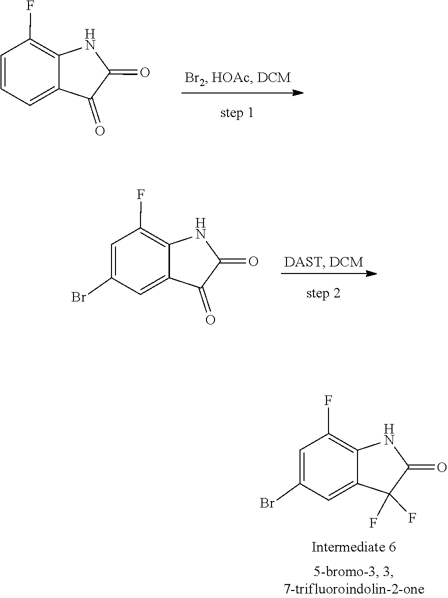 [SMILES: O=C1Nc2c(F)cccc2C1=O ; O=C1Nc2c(F)cc(Br)cc2C1=O ; O=C1Nc2c(F)cc(Br)cc2C1(F)F]

Step 1: Into a 40 mL sealed tube was added 7-fluoro-1H-indole-2,3-dione (5 g, 30.3 mmol, 1 equiv), dichloromethane (DCM) (15 mL) and acetic acid (AcOH) (3 mL) at room temperature. To the above solution was added Br₂ (9.68 g, 60.6 mmol, 2 equiv) at 0° C. The resulting mixture was stirred for 16 h at 40° C. under nitrogen atmosphere. The reaction was monitored by LCMS. The reaction was quenched by the addition of sodium thiosulfate (aq.) (100 mL) at room temperature. The resulting mixture was extracted with ethyl acetate (EtOAc) (3×100 mL). The combined organic layers were washed with water (100 mL), dried over anhydrous Na₂SO₄. After filtration, the filtrate was concentrated under reduced pressure. This resulted in 5-bromo-7-fluoro-1H-indole-2,3-dione (5.60 g, 76% yield).

LCMS:(ES, m/z): RT=0.598 min, m/z=243.9[M+H]⁺.

Step 2: Into a 250 mL three necks bottom flask was added 5-bromo-7-fluoro-1H-indole-2,3-dione (10.0 g, 41.0 mmol, 1 equiv) and dichloromethane (DCM) (100 mL) at room temperature. To the above solution was added diethylaminosulfurtrifluoride (DAST) (16.5 g, 102 mmol, 2.50 equiv) at 0° C. The resulting mixture was stirred for 16 h at room temperature. The reaction was monitored by LCMS. The reaction was quenched with water (200 mL) at room temperature. The resulting mixture was extracted with ethyl acetate (EtOAc) (3×200 mL). The combined organic layers were washed with water (100 mL), dried over anhydrous Na₂SO₄. After filtration, the filtrate was concentrated under reduced pressure. The residue was purified by silica gel column chromatography, eluting with petroleum ether/ethyl acetate (1:1), to afford 5-bromo-3,3,7-trifluoroindolin-2-one (Intermediate 6) (8.50 g, 78% yield).

LCMS:(ES, m/z): RT=0.745 min, m/z=266.9[M+H]⁺.

### Intermediate Example 6. 5-bromo-7-iodo-3,3-dimethylindolin-2-one (Intermediate 7) and 5-bromo-3,3-dimethylindolin-2-one (Intermediate 19)

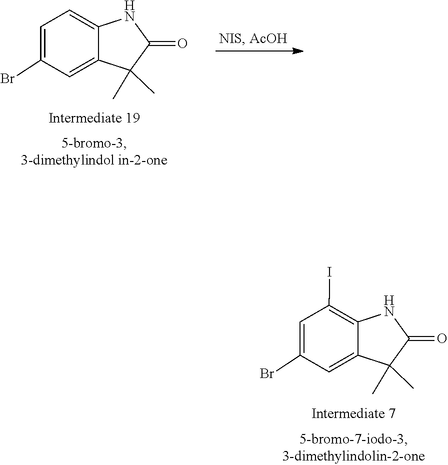

To an ice-cold solution of 5-bromo-3,3-dimethylindolin-2-one (Intermediate 19) (400 mg, 1.66 mmol, 1 equiv) (Compound 24a of Fensome et al., J. Med. Chem. (2008) 1861-1873) in acetic acid (AcOH) (4 mL) was added N-iodosuccinimide (NIS) (450 mg, 1.99 mmol, 1.2 equiv) portion wise followed by stirring for 3 h at 50° C. Then conc. H₂SO₄ (49.01 mg, 0.50 mmol, 0.3 equiv) was added at room temperature. The resulting mixture was stirred for additional 2 h at 50° C. The reaction mixture was quenched by ice/water (10 mL). The resulting mixture was extracted with ethyl acetate (EtOAc) (5×10 mL). The combined organic layers were dried over anhydrous Na₂SO₄. After filtration, the filtrate was concentrated under reduced pressure. The residue was purified by silica gel column chromatography, eluting with petroleum ether/ethyl acetate (5:1), to afford 5-bromo-7-iodo-3,3-dimethylindolin-2-one (Intermediate 7) (500 mg, 82% yield). LCMS:(ES, m/z): RT=0.85 min, m/z=366 [M+H]⁺.

### Intermediate Example 7. 5-bromo-6-methoxy-3,3-dimethylindolin-2-one (Intermediate 8)

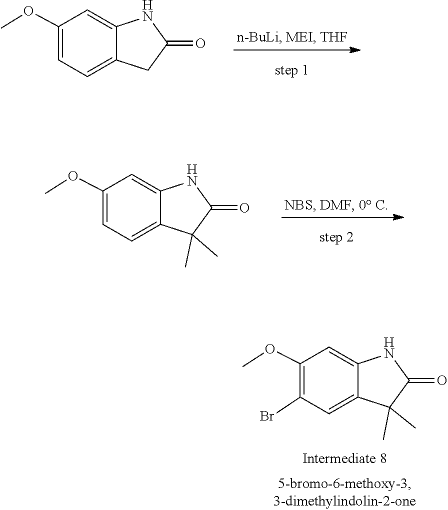

Step 1: To a stirred solution of 6-methoxy-1,3-dihydroindol-2-one (1 g, 6.12 mmol, 1 equiv) and LiCl (649 mg, 15.32 mmol, 2.5 equiv) in tetrahydrofuran (THF) (100 mL) was added n-butyl lithium (n-BuLi) (2.5N) (6.13 mL, 15.32 mmol, 2.5 equiv) dropwise at 0° C. under nitrogen atmosphere.

The resulting mixture was stirred for 30 min at 0° C. under nitrogen atmosphere. To the above mixture was added Mel (1739.70 mg, 12.25 mmol, 2 equiv) dropwise over 20 min at 0° C. The resulting mixture was stirred for additional 1 h at 0° C. The reaction was quenched by the addition of water/ice (20 mL) at 0° C. The aqueous layer was extracted with ethyl acetate (EtOAc) (3×200 mL) and dried over anhydrous Na₂SO₄. After filtration, the filtrate was concentrated under reduced pressure. The residue was purified by silica gel column chromatography, eluting with petroleum ether/ethyl acetate (8:1), to afford 6-methoxy-3,3-dimethyl-1H-indol-2-one (200 mg, 17% yield).

Step 2: Into a 20 mL vial was added 6-methoxy-3,3-dimethyl-1H-indol-2-one (200 mg, 1.04 mmol, 1 equiv), N,N-dimethyl formamide (DMF) (8 mL), and N-bromosuccinimide (NBS) (186 mg, 1.046 mmol, 1 equiv) at room temperature. The resulting mixture was stirred for 2 h at room temperature under nitrogen atmosphere. The reaction was quenched by the addition of water/ice (20 mL) at 0° C. The aqueous layer was extracted with ethyl acetate (EtOAc) (3×20 mL) and dried over anhydrous Na₂SO₄. After filtration, the filtrate was concentrated under reduced pressure. The residue was purified by silica gel column chromatography, eluting with petroleum ether/ethyl acetate (2:1), to afford 5-bromo-6-methoxy-3,3-dimethylindolin-2-one (Intermediate 8) (190 mg, 67% yield). LCMS (LCMS, ESI): RT=0.735 min, m/z 270[M+H]⁺.

### Intermediate Example 8. 7-iodo-3,3-dimethyl-5-(trifluoromethyl)indolin-2-one (Intermediate 17) and 3,3-dimethyl-5-(trifluoromethyl)indolin-2-one (Intermediate 11)

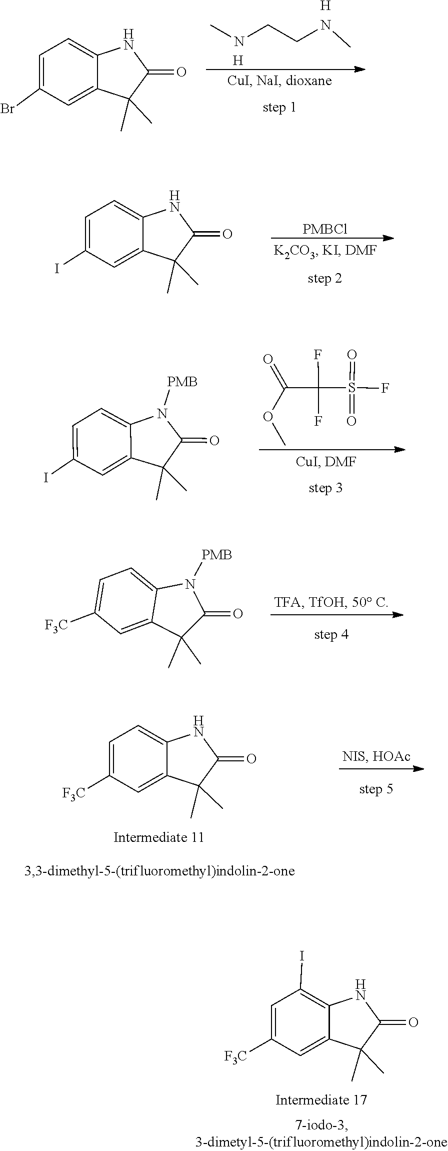

Step 1: Into a 40 mL sealed tube was added 5-bromo-3,3-dimethyl-1H-indol-2-one (4 g, 16.7 mmol, 1 equiv), methyl[2-(methylamino)ethyl]amine (2.94 g, 33.3 mmol, 2 equiv), CuI (6.35 g, 33.3 mmol, 2 equiv), NaI (4.99 g, 33.3 mmol, 2 equiv) and dioxane (20 mL) at room temperature. The resulting mixture was stirred for 2 h at 120° C. under nitrogen atmosphere, then quenched with water (100 mL) at room temperature. The resulting mixture was extracted with ethyl acetate (EtOAc) (3×150 mL), and the combined organic layers were washed with water (100 mL), and dried over anhydrous Na₂SO₄. After filtration, the filtrate was concentrated under reduced pressure. The residue was purified by reversed-phase flash chromatography (C18 silica gel; mobile phase, acetonitrile in water, 0% to 100% gradient in 30 min; detector, UV 254/220 nm) to provide 5-iodo-3,3-dimethyl-1H-indol-2-one (2.50 g, 52% yield). LCMS:(ES, m/z): RT=0.507 min, m/z=288.0[M+H]⁺.

Step 2: Into a 40 mL sealed tube was added 5-iodo-3,3-dimethyl-1H-indol-2-one (2.50 g, 8.70 mmol, 1 equiv), K₂CO₃ (3.61 g, 26.1 mmol, 3 equiv), KI (1.45 g, 8.70 mmol, 1 equiv), N,N-dimethyl formamide (DMF) (20 mL) and p-methoxybenzyl chloride (PMBCl) (4.09 g, 26.1 mmol, 3 equiv) at room temperature. The resulting mixture was stirred for 2 h at 100° C., then quenched with water (80 mL) at room temperature. The resulting mixture was extracted with ethyl acetate (EtOAc) (3×120 mL), the combined organic layers were washed with water (80 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254/220 nm) to provide 5-iodo-1-[(4-methoxyphenyl)methyl]-3,3-dimethylindol-2-one (2 g, 56% yield). LCMS:(ES, m/z): RT=0.857 min, m/z=408.0[M+H]⁺.

Step 3: Into a 40 mL sealed tube was added 5-iodo-1-[(4-methoxyphenyl)methyl]-3,3-dimethylindol-2-one (2 g, 4.91 mmol, 1 equiv), methyl 2,2-difluoro-2-sulfoacetate (1.89 g, 9.82 mmol, 2 equiv), CuI (2.81 g, 14.7 mmol, 3 equiv) and N,N-dimethyl formamide (DMF) (15 mL) at room temperature. The resulting mixture was stirred for 2 h at 100° C. under nitrogen atmosphere, then quenched with water (50 mL) at room temperature. The resulting mixture was extracted with ethyl acetate (EtOAc) (3×100 mL), and the combined organic layers were washed with water (50 mL), and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254/220 nm) to provide 1-[(4-methoxyphenyl)methyl]-3,3-dimethyl-5-(trifluoromethyl)indol-2-one (1 g, 58% yield). LCMS:(ES, m/z): RT=0.820 min, m/z=350.1[M+H]⁺.

Step 4: Into a 40 mL sealed tube was added 1-[(4-methoxyphenyl)methyl]-3,3-dimethyl-5-(trifluoromethyl)indol-2-one (1 g, 2.86 mmol, 1 equiv), trifluoroacetic acid (TFA) (9 mL) and trifluoromethanesulfonic acid (3 mL) at room temperature. The resulting mixture was stirred overnight at 50° C., then quenched with water (50 mL) at room temperature. The resulting mixture was extracted with dichloromethane (DCM) (3×100 mL), and the combined organic layers were washed with water (50 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254/220 nm) to provide 3,3-dimethyl-5-(trifluoromethyl)indolin-2-one (Intermediate 11) (520.0 mg, 79% yield). LCMS:(ES, m/z): RT=0.675 min, m/z=230.1[M+H]⁺.

Step 5: Into a 40 mL sealed tube was added Intermediate 11 (520.0 mg, 2.26 mmol, 1 equiv), acetic acid (AcOH) (8 mL) and N-iodosuccinimide (NIS) (1.02 g, 4.53 mmol, 2 equiv) at room temperature. The resulting mixture was stirred for 2 h at 80° C. under nitrogen atmosphere, then the reaction was quenched with Na₂S₂O₃(aq) (50 mL) at room temperature. The resulting mixture was extracted with ethyl acetate (EtOAc) (3×60 mL), and the combined organic layers were washed with water (50 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254/220 nm) to provide 7-iodo-3,3-dimethyl-5-(trifluoromethyl)indolin-2-one (Intermediate 17) (420.0 mg, 52% yield). LCMS:(ES, m/z): RT=0.843 min, m/z=356.1[M+H]⁺.

### Intermediate Example 9. 5-chloro-4,7-difluoro-3,3-dimethylindolin-2-one (Intermediate 23)

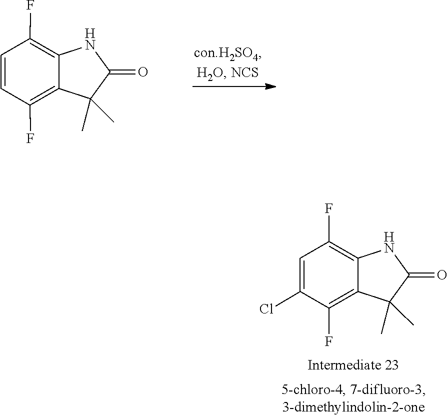

Into a 20 mL vial was added 4,7-difluoro-3,3-dimethyl-1H-indol-2-one (product of Intermediate Example 3, step 2) (300 mg, 1.52 mmol, 1 equiv), con. H₂SO₄(3.0 mL), water (0.30 mL) and N-chlorosuccinimide (NCS) (607 mg, 4.56 mmol, 3 equiv) at room temperature. The resulting mixture was stirred for 4 h at room temperature, then diluted with water (20 mL) and extracted with ethyl acetate (EtOAc) (3×30 mL). The combined organic layers were washed with brine (20 mL), dried over anhydrous Na₂SO₄. After filtration, the filtrate was concentrated under reduced pressure to provide a residue, which was purified by silica gel column chromatography, eluting with petroleum ether/EtOAc (1:1), to afford 5-chloro-4,7-difluoro-3,3-dimethyl-1H-indol-2-one (Intermediate 23) (300 mg, 85% yield). LCMS (LCMS, ESI): RT=0.72 min, m/z=232.1[M+H]⁺;¹H NMR (400 MHz, DMSO-d₆) δ 11.23 (s, 1H), 7.55 (dd, J=9.2 Hz, 1H), 1.36 (d, J=6.4 Hz, 6H).

### Intermediate Example 10. 4,7-difluoro-3,3-dimethyl-5-(trifluoromethyl)indolin-2-one (Intermediate 9) and 4,7-difluoro-5-iodo-3,3-dimethylindolin-2-one (Intermediate 24)

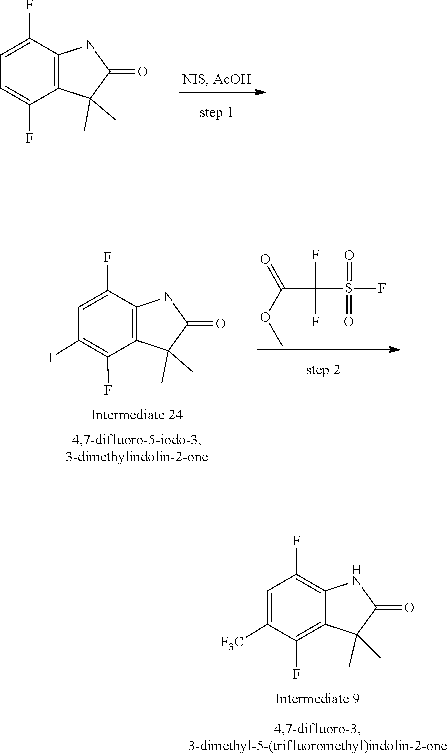

Step 1: Into an 8 mL vial was added 4,7-difluoro-3,3-dimethyl-1H-indol-2-one (product of Intermediate Example 3, step 2) (360.00 mg, 1.82 mmol, 1 equiv), N-iodosuccinimide (NIS) (821.51 mg, 3.65 mmol, 2 equiv) and acetic acid (AcOH) (4 mL) at room temperature. The resulting mixture was stirred for 16 h at 60° C., then quenched by the addition of water (20 mL) at room temperature. The resulting mixture was extracted with CH₂Cl₂ (3×20 mL), and the combined organic layers were dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254/220 nm) to provide 4,7-difluoro-5-iodo-3,3-dimethylindolin-2-one (Intermediate 24) (350 mg, 59% yield). LCMS:(ES, m/z): RT=0.775 min, m/z=324.1[M+H]⁺.

Step 2: Into a 20 mL vial were added Intermediate 24 (350 mg, 1.08 mmol, 1 equiv), CuI (1.03 g, 5.41 mmol, 5 equiv), DMF (3 mL) and methyl 2,2-difluoro-2-sulfoacetate (1.04 g, 5.41 mmol, 5 equiv) at room temperature. The resulting mixture was stirred for 16 h at 100° C. under nitrogen atmosphere, then quenched by the addition of water (20 mL) at room temperature. The resulting mixture was extracted with CH₂Cl₂ (3×20 mL), and the combined organic layers were dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254/220 nm) to provide 4,7-difluoro-3,3-dimethyl-5-(trifluoromethyl)-1H-indol-2-one (Intermediate 9) (140 mg, 49% yield). LCMS:(ES, m/z): RT=0.810 min, m/z=266.2[M+H]⁺.

Additional intermediates may be prepared as provided in below Table A.

TABLE A
Additional Intermediates

| Intermediate | Procedure |
|---|---|
| 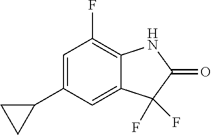 [SMILES: O=C1Nc2c(F)cc(C3CC3)cc2C1(F)F] | May be prepared by protecting Intermediate 6 with a Boc protecting group (e.g., using ditertbutyldicarbonate (Boc₂O) and triethylamine), followed by cross-coupling with cyclopropylboronic acid to provide the corresponding cyclopropyl material, followed by Boc deprotection (e.g., using TFA/DCM). |
| Intermediate 10 | |
| 5-cyclopropyl-3,3,7- | |
| trifluoroindolin-2-one | |
| 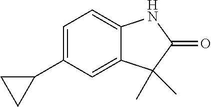 | May be prepared by protecting commercially available 5-bromo- 3,3-dimethylindolin-2-one with a Boc protecting group (e.g., using ditertbutyldicarbonate (Boc₂O) and triethylamine), followed by cross-coupling with cyclopropylboronic acid to provide the corresponding cyclopropyl material, followed by Boc deprotection (e.g., using TFA/DCM). |
| Intermediate 12 | |
| 5-cyclopropyl-3,3- | |
| dimethylindolin-2-one | |
| 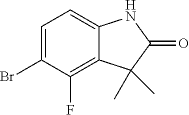 | May be prepared according to Fensome et al., J. Med. Chem (2008) 1861-1873 (Compound 36a). |
| Intermediate 13 | |
| 5-bromo-4-fluoro-3,3- | |
| dimethylindolin-2-one | |
| 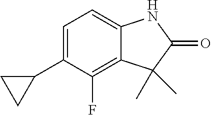 [SMILES: CC1(C)C(=O)Nc2ccc(C3CC3)c(F)c21] | May be prepared by protecting Intermediate 13 with a Boc protecting group (e.g., using ditertbutyldicarbonate (Boc₂O) and triethylamine), followed by cross-coupling with cyclopropylboronic acid to provide the corresponding cyclopropyl material, followed by Boc deprotection (e.g., using TFA/DCM). |
| Intermediate 14 | |
| 5-cyclopropyl-4-fluoro-3,3- | |
| dimethylindolin-2-one | |
| 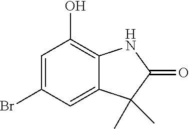 | May be prepared by protecting Intermediate 7 with a Boc protecting group (e.g., using ditertbutyldicarbonate (Boc₂O) and triethylamine), followed by oxidation (Cu₂O, NaOH, pyridine, and H₂O) conditions to provide the corresponding hydroxylated material, followed by Boc deprotection (e.g., using TFA/DCM). |
| Intermediate 15 | |
| 5-bromo-7-hydroxy-3,3- | |
| dimethylindolin-2-one | |
| 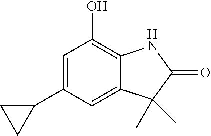 [SMILES: CC1(C)C(=O)Nc2c(O)cc(C3CC3)cc21] | May be prepared by protecting Intermediate 7 with a Boc protecting group (e.g., using ditertbutyldicarbonate (Boc₂O) and triethylamine), followed by oxidation (e.g., using Cu₂O, NaOH, pyridine, and H₂O) conditions to provide the corresponding hydroxylated material, followed by cross-coupling with cyclopropylboronic acid, then Boc deprotection (e.g., using TFA/DCM). |
| Intermediate 16 | |
| 5-cyclopropyl-7-hydroxy-3,3- | |
| dimethylindolin-2-one | |
| 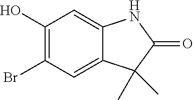 | May be prepared by deprotecting Intermediate 8 under demethylation conditions (e.g., using BBr₃/DCM) |
| Intermediate 20 | |
| 5-bromo-6-hydroxy-3,3- | |
| dimethylindolin-2-one | |
| 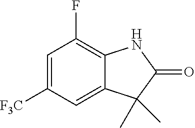 [SMILES: CC1(C)C(=O)Nc2c(F)cc(C(F)(F)F)cc21] | May be prepared by protecting Intermediate 1 with a Boc protecting group (e.g., using ditertbutyldicarbonate (Boc₂O) and triethylamine), followed by CF₃-coupling (e.g., using methyl 2,2- difluoro-2-(fluorosulfonyl)acetate, CuI, KF in DMF) to provide the corresponding trifluoromethyl material, followed by Boc deprotection (e.g., using TFA/DCM). |
| Intermediate 21 | |
| 7-fluoro-3,3-dimethyl-5- | |
| (trifluoromethyl)indolin-2-one | |
| 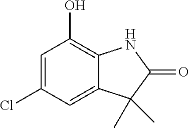 | May be prepared by protecting Intermediate 4 with a Boc protecting group (e.g., using ditertbutyldicarbonate (Boc₂O) and triethylamine), followed by oxidation (e.g., using Cu₂O, NaOH, pyridine, and H₂O) conditions to provide the corresponding hydroxylated material, followed by Boc deprotection (e.g., using TFA/DCM). |
| Intermediate 22 | |
| 5-chloro-7-hydroxy-3,3- | |
| dimethylindolin-2-one | |

### Final Products

### Example 1. 4-(3-(5-bromo-7-fluoro-3,3-dimethyl-2-oxoindolin-1-yl)-2-oxopyridin-1(2H)-yl)butanoic acid (Compound 1) and tert-butyl 4-(3-(5-bromo-7-fluoro-3,3-dimethyl-2-oxoindolin-1-yl)-2-oxopyridin-1(2H)-yl)butanoate (Compound 1-OtBu)

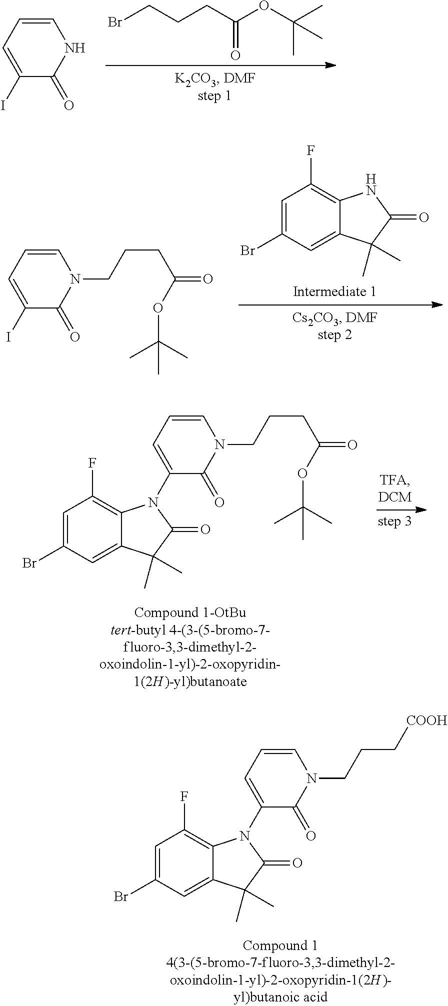

Step 1: Into an 8 mL vial was added 3-iodo-1H-pyridin-2-one (500.0 mg, 2.26 mmol, 1 equiv), K₂CO₃ (938.1 mg, 6.79 mmol, 3 equiv) and N,N-dimethyl formamide (DMF) (4 mL). The resulting mixture was stirred for 2 h at 80° C., then diluted with water (40 mL). The aqueous layer was extracted with ethyl acetate (EtOAc) (3×30 mL), and the organic layer concentrated under reduced pressure to provide a residue, which was purified by reverse flash chromatography (C18 silica gel; mobile phase, acetonitrile in water (0.1% TFA), 10% to 50% gradient in 10 min; detector, UV 254 nm) to provide tert-butyl 4-(3-iodo-2-oxopyridin-1-yl)butanoate (480.0 mg, 56% yield). LCMS: (ES, m/z): RT=1.02 min, m/z=308[M+H]⁺.

Step 2: Into a 20 mL vial was added tert-butyl 4-(3-iodo-2-oxopyridin-1-yl)butanoate (337.7 mg, 0.93 mmol, 1.20 equiv), 5-bromo-7-fluoro-3,3-dimethyl-1H-indol-2-one (Intermediate 1) 200 mg, 0.78 mmol, 1 equiv.), Cs₂CO₃ (757.5 mg, 2.32 mmol, 3 equiv) and N,N-dimethyl formamide (DMF) (5 mL, 0.12 mmol). The resulting mixture was stirred for 2 h at 120° C., and then the resulting mixture was diluted with water (60 mL). The aqueous layer was extracted with ethyl acetate (EtOAc) (3×50 mL) and the organic layer was concentrated under reduced pressure to provide a residue, which was purified by silica gel column chromatography, eluting with petroleum ether/ethyl acetate (4:1), to afford tert-butyl 4-(3-(5-bromo-7-fluoro-3,3-dimethyl-2-oxoindolin-1-yl)-2-oxopyridin-1(2H)-yl)butanoate (Compound 1-OtBu) (160.0 mg, 38% yield). LCMS: (ES, m/z): RT=1.039 min, m/z=438[M+H]⁺.

Step 3: Into an 8 mL vial was added Compound 1-OtBu (130.0 mg, 0.26 mmol, 1 equiv), trifluoroacetic acid (TFA) (0.60 mL) and dichloromethane (DCM) (3 mL). The resulting mixture was stirred for 2 h at room temperature, and then was concentrated under reduced pressure to provide a crude product, which was purified by Prep-HPLC (XBridge Prep OBD C18 Column, 30*150 mm, 5 m; Mobile Phase A: Water (10 mmol/L NH₄HCO₃), Mobile Phase B: acetonitrile; Flow rate: 60 mL/min; Gradient: 22% B to 30% B in 9 min, 30% B; Wave Length: 220 nm) to provide 4-(3-(5-bromo-7-fluoro-3,3-dimethyl-2-oxoindolin-1-yl)-2-oxopyridin-1(2H)-yl)butanoic acid (Compound 1) (72.2 mg, 63% yield).

Compound 1: LCMS: (ES, m/z): RT=1.22 min, m/z=438[M+H]⁺. ¹H NMR (400 MHz, DMSO-d₆) δ 7.81 (dd, J=6.8, 2.0 Hz, 1H), 7.67 (dd, J=7.1, 2.0 Hz, 1H), 7.58 (d, J=1.8 Hz, 1H), 7.39 (dd, J=10.8, 1.8 Hz, 1H), 6.37 (t, J=6.9 Hz, 1H), 4.04 (m, 1H), 3.88 (m, 1H), 2.17 (t, J=7.4 Hz, 2H), 1.85 (p, J=7.4 Hz, 2H), 1.41 (s, 3H), 1.37 (s, 3H).

### Example 2. 4-(3-(5-cyclopropyl-4,7-difluoro-3,3-dimethyl-2-oxoindolin-1-yl)-4-methyl-2-oxopyridin-1(2H)-yl)butanoic acid (Compound 4, rac-4), methyl 4-(3-(5-cyclopropyl-4,7-difluoro-3,3-dimethyl-2-oxoindolin-1-yl)-4-methyl-2-oxopyridin-1(2H)-yl)butanoate (Compound 4-OMe), (Sa)-4-(3-(5-cyclopropyl-4,7-difluoro-3,3-dimethyl-2-oxoindolin-1-yl)-4-methyl-2-oxopyridin-1(2H)-yl)butanoic acid (Compound 4A*), and (Ra)-4-(3-(5-cyclopropyl-4,7-difluoro-3,3-dimethyl-2-oxoindolin-1-yl)-4-methyl-2-oxopyridin-1(2H)-yl)butanoic acid (Compound 4B*)

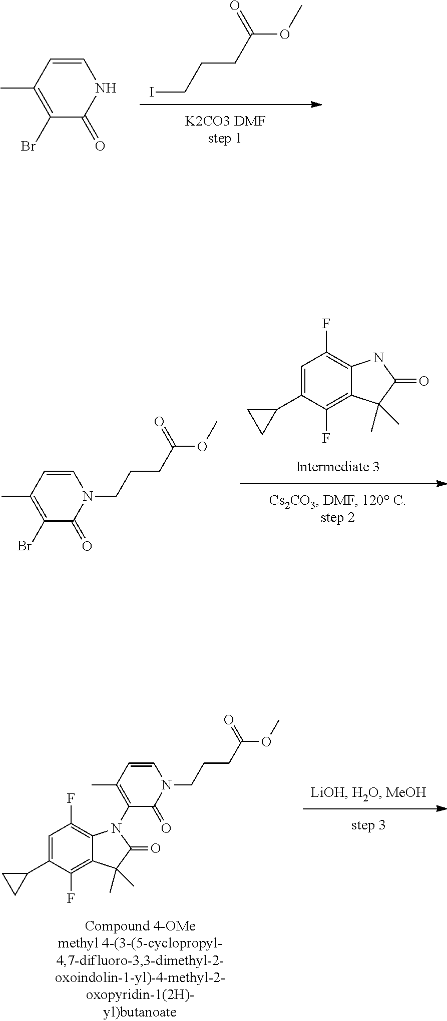

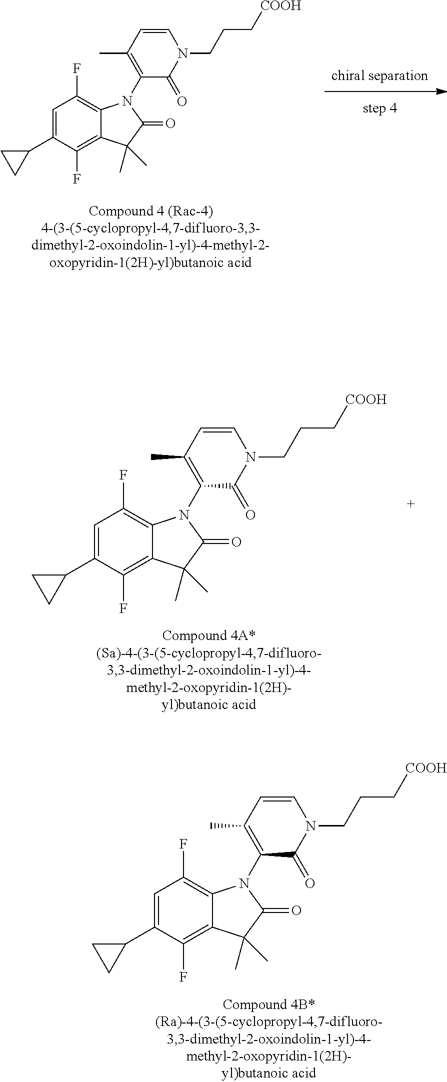

Step 1: Into a 20 mL vial was added 3-bromo-4-methyl-1,2-dihydropyridin-2-ol (400.0 mg, 2.11 mmol, 1 equiv), methyl 4-iodobutanoate (959.9 mg, 4.21 mmol, 2 equiv), K₂CO₃ (855.6 mg, 6.33 mmol, 3 equiv) and N,N-dimethyl formamide (DMF) (5 mL) at room temperature. The resulting mixture was stirred for 1 h at 100° C. under nitrogen atmosphere, then the reaction was quenched by the addition of water (100 mL) at room temperature. The resulting mixture was extracted with ethyl acetate (EtOAc) (3×80 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 30% to 60% gradient in 25 min; detector, UV 220 nm) to provide methyl 4-(3-bromo-4-methyl-2-oxopyridin-1-yl)butanoate (450 mg, 74% yield). LCMS (LCMS, ESI): RT=0.56 min, m/z=228.1 [M+H]⁺.

Step 2: Into an 8 mL sealed tube was added methyl 4-(3-bromo-4-methyl-2-oxopyridin-1-yl)butanoate (100.0 mg, 0.35 mmol, 1 equiv), 5-cyclopropyl-4,7-difluoro-3,3-dimethylindolin-2-one (Intermediate 3) (164.7 mg, 0.70 mmol, 2 equiv), Cs₂CO₃ (339.2 mg, 1.04 mmol, 3 equiv) and N,N-dimethyl formamide (DMF) (4 mL) at room temperature. The resulting mixture was stirred for 2 h at 120° C., then quenched by the addition of water (150 mL) at room temperature. The resulting mixture was extracted with ethyl acetate (EtOAc) (3×100 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 30% to 50% gradient in 20 min; detector, UV 254 nm/220 nm) to provide methyl 4-(3-(5-cyclopropyl-4,7-difluoro-3,3-dimethyl-2-oxoindolin-1-yl)-4-methyl-2-oxopyridin-1(2H)-yl)butanoate (Compound 4-OMe) (70 mg, 43% yield).

Step 3: Into an 8 mL vial was added Compound 4-OMe (60.0 mg, 0.14 mmol, 1 equiv), LiOH (16.2 mg, 0.66 mmol, 5 equiv), H₂O (2 mL) and MeOH (2 mL) at room temperature. The resulting mixture was stirred for 2 h at room temperature, then the pH of the mixture was adjusted to pH 4 with HCl (4M). The resulting mixture was extracted with ethyl acetate (EtOAc) (3×20 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 20% to 40% gradient in 20 min; detector, UV 254 nm/220 nm) to provide a crude product (50 mg), which was purified by Prep-HPLC (XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 25% B to 35% B in 10 min; Wave Length: 254 nm/220 nm; RT(min): 8.53) to provide 4-(3-(5-cyclopropyl-4,7-difluoro-3,3-dimethyl-2-oxoindolin-1-yl)-4-methyl-2-oxopyridin-1(2H)-yl)butanoic acid (Compound 4, rac-4) (32 mg, 54% yield) as a mixture of separable atropisomers.

LCMS (LCMS, ESI): RT=0.91 min, m/z=431.1 [M+H]⁺; ¹H NMR (400 MHz, Methanol-d4) δ 7.68 (d, J=7.2 Hz, 1H), 6.64 (dd, J=12.0, 5.6 Hz, 1H), 6.41 (d, J=7.2 Hz, 1H), 4.06 (dd, J=45.2, 13.2 Hz, 2H), 2.29 (t, J=7.2 Hz, 2H), 2.14 (s, 3H), 2.04 (d, J=14.4 Hz, 3H), 1.59 (d, J=10.4 Hz, 6H), 0.99 (d, J=9.6 Hz, 2H), 0.76-0.62 (m, 2H).

Step 4: Compound 4 mixture from step 3 (32 mg) was separated by Prep-Chiral HPLC (CHIRALPAK ID, 2*25 cm, 5 m; Mobile Phase A: Hexanes (0.1% TFA), Mobile Phase B: ethanol:dichloromethane=1:1; Flow rate: 20 mL/min; Gradient: 30% B to 30% B in 7.5 min; Wave Length: 220/254 nm); to afford (Sa)-4-(3-(5-cyclopropyl-4,7-difluoro-3,3-dimethyl-2-oxoindolin-1-yl)-4-methyl-2-oxopyridin-1(2H)-yl)butanoic acid (Compound 4A*) (6.2 mg, 19% yield; RT(min): 4.37) as the first eluting peak, and (Ra)-4-(3-(5-cyclopropyl-4,7-difluoro-3,3-dimethyl-2-oxoindolin-1-yl)-4-methyl-2-oxopyridin-1(2H)-yl)butanoic acid (Compound 4B*) (6.7 mg, 21% yield; RT(min): 5.77) as the second eluting peak. *Stereochemistry arbitrarily assigned.

Compound 4A*: LCMS (LCMS, ESI): RT=0.89 min, m/z=431.1 [M+H]⁺; H NMR (400 MHz, Methanol-d4) δ 7.67 (d, J=7.2 Hz, 1H), 6.64 (dd, J=12.0, 5.8 Hz, 1H), 6.41 (d, J=7.2 Hz, 1H), 4.12 (d, J=7.2 Hz, 1H), 4.01 (t, J=13.2 Hz, 1H), 2.28 (t, J=7.2 Hz, 2H), 2.14 (s, 3H), 2.10-1.96 (m, 3H), 1.59 (d, J=9.6 Hz, 6H), 1.03-0.96 (m, 2H), 0.68 (q, J=4.8 Hz, 2H).

Compound 4B*: LCMS (LCMS, ESI): RT=0.90 min, m/z=431.1 [M+H]⁺; H NMR (400 MHz, Methanol-d4) δ 7.68 (d, J=7.2 Hz, 1H), 6.63 (d, J=6.0 Hz, 2H), 2.14 (s, 3H), 2.03 (d, J=18.4 Hz, 3H), 1.59 (d, J=9.6 Hz, 6H), 1.05-0.92 (m, 2H), 0.72-0.61 (m, 2H).

### Example 3. 4-(3-(5-cyclopropyl-4,7-difluoro-3,3-dimethyl-2-oxoindolin-1-yl)-5-methyl-2-oxopyridin-1(2H)-yl)butanoic acid (Compound 5) and methyl 4-(3-(5-cyclopropyl-4,7-difluoro-3,3-dimethyl-2-oxoindolin-1-yl)-5-methyl-2-oxopyridin-1(2H)-yl)butanoate (Compound 5-OMe)

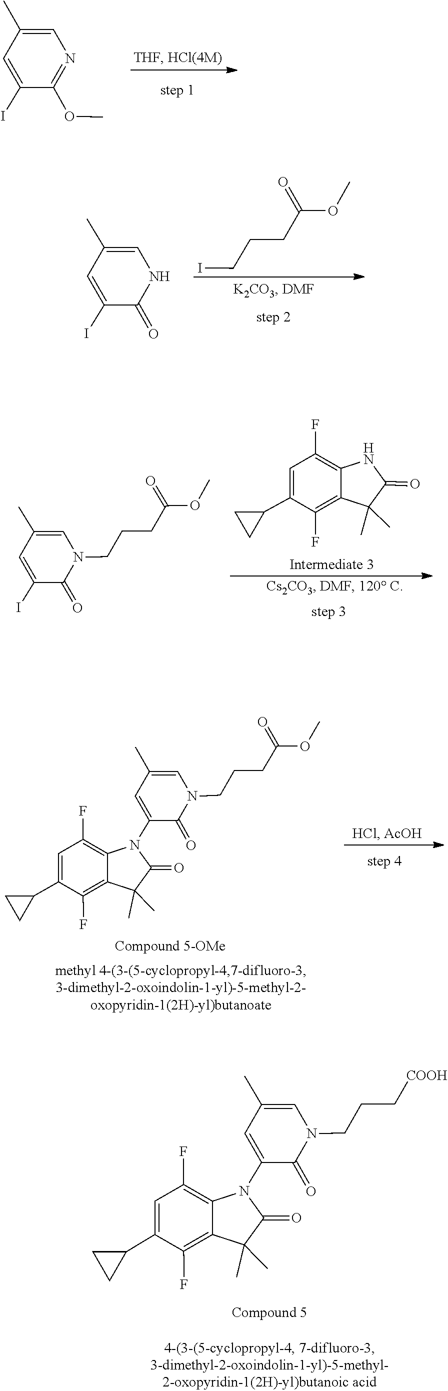

Step 1: Into an 8 mL vial was added 3-iodo-2-methoxy-5-methyl-1,2-dihydropyridine (200.0 mg, 0.79 mmol, 1 equiv), tetrahydrofuran (THF) (0.3 mL), and HCl (4M, 3 mL) at room temperature. The resulting mixture was stirred for 1 h at 90° C. under nitrogen atmosphere. The reaction was monitored by LCMS. The resulting mixture was concentrated under reduced pressure to provide 3-iodo-5-methyl-1H-pyridin-2-one (200 mg, 92% yield), which was used in the next step directly without purification. LCMS: (ES, m/z): RT=0.576 min, m/z=236.1[M+H]⁺.

Step 2: Into an 8 mL vial was added 3-iodo-5-methyl-1H-pyridin-2-one (200.0 mg, 0.85 mmol, 1 equiv), methyl 4-bromobutanoate (308.10 mg, 1.70 mmol, 2 equiv), K₂CO₃ (352.8 mg, 2.55 mmol, 3 equiv), and N,N-dimethyl formamide (DMF) (2 mL) at room temperature. The resulting mixture was stirred for 1 h at 90° C. under nitrogen atmosphere, then quenched by the addition of water (20 mL) at room temperature. The resulting mixture was extracted with ethyl acetate (EtOAc) (3×40 mL), and the combined organic layers were washed with water (3×20 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 20% to 30% gradient in 12 min; detector, UV 220 nm) to provide methyl 4-(3-iodo-5-methyl-2-oxopyridin-1-yl)butanoate (140 mg, 49% yield). LCMS: (ES, m/z): RT=0.65 min, m/z=336.1[M+H]⁺.

Step 3: A solution of methyl 4-(3-iodo-5-methyl-2-oxopyridin-1-yl)butanoate (100.0 mg, 0.29 mmol, 1 equiv), 5-cyclopropyl-4,7-difluoro-3,3-dimethylindolin-2-one (Intermediate 3) (70.8 mg, 0.29 mmol, 1 equiv), Cs₂CO₃ (291.7 mg, 0.89 mmol, 3 equiv) in N,N-dimethyl formamide (DMF) (3 mL) was stirred for 1 h at 120° C. under nitrogen atmosphere, then reaction was quenched with water (15 mL) at room temperature. The resulting mixture was extracted with ethyl acetate (EtOAc) (3×30 mL). The combined organic layers were washed with water (2×10 mL), dried over anhydrous Na₂SO₄. After filtration, the filtrate was concentrated under reduced pressure to provide a residue, which was purified by reversed-phase flash chromatography (C18 silica gel; mobile phase, acetonitrile in water, 40% to 50% gradient in 10 min; detector, UV 254 nm/UV 220 nm) to provide methyl 4-(3-(5-cyclopropyl-4,7-difluoro-3,3-dimethyl-2-oxoindolin-1-yl)-5-methyl-2-oxopyridin-1(2H)-yl)butanoate (Compound 5-OMe) (50 mg, 38% yield). LCMS: (ES, m/z): RT=0.94 min, m/z=445.2[M+H]⁺.

Step 4: A solution of Compound 5-OMe (40.0 mg, 0.09 mmol, 1 equiv) and acetic acid (AcOH) (0.5 mL) in HCl (6M, 0.5 mL) was stirred overnight at 40° C., then quenched with water (10 mL) at room temperature. The resulting mixture was extracted with ethyl acetate (EtOAc) (3×20 mL), and the combined organic layers were washed with water (2×10 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 35% to 45% gradient in 10 min; detector, UV 254 nm/UV 220 nm) to provide a crude product (20 mg), which was purified by Prep-HPLC (XBridge Prep OBD C18 Column, 30*150 mm, 5 m; Mobile Phase A: water (10 mmol/L NH₄HCO₃), Mobile Phase B: acetonitrile; Flow rate: 60 mL/min; Gradient: 25% B to 35% B in 10 min; Wave Length: 254 nm/220 nm; RT(min): 8.98) to afford 4-(3-(5-cyclopropyl-4,7-difluoro-3,3-dimethyl-2-oxoindolin-1-yl)-5-methyl-2-oxopyridin-1(2H)-yl)butanoic acid (Compound 5) (14.4 mg, 37% yield).

Compound 5: LCMS: (ES, m/z): RT=1.497 min, m/z=431.1[M+H]⁺. ¹H NMR (400 MHz, Methanol-d4) δ 7.63-7.56 (m, 2H), 6.63 (d, J=11.8 Hz, 1H), 4.14 (d, J=12.8 Hz, 1H), 4.01 (d, J=13.4 Hz, 1H), 2.29 (t, J=7.2 Hz, 2H), 2.21 (s, 3H), 2.04 (q, J=7.6 Hz, 3H), 1.58 (d, J=4.6 Hz, 6H), 1.00 (d, J=9.8 Hz, 2H), 0.68 (d, J=4.0 Hz, 2H).

### Example 4. 4-(3-(5-cyclopropyl-4,7-difluoro-3,3-dimethyl-2-oxoindolin-1-yl)-6-methyl-2-oxopyridin-1(2H)-yl)butanoic acid (Compound 6) and methyl 4-(3-(5-cyclopropyl-4,7-difluoro-3,3-dimethyl-2-oxoindolin-1-yl)-6-methyl-2-oxopyridin-1(2H)-yl)butanoate (Compound 6-OMe)

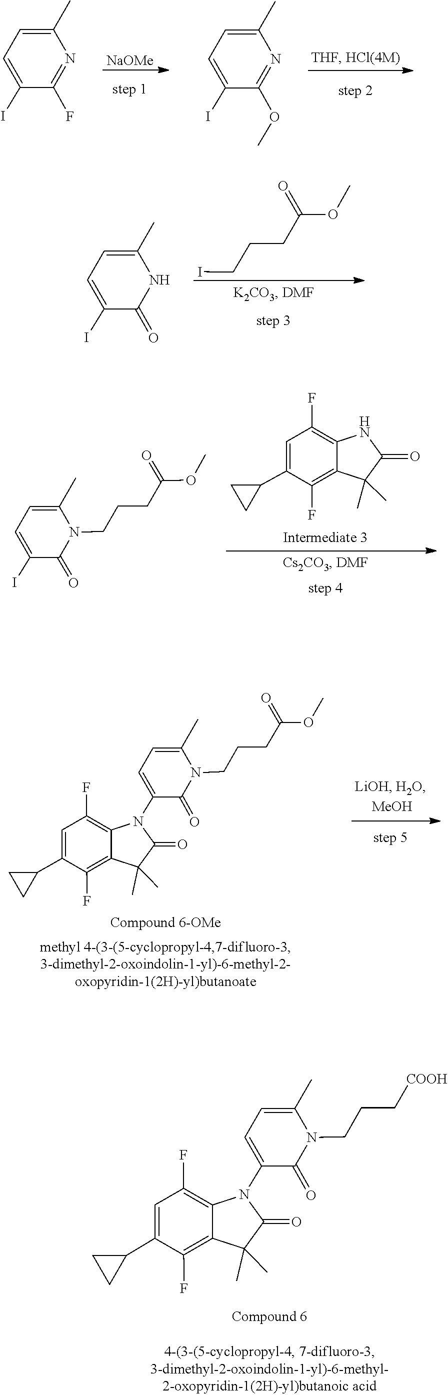

Step 1: Into a 40 mL vial was added 2-fluoro-3-iodo-6-methylpyridine (2 g, 8.43 mmol, 1 equiv) and sodium methoxide (60% Na in MeOH) (20 mL) at room temperature. The resulting mixture was stirred for 1 h at 80° C., then the reaction was quenched by the addition of water (100 mL) at room temperature. The resulting mixture was extracted with ethyl acetate (EtOAc) (3×200 mL), and the combined organic layers were washed with water (3×50 mL) and dried over anhydrous Na₂SO₄. After filtration, the filtrate was concentrated under reduced pressure to provide a residue, which was purified by silica gel column chromatography, eluting with petroleum ether/ethyl acetate (1:1), to afford 3-iodo-2-methoxy-6-methylpyridine (800.0 mg, 38% yield). LCMS: (ES, m/z): RT=1.09 min, m/z=250 [M+H]⁺.

Step 2: Into a 40 mL vial was added 3-iodo-6-methylpyridin-2-ol (700 mg, 2.97 mmol, 1 equiv), tetrahydrofuran (THF) (0.8 mL), and 4M HCl (8 mL) at room temperature. The resulting mixture was stirred for 1 h at 90° C. under nitrogen atmosphere, then quenched by the addition of water (100 mL) at room temperature. The resulting mixture was extracted with ethyl acetate (EtOAc) (3×140 mL) and the combined organic layers were washed with water (3×50 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 20% to 30% gradient in 13 min; detector, UV 220 nm) to provide 3-iodo-6-methyl-1H-pyridin-2-one (600.0 mg, 86% yield). LCMS: (ES, m/z): RT=0.60 min, m/z=236 [M+H]⁺.

Step 3: Into a 40 mL vial was added 3-iodo-6-methyl-1H-pyridin-2-one (550.0 mg, 2.34 mmol, 1 equiv), methyl 4-iodobutanoate (1067 mg, 4.68 mmol, 2 equiv), K₂CO₃ (970.3 mg, 7.02 mmol, 3 equiv), and N,N-dimethyl formamide (DMF) (6 mL) at room temperature. The resulting mixture was stirred for 1 h at 100° C. under nitrogen atmosphere, then quenched by the addition of water (100 mL) at room temperature. The resulting mixture was extracted with ethyl acetate (EtOAc) (3×100 mL). The combined organic layers were washed with water (3×50 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 10% to 20% gradient in 15 min; detector, UV 220 nm) to provide methyl 4-(3-iodo-6-methyl-2-oxopyridin-1-yl)butanoate (130.0 mg, 17% yield). LCMS: (ES, m/z): RT=0.71 min, m/z=336 [M+H]⁺.

Step 4: Into a 4 mL sealed tube was added methyl 4-(3-iodo-6-methyl-2-oxopyridin-1-yl)butanoate (120.0 mg, 0.35 mmol, 1 equiv), 5-cyclopropyl-4,7-difluoro-3,3-dimethylindolin-2-one (Intermediate 3) (42.5 mg, 0.17 mmol, 0.50 equiv), Cs₂CO₃ (349.99 mg, 1.07 mmol, 3 equiv), and N,N-dimethyl formamide (DMF) (1 mL) at room temperature. The reaction mixture was irradiated with microwave radiation for 2 h at 120° C., then quenched by the addition of water (20 mL) at room temperature. The resulting mixture was extracted with ethyl acetate (EtOAc) (3×20 mL), and the combined organic layers were washed with water (2×10 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 40% to 50% gradient in 12 min; detector, UV 220 nm) to provide methyl 4-(3-(5-cyclopropyl-4,7-difluoro-3,3-dimethyl-2-oxoindolin-1-yl)-6-methyl-2-oxopyridin-1(2H)-yl)butanoate (Compound 6-OMe) (40.0 mg, 25% yield). LCMS: (ES, m/z): RT=0.83 min, m/z=445 [M+H]⁺.

Step 5: Into an 8 mL vial was added Compound 6-OMe (35.0 mg, 0.08 mmol, 1 equiv), LiOH (5.7 mg, 0.24 mmol, 3 equiv), H₂O (0.3 mL), and methanol (MeOH) (0.3 mL) at room temperature. The resulting mixture was stirred for 1 h at room temperature, then the pH of the mixture was adjusted to pH 6 with 6M HCl (aq.). The resulting mixture was extracted with ethyl acetate (EtOAc) (3×15 mL), and the combined organic layers were washed with water (2×10 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 30% to 40% gradient in 11 min; detector, UV 220 nm) to provide a crude product (30 mg), which was purified by Prep-HPLC (Xselect CSH OBD Column 30*150 mm, 5 um; Mobile Phase A: water (0.05% TFA), Mobile Phase B: acetonitrile; Flow rate: 60 mL/min; Gradient: 5% B to 5% B in 2 min, 35% B to 45% B in 10 min; Wave Length: 254 nm/220 nm; RT(min): 9.4-10.4) to afford 4-(3-(5-cyclopropyl-4,7-difluoro-3,3-dimethyl-2-oxoindolin-1-yl)-6-methyl-2-oxopyridin-1(2H)-yl)butanoic acid (Compound 6) (4.2 mg, 12% yield).

Compound 6: LCMS: (ES, m/z): RT=0.78 min, m/z=431.2[M+H]⁺. ¹H NMR (400 MHz, DMSO-d6) δ 7.54 (d, J=7.4 Hz, 1H), 6.73 (d, J=11.8 Hz, 1H), 6.25 (d, J=7.4 Hz, 1H), 4.05 (d, J=15.2 Hz, 1H), 3.97-3.87 (m, 1H), 2.47 (s, 3H), 2.30 (t, J=7.2 Hz, 2H), 2.04 (d, J=29.3 Hz, 1H), 1.80 (q, J=7.4 Hz, 2H), 1.48 (s, 3H), 1.43 (s, 3H), 0.97-0.91 (m, 2H), 0.70 (s, 2H).

### Example 5. 4-(3-(5-chloro-7-hydroxy-3,3-dimethyl-2-oxoindolin-1-yl)-2-oxopyridin-1(2H)-yl)butanoic acid (Compound 7) and tert-butyl 4-(3-(5-chloro-7-iodo-3,3-dimethyl-2-oxoindolin-1-yl)-2-oxopyridin-1(2H)-yl)butanoate (Compound 7-OtBu)

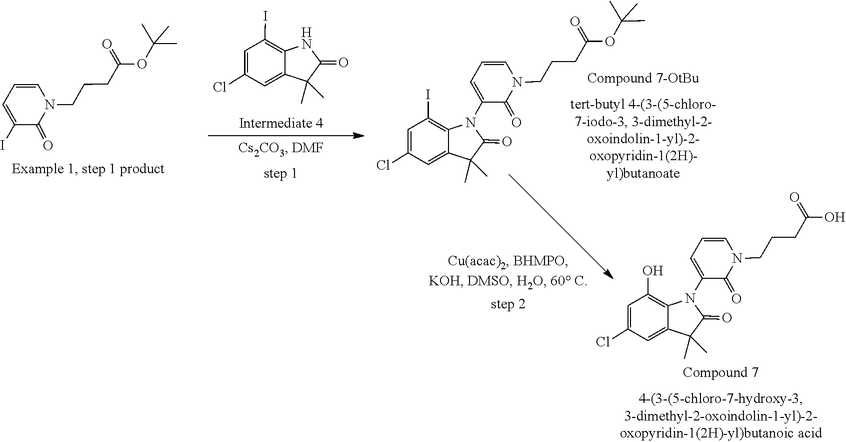 [SMILES: CC(C)(C)OC(=O)CCCn1cccc(I)c1=O ; CC1(C)C(=O)Nc2c(I)cc(Cl)cc21 ; CC(C)(C)OC(=O)CCCn1cccc(N2C(=O)C(C)(C)c3cc(Cl)cc(I)c32)c1=O ; CC1(C)C(=O)N(c2cccn(CCCC(=O)O)c2=O)c2c(O)cc(Cl)cc21]

Step 1: Into an 8 mL vial was added tert-butyl 4-(3-iodo-2-oxopyridin-1-yl)butanoate (product of Example 1, step 1) (2.03 g, 5.59 mmol, 2 equiv), 5-chloro-7-iodo-3,3-dimethylindolin-2-one (Intermediate 4) (900.0 mg, 2.79 mmol, 1 equiv), Cs₂CO₃ (2.74 g, 8.39 mmol, 3 equiv) and N,N-dimethyl formamide (DMF) (6 mL) at room temperature. The resulting mixture was stirred for 24 h at 120° C., then quenched with 20 mL of water at room temperature. The aqueous phase was extracted with dichloromethane (DCM) (3×100 mL), and the organic phase was washed with water (100 mL), then dried over Na₂SO₄, filtered, and concentrated to provide a residue, which was purified by reversed-phase flash chromatography (C18 silica gel; mobile phase, acetonitrile in water, 0% to 100% gradient in 40 min; detector, UV 254/220 nm) to provide tert-butyl 4-(3-(5-chloro-7-iodo-3,3-dimethyl-2-oxoindolin-1-yl)-2-oxopyridin-1(2H)-yl)butanoate (Compound 7-OtBu) (75.0 mg, 5% yield). LCMS: (ES, m/z): RT=0.965 min, m/z=557.1[M+H]⁺.

Step 2: Into an 8 mL sealed tube was added Compound 7-OtBu (75.0 mg, 0.14 mmol, 1 equiv) N,N-bis(4-hydroxy-2,6-dimethylphenyl)oxamide (BHMPO) (44.23 mg, 0.14 mmol, 1 equiv), copper(II) acetylacetonate (35.3 mg, 0.14 mmol, 1 equiv), dimethylsulfoxide (DMSO) (3 mL), H₂O (1 mL) and KOH (151.1 mg, 2.70 mmol, 20 equiv) at room temperature. The reaction was stirred for 24 h at 60° C., then the reaction was adjusted to pH=4 with HCl (1M), extracted with dichloromethane (DCM) (3×15 mL), the organic phase was dried over Na₂SO₄, then the organic phase was concentrated under reduce pressure, and the residue was purified by reversed-phase flash chromatography (C18 silica gel; mobile phase, acetonitrile in water (5 mmol NH₄HCO₃), 0% to 100% gradient in 30 min; detector, UV 254/220 nm) to provide a crude product (15 mg) which was further purified by Prep HPLC (Xselect CSH OBD Column 30*150 mm, 5 m; Mobile Phase A: water (0.05% TFA), Mobile Phase B: acetonitrile; Flow rate: 60 mL/min; Gradient: 5% B to 5% B in 2 min, 22% B to 32% B in 10 min; Wave Length: 254/220 nm; RT(min): 8.3) to provide 4-(3-(5-chloro-7-hydroxy-3,3-dimethyl-2-oxoindolin-1-yl)-2-oxopyridin-1(2H)-yl)butanoic acid (Compound 7) (5.90 mg, 11% yield).

Compound 7: LCMS: (ES, m/z): RT=0.698 min, m/z=391.1[M+H]⁺. ¹H NMR (400 MHz, DMSO-d₆) δ 12.16 (s, 1H), 9.66 (s, 1H), 7.78-7.62 (m, 1H), 7.50-7.40 (m, 1H), 6.99 (d, J=2.0 Hz, 1H), 6.66 (d, J=2.0 Hz, 1H), 6.29 (t, J=6.8 Hz, 1H), 4.06-3.90 (m, 1H), 3.88-3.77 (m, 1H), 2.24 (t, J=7.6 Hz, 2H), 1.87 (p, J=7.2 Hz, 2H), 1.33 (s, 6H).

### Example 6. 4-(5-(5-cyclopropyl-4,7-difluoro-3,3-dimethyl-2-oxoindolin-1-yl)-6-oxopyrimidin-1(6H)-yl)butanoic acid (Compound 8) and methyl 4-(5-(5-cyclopropyl-4,7-difluoro-3,3-dimethyl-2-oxoindolin-1-yl)-6-oxopyrimidin-1(6H)-yl)butanoate (Compound 8-OMe)

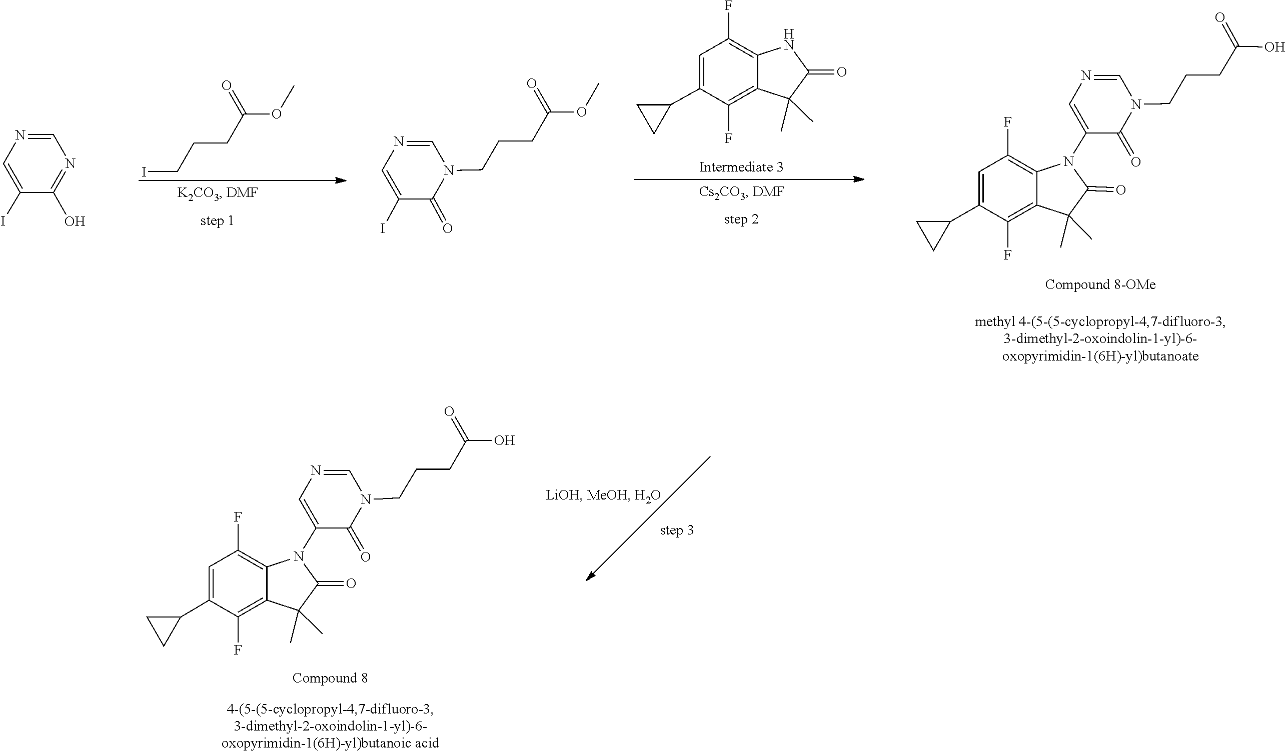

Step 1: A solution of 5-iodopyrimidin-4-ol (1 g, 4.50 mmol, 1 equiv), methyl 4-iodobutanoate (2.05 g, 9.01 mmol, 2 equiv), and K₂CO₃ (1.87 g, 13.5 mmol, 3 equiv) in N,N-dimethyl formamide (DMF) (10 mL) was stirred for 1 h at 100° C., then quenched by the addition of water (1 mL) at room temperature. The resulting mixture was concentrated under vacuum to provide a residue, which was purified by reversed-phase flash chromatography (C18 silica gel; mobile phase, acetonitrile in water, 15% to 25% gradient in 10 min; detector, UV 254/220 nm) to provide methyl 4-(5-iodo-6-oxopyrimidin-1-yl)butanoate (800 mg, 55% yield). LCMS: (ES, m/z): RT=0.57 min, m/z=323.1 [M+H]⁺.

Step 2: A solution of methyl 4-(5-iodo-6-oxopyrimidin-1-yl)butanoate (325.8 mg, 1.01 mmol, 3 equiv), 5-cyclopropyl-4,7-difluoro-3,3-dimethylindolin-2-one (Intermediate 3) (80.0 mg, 0.33 mmol, 1 equiv), and Cs₂CO₃ (329.6 mg, 1.01 mmol, 3 equiv) in N,N-dimethyl formamide (DMF) (1 mL) was stirred for 1 h at 120° C., then quenched with water (150 mL) at room temperature. The resulting mixture was extracted with ethyl acetate (EtOAc) (3×100 mL), and the combined organic layers were washed with water (3×30 mL), and dried over anhydrous Na₂SO₄. After filtration, the filtrate was concentrated under reduced pressure to provide a residue, which was purified by reversed-phase flash chromatography (C18 silica gel; mobile phase, acetonitrile in water, 40% to 50% gradient in 15 min; detector, UV 220 nm) to provide methyl 4-(5-(5-cyclopropyl-4,7-difluoro-3,3-dimethyl-2-oxoindolin-1-yl)-6-oxopyrimidin-1(6H)-yl)butanoate (Compound 8-OMe) (70.0 mg, 48% yield). LCMS: (ES, m/z): RT=0.85 min, m/z=432.1 [M+H]⁺.

Step 3: A solution of Compound 8-OMe (60.0 mg, 0.13 mmol, 1 equiv), LiOH (16.7 mg, 0.69 mmol, 5 equiv), H₂O (0.5 mL) in methanol (MeOH) (0.5 mL) was stirred for 1 h at 40° C., then the pH of the mixture adjusted to pH 5 with 6M HCl (aq.). The resulting mixture was extracted with ethyl acetate (EtOAc) (3×80 mL), and the combined organic layers were washed with water (2×20 mL), and dried over anhydrous Na₂SO₄. After filtration, the filtrate was concentrated under reduced pressure to provide a residue, which was purified by reversed-phase flash chromatography (C18 silica gel; mobile phase, acetonitrile in water, 20% to 30% gradient in 12 min; detector, UV 220 nm) to provide a crude product (34 mg), which was further purified by Prep-HPLC (Xselect CSH C18 OBD Column 30*150 mm 5 m; Mobile Phase A: water (0.05% TFA), Mobile Phase B: acetonitrile; Flow rate: 60 mL/min; Gradient: 5% B to 5% B in 2 min, 30% B to 40% B in 10 min; Wave Length: 254/220 nm; RT(min): 9.2) to afford 4-(5-(5-cyclopropyl-4,7-difluoro-3,3-dimethyl-2-oxoindolin-1-yl)-6-oxopyrimidin-1(6H)-yl)butanoic acid (Compound 8) (12.2 mg).

Compound 8: LCMS: (ES, m/z): RT=1.31 min, m/z=418.1 [M+H]⁺. ¹H NMR (400 MHz, DMSO-d6) δ 8.60 (s, 1H), 8.23 (s, 1H), 6.80 (d, J=11.8 Hz, 1H), 3.99 (d, J=47.8 Hz, 2H), 2.25 (t, J=7.4 Hz, 2H), 2.23-1.97 (m, 1H), 1.90 (t, J=7.2 Hz, 2H), 1.48 (d, J=22.6 Hz, 6H), 0.98-0.90 (m, 2H), 0.75-0.67 (m, 2H).

### Example 7. 4-(3-(5-cyclopropyl-4,7-difluoro-3,3-dimethyl-2-oxoindolin-1-yl)-2-oxoquinolin-1(2H)-yl)butanoic acid (Compound 9) and methyl 4-(3-(5-cyclopropyl-4,7-difluoro-3,3-dimethyl-2-oxoindolin-1-yl)-2-oxoquinolin-1(2H)-yl)butanoate (Compound 9-OMe) PP-3

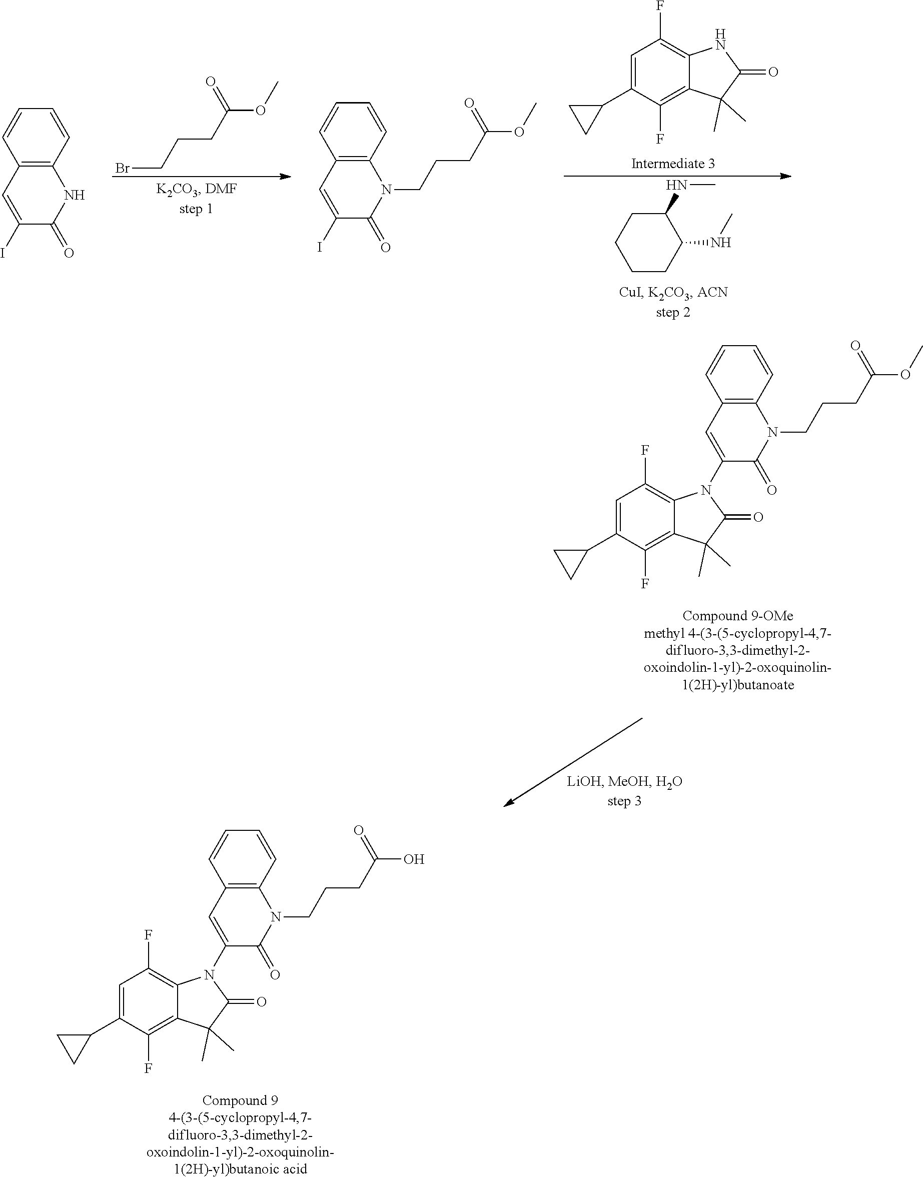

Step 1: A solution of 3-iodo-1H-quinolin-2-one (500.0 mg, 1.84 mmol, 1 equiv) and methyl 4-bromobutanoate (667.9 mg, 3.69 mmol, 2 equiv), K₂CO₃ (764.8 mg, 5.53 mmol, 3 equiv) in N,N-dimethyl formamide (DMF) (5 mL) was stirred for 1 h at 100° C., then quenched by the addition of water (1 mL) at room temperature. The resulting mixture was concentrated under vacuum to provide a residue, which was purified by reversed-phase flash chromatography (C18 silica gel; mobile phase, acetonitrile in water, 10% to 20% gradient in 12 min; detector, UV 220 nm) to provide methyl 4-(3-iodo-2-oxoquinolin-1-yl)butanoate (380.0 mg, 56% yield). LCMS: (ES, m/z): RT=0.79 min, m/z=372.1 [M+H]⁺.

Step 2: A solution of methyl 4-(3-iodo-2-oxoquinolin-1-yl)butanoate (156.5 mg, 0.42 mmol, 1 equiv), 5-cyclopropyl-4,7-difluoro-3,3-dimethylindolin-2-one (Intermediate 3) (100.0 mg, 0.42 mmol, 1 equiv), (1R,2R)—N1,N2-dimethylcyclohexane-1,2-diamine (18.0 mg, 0.12 mmol, 0.30 equiv), CuI (24.1 mg, 0.12 mmol, 0.30 equiv), and K₂CO₃ (174.8 mg, 1.26 mmol, 3 equiv) in acetonitrile (1.5 mL) was stirred for overnight at 80° C. under nitrogen atmosphere, then quenched by the addition of water (100 mL) at room temperature. The resulting mixture was extracted with ethyl acetate (EtOAc) (3×100 mL), and the combined organic layers were washed with water (2×30 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 60% to 70% gradient in 13 min; detector, UV 220 nm) to provide methyl 4-(3-(5-cyclopropyl-4,7-difluoro-3,3-dimethyl-2-oxoindolin-1-yl)-2-oxoquinolin-1(2H)-yl)butanoate (Compound 9-OMe) (26.0 mg, 13% yield). LCMS: (ES, m/z): RT=1.00 min, m/z=481.3 [M+H]⁺.

Step 3: A solution of Compound 9-OMe (20.0 mg, 0.04 mmol, 1 equiv), LiOH (4.98 mg, 0.20 mmol, 5 equiv), H₂O (0.5 mL) in MeOH (0.5 mL) was stirred for 1 h at 40° C., then the pH of the mixture was adjusted to pH 5 with 6M HCl (aq.). The resulting mixture was extracted with ethyl acetate (EtOAc) (3×50 mL), and the combined organic layers were washed with water (2×10 mL), and dried over anhydrous Na₂SO₄. After filtration, the filtrate was concentrated under reduced pressure to provide a crude product (12.0 mg), which was purified by Prep-HPLC (Xselect CSH C18 OBD Column 30*150 mm 5 m; Mobile Phase A: Water (0.05% TFA), Mobile Phase B: acetonitrile; Flow rate: 60 mL/min; Gradient: 5% B to 5% B in 2 min, 6% B to 16% B in 10 min; Wave Length: 254/220 nm; RT(min): 9.2) to afford 4-(3-(5-cyclopropyl-4,7-difluoro-3,3-dimethyl-2-oxoindolin-1-yl)-2-oxoquinolin-1(2H)-yl)butanoic acid (Compound 9) (5.60 mg).

Compound 9: LCMS: (ES, m/z): RT=1.21 min, m/z=467.1 [M+H]⁺. ¹H NMR (400 MHz, DMSO-d6) δ 8.27 (s, 1H), 7.86 (d, J=7.8 Hz, 1H), 7.75 (d, J=7.6 Hz, 2H), 7.38 (t, J=7.4 Hz, 1H), 6.79-6.66 (m, 1H), 4.31 (d, J=7.0 Hz, 2H), 2.36 (q, J=7.4 Hz, 2H), 2.15-1.92 (m, 1H), 1.85 (s, 2H), 1.51 (d, J=21.4 Hz, 6H), 0.96 (s, 2H), 0.73 (s, 2H).

### Example 8. 4-(3-(5-cyclopropyl-4,7-difluoro-3,3-dimethyl-2-oxoindolin-1-yl)-2-oxopyrazin-1(2H)-yl)butanoic acid (Compound 10) and tert-butyl 4-(3-(5-cyclopropyl-4,7-difluoro-3,3-dimethyl-2-oxoindolin-1-yl)-2-oxopyrazin-1(2H)-yl)butanoate (Compound 10-OtBu)

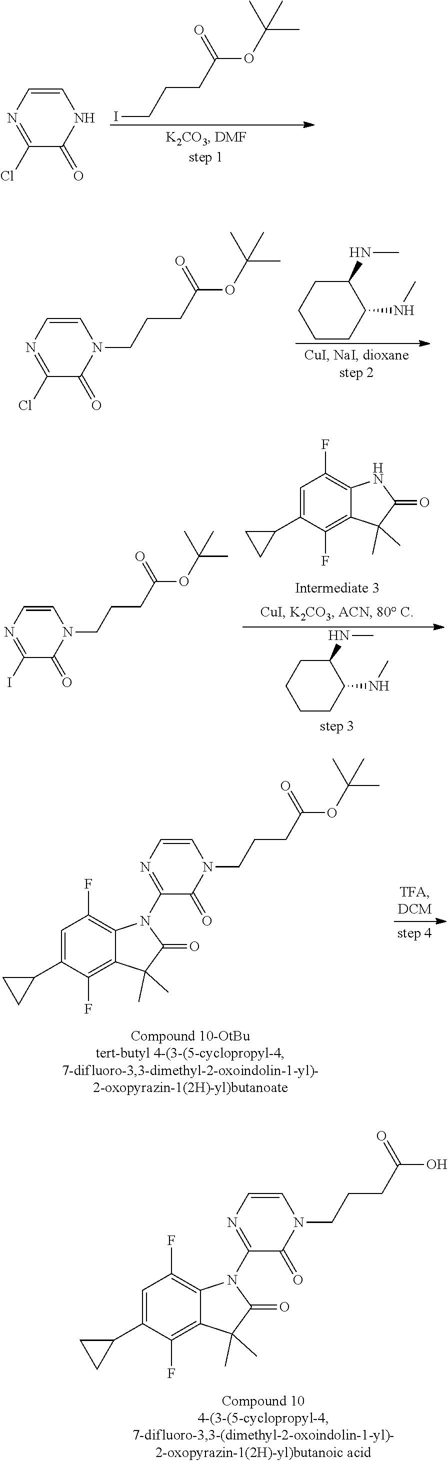

Step 1: A solution of bis(3-chloro-1H-pyrazin-2-one) (1.70 g, 6.51 mmol, 1 equiv), tert-butyl 4-bromobutanoate (2.91 g, 13.0 mmol, 2 equiv), and K₂CO₃ (2.70 g, 19.5 mmol, 3 equiv) in N,N-dimethyl formamide (DMF) (20 mL) was stirred for 1 h at 100° C., then reaction was quenched with water (800 mL) at room temperature. The resulting mixture was extracted with ethyl acetate (EtOAc) (3×800 mL), and the combined organic layers were washed with water (3×100 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 30% to 40% gradient in 15 min; detector, UV 220 nm) to provide tert-butyl 4-(3-chloro-2-oxopyrazin-1(2H)-yl)butanoate (2 g, 56% yield). LCMS: (ES, m/z): RT=0.72 min, m/z=217.1 [M+H]⁺.

Step 2: A solution of tert-butyl 4-(3-chloro-2-oxopyrazin-1-yl)butanoate (500.0 mg, 1.83 mmol, 1 equiv), (1R,2R)—N1,N2-dimethylcyclohexane-1,2-diamine (782.4 mg, 5.49 mmol, 3 equiv), NaI (549.6 mg, 3.66 mmol, 2 equiv), and CuI (698.3 mg, 3.66 mmol, 2 equiv) in dioxane (5 mL) was stirred for 2 h at 100° C. under nitrogen atmosphere, then quenched by the addition of water (200 mL) at room temperature. The resulting mixture was extracted with ethyl acetate (EtOAc) (3×200 mL), and the combined organic layers were washed with water (2×50 mL) and dried over anhydrous Na₂SO₄. After filtration, the filtrate was concentrated under reduced pressure and the residue was purified by reversed-phase flash chromatography (C18 silica gel; mobile phase, acetonitrile in water, 40% to 50% gradient in 12 min; detector, UV 220 nm) to provide tert-butyl 4-(3-iodo-2-oxopyrazin-1-yl)butanoate (260.0 mg, 39% yield). LCMS: (ES, m/z): RT=0.69 min, m/z=309.1 [M+H]⁺.

Step 3: A solution of tert-butyl 4-(3-iodo-2-oxopyrazin-1-yl)butanoate (214.9 mg, 0.59 mmol, 2 equiv), 5-cyclopropyl-4,7-difluoro-3,3-dimethylindolin-2-one (Intermediate 3) (70.0 mg, 0.29 mmol, 1 equiv), (1R,2R)—N1,N2-dimethylcyclohexane-1,2-diamine (12.6 mg, 0.08 mmol, 0.30 equiv), CuI (16.9 mg, 0.08 mmol, 0.30 equiv), and K₂CO₃ (122.3 mg, 0.88 mmol, 3 equiv) in acetonitrile (1 mL) was stirred for 2 h at 80° C. under nitrogen atmosphere, then quenched by the addition of water (50 mL) at room temperature. The resulting mixture was extracted with ethyl acetate (EtOAc) (3×50 mL), and the combined organic layers were washed with water (2×10 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 50% to 60% gradient in 13 min; detector, UV 220 nm) to provide tert-butyl 4-(3-(5-cyclopropyl-4,7-difluoro-3,3-dimethyl-2-oxoindolin-1-yl)-2-oxopyrazin-1(2H)-yl)butanoate (Compound 10-OtBu) (46.0 mg, 33% yield). LCMS: (ES, m/z): RT=0.94 min, m/z=418.2 [M+H]⁺.

Step 4: A solution of Compound 10-OtBu (40.0 mg, 0.08 mmol, 1 equiv) and trifluoroacetic acid (TFA) (0.5 mL) in dichloromethane (DCM) (0.5 mL) was stirred for 1 h at room temperature. The reaction was monitored by LCMS. The resulting mixture was concentrated under vacuum. The crude product (35 mg) was purified by Prep-HPLC (Xselect CSH C18 OBD Column 30*150 mm, 5 wm; Mobile Phase A: Water (0.1% FA), Mobile Phase B: acetonitrile; Flow rate: 25 mL/min; Gradient: 38% B to 48% B in 10 min; Wave Length: 254/220 nm; RT(min): 12) to afford 4-(3-(5-cyclopropyl-4,7-difluoro-3,3-dimethyl-2-oxoindolin-1-yl)-2-oxopyrazin-1(2H)-yl)butanoic acid (Compound 10) (18.1 mg).

Compound 10: LCMS: (ES, m/z): RT=0.66 min, m/z=418.1 [M+H]⁺. ¹H NMR (400 MHz, DMSO-d6) δ 12.17 (s, 1H), 7.91 (d, J=4.2 Hz, 1H), 7.40 (d, J=4.2 Hz, 1H), 6.81 (d, J=11.8 Hz, 1H), 4.11-3.77 (m, 1H), 2.25 (t, J=7.4 Hz, 2H), 2.02 (d, J=4.4 Hz, 1H), 1.89 (d, J=7.4 Hz, 2H), 1.49 (s, 6H), 0.96 (d, J=8.2 Hz, 2H), 0.79-0.63 (m, 2H).

### Example 9. 4-(5-(5-cyclopropyl-4,7-difluoro-3,3-dimethyl-2-oxoindolin-1-yl)-6-oxopyridazin-1(6H)-yl)butanoic acid (Compound 11) and tert-butyl 4-(5-(5-cyclopropyl-4,7-difluoro-3,3-dimethyl-2-oxoindolin-1-yl)-6-oxopyridazin-1(6H)-yl)butanoate (Compound 11-OtBu)

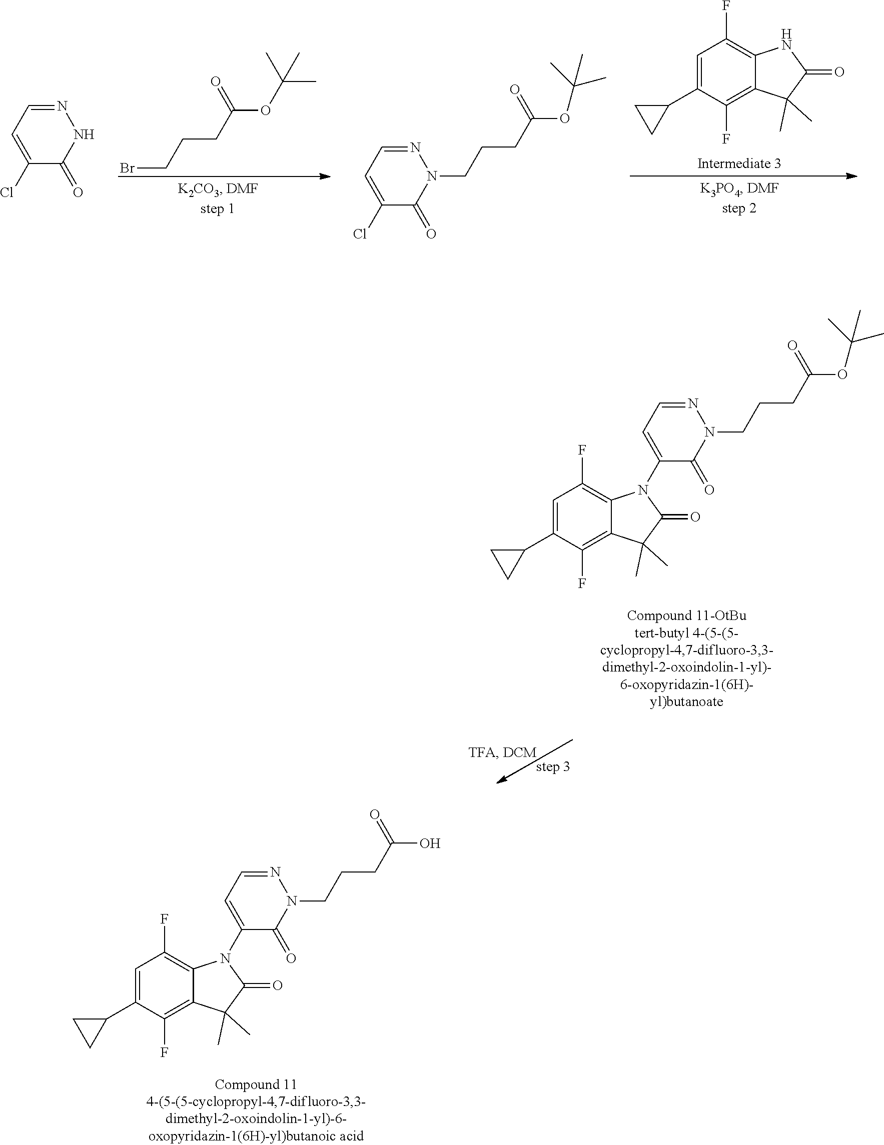

Step 1: A solution of 4-chloro-2H-pyridazin-3-one (1 g, 7.66 mmol, 1 equiv), tert-butyl 4-bromobutanoate (3.41 g, 15.4 mmol, 2 equiv), and K₂CO₃ (3.17 g, 223.0 mmol, 3 equiv) in N,N-dimethyl formamide (DMF) (10 mL) was stirred for 1 h at 100° C., then quenched by the addition of water (300 mL) at room temperature. The resulting mixture was extracted with ethyl acetate (EtOAc) (3×300 mL), and the combined organic layers were washed with water (2×100 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 40% to 50% gradient in 10 min; detector, UV 220 nm) to provide tert-butyl 4-(5-chloro-6-oxopyridazin-1-yl)butanoate (950 mg, 46% yield). LCMS: (ES, m/z): RT=0.79 min, m/z=372.1 [M+H]⁺.

Step 2: A solution of tert-butyl 4-(5-chloro-6-oxopyridazin-1-yl)butanoate (517.3 mg, 1.89 mmol, 3 equiv), 5-cyclopropyl-4,7-difluoro-3,3-dimethylindolin-2-one (Intermediate 3) (150.0 mg, 0.63 mmol, 1 equiv), and K₃PO₄ (402.6 mg, 1.89 mmol, 3 equiv) in N,N-dimethyl formamide (DMF) (1.5 mL) was stirred for overnight at 120° C., then the reaction was quenched by the addition of water (100 mL) at room temperature. The resulting mixture was extracted with ethyl acetate (EtOAc) (3×100 mL), and the combined organic layers were washed with water (2×30 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 50% to 60% gradient in 15 min; detector, UV 220 nm) to provide tert-butyl 4-(5-(5-cyclopropyl-4,7-difluoro-3,3-dimethyl-2-oxoindolin-1-yl)-6-oxopyridazin-1(6H)-yl)butanoate (Compound 11-OtBu) (25.0 mg, 8% yield). LCMS: (ES, m/z): RT=1.00 min, m/z=481.3 [M+H]⁺.

Step 3: A solution of Compound 11-OtBu (20.0 mg, 0.04 mmol, 1 equiv) and trifluoroacetic acid (TFA) (0.5 mL) in dichloromethane (DCM) (0.5 mL) was stirred for 1 h at room temperature, then the resulting mixture was concentrated under vacuum to provide a residue, which was purified by reversed-phase flash chromatography (C18 silica gel; mobile phase, acetonitrile in water, 40% to 50% gradient in 12 min; detector, UV 220 nm) to provide a crude product (15 mg), which was further purified by Prep-HPLC (XSelect CSH F-phenyl OBD Column 19*250 mm, 5 m; Mobile Phase A: water (0.05% TFA), Mobile Phase B: acetonitrile; Flow rate: 25 mL/min; Gradient: 28% B to 38% B in 10 min; Wave Length: 254/220 nm; RT(min): 15) to provide 4-(5-(5-cyclopropyl-4,7-difluoro-3,3-dimethyl-2-oxoindolin-1-yl)-6-oxopyridazin-1(6H)-yl)butanoic acid (Compound 11) (5.2 mg).

Compound 11: LCMS: (ES, m/z): RT=0.68 min, m/z=418.1 [M+H]⁺. ¹H NMR (400 MHz, DMSO-d6) δ 12.13 (s, 1H), 8.12 (d, J=4.2 Hz, 1H), 7.68 (d, J=4.2 Hz, 1H), 6.81 (d, J=11.8 Hz, 1H), 4.23 (d, J=13.6 Hz, 1H), 4.08-3.97 (m, 1H), 2.35-2.19 (m, 2H), 2.04-1.80 (m, 3H), 1.52 (s, 3H), 1.46 (s, 3H), 1.00-0.93 (m, 2H), 0.73 (d, J=5.6 Hz, 2H).

### Example 10. 4-(3-(5-cyclopropyl-4,7-difluoro-3,3-dimethyl-2-oxoindolin-1-yl)-2-oxopyrrolidin-1-yl)butanoic acid (Compound 16, rac-16), tert-butyl 4-(3-(5-cyclopropyl-4,7-difluoro-3,3-dimethyl-2-oxoindolin-1-yl)-2-oxopyrrolidin-1-yl)butanoate (Compound 16-OtBu), tert-butyl (S)-4-(3-(5-cyclopropyl-4,7-difluoro-3,3-dimethyl-2-oxoindolin-1-yl)-2-oxopyrrolidin-1-yl)butanoate (Compound 16B-OtBu), tert-butyl (R)-4-(3-(5-cyclopropyl-4,7-difluoro-3,3-dimethyl-2-oxoindolin-1-yl)-2-oxopyrrolidin-1-yl)butanoate (Compound 16A-OtBu), (S)-4-(3-(5-cyclopropyl-4,7-difluoro-3,3-dimethyl-2-oxoindolin-1-yl)-2-oxopyrrolidin-1-yl)butanoic acid (Compound 16B), and (R)-4-(3-(5-cyclopropyl-4,7-difluoro-3,3-dimethyl-2-oxoindolin-1-yl)-2-oxopyrrolidin-1-yl)butanoic acid (Compound 16A)

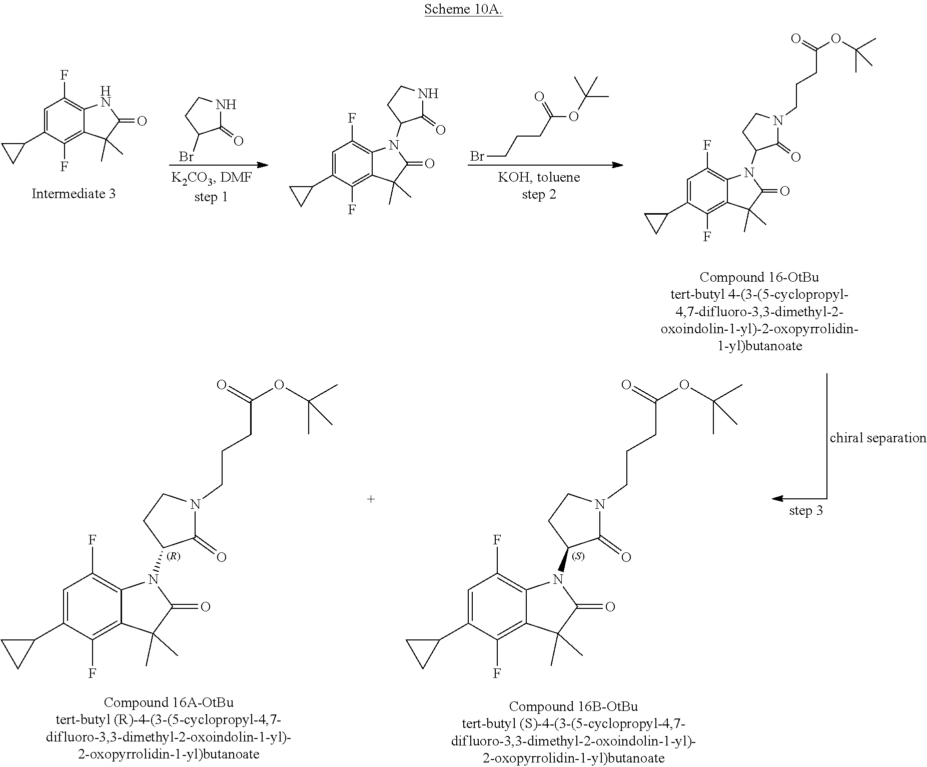

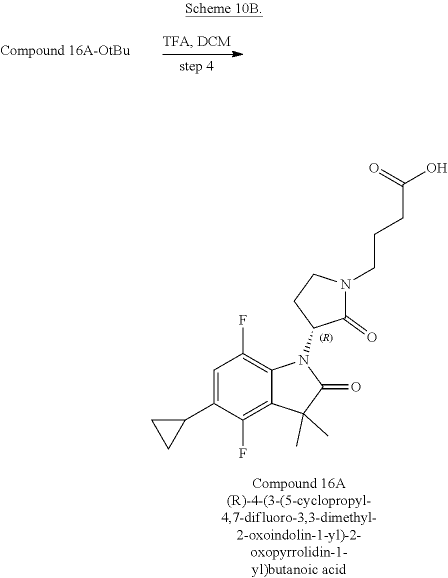

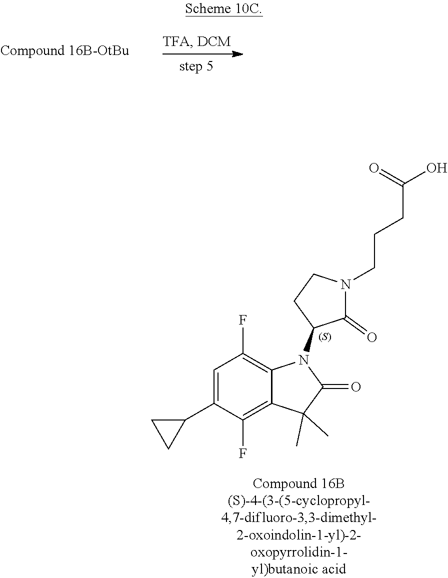

Step 1: A solution of 5-cyclopropyl-4,7-difluoro-3,3-dimethylindolin-2-one (Intermediate 3) (100.0 mg, 0.42 mmol, 1 equiv), 3-bromopyrrolidin-2-one (76.0 mg, 0.46 mmol, 1.10 equiv) and K₂CO₃ (174.8 mg, 1.26 mmol, 3 equiv) in N,N-dimethyl formamide (DMF) (1 mL) was stirred for 1 h at 80° C. under nitrogen atmosphere, then quenched by the addition of water (5 mL) at room temperature. The resulting mixture was extracted with ethyl acetate (EtOAc) (3×20 mL), and the combined organic layers were washed with water (1×5 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 30% to 40% gradient in 10 min; detector, UV 220/254 nm) to provide 5-cyclopropyl-4,7-difluoro-3,3-dimethyl-1-(2-oxopyrrolidin-3-yl)indol-2-one (80 mg, 49% yield). LCMS: (ES, m/z): RT=0.714 min, m/z=321.1[M+H]⁺.

Step 2: A solution of 5-cyclopropyl-4,7-difluoro-3,3-dimethyl-1-(2-oxopyrrolidin-3-yl)indol-2-one (50.0 mg, 0.15 mmol, 1 equiv), tert-butyl 4-bromobutanoate (41.8 mg, 0.18 mmol, 1.20 equiv), and KOH (26.3 mg, 0.46 mmol, 3 equiv) in toluene (1.5 mL) was stirred for 2 h at 100° C., then quenched by the addition of water (5 mL) at room temperature. The resulting mixture was extracted with ethyl acetate (EtOAc) (3×20 mL), and the combined organic layers were washed with water (1×5 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 40% to 50% gradient in 10 min; detector, UV 220 nm) to provide a crude product (45 mg), which was further purified by Prep-HPLC (XBridge Prep OBD C18 Column, 30*150 mm, 5 m; Mobile Phase A: water (10 mmol/L NH₄HCO₃), Mobile Phase B: acetonitrile; Flow rate: 60 mL/min; Gradient: 68% B to 83% B in 10 min; Wave Length: 254/220 nm; RT(min): 6.89) to afford tert-butyl 4-(3-(5-cyclopropyl-4,7-difluoro-3,3-dimethyl-2-oxoindolin-1-yl)-2-oxopyrrolidin-1-yl)butanoate (Compound 16-OtBu) (30 mg, 41% yield), provided as a mixture of stereoisomers. LCMS: (ES, m/z): RT=0.941 min, m/z=463.2[M+H]⁺.

Step 3: The Compound 16-OtBu mixture product (30 mg) was purified by Prep-Chiral-HPLC (CHIRALPAK IC, 2*25 cm, 5 m; Mobile Phase A: Hexanes (0.5% 2M NH₃-MeOH), Mobile Phase B: MeOH: DCM=1:1; Flow rate: 20 mL/min; Gradient: 20% B to 20% B in 11 min; Wave Length: 220/254 nm) to provide tert-butyl (R)-4-(3-(5-cyclopropyl-4,7-difluoro-3,3-dimethyl-2-oxoindolin-1-yl)-2-oxopyrrolidin-1-yl)butanoate (Compound 16A-OtBu) (10.1 mg, 33% yield, RT(min): 8.19) as the first eluting peak, and tert-butyl (S)-4-(3-(5-cyclopropyl-4,7-difluoro-3,3-dimethyl-2-oxoindolin-1-yl)-2-oxopyrrolidin-1-yl)butanoate (Compound 16B-OtBu) (9.4 mg, 31% yield, RT(min): 9.87) as the second eluting peak. Compound 16B-OtBu: LCMS: (ES, m/z): RT=0.943 min, m/z=463.2[M+H]⁺. Compound 16A-OtBu: LCMS: (ES, m/z): RT=0.940 min, m/z=463.2[M+H]⁺. Absolute stereochemistry of 16A-OtBu and 16B-OtBu known based on X-ray crystallographic confirmation of the absolute stereochemistry of Compound 16A.

Step 4: A solution of Compound 16A-OtBu (10.1 mg, 0.02 mmol, 1 equiv) and trifluoroacetic acid (TFA) (0.2 mL) in dichloromethane (DCM) (0.3 mL) was stirred for 1 h at room temperature, then the resulting mixture was concentrated under reduced pressure. The residue was purified by reversed-phase flash chromatography (C18 silica gel; mobile phase, acetonitrile in water, 30% to 40% gradient in 10 min; detector, UV220/254 nm) to provide (R)-4-(3-(5-cyclopropyl-4,7-difluoro-3,3-dimethyl-2-oxoindolin-1-yl)-2-oxopyrrolidin-1-yl)butanoic acid (Compound 16A) (6.3 mg, 70% yield). Absolute stereochemistry of Compound 16A was confirmed by X-ray crystallography.

Step 5: A solution of Compound 16B-OtBu (9.40 mg, 0.02 mmol, 1 equiv) and trifluoroacetic acid (TFA) (0.2 mL) in dichloromethane (DCM) (0.3 mL) was stirred for 1 h at room temperature, then the resulting mixture was concentrated under reduced pressure. The residue was purified by reversed-phase flash chromatography (C18 silica gel; mobile phase, acetonitrile in water, 30% to 40% gradient in 10 min; detector, UV 220/254 nm) to provide (S)-4-(3-(5-cyclopropyl-4,7-difluoro-3,3-dimethyl-2-oxoindolin-1-yl)-2-oxopyrrolidin-1-yl)butanoic acid (Compound 16B) (5.9 mg, 71% yield). Absolute stereochemistry of Compound 16B known based on X-ray crystallographic confirmation of the absolute stereochemistry of Compound 16A.

Compound 16A: LCMS: (ES, m/z): RT=1.414 min, m/z=405.1[M+H]⁺. ¹H NMR (400 MHz, DMSO-d6) δ 12.66-11.44 (m, 1H), 6.98-6.67 (m, 1H), 6.83-6.78 (m, 1H), 5.27-5.22 (m, 1H), 3.54-3.38 (m, 2H), 3.26-3.14 (m, 2H), 2.34 (s, 1H), 2.25-2.21 (m, 2H), 2.10-1.95 (m, 2H), 1.82-1.63 (m, 2H), 1.44-1.32 (m, 6H), 0.99-0.88 (m, 2H), 0.72-0.70 (m, 2H).

Compound 16B: LCMS: (ES, m/z): RT=1.414 min, m/z=405.1[M+H]⁺. ¹H NMR (400 MHz, DMSO-d6) δ 12.66-11.44 (bar, 1H), 6.96-6.69 (m, 1H), 5.27-5.22 (m, 1H), 3.49-3.31 (m, 2H), 3.20-3.18 (m, 2H), 2.34 (s, 1H), 2.20-2.10 (m, 2H), 2.08-1.95 (m, 2H), 1.82-1.63 (m, 2H), 1.44-1.32 (m, 6H), 0.95-0.93 (m, 2H), 0.71-0.70 (m, 2H).

### Example 11. 2-(2-(3-(5-cyclopropyl-4,7-difluoro-3,3-dimethyl-2-oxoindolin-1-yl)-2-oxopyrazin-1(2H)-yl)cyclopentyl)acetic acid (Compound 20, rac-20), 2-((1S,2S)-2-(3-(5-cyclopropyl-4,7-difluoro-3,3-dimethyl-2-oxoindolin-1-yl)-2-oxopyrazin-1(2H)-yl)cyclopentyl)acetic acid (Compound 20A*), 2-((1R,2R)-2-(3-(5-cyclopropyl-4,7-difluoro-3,3-dimethyl-2-oxoindolin-1-yl)-2-oxopyrazin-1(2H)-yl)cyclopentyl)acetic acid (Compound 20B*), 2-((1R,2S)-2-(3-(5-cyclopropyl-4,7-difluoro-3,3-dimethyl-2-oxoindolin-1-yl)-2-oxopyrazin-1(2H)-yl)cyclopentyl)acetic acid (Compound 20C*), and 2-((1S,2R)-2-(3-(5-cyclopropyl-4,7-difluoro-3,3-dimethyl-2-oxoindolin-1-yl)-2-oxopyrazin-1(2H)-yl)cyclopentyl)acetic acid (Compound 20D*), and Compounds 20A*-OEt, 20B*-OEt, 20A*-OtBu, 20B*-OtBu, 20C*-OtBu, and 20D*-OtBu

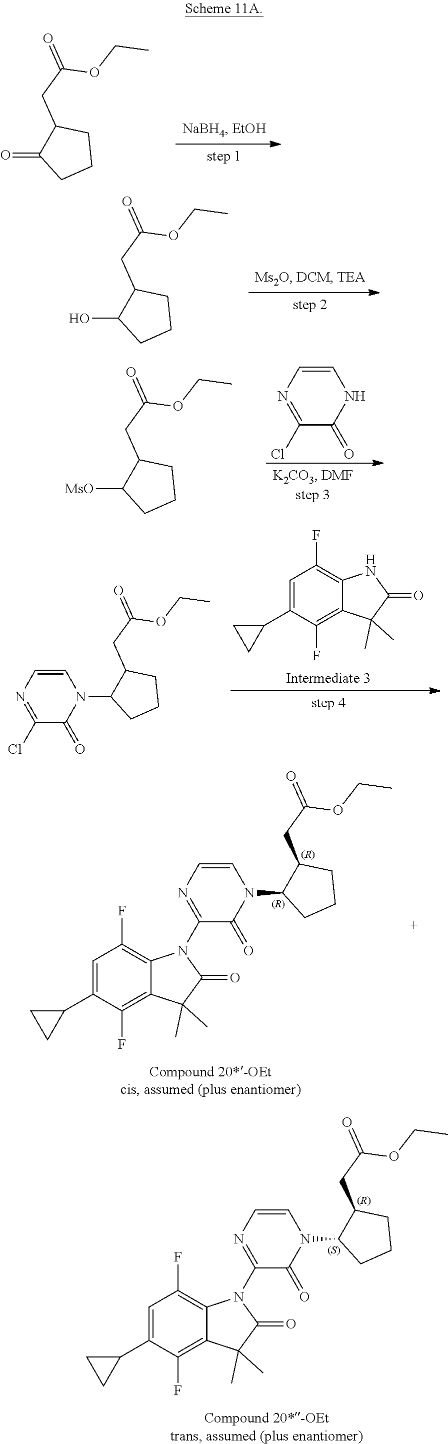

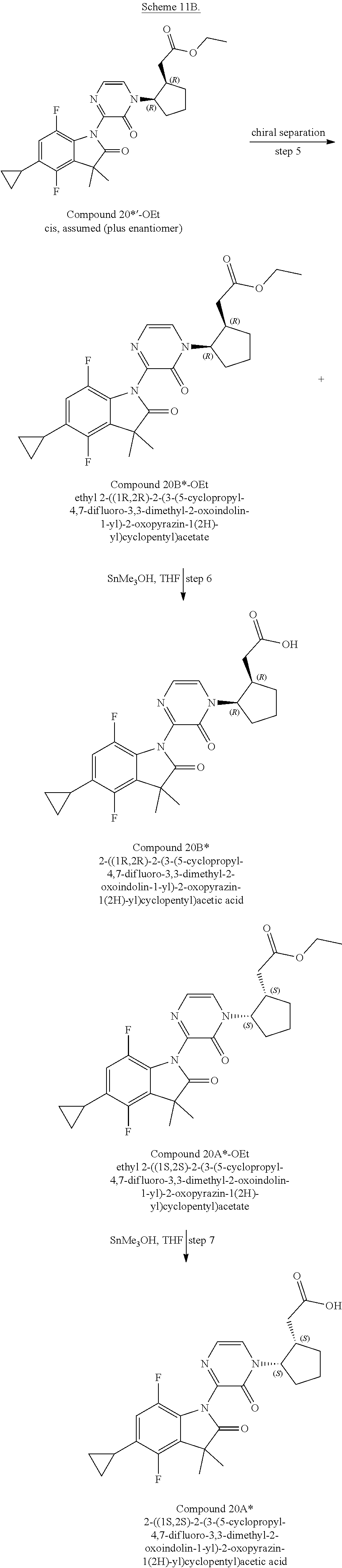

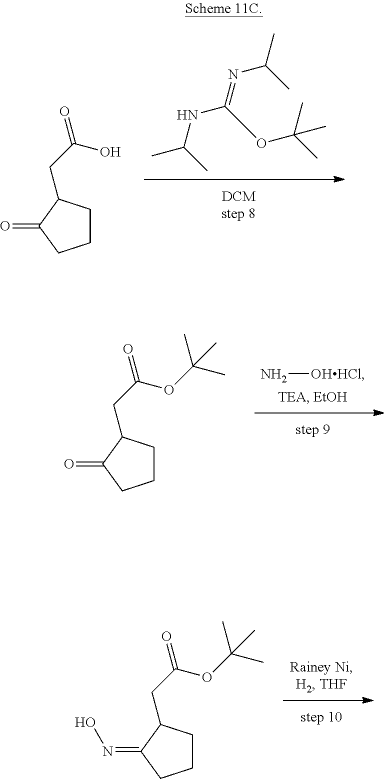

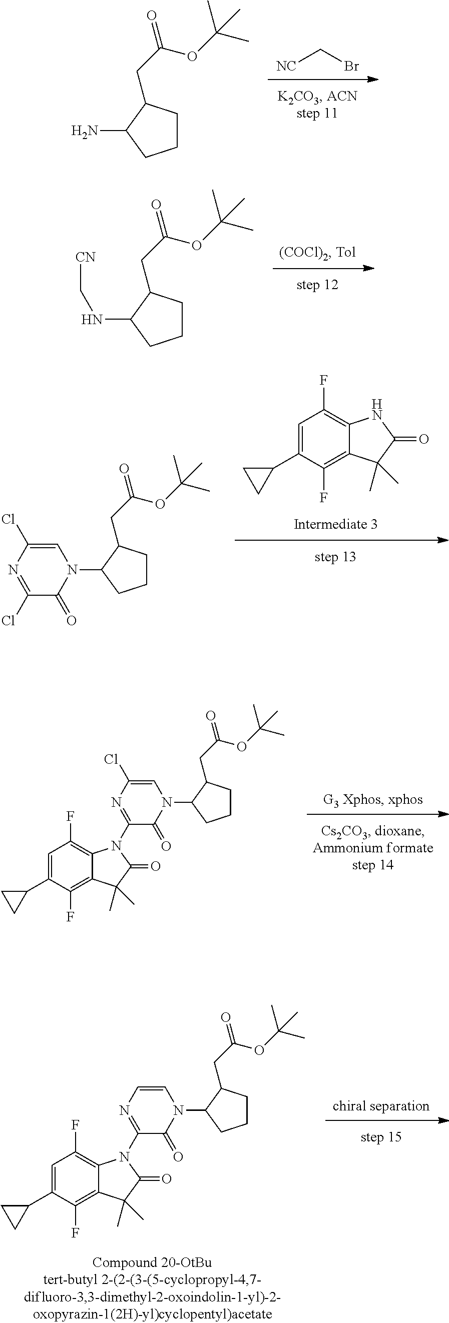

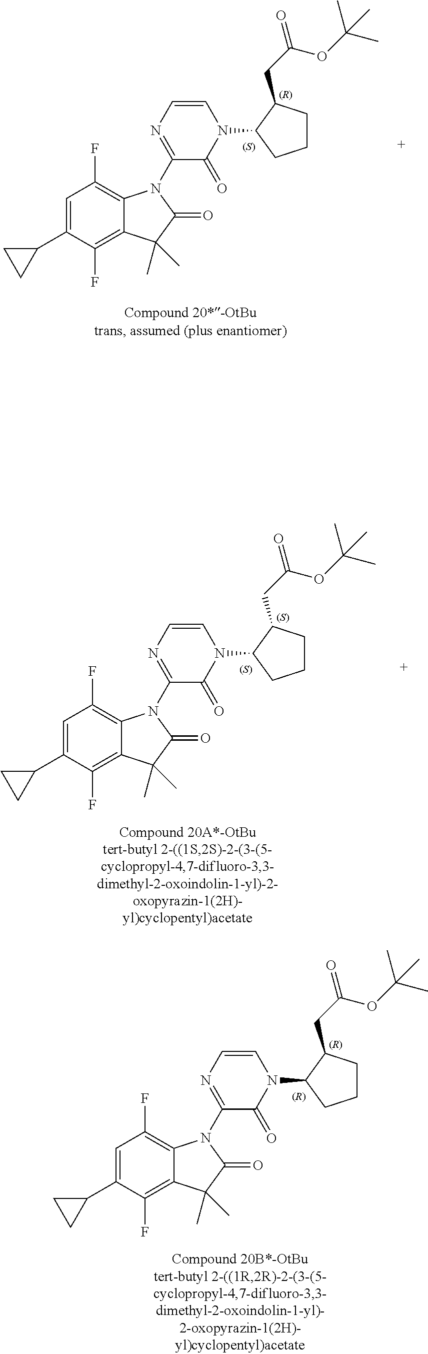

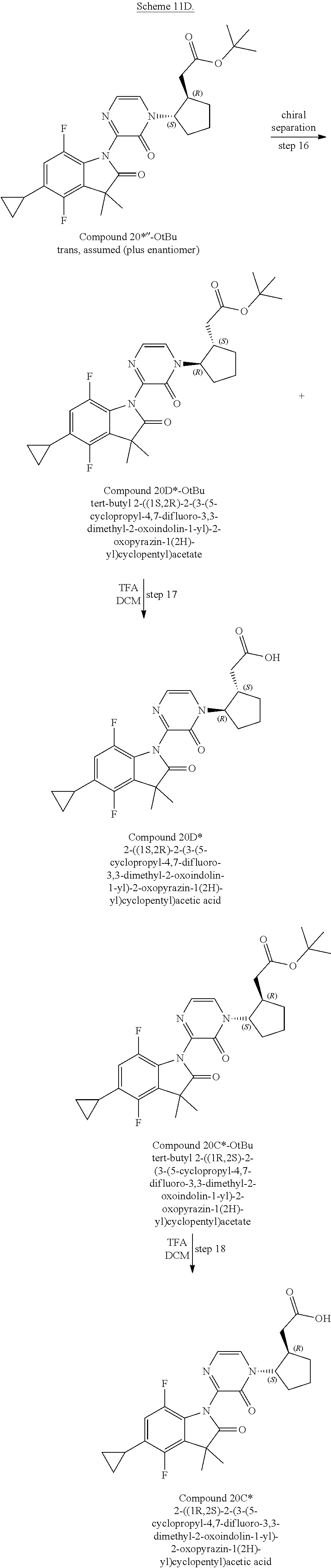

Step 1: Into a 500 mL round-bottom flask was added ethyl-2-(2-oxocyclopentyl)acetate (20.0 g, 118 mmol, 1 equiv) and ethanol (EtOH) (200 mL). NaBH₄ (8.89 g, 235 mmol, 2 equiv) was added at 0° C. The resulting mixture was stirred for 1 h at room temperature under nitrogen atmosphere. The reaction was monitored by GCMS. The mixture was adjusted to pH 3 with HCl (2M). The resulting mixture was extracted with ethyl acetate (EtOAc) (3×500 mL). The combined organic layers were washed with brine (2×500 mL) and dried over anhydrous Na₂SO₄. After filtrated, the filtrate was concentrated under reduced pressure to provide a residue, which was purified by silica gel column chromatography, eluting with petroleum ether/ethyl acetate (1:1), to afford ethyl-2-(2-hydroxycyclopentyl)acetate (8 g, 40% yield). LCMS:(ES, m/z): RT=0.518 min, m/z=173.1[M+H]⁺.

Step 2: Into a 500 mL round-bottom flask was added ethyl-2-(2-hydroxycyclopentyl)acetate (8 g, 46.5 mmol, 1 equiv), triethylamine (14.1 g, 139 mmol, 3 equiv), dichloromethane (DCM) (200 mL) and methanesulfonyl methanesulfonate (16.2 g, 92.9 mmol, 2 equiv) at room temperature. The resulting mixture was stirred for 1 h at room temperature under nitrogen atmosphere, then quenched by the addition of water (200 mL) at room temperature, and the resulting mixture was extracted with CH₂Cl₂ (3×500 mL). The combined organic layers were washed with brine (2×500 mL), dried over anhydrous Na₂SO₄. After filtration, the filtrate was concentrated under reduced pressure to provide ethyl-2-[2-(methanesulfonyloxy)cyclopentyl]acetate (5 g, 43% yield).

Step 3: Into a 250 mL round-bottom flask was added ethyl-2-[(1R,2S)-2-(methanesulfonyloxy)cyclopentyl]acetate (5 g, 20.0 mmol, 1 equiv) and 3-chloro-1H-pyrazin-2-one (1.56 g, 12.0 mmol, 0.6 equiv), K₂CO₃ (8.28 g, 59.9 mmol, 3 equiv), dimethylformamide (DMF) (50 mL) at room temperature. The resulting mixture was stirred at 80° C. for 16 h under nitrogen atmosphere, then quenched by the addition of water (100 mL) at room temperature. The resulting mixture was extracted with ethyl acetate (EtOAc) (3×100 mL), and the combined organic layers were washed with brine (2×100 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254 nm/220 nm) to provide ethyl-2-[(1R,2R)-2-(3-chloro-2-oxopyrazin-1-yl)cyclopentyl]acetate (240 mg, 4% yield). LCMS:(ES, m/z):RT=0.620 min, m/z=285.1[M+H]V.

Step 4: Into a 20 mL vial was added ethyl 2-(2-(3-chloro-2-oxopyrazin-1(2H)-yl)cyclopentyl)acetate (240 mg, 0.840 mmol, 1 equiv) and 5-cyclopropyl-4,7-difluoro-3,3-dimethylindolin-2-one (Intermediate 3) (100 mg, 0.42 mmol, 0.5 equiv), (1R,2R)—N1,N2-dimethylcyclohexane-1,2-diamine (24.0 mg, 0.16 mmol, 0.20 equiv), CuI (16.1 mg, 0.08 mmol, 0.10 equiv), acetonitrile (3 mL), and K₂CO₃ (350 mg, 2.52 mmol, 3 equiv) at room temperature. The resulting mixture was stirred at 80° C. for 16 h under nitrogen atmosphere, then quenched by the addition of water (10 mL) at room temperature. The resulting mixture was extracted with CH₂Cl₂ (3×20 mL), and the combined organic layers were washed with water (2×20 mL) and dried over anhydrous Na₂SO₄.

After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254 nm/220 nm) to provide as the first eluting peak Compound 20*′-OEt (180 mg, 43.9% yield) as a mixture of two cis stereoisomers, and as the second eluting peak Compound 20*″-OEt (0.3 mg) as a mixture of two trans stereoisomers. *Cis and trans stereochemistry arbitrarily assigned. Compound 20*′-OEt (cis, assumed) was repurified by (i) Prep-HPLC (XSelect CSH Prep C18 OBD Column, 19*250 mm, 5 m; Mobile Phase A: Water (10 mmol/L NH₄HCO₃), Mobile Phase B: acetonitrile; Flow rate: 20 mL/min; Gradient: 55% B to 65% B in 10 min; Wave Length: 254 nm/220 nm; RT(min): 15.7), then further purified by (ii) Prep-Chiral-HPLC (CHIRALPAK IG, 2*25 cm, 5 m; Mobile Phase A: Hexanes (0.5% 2M NH₃-MeOH), Mobile Phase B: EtOH: DCM=1:1; Flow rate: 20 mL/min; Gradient: 20% B to 20% B in 12 min; Wave Length: 220 nm/254 nm; RT(min): 7.33) provide Compound 20*′-OEt (60 mg, 14.7% yield) as a mixture of two cis stereoisomers.

Step 5: The Compound 20*′-OEt mixture of Step 4 (60.0 mg) was separated by Prep-Chiral-HPLC (CHIRALPAK IC, 2*25 cm, 5 m; Mobile Phase A: Hexanes (0.1% TFA), Mobile Phase B: EtOH: DCM=1:1; Flow rate: 20 mL/min; Gradient: 50% B to 50% B in 10 min; Wave Length: 220 nm/254 nm;) to afford as the first eluting peak Compound 20B*-OEt (20.0 mg, 33.3% yield, RT(min): 5.97) and as the second eluting peak Compound 20A*-OEt (25.0 mg, 41.7% yield, RT(min): 8.03). * Stereochemistry arbitrarily assigned.

Compound 20A*-OEt: LCMS:(ES, m/z): RT=1.619 min, m/z=486.1 [M+H]⁺. ¹H NMR (400 MHz, Methanol-d₄) δ 7.68 (d, J=5.6 Hz, 1H), 7.44 (d, J=4.8 Hz, 1H), 6.72-6.63 (m, 1H), 5.25-5.15 (m, 1H), 4.05 (s, 2H), 2.84 (d, J=8.4 Hz, 1H), 2.37-1.98 (m, 7H), 1.84-1.74 (m, 1H), 1.64-1.56 (m, 7H), 1.23-1.12 (m, 3H), 1.05-0.97 (m, 2H), 0.74-0.66 (m, 2H).

Compound 20B*-OEt: LCMS:(ES, m/z): RT=1.617 min, m/z=486.1 [M+H]⁺. H NMR (400 MHz, Methanol-d₄) δ 7.68 (d, J=5.6 Hz, 1H), 7.44 (d, J=4.8 Hz, 1H), 6.72-6.64 (m, 1H), 5.25-5.15 (m, 1H), 4.05 (s, 2H), 2.84 (d, J=8.4 Hz, 1H), 2.34-1.97 (m, 7H), 1.82 (s, 1H), 1.68-1.39 (m, 7H), 1.23-1.12 (m, 3H), 1.06-0.97 (m, 2H), 0.74-0.66 (m, 2H).

Step 6: Into an 8 mL vial was added Compound 20B*-OEt (20 mg, 0.04 mmol, 1 equiv) and trimethylstannanol (74.5 mg, 0.40 mmol, 10 equiv) and tetrahydrofuran (THF) (1.0 mL) at room temperature. The resulting mixture was stirred at 80° C. for 16 h under nitrogen atmosphere, then quenched by the addition of water (5.00 mL) at room temperature. The resulting mixture was extracted with EtOAc (3×5 mL), and the combined organic layers were washed with water (2×5 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254 nm/220 nm), and further purified by Prep-HPLC (XBridge Prep Phenyl OBD Column19*250 mm; 5 m;Mobile Phase A: Water(0.05% TFA), Mobile Phase B: acetonitrile; Flow rate: 25 mL/min; Gradient: 40% B to 50% B in 10 min; Wave Length: 254 nm/220 nm; RT(min): 12.3) to afford Compound 20B* (6.8 mg, 36% yield). * Stereochemistry arbitrarily assigned.

Step 7: Into an 8 mL vial was added Compound 20A*-OEt (25.0 mg, 0.05 mmol, 1 equiv), tetrahydrofuran (THF) (1 mL) and trimethylstannanol (93.10 mg, 0.50 mmol, 10 equiv) at room temperature. The resulting mixture was stirred at 80° C. for 16 h under nitrogen atmosphere, then quenched by the addition of water (5 mL) at room temperature. The resulting mixture was extracted with EtOAc (3×5 mL), and the combined organic layers were washed with water (2×5 mL), and dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 100% gradient in 30 min; detector, UV 254 nm/220 nm), and further purified by Prep-HPLC (Xselect CSH C18 OBD Column 30*150 mm 5 m; Mobile Phase A: Water (0.05% TFA), Mobile Phase B: acetonitrile; Flow rate: 60 mL/min; Gradient: 5% B to 5% B in 2 min, 32% B to 52% B in 15 min; Wave Length: 254 nm/220 nm; RT(min): 13.5) to afford Compound 20A* (9.8 mg, 40% yield). *Stereochemistry arbitrarily assigned.

Compound 20A*: LCMS:(ES, m/z): RT=1.485 min, m/z=458.1 [M+H]⁺. ¹H NMR (400 MHz, Methanol-d₄) δ 7.69 (d, J=4.4 Hz, 1H), 7.45 (d, J=4.8 Hz, 1H), 6.73-6.64 (m, 1H), 5.28-5.14 (m, 1H), 2.82 (d, J=7.2 Hz, 1H), 2.34-1.96 (m, 7H), 1.86-1.73 (m, 1H), 1.70-1.56 (m, 7H), 1.05-0.94 (m, 2H), 0.75-0.64 (m, 2H).

Compound 20B*: LCMS:(ES, m/z):RT=1.030 min, m/z=458.1 [M+H]⁺. ¹H NMR (400 MHz, Methanol-d₄) δ 7.70 (d, J=4.4 Hz, 1H), 7.45 (d, J=4.8 Hz, 1H), 6.73-6.64 (m, 1H), 5.29-5.14 (m, 1H), 2.82 (d, J=7.2 Hz, 1H), 2.35-1.93 (m, 7H), 1.81-1.77 (m, 1H), 1.68-1.53 (m, 7H), 1.05-0.96 (m, 2H), 0.75-0.66 (m, 2H).

Step 8: Into a 250 mL round-bottom flask was added (2-oxocyclopentyl)acetic acid (10.0 g, 70.3 mmol, 1 equiv) and dichloromethane (DCM) (100 mL) at room temperature. To the above mixture was added (Z)-N,N′-diisopropyltert-butoxymethanimidamide (141 g, 703 mmol, 10 equiv) at 0° C. The resulting mixture was stirred for 2 h at room temperature, then the reaction was quenched by the addition of water (500 mL) at room temperature. The resulting mixture was extracted with DCM (3×500 mL), and the combined organic layers were washed with brine (2×500 mL) and dried over anhydrous Na₂SO₄.

After filtration, the filtrate was concentrated under reduced pressure to provide a residue, which was purified by silica gel column chromatography, eluting with petroleum ether/ethyl acetate (3:1), to afford tert-butyl 2-(2-oxocyclopentyl)acetate (10 g, 72% yield). LCMS:(ES, m/z): RT=0.707 min, m/z=199.1[M+H]⁺.

Step 9: Into a 250 mL round-bottom flask was added tert-butyl 2-(2-oxocyclopentyl)acetate (10 g, 50.4 mmol, 1 equiv) and triethylamine (TEA) (15.3 g, 151 mmol, 3 equiv), ethanol (EtOH) (50 mL), and hydroxylamine hydrochloride (10.5 g, 151 mmol, 3 equiv) at room temperature. The resulting mixture was stirred at 20° C. for 2 h, then quenched by the addition of water (100 mL) at room temperature. The resulting mixture was extracted with ethyl acetate (EtOAc) (3×200 mL) and the combined organic layers were washed with H₂O (100 mL) and dried over anhydrous Na₂SO₄. After filtration, the filtrate was concentrated under reduced pressure to provide tert-butyl 2-[(2Z)-2-(hydroxyimino)cyclopentyl]acetate (7 g, 65% yield). LCMS:(ES, m/z): RT=0.682 min, m/z=214.1[M+H]⁺.

Step 10: Into a 250 mL round-bottom flask was added tert-butyl 2-[(2Z)-2-(hydroxyimino)cyclopentyl]acetate (7 g, 32.8 mmol, 1 equiv), tetrahydrofuran (THF) (50 mL), and Raney Ni (9.63 g, 164 mmol, 5 equiv) at room temperature. The mixture was stirred at room temperature for overnight under hydrogen atmosphere, then the resulting mixture was filtered, the filter cake was washed with THF (50 mL), and the filtrate was concentrated under reduced pressure to provide tert-butyl 2-(2-aminocyclopentyl)acetate (3 g, 46% yield). LCMS:(ES, m/z): RT=0.392 min, m/z=200.2[M+H]⁺.

Step 11: Into a 100 mL vial was added tert-butyl 2-(2-aminocyclopentyl)acetate (3 g, 15.1 mmol, 1 equiv) and 2-bromoacetonitrile (5.42 g, 45.2 mmol, 3 equiv), and acetonitrile (10 mL) at room temperature. The mixture was stirred at 80° C. for 2 h, then quenched by the addition of water (20 mL) at room temperature. The resulting mixture was extracted with ethyl acetate (EtOAc) (3×100 mL), and the combined organic layers were washed with water (2×20 mL) and dried over anhydrous Na₂SO₄. After filtration, the filtrate was concentrated under reduced pressure to provide a residue, which was purified by silica gel column chromatography, eluting with petroleum ether/ethyl acetate (5:1), to afford tert-butyl 2-{2-[(cyanomethyl)amino]cyclopentyl}acetate (3 g, 84% yield). LCMS:(ES, m/z): RT=0.535 min, m/z=239.2[M+H]⁺.

Step 12: To a 40 mL vial was added tert-butyl 2-{2-[(cyanomethyl)amino]cyclopentyl}acetate (1 g, 12.6 mmol, 1 equiv), (COCl)₂ (2.7 g, 62.9 mmol, 5 equiv), and toluene (3 mL) at room temperature, and the reaction was stirred at 80° C. for 2 h, then quenched by the addition of water (7 mL) at room temperature. The reaction was repeated two times, the batches were combined and extracted with ethyl acetate (EtOAc) (3×10 mL), and the combined organic layers were washed with water (10 mL) and dried over anhydrous Na₂SO₄. After filtration, the filtrate was concentrated under reduced pressure to provide a residue, which was purified by reversed-phase flash chromatography (C18 silica gel; mobile phase, acetonitrile in water, 10% to 50% gradient in 10 min; detector, UV 254/220 nm) to provide tert-butyl 2-[2-(3,5-dichloro-2-oxopyrazin-1-yl)cyclopentyl]acetate (1 g, 23% yield). LCMS:(ES, m/z): RT=0.964 min, m/z=347.1[M+H]⁺.

Step 13: Into a 40 mL vial was added tert-butyl 2-[2-(3,5-dichloro-2-oxopyrazin-1-yl)cyclopentyl]acetate (400 mg, 1.15 mmol, 1 equiv), 5-cyclopropyl-4,7-difluoro-3,3-dimethylindolin-2-one (Intermediate 3) (137 mg, 0.57 mmol, 0.50 equiv), CuI (44 mg, 0.23 mmol, 0.20 equiv), K₂CO₃ (478 mg, 3.45 mmol, 3 equiv), acetonitrile (4 mL) and (1R,2R)—N1,N2-dimethylcyclohexane-1,2-diamine (33 mg, 0.23 mmol, 0.20 equiv) at room temperature. The mixture was stirred at 80° C. for 2 h under nitrogen atmosphere, then quenched by the addition of water (5 mL) at room temperature. The resulting mixture was extracted with ethyl acetate (EtOAc) (3×5 mL), and the combined organic layers were washed with water (2×10 mL), dried over anhydrous Na₂SO₄. After filtration, the filtrate was concentrated under reduced pressure to provide a residue, which was purified by reversed-phase flash chromatography (C18 silica gel; mobile phase, acetonitrile in water, 10% to 50% gradient in 10 min; detector, UV 254 nm) to provide tert-butyl 2-(2-[5-chloro-3-(5-cyclopropyl-4,7-difluoro-3,3-dimethyl-2-oxoindol-1-yl)-2-oxopyrazin-1-yl]cyclopentylacetate (115 mg, 18% yield). LCMS:(ES, m/z): RT=0.833 min, m/z=548.2[M+H]⁺.

Step 14: Into a 40 mL vial was added tert-butyl 2-(2-[5-chloro-3-(5-cyclopropyl-4,7-difluoro-3,3-dimethyl-2-oxoindol-1-yl)-2-oxopyrazin-1-yl]cyclopentylacetate (115 mg, 0.21 mmol, 1 equiv), 1,4-dioxane (3 mL), 2-dicyclohexylphosphino-2′,4′,6′-triisopropylbiphenyl (Xphos) (40 mg, 0.080 mmol, 0.40 equiv), (2-dicyclohexylphosphino-2′,4′,6′-triisopropyl-1,1′-biphenyl)[2-(2′-amino-1,1′-biphenyl)]palladium(II) methanesulfonate (XPhos Pd G3) (35 mg, 0.040 mmol, 0.20 equiv), ammonium formate (27 mg, 0.42 mmol, 2 equiv) and Cs₂CO₃ (210 mg, 0.63 mmol, 3 equiv) at room temperature. The mixture was stirred at 80° C. for 2 h. The reaction progress was monitored by LCMS. The reaction was quenched by the addition of water (5 mL) at room temperature. The resulting mixture was extracted with ethyl acetate (EtOAc) (3×10 mL). The combined organic layers were washed with water (20 mL), dried over anhydrous Na₂SO₄. After filtration, the filtrate was concentrated under reduced pressure to provide a residue, which was purified by reversed-phase flash chromatography (C18 silica gel; mobile phase, acetonitrile in water, 10% to 50% gradient in 10 min; detector, UV 254/220 nm) to afford tert-butyl 2-(2-1[3-(5-cyclopropyl-4,7-difluoro-3,3-dimethyl-2-oxoindol-1-yl)-2-oxopyrazin-1-yl]cyclopentylacetate (45 mg, 42% yield) as a mixture of four cis and trans stereoisomers. LCMS:(ES, m/z): RT=0.955 min, m/z=514.2[M+H]⁺.

Step 15: The tert-butyl 2-(2-(3-(5-cyclopropyl-4,7-difluoro-3,3-dimethyl-2-oxoindolin-1-yl)-2-oxopyrazin-1(2H)-yl)cyclopentyl)acetate mixture of Step 14 (45 mg) was separated by Prep-Chiral-HPLC (CHIRAL ART Cellulose-SC, 2*25 cm, 5 μm; Mobile Phase A: Hexanes (0.1% TFA), Mobile Phase B: EtOH: DCM=1:1; Flow rate: 20 mL/min; Gradient: 40% B to 40% B in 14 min; Wave Length: 220/254 nm) to afford as the first eluting peak a mixture of Compound 20C*-OtBu and Compound 20D*-OtBu (20 mg, 44% yield, RT(min): 8.07); as the second eluting peak Compound 20A*-OtBu (5.0 mg, 11I % yield, RT(min): 12.39); and as the third eluting peak Compound 20B*-OtBu (5.0 mg, 11% yield, RT(min): 15.28). *Stereochemistry arbitrarily assigned.

Step 16: The mixture of Compound 20C*-OtBu and Compound 20D*-OtBu from Step 15 (20 mg) was separated by Prep-Chiral-HPLC (CHIRALPAK ID, 2*25 cm, 5 m; Mobile Phase A: Hexanes (0.5% 2M NH₃-MeOH), Mobile Phase B: EtOH; Flow rate: 20 mL/min; Gradient: 10% B to 10% B in 15 min; Wave Length: 220/254 nm) to afford as the first eluting peak Compound 20D*-OtBu (10 mg, 50% yield, RT(min): 8.75), and as the second eluting peak Compound 20C*-OtBu (9.8 mg, 49% yield, RT(min): 11.38). Compound 20C*-OtBu: LCMS:(ES, m/z): RT=1.497 min, m/z=514.2[M+H]⁺. Compound 20D*-OtBu: LCMS:(ES, m/z): RT=1.537 min, m/z=514.2[M+H]⁺.

Step 17: Into an 8 mL vial was added Compound 20D*-OtBu (10 mg, 0.02 mmol, 1 equiv) and trifluoracetic acid (TFA) (0.2 mL) in dichloromethane (DCM) (0.4 mL) at room temperature. The mixture was stirred at room temperature for 1 h, then concentrated under reduced pressure and lyophilized to afford Compound 20D* (5.2 mg 58% yield).

Step 18: Into an 8 mL vial was added Compound 20C*-OtBu (9.8 mg, 0.02 mmol, 1 equiv) and trifluoracetic acid (TFA) (0.2 mL) in dichloromethane (DCM) (0.4 mL) at room temperature. The mixture was stirred at room temperature for 1 h, then concentrated under reduced pressure and lyophilized to afford Compound 20C* (5.2 mg 65% yield).

Compound 20C*: LCMS:(ES, m/z): RT=1.598 min, m/z=458.2[M+H]⁺. ¹H NMR (400 MHz, Methanol-d₄) δ 7.88-7.83 (m, 1H), 7.47 (d, J=4.4 Hz, 1H), 6.72-6.65 (m, 1H), 2.66 (s, 1H), 2.52-2.16 (m, 4H), 2.13-1.79 (m, 4H), 1.70-1.40 (m, 8H), 1.06-0.97 (m, 2H), 0.71 (d, J=6.0 Hz, 2H).

Compound 20D*: LCMS:(ES, m/z): RT=1.577 min, m/z=458.2[M+H]⁺. ¹H NMR (400 MHz, Methanol-d₄) δ 7.85 (d, J=4.4 Hz, 1H), 7.47 (d, J=4.4 Hz, 1H), 6.72-6.65 (m, 1H), 2.65 (s, 1H), 2.52-2.16 (m, 4H), 2.13-1.78 (m, 4H), 1.69-1.42 (m, 8H), 1.01 (d, J=8.8 Hz, 2H), 0.71 (d, J=5.2 Hz, 2H).

### Example 12. 4-(3-(4,7-difluoro-3,3-dimethyl-2-oxo-5-(trifluoromethyl)indolin-1-yl)-2-oxopyrrolidin-1-yl)butanoic acid (Compound 19, rac-19), tert-butyl (R)-4-(3-(4,7-difluoro-3,3-dimethyl-2-oxo-5-(trifluoromethyl)indolin-1-yl)-2-oxopyrrolidin-1-yl)butanoate (Compound 19A*-OtBu), tert-butyl (S)-4-(3-(4,7-difluoro-3,3-dimethyl-2-oxo-5-(trifluoromethyl)indolin-1-yl)-2-oxopyrrolidin-1-yl)butanoate (Compound 19B*-OtBu), (R)-4-(3-(4,7-difluoro-3,3-dimethyl-2-oxo-5-(trifluoromethyl)indolin-1-yl)-2-oxopyrrolidin-1-yl)butanoic acid (Compound 19A*), and (S)-4-(3-(4,7-difluoro-3,3-dimethyl-2-oxo-5-(trifluoromethyl)indolin-1-yl)-2-oxopyrrolidin-1-yl)butanoic acid (Compound 19B*)

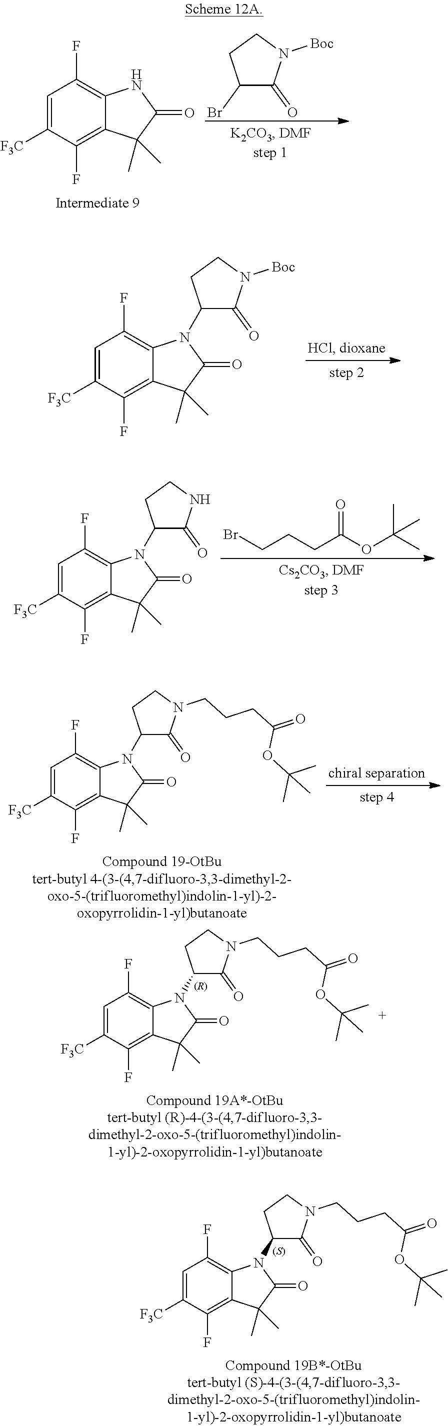

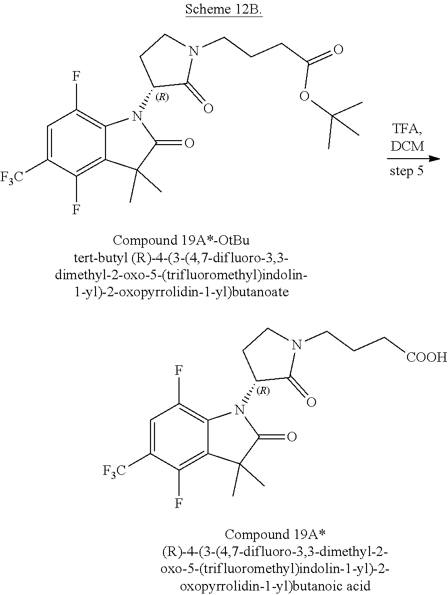

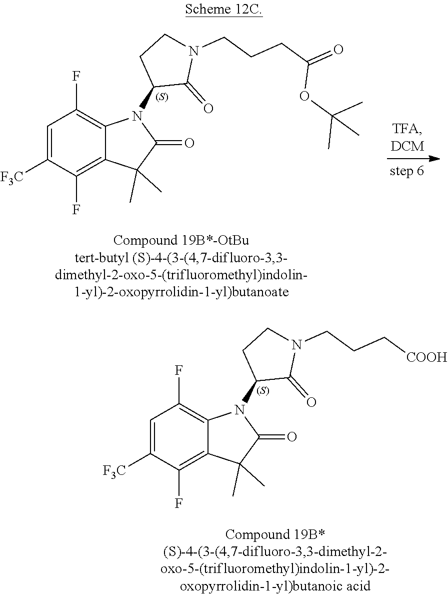

Step 1: Into an 8 mL vial was added 4,7-difluoro-3,3-dimethyl-5-(trifluoromethyl)indolin-2-one (Intermediate 9) (300 mg, 1.13 mmol, 1 equiv), tert-butyl 3-bromo-2-oxopyrrolidine-1-carboxylate (448 mg, 1.70 mmol, 1.50 equiv), K₂CO₃ (469 mg, 3.39 mmol, 3 equiv) and dimethylformamide (DMF) (2.5 mL). The resulting mixture was stirred at 80° C. overnight, then diluted with water (30 mL). The aqueous layer was extracted with ethyl acetate (EtOAc) (3×30 mL) and concentrated under reduced pressure to provide a residue, which was purified by reversed-phase flash chromatography (C18 silica gel; mobile phase, acetonitrile in water, 45% to 55% gradient in 10 min; detector, UV 254 nm) to afford tert-butyl 3-[4,7-difluoro-3,3-dimethyl-2-oxo-5-(trifluoromethyl)indol-1-yl]-2-oxopyrrolidine-1-carboxylate (430 mg, 85% yield). LCMS:(ES, m/z): RT=1.09 min, m/z=449.1 [M+H]⁺.

Step 2: Into a 20 mL vial was added tert-butyl 3-[4,7-difluoro-3,3-dimethyl-2-oxo-5-(trifluoromethyl)indol-1-yl]-2-oxopyrrolidine-1-carboxylate (410 mg, 0.910 mmol, 1 equiv) and HCl(gas) in dioxane (4.0 M in 1,4-dioxane) (5.0 mL). The resulting mixture was stirred at room temperature overnight and then concentrated under reduced pressure to afford 4,7-difluoro-3,3-dimethyl-1-(2-oxopyrrolidin-3-yl)-5-(trifluoromethyl)indol-2-one (300 mg, 94% yield). LCMS:(ES, m/z): RT=0.71 min, m/z=349.1 [M+H]⁺.

Step 3: Into a 20 mL vial was added 4,7-difluoro-3,3-dimethyl-1-(2-oxopyrrolidin-3-yl)-5-(trifluoromethyl)indol-2-one (140 mg, 0.4 mmol, 1 equiv), tert-butyl 4-bromobutanoate (448 mg, 2.01 mmol, 5 equiv), Cs₂CO₃ (1310 mg, 4.02 mmol, 10 equiv) and dimethylformamide (DMF) (2.5 mL). The resulting mixture was stirred at 100° C. for 3 h, then the mixture was diluted with water (30 mL), and the aqueous layer was extracted with EtOAc (4×30 mL) and concentrated under reduced pressure to provide a residue, which was purified by reversed-phase flash chromatography (C18 silica gel; mobile phase, acetonitrile in water, 60% to 70% gradient in 10 min; detector, UV 254 nm) to afford Compound 19-OtBu (120 mg, 61% yield) as a mixture of two stereoisomers. LCMS:(ES, m/z): RT=0.92 min, m/z=491.1 [M+H]⁺.

Step 4: The mixture of Compound 19-OtBu (120 mg) was separated by Prep-Chiral-HPLC (CHIRALPAK IF, 2*25 cm, 5 m; Mobile Phase A: Hexanes (0.5% 2M NH₃-MeOH), Mobile Phase B: EtOH: DCM=1:1--HPLC; Flow rate: 20 mL/min; Gradient: 20% B to 20% B in 10 min; Wave Length: 220/254 nm) to afford Compound 19A*-OtBu (40 mg, 20% yield, RT (min): 5.92) as the first eluting peak and Compound 19B*-OtBu (40 mg, 20% yield, RT (min): 6.96) as the second eluting peak. *(R) stereochemistry rationally assigned to Compounds 19A* and 19A*-OtBu based on (i) the hWB NLRP3 inhibitory activity of the more active isomer Compound 19A* (compared to (S)-isomer 19B*) and (ii) the known absolute (R) stereochemistry of the more active isomer, Compound 16A (compared to (S)-isomer 16B).

Compound 19A*-OtBu: LCMS:(ES, m/z): RT=1.54 min, m/z=491.1 [M+H]⁺; H NMR (400 MHz, DMSO-d₆) δ 7.86-7.52 (m, 1H), 5.37-4.93 (m, 1H), 3.54-3.37 (m, 2H), 3.30-3.14 (m, 2H), 2.44-2.02 (m, 4H), 1.78-1.65 (m, 2H), 1.43 (d, J=17.2 Hz, 15H).

Compound 19B*-OtBu: LCMS:(ES, m/z): RT=1.54 min, m/z=491.1 [M+H]⁺; H NMR (400 MHz, DMSO-d₆) δ 7.83-7.70 (m, 1H), 5.36-4.94 (m, 1H), 3.54-3.38 (m, 2H), 3.30-3.15 (m, 2H), 2.45-2.00 (m, 4H), 1.78-1.65 (m, 2H), 1.43 (d, J=17.2 Hz, 15H).

Step 5: Into an 8 mL vial was added Compound 19A*-OtBu (35 mg, 0.070 mmol, 1 equiv), trifluoroacetic acid (TFA) (0.40 mL) and dichloromethane (DCM) (2.0 mL). The resulting mixture was stirred at room temperature for 2 h, then diluted with water (15 mL) and dried by lyophilization to provide Compound 19A* (21.2 mg, 68% yield).

Step 6: Into an 8 mL vial was added Compound 19B*-OtBu (35 mg, 0.070 mmol, 1 equiv), trifluoroacetic acid (TFA) (0.40 mL) and dichloromethane (DCM) (2.0 mL). The resulting mixture was stirred at room temperature overnight, then diluted with water (15 mL), and dried by lyophilization to provide Compound 19B* (25.6 mg, 83% yield).

Compound 19A*: LCMS:(ES, m/z): RT=1.27 min, m/z=435.2 [M+H]⁺; ¹H NMR (400 MHz, Methanol-d₄) δ 7.56-7.47 (m, 1H), 5.53-5.12 (m, 1H), 3.68-3.53 (m, 2H), 3.53-3.36 (m, 2H), 2.63-2.17 (m, 4H), 1.99-1.84 (m, J=6.8 Hz, 2H), 1.59 (s, 2H), 1.56-1.48 (m, 4H).

Compound 19B*: LCMS:(ES, m/z): RT=1.27 min, m/z=435.2 [M+H]⁺; H NMR (400 MHz, Methanol-d₄) δ 7.56-7.47 (m, 1H), 5.52-5.12 (m, 1H), 3.68-3.53 (m, 2H), 3.53-3.36 (m, 2H), 2.64-2.16 (m, 4H), 1.99-1.85 (m, J=6.8 Hz, 2H), 1.59 (s, 2H), 1.56-1.48 (m, 4H).

### Example 13. 4-(3-(5-cyclopropyl-7-fluoro-3,3-dimethyl-2-oxoindolin-1-yl)-2-oxopyrrolidin-1-yl)butanoic acid (Compound 22, rac-22), tert-butyl 4-(3-(5-cyclopropyl-7-fluoro-3,3-dimethyl-2-oxoindolin-1-yl)-2-oxopyrrolidin-1-yl)butanoate (Compound 22-OtBu), (R)-4-(3-(5-cyclopropyl-7-fluoro-3,3-dimethyl-2-oxoindolin-1-yl)-2-oxopyrrolidin-1-yl)butanoic acid (Compound 22A*), and (S)-4-(3-(5-cyclopropyl-7-fluoro-3,3-dimethyl-2-oxoindolin-1-yl)-2-oxopyrrolidin-1-yl)butanoic acid (Compound 22B*)

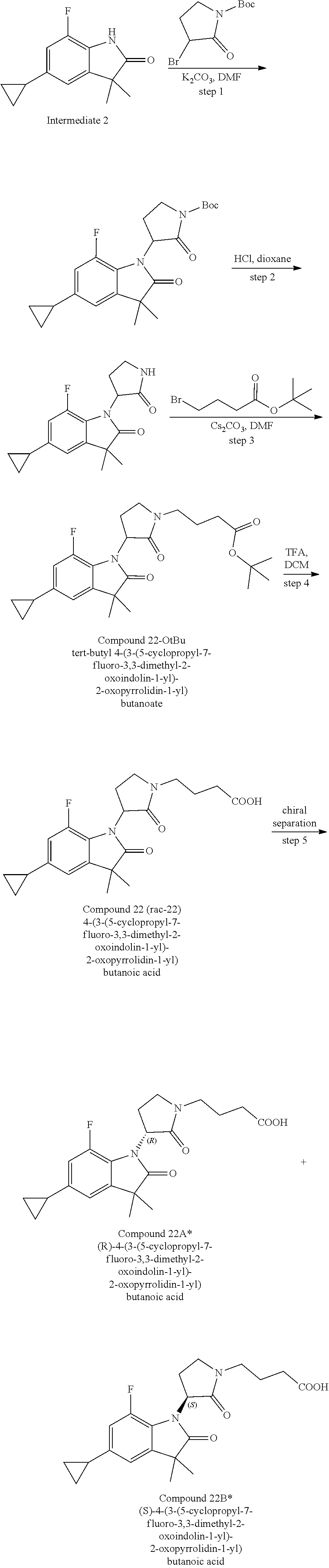

Step 1: A solution of 5-cyclopropyl-7-fluoro-3,3-dimethylindolin-2-one (Intermediate 2) (160 mg, 0.73 mmol, 1 equiv) and tert-butyl 3-bromo-2-oxopyrrolidine-1-carboxylate (385 mg, 1.46 mmol, 2 equiv), K₂CO₃ (302 mg, 2.19 mmol, 3 equiv) in dimethylformamide (DMF) (2 mL) was stirred at 80° C. overnight, then quenched by the addition of water (3 mL) at room temperature. The resulting mixture was concentrated under reduced pressure and the resulting residue was purified by reversed-phase flash chromatography (C18 silica gel; mobile phase, acetonitrile in water, 35% to 45% gradient in 10 min; detector, UV 220 nm) to provide tert-butyl 3-(5-cyclopropyl-7-fluoro-3,3-dimethyl-2-oxoindol-1-yl)-2-oxopyrrolidine-1-carboxylate (160 mg, 54% yield). LCMS: (ES, m/z): RT=0.87 min, m/z=303.1[M+H]⁺.

Step 2: A solution of tert-butyl 3-(5-cyclopropyl-7-fluoro-3,3-dimethyl-2-oxoindol-1-yl)-2-oxopyrrolidine-1-carboxylate (150 mg, 0.37 mmol, 1 equiv) and HCl(gas) in 1,4-dioxane (2 mL, 4M) was stirred at room temperature for 1 h, then the resulting mixture was concentrated under reduced pressure to provide 5-cyclopropyl-7-fluoro-3,3-dimethyl-1-(2-oxopyrrolidin-3-yl)indol-2-one (120 mg, 96% yield) which was used directly in the next step. LCMS: (ES, m/z): RT=0.65 min, m/z=303.1 [M+H]⁺.

Step 3: A solution of 5-cyclopropyl-7-fluoro-3,3-dimethyl-1-(2-oxopyrrolidin-3-yl)indol-2-one (90.0 mg, 0.29 mmol, 1 equiv), tert-butyl 4-bromobutanoate (133 mg, 0.59 mmol, 2 equiv), Cs₂CO₃ (970 mg, 2.98 mmol, 10 equiv) in dimethylformamide (DMF) (1 mL) was stirred at 100° C. for 1 h, then diluted with water (5 mL), and the resulting mixture was concentrated under vacuum. The resulting residue was purified by reversed-phase flash chromatography (C18 silica gel; mobile phase, acetonitrile in water, 40% to 50% gradient in 10 min; detector, UV 220 nm) to provide tert-butyl 4-(3-(5-cyclopropyl-7-fluoro-3,3-dimethyl-2-oxoindolin-1-yl)-2-oxopyrrolidin-1-yl)butanoate (Compound 22-OtBu), (80 mg, 60% yield) as a mixture of two stereoisomers. LCMS: (ES, m/z): RT=0.89 min, m/z=445.2[M+H]⁺.

Step 4: A solution of Compound 22-OtBu (70 mg, 0.15 mmol, 1 equiv), trifluoroacetic acid (TFA) (1 mL) in dichloromethane (DCM) (1 mL) was stirred at room temperature for 1 h, then was concentrated under reduced pressure to provide a crude residue (50 mg) which was purified by Prep-HPLC (XSelect CSH Prep C18 OBD Column, 19*250 mm, 5 m; Mobile Phase A: water (0.05% TFA), Mobile Phase B: acetonitrile; Flow rate: 25 mL/min; Gradient: 28% B to 38% B in 20 min; Wave Length: 254/220 nm; RT(min): 18) to afford 4-(3-(5-cyclopropyl-7-fluoro-3,3-dimethyl-2-oxoindolin-1-yl)-2-oxopyrrolidin-1-yl)butanoic acid (Compound 22, rac-22) (35 mg, 57% yield) as a mixture of two stereoisomers. LCMS: (ES, m/z): RT=1.08 min, m/z=389.1[M+H]⁺.

Step 5: The Compound 22 mixture (35 mg) was separated by Prep-Chiral HPLC (CHIRALPAK IF, 2*25 cm, 5 m; Mobile Phase A: Hexanes (0.1% TFA), Mobile Phase B: isopropanol: DCM=1:1; Flow rate: 20 mL/min; Gradient: 30% B to 30% B in 23 min; Wave Length: 220/254 nm) to provide Compound 22-A*: (10.5 mg, 30% yield, RT (min): 14.53) as the first eluting peak, and Compound 22-B* (11.8 mg, 34% yield, RT (min): 18.81) as the second eluting peak. *(R) stereochemistry rationally assigned to Compound 22A* based on (i) the hWB NLRP3 inhibitory activity of the more active isomer Compound 22A* (compared to (S)-isomer 22B*) and (ii) the known absolute (R) stereochemistry of the more active isomer, Compound 16A (compared to (S)-isomer 16B).

Compound 22A*: LCMS: (ES, m/z): RT=1.403 min, m/z=389.2[M+H]¹H NMR (400 MHz, DMSO-d6) δ 12.06 (s, 1H), 7.07-6.98 (m, 1H), 6.89-6.75 (m, 1H), 5.25 (d, J=10.2 Hz, 1H), 3.44 (d, J=16.2 Hz, 2H), 3.31-3.17 (m, J=6.7 Hz, 2H), 2.33-2.21 (m, 4H), 1.92 (d, J=13.4 Hz, 1H), 1.82-1.64 (m, J=6.9 Hz, 2H), 1.28-1.14 (m, 6H), 0.97-0.82 (m, 2H), 0.75-0.63 (m, 2H).

Compound 22B*: LCMS: (ES, m/z): RT=1.395 min, m/z=389.2[M+H]⁺. ¹H NMR (400 MHz, DMSO-d6) δ 12.07 (s, 1H), 7.07-6.98 (m, 1H), 6.89-6.76 (m, 1H), 5.25 (d, J=10.2 Hz, 1H), 3.43 (d, J=18.8 Hz, 2H), 3.23 (d, J=7.2 Hz, 2H), 2.25 (d, J=7.3 Hz, 3H), 2.12-1.99 (m, 1H), 1.92 (d, J=13.4 Hz, 1H), 1.81-1.64 (m, 2H), 1.28-1.14 (m, 6H), 0.93 (d, J=8.4 Hz, 2H), 0.76-0.63 (m, 2H).

### Example 14. 4-(3-(5-cyclopropyl-4,7-difluoro-3,3-dimethyl-2-oxoindolin-1-yl)-2-oxopyrrolidin-1-yl)pentanoic acid (Compound 24, rac-24), ethyl 4-(3-(5-cyclopropyl-4,7-difluoro-3,3-dimethyl-2-oxoindolin-1-yl)-2-oxopyrrolidin-1-yl)pentanoate (Compound 24-OEt), (S)-4-((R)-3-(5-cyclopropyl-4,7-difluoro-3,3-dimethyl-2-oxoindolin-1-yl)-2-oxopyrrolidin-1-yl)pentanoic acid (Compound 24A*), (R)-4-((S)-3-(5-cyclopropyl-4,7-difluoro-3,3-dimethyl-2-oxoindolin-1-yl)-2-oxopyrrolidin-1-yl)pentanoic acid (Compound 24B*), (S)-4-((S)-3-(5-cyclopropyl-4,7-difluoro-3,3-dimethyl-2-oxoindolin-1-yl)-2-oxopyrrolidin-1-yl)pentanoic acid (Compound 24C*), and (R)-4-((R)-3-(5-cyclopropyl-4,7-difluoro-3,3-dimethyl-2-oxoindolin-1-yl)-2-oxopyrrolidin-1-yl)pentanoic acid (Compound 24D*)

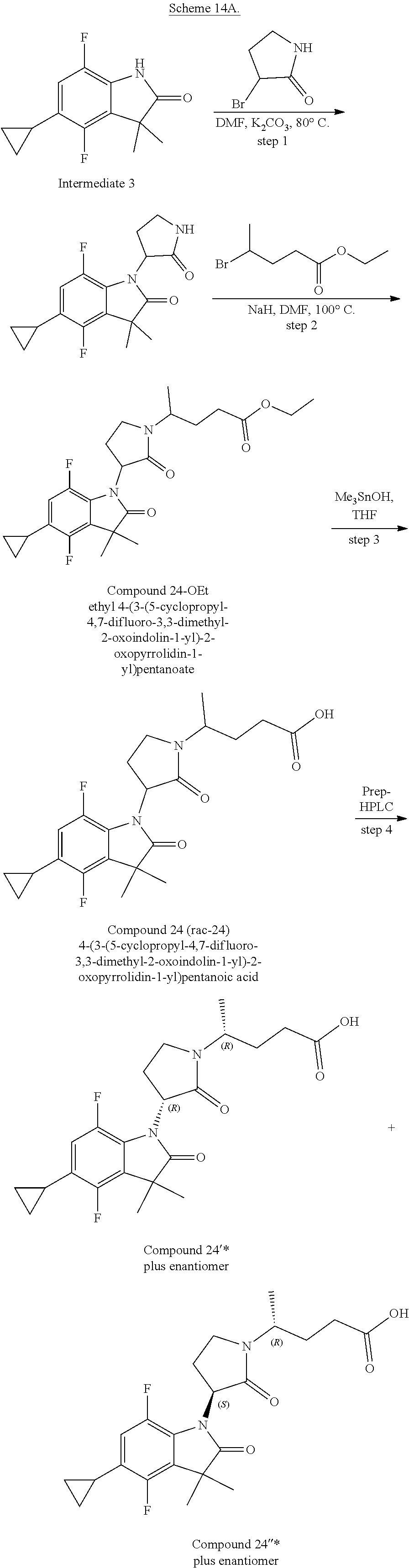

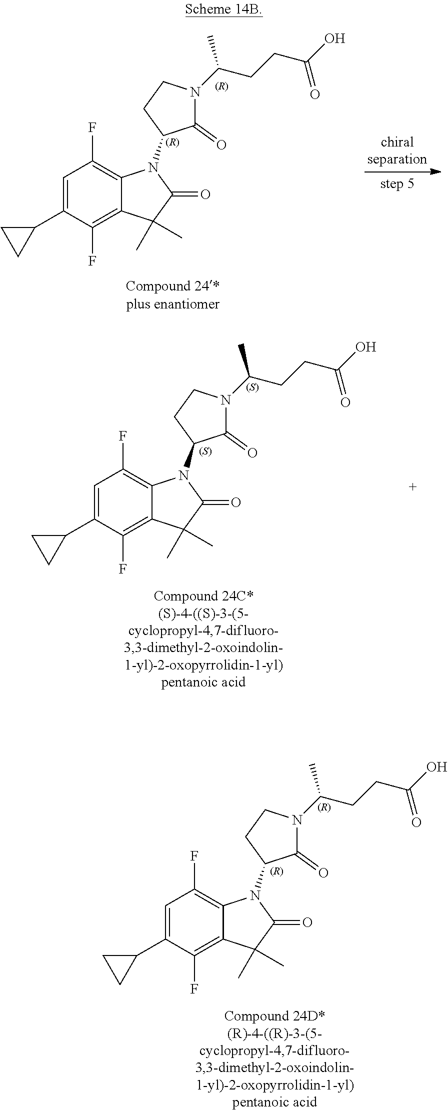

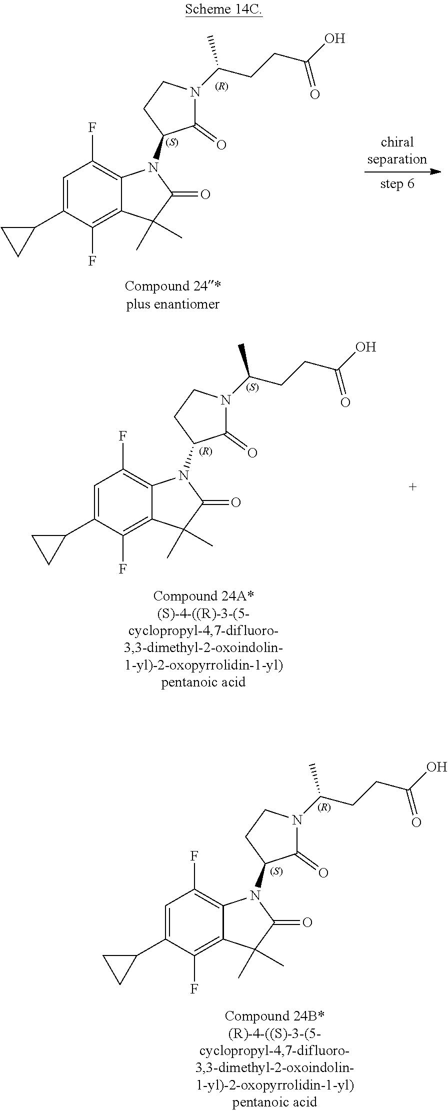

Step 1: Into a 20 mL vial was added 5-cyclopropyl-4,7-difluoro-3,3-dimethylindolin-2-one (Intermediate 3) (500 mg, 2.10 mmol, 1 equiv), dimethylformamide (DMF) (5 mL), 3-bromopyrrolidin-2-one (415 mg, 2.52 mmol, 1.20 equiv) and K₂CO₃ (874 mg, 6.32 mmol, 3 equiv) at room temperature. The resulting mixture was stirred overnight at 80° C., then quenched by the addition of water (20 mL) at room temperature. The resulting mixture was extracted with CH₂Cl₂ (3×20 mL). The combined organic layers were dried over anhydrous Na₂SO₄. After filtration, the filtrate was concentrated under reduced pressure and the residue was purified by reversed-phase flash chromatography (C18 silica gel; mobile phase, acetonitrile in water, 0% to 100% gradient in 30 min; detector, UV 254 nm/220 nm) to provide 5-cyclopropyl-4,7-difluoro-3,3-dimethyl-1-(2-oxopyrrolidin-3-yl)indol-2-one (400 mg, 53% yield). LCMS:(ES, m/z): RT=0.705 min, m/z=321.2[M+H]⁺.

Step 2: Into an 8 mL vial was added 5-cyclopropyl-4,7-difluoro-3,3-dimethyl-1-(2-oxopyrrolidin-3-yl)indol-2-one (20 mg, 0.062 mmol, 1 equiv) and dimethylformamide (DMF) (0.5 mL) at room temperature. Then NaH (60% in mineral oil, 5.0 mg, 0.12 mmol, 2 equiv) was added at 0° C. The resulting mixture was stirred for 30 min at room temperature. Then ethyl 4-bromopentanoate (39 mg, 0.19 mmol, 3 equiv) was added at room temperature, and the resulting mixture was stirred for 2 h at 100° C. under nitrogen atmosphere. The reaction was repeated 20 times, then worked up together as follows: the reaction was quenched by water (20 mL) at room temperature, the mixture extracted with CH₂Cl₂ (3×20 mL), and the combined organic layers were dried over anhydrous Na₂SO₄. After filtration, the filtrate was concentrated under reduced pressure to provide a residue, which was purified by reversed-phase flash chromatography (C18 silica gel, mobile phase: acetonitrile in water, 0% to 100% gradient in 30 min; detector, UV 254/220 nm) to provide ethyl 4-(3-(5-cyclopropyl-4,7-difluoro-3,3-dimethyl-2-oxoindolin-1-yl)-2-oxopyrrolidin-1-yl)pentanoate (Compound 24-OEt) (80 mg, 11% yield) as amixture of two stereoisomers. LCMS:(ES, m/z): RT=0.884 min, m/z=449.2[M+H]⁺.

Step 3: Into an 8 mL vial was added Compound 24-OEt (80.00 mg, 0.17 mmol, 1 equiv), tetrahydrofuran (THF) (2 mL) and (CH₃)₃SnOH (322.53 mg, 1.78 mmol, 10 equiv) at room temperature. The resulting mixture was stirred at 80° C. for 16 h, then was quenched by water (10 mL) at room temperature. The resulting mixture was extracted with CH₂Cl₂ (3×10 mL), and the combined organic layers were dried over anhydrous Na₂SO₄. After filtration, the filtrate was concentrated under reduced pressure to provide a residue, which was purified by reversed-phase flash chromatography (C18 silica gel; mobile phase, acetonitrile in water, 0% to 30% gradient in 30 min; detector, UV 254/220 nm) to provide 4-(3-(5-cyclopropyl-4,7-difluoro-3,3-dimethyl-2-oxoindolin-1-yl)-2-oxopyrrolidin-1-yl)pentanoic acid (Compound 24, rac-24) (70 mg, 70% yield) as a mixture of four stereoisomers. LCMS:(ES, m/z): RT=0.721 min, m/z=421.2[M+H]⁺.

Step 4: The Compound 24 mixture of Step 3 (70 mg) was separated by Prep-HPLC (XSelect CSH Prep C18 OBD Column, 19*250 mm; 5 m; Mobile Phase A: water (0.05% TFA), Mobile Phase B: acetonitrile; Flow rate: 25 mL/min; Gradient: 38% B to 48% B in 15 min; Wave Length: 254/220 nm) to afford as the first eluting peak Compound 24′* (13 mg, 25% yield, RT (min): 12.1) as a mixture of two stereoisomers, and as the second eluting peak Compound 24″* (8 mg, 15% yield, RT (min): 13.6) as a mixture of two stereoisomers. * Stereochemistry arbitrarily assigned.

Compound 24′*: LCMS:(ES, m/z): RT=0.711 min, m/z=421.2[M+H]. ¹H NMR (400 MHz, Methanol-d₄) δ 6.74-6.69 (m, 1H), 5.41-5.36 (m, 1H), 4.25-4.11 (m, 1H), 3.58-3.44 (m, 2H), 2.42 (s, 1H), 2.33-2.12 (m, 3H), 2.06-1.77 (m, 3H), 1.58-1.40 (m, 6H), 1.22 (d, J=6.8 Hz, 3H), 1.05-0.92 (m, 2H), 0.73-0.63 (m, 2H).

Compound 24″*: LCMS:(ES, m/z): RT=0.731 min, m/z=421.2[M+H]. ¹H NMR (400 MHz, Methanol-d₄) δ 6.71-6.67 (m, 1H), 5.48 (t, J=10.4 Hz, 1H), 4.20 (q, J=6.8 Hz, 1H), 3.57-3.41 (m, 2H), 2.46 (s, 1H), 2.19 (t, J=7.6 Hz, 3H), 2.08-1.99 (m, 1H), 1.83 (q, J=7.6 Hz, 2H), 1.58-1.42 (m, 6H), 1.29-1.23 (m, 3H), 1.04-0.96 (m, 2H), 0.73-0.64 (m, 2H).

Step 5: Compound 24′* (13 mg) was separated by Chiral HPLC (CHIRALPAK IC, 2*25 cm, 5 μm; Mobile Phase A: Hexanes (0.1% TFA), Mobile Phase B: isopropanol (IPA): DCM=1:1; Flow rate: 20 mL/min; Gradient: 40% B to 40% B in 12 min; Wave Length: 220/254 nm) to afford (S)-4-((S)-3-(5-cyclopropyl-4,7-difluoro-3,3-dimethyl-2-oxoindolin-1-yl)-2-oxopyrrolidin-1-yl)pentanoic acid (Compound 24C*) (5.8 mg, 45% yield, RT (min): 6.64) as the first eluting peak and (R)-4-((R)-3-(5-cyclopropyl-4,7-difluoro-3,3-dimethyl-2-oxoindolin-1-yl)-2-oxopyrrolidin-1-yl)pentanoic acid (Compound 24D*) (4.7 mg, 36% yield, RT (min): 9.93) as the second eluting peak. * Stereochemistry arbitrarily assigned.

Compound 24C*: LCMS: (ES, m/z): RT=0.820 min, m/z=421.1[M+H]. ¹H NMR (400 MHz, Methanol-d₄) δ 6.81-6.69 (m, 1H), 5.45-5.06 (m, 1H), 4.27-4.09 (m, 1H), 3.52-3.39 (m, 2H), 2.56-2.31 (m, 3H), 2.26-2.10 (m, 1H), 2.11-2.02 (m, 1H), 1.90 (m, 2H), 1.55 (s, 2H), 1.47 (d, J=12.8 Hz, 4H), 1.23-1.17 (m, 3H), 1.04-0.94 (m, 2H), 0.69 (t, J=5.6 Hz, 2H).

Compound 24D*: LCMS: (ES, m/z): RT=0.818 min, m/z=421.1[M+H]. ¹H NMR (400 MHz, Methanol-d₄) δ 6.74-6.69 (m, 1H), 5.46-5.06 (m, 1H), 4.27-4.07 (m, 1H), 3.55-3.41 (m, 2H), 2.55-2.30 (m, 3H), 2.27-2.12 (m, 1H), 2.07-2.01 (m, 1H), 1.99-1.82 (m, 2H), 1.55 (s, 2H), 1.46 (d, J=12.8 Hz, 4H), 1.23-1.16 (m, 3H), 1.04-0.94 (m, 2H), 0.69 (t, J=5.6 Hz, 2H).

Step 6: Compound 24″* (8 mg) was separated by Chiral HPLC (CHIRALPAK IG, 2*25 cm, 5 μm; Mobile Phase A: Hexanes (0.1% TFA), Mobile Phase B: isopropanol (IPA): DCM=1:1; Flow rate: 20 mL/min; Gradient: 40% B to 40% B in 10 min; Wave Length: 220/254 nm) to afford (S)-4-((R)-3-(5-cyclopropyl-4,7-difluoro-3,3-dimethyl-2-oxoindolin-1-yl)-2-oxopyrrolidin-1-yl)pentanoic acid (Compound 24A*) (2.9 mg, 36% yield, RT (min): 5.53) as the first eluting peak and (R)-4-((S)-3-(5-cyclopropyl-4,7-difluoro-3,3-dimethyl-2-oxoindolin-1-yl)-2-oxopyrrolidin-1-yl)pentanoic acid (Compound 24B*) (2.8 mg, 35% yield, RT (min): 7.18) as the second eluting peak. *Stereochemistry arbitrarily assigned.

Compound 24A*: LCMS: (ES, m/z): RT=1.615 min, m/z=421.2[M+H]⁺. ¹H NMR (400 MHz, Methanol-d₄) δ 6.75-6.67 (m, 1H), 5.51-5.02 (m, 1H), 4.28-4.13 (m, 1H), 3.60-3.40 (m, 2H), 2.50-2.42 (m, 1H), 2.32 (t, J=7.2 Hz, 2H), 2.21-2.13 (m, 1H), 2.07-2.01 (m, 1H), 1.94-1.79 (m, 2H), 1.61-1.43 (m, 6H), 1.33-1.21 (m, 3H), 1.03-0.95 (m, 2H), 0.70-0.67 (m, 2H).

Compound 24B*: LCMS: (ES, m/z): RT=1.605 min, m/z=421.1[M+H]. ¹H NMR (400 MHz, Methanol-d₄) δ 6.75-6.68 (m, 1H), 5.51-5.05 (m, 1H), 4.20 (d, J=9.2 Hz, 1H), 3.59-3.41 (m, 2H), 2.46 (d, J=9.6 Hz, 1H), 2.32 (t, J=7.2 Hz, 2H), 2.22-2.13 (m, 1H), 2.08-2.02 (m, 1H), 1.95-1.80 (m, 2H), 1.58-1.41 (m, 6H), 1.33-1.25 (m, 3H), 1.04-0.95 (m, 2H), 0.70-0.67 (m, 2H).

### Example 15. 4-(3-(5-cyclopropyl-4,7-difluoro-3,3-dimethyl-2-oxoindolin-1-yl)-2-oxopyrrolidin-1-yl)-3-methylbutanoic acid (Compound 26, rac-26), (S)-4-((R)-3-(5-cyclopropyl-4,7-difluoro-3,3-dimethyl-2-oxoindolin-1-yl)-2-oxopyrrolidin-1-yl)-3-methylbutanoic acid (Compound 26A*), (S)-4-((S)-3-(5-cyclopropyl-4,7-difluoro-3,3-dimethyl-2-oxoindolin-1-yl)-2-oxopyrrolidin-1-yl)-3-methylbutanoic acid (Compound 26B*), (R)-4-((R)-3-(5-cyclopropyl-4,7-difluoro-3,3-dimethyl-2-oxoindolin-1-yl)-2-oxopyrrolidin-1-yl)-3-methylbutanoic acid (Compound 26C*), and (R)-4-((S)-3-(5-cyclopropyl-4,7-difluoro-3,3-dimethyl-2-oxoindolin-1-yl)-2-oxopyrrolidin-1-yl)-3-methylbutanoic acid (Compound 26D*) and ethyl 4-(3-(5-cyclopropyl-4,7-difluoro-3,3-dimethyl-2-oxoindolin-1-yl)-2-oxopyrrolidin-1-yl)-3-methylbutanoate (Compound 26-OEt)

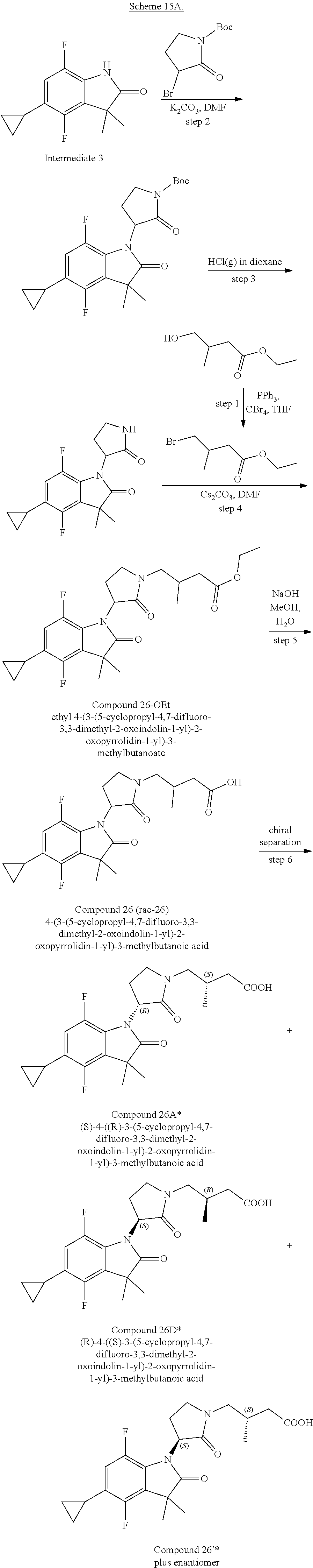

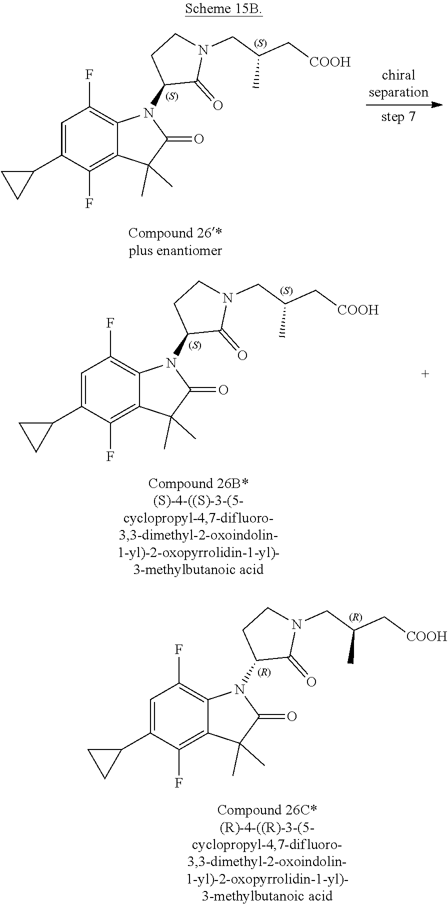

Step 1: Into a 250 mL 3-necked round-bottom flask was added ethyl 4-hydroxy-3-methylbutanoate (6.00 g, 41.004 mmol, 1 equiv), PPh₃ (32.3 g, 123 mmol, 3 equiv), CBr₄ (40.8 g, 123 mmol, 3 equiv), and tetrahydrofuran (THF) (60 mL), and the resulting mixture was stirred at room temperature for 3 h under nitrogen atmosphere. The reaction was then poured into sat. NaHCO₃ (aq.) at 0° C., extracted with EtOAc (3×100 mL), and the combined organic layers were washed with brine (100 mL), and dried over anhydrous Na₂SO₄. After filtration, the filtrate was concentrated under reduced pressure to provide a residue, which was purified by silica gel column chromatography, eluting with CH₂Cl₂/petroleum ether (5:1), to afford ethyl 4-bromo-3-methylbutanoate (3.3 g, 38% yield).

LCMS: (ES, m/z): RT=0.71 min, m/z=209.1[M+H]⁺.

Step 2: Into a 20 mL vial was added 5-cyclopropyl-4,7-difluoro-3,3-dimethylindolin-2-one (Intermediate 3) (500 mg, 2.10 mmol, 1 equiv), tert-butyl 3-bromo-2-oxopyrrolidine-1-carboxylate (1.11 g, 4.20 mmol, 2 equiv), K₂CO₃ (874 mg, 6.32 mmol, 3 equiv), and dimethylformamide (DMF) (5 mL).

The resulting mixture was stirred at 80° C. for 1 h, then the reaction was quenched with water (20 mL) at room temperature. The resulting mixture was extracted with EtOAc (3×30 mL), and the combined organic layers were washed with brine (1×30 mL) and dried over anhydrous Na₂SO₄. After filtration, the filtrate was concentrated under reduced pressure to provide a residue, which was purified by silica gel column chromatography, eluting with CH₂Cl₂/petroleum ether (6:1), to afford tert-butyl 3-(5-cyclopropyl-4,7-difluoro-3,3-dimethyl-2-oxoindol-1-yl)-2-oxopyrrolidine-1-carboxylate (700 mg, 79% yield). LCMS: (ES, m/z): RT=0.91 min, m/z=421.1[M+H]⁺.

Step 3: Into a 40 mL vial was added tert-butyl 3-(5-cyclopropyl-4,7-difluoro-3,3-dimethyl-2-oxoindol-1-yl)-2-oxopyrrolidine-1-carboxylate (700 mg, 1.66 mmol, 1 equiv) and HCl(g) in 1,4-dioxane (4.0 M, 7 mL). The resulting mixture was stirred at room temperature for 1 h, then concentrated under reduced pressure to provide 5-cyclopropyl-4,7-difluoro-3,3-dimethyl-1-(2-oxopyrrolidin-3-yl)indol-2-one hydrochloride (510 mg, 96% yield) which was used in the next step directly. LCMS: (ES, m/z): RT=0.45 min, m/z=321.1[M+H]⁺.

Step 4: Into a 40 mL vial was added 5-cyclopropyl-4,7-difluoro-3,3-dimethyl-1-(2-oxopyrrolidin-3-yl)indol-2-one hydrochloride (500 mg, 1.56 mmol, 1 equiv), ethyl 4-bromo-3-methylbutanoate (from Step 1 of this Example) (3.26 g, 15.6 mmol, 10 equiv), Cs₂CO₃ (1.53 g, 4.68 mmol, 3 equiv), and dimethylformamide (DMF) (5.0 mL). The resulting mixture was stirred at 80° C. for 2 h, then the resulting mixture was filtered, the filter cake was washed with MeOH (3×30 mL), and the filtrate concentrated under reduced pressure to provide a residue, which was purified by reversed-phase flash chromatography (C18 silica gel; mobile phase, acetonitrile in water (0.1% TFA), 60% to 80% gradient in 30 min; detector, UV 220/254 nm) to provide ethyl 4-(3-(5-cyclopropyl-4,7-difluoro-3,3-dimethyl-2-oxoindolin-1-yl)-2-oxopyrrolidin-1-yl)-3-methylbutanoate (Compound 26-OEt) (120 mg, 18% yield) as a mixture of four stereoisomers. LCMS: (ES, m/z): RT=0.88 min, m/z=449.1[M+H]⁺.

Step 5: Into an 8 mL vial was added Compound 26-OEt (120 mg, 0.15 mmol, 1 equiv), NaOH (18.7 mg, 0.450 mmol, 3 equiv), MeOH (1 mL), and H₂O (0.2 mL). The resulting mixture was stirred at room temperature for 2 h, then the mixture was adjusted pH=3 with 1M HCl (aq.). The resulting mixture was extracted with EtOAc (3×30 mL), the combined organic layers were washed with brine (30 mL), dried over anhydrous Na₂SO₄, and after filtration, the filtrate was concentrated under reduced pressure to provide a residue, which was purified by reversed-phase chromatography (C18 silica gel; mobile phase, acetonitrile in water, 20% to 50% gradient in 30 min; detector, UV 220/254 nm, RT(min): 12) to provide 4-(3-(5-cyclopropyl-4,7-difluoro-3,3-dimethyl-2-oxoindolin-1-yl)-2-oxopyrrolidin-1-yl)-3-methylbutanoic acid (Compound 26) (70 mg, 58% yield) as a mixture of four stereoisomers. LCMS: (ES, m/z): RT=0.69 min, m/z=421.1[M+H]⁺.

Step 6: The Compound 26 mixture (70 mg) was purified by Prep-Chiral-HPLC (CHIRALPAK IA, 2*25 cm, 5 m; Mobile Phase A: Hexanes (0.1% TFA), Mobile Phase B: isopropanol (IPA): DCM=1:1; Flow rate: 20 mL/min; Gradient: 20% B to 20% B in 12 min; Wave Length: 220/254 nm) to afford as the first eluting peak (S)-4-((R)-3-(5-cyclopropyl-4,7-difluoro-3,3-dimethyl-2-oxoindolin-1-yl)-2-oxopyrrolidin-1-yl)-3-methylbutanoic acid (Compound 26A*) (5.6 mg, 8% yield, RT (min): 5.83), as the second eluting peak Compound 26′* (19 mg, 27% yield, RT (min): 7.56) as a mixture of two stereoisomers, and as the third eluting peak (R)-4-((S)-3-(5-cyclopropyl-4,7-difluoro-3,3-dimethyl-2-oxoindolin-1-yl)-2-oxopyrrolidin-1-yl)-3-methylbutanoic acid (Compound 26D*) (4.9 mg, 7% yield, RT (min): 9.61). *Stereochemistry arbitrarily assigned.

Compound 26A*: LCMS: (ES, m/z): RT=1.39 min, m/z=421.2[M+H]⁺. ¹H NMR (400 MHz, Methanol-d₄) δ 6.71 (m, 1H), 5.45 (dd, J=11.2, 9.4 Hz, 1H), 3.68-3.50 (m, 2H), 3.28-3.08 (m, 2H), 2.62-2.29 (m, 4H), 2.19-2.10 (m, 1H), 2.08-1.98 (m, 1H), 1.55 (s, 2H), 1.53-1.42 (m, 4H), 1.04-0.90 (m, 5H), 0.74-0.65 (m, 2H).

Compound 26′*: LCMS: (ES, m/z): RT=1.32 min, m/z=421.2[M+H]. ¹H NMR (400 MHz, Methanol-d₄) δ 6.78-6.64 (m, 1H), 5.47-5.04 (m, 1H), 3.73-3.46 (m, 2H), 3.31-3.12 (m, 2H), 2.59-2.31 (m, 3H), 2.29-2.11 (m, 2H), 2.09-1.98 (m, 1H), 1.55 (s, 2H), 1.52-1.41 (m, 4H), 1.10-0.94 (m, 5H), 0.74-0.65 (m, 2H).

Compound 26D*: LCMS: (ES, m/z): RT=1.25 min, m/z=421.2[M+H]⁺. ¹H NMR (400 MHz, Methanol-d₄) δ 6.77-6.64 (m, 1H), 5.45 (dd, J=11.0, 9.4 Hz, 1H), 3.68-3.48 (m, 2H), 3.27-3.10 (m, 2H), 2.64-2.24 (m, 4H), 2.18-2.10 (m, 1H), 2.08-2.00 (m, 1H), 1.55 (s, 2H), 1.52-1.32 (m, 4H), 1.08-0.87 (m, 5H), 0.74-0.63 (m, 2H).

Step 7: The Compound 26′* mixture product of Step 6 (19 mg) was separated by Prep-Chiral-HPLC (CHIRALPAK IH, 2*25 cm, 5 m; Mobile Phase A: Hexanes (0.1% TFA), Mobile Phase B: isopropanol (IPA): DCM=1:1; Flow rate: 20 mL/min; Gradient: 20% B to 20% B in 18 min; Wave Length: 220/254 nm) to afford as the first eluting peak (S)-4-((S)-3-(5-cyclopropyl-4,7-difluoro-3,3-dimethyl-2-oxoindolin-1-yl)-2-oxopyrrolidin-1-yl)-3-methylbutanoic acid (Compound 26B*) (4.1 mg, 22% yield, RT (min): 7.67), and as the second eluting peak (R)-4-((R)-3-(5-cyclopropyl-4,7-difluoro-3,3-dimethyl-2-oxoindolin-1-yl)-2-oxopyrrolidin-1-yl)-3-methylbutanoic acid (Compound 26C*) (6.6 mg, 35% yield, RT (min): 13.84). *Stereochemistry arbitrarily assigned.

Compound 26B*: LCMS: (ES, m/z): RT=0.68 min, m/z=421.1[M+H]⁺. H NMR (400 MHz, Methanol-d₄) δ 6.80-6.62 (m, 1H), 5.43 (dd, J=11.2, 9.4 Hz, 1H), 3.70-3.59 (m, 1H), 3.59-3.47 (m, 1H), 3.29-3.13 (m, 2H), 2.62-2.25 (m, 4H), 2.17 (dd, J=17.4, 9.6 Hz, 1H), 2.08-1.97 (m, 1H), 1.55 (s, 2H), 1.52-1.32 (m, 4H), 1.10-0.86 (m, 5H), 0.76-0.61 (m, 2H).

Compound 26C*: LCMS: (ES, m/z): RT=1.32 min, m/z=421.2[M+H]⁺. ¹H NMR (400 MHz, Methanol-d₄) δ 6.78-6.63 (m, 1H), 5.43 (t, J=10.3 Hz, 1H), 3.69-3.50 (m, 2H), 3.30-3.12 (m, 2H), 2.59-2.11 (m, 5H), 2.09-1.99 (m, 1H), 1.55 (s, 2H), 1.52-1.31 (m, 4H), 1.14-0.87 (m, 5H), 0.75-0.61 (m, 2H).

Additional compounds provided in the below Table B were prepared following General Method Scheme A as described in the Methods of Preparation, in the Examples as described above, and/or in the described Procedure as provided in the below Table. NMR and LCMS data is provided for each compound synthesized. Dashed lines (--) indicate no data is available. Asterix (*) indicates arbitrary or rational stereochemical assignment.

TABLE B
Additional Examples

| | | LCMS | | |
|---|---|---|---|---|
| | | (m/z) | | |
| # | Compound | [M + 1]⁺ | ¹HNMR | Procedure |
| 2 | 4-(3-(5-cyclopropyl-7- | 399.10 | ¹H NMR (400 MHz, | Compound 2 was prepared |
| | fluoro-3,3-dimethyl-2- | | DMSO-d₆) δ 7.78 (dd, | following Example 1 using |
| | oxoindolin-1-yl)-2- | | J = 6.8, 2.0 Hz, 1H), | 5-cyclopropyl-7-fluoro-3,3- |
| | oxopyridin-1(2H)- | | 7.62 (dd, J = 7.1, 2.0 | dimethylindolin-2-one |
| | yl)butanoic acid | | Hz, 1H), 7.04 (d, J = | (Intermediate 2) as the |
| | | | 1.6 Hz, 1H), 6.73 (dd, | indolinone (i) reagent and |
| | | | J = 12.8, 1.6 Hz, 1H), | tert-butyl 4-(3-iodo-2- |
| | | | 6.35 (t, J = 7.0 Hz, | oxopyridin-1(2H)- |
| | | | 1H), 4.08-4.01 (m, | yl)butanoate as the lactam |
| | | | 1H), 3.91-3.84 (m, | (ii) reagent. |
| | | | 1H), 2.23 (t, J = 7.5 | |
| | | | Hz, 2H), 1.98-1.81 | |
| | | | (m, 3H), 1.36 (d, J = | |
| | | | 15.6 Hz, 6H), 1.02- | |
| | | | 0.84 (m, 2H), 0.75- | |
| | | | 0.62 (m, 2H). | |
| 3 | 4-(3-(5-cyclopropyl-4,7- | 417.10 | ¹H NMR (400 MHz, | Compound 3 was prepared |
| | difluoro-3,3-dimethyl-2- | | DMSO-d₆) δ 12.15 (s, | following Example 1 using |
| | oxoindolin-1-yl)-2- | | 1H), 7.80 (d, J = 6.8 | 5-cyclopropyl-4,7-difluoro- |
| | oxopyridin-1(2H)- | | Hz, 1H), 7.66 (d, J = | 3,3-dimethylindolin-2-one |
| | yl)butanoic acid | | 7.1 Hz, 1H), 6.75 (d, | (Intermediate 3) as the |
| | | | J = 12.0, 5.8 Hz, 1H), | indolinone (i) reagent and |
| | | | 6.37 (d, J = 6.9 Hz, | tert-butyl 4-(3-iodo-2- |
| | | | 1H), 4.05 (d, J = 13.8, | oxopyridin-1(2H)- |
| | | | 7.0 Hz, 1H), 3.87 (d, | yl)butanoate as the lactam |
| | | | J = 13.9, 7.2 Hz, 1H), | (ii) reagent. |
| | | | 2.22 (d, J = 7.4 Hz, | |
| | | | 2H), 2.01 (s, 1H), 1.87 | |
| | | | (d, J = 7.5 Hz, 2H), | |
| | | | 1.49 (s, 3H), 1.44 (s, | |
| | | | 3H), 0.94 (d, J = 9.4 | |
| | | | Hz, 2H), 0.71 (d, J = | |
| | | | 5.4 Hz, 2H). | |
| 12 | 4-(3-(5-cyclopropyl-4,7- | 485.15 | ¹H NMR (400 MHz, | Compound 12 was prepared |
| | difluoro-3,3-dimethyl-2- | | Methanol-d₄) δ 8.38 | following Example 1 using |
| | oxoindolin-1-yl)-2-oxo- | | (d, J = 2.4 Hz, 1H), | 5-cyclopropyl-4,7-difluoro- |
| | 5-(trifluoromethyl)pyridin- | | 7.95 (d, J = 2.4 Hz, | 3,3-dimethylindolin-2-one |
| | 1(2H)-yl)butanoic acid | | 1H), 6.66 (d, J = 11.6 | (Intermediate 3) as the |
| | | | Hz, 1H), 4.23 (t, J = | indolinone (i) reagent and |
| | | | 7.2 Hz, 1H), 4.11 (d, | tert-butyl 4-((3-iodo-5- |
| | | | J = 7.2 Hz, 1H), 2.38 (t, | (trifluoromethyl)pyridin-2- |
| | | | J = 7.2 Hz, 2H), 2.13- | yl)oxy)butanoate as the |
| | | | 2.03 (m, 2H), 1.59 (d, | lactam (ii) reagent, using 3- |
| | | | J = 4.5 Hz, 6H), 1.05- | iodo-5- |
| | | | 0.94 (m, 2H), 0.70 (t, | (trifluoromethyl)pyridin- |
| | | | J = 2.4 Hz, 2H). | 2(1H)-one as the step 1 |
| | | | | reagent. |
| 13 | 4-(3-(5-cyclopropyl-4,7- | 493.25 | ¹H NMR (400 MHz, | Compound 13 was prepared |
| | difluoro-3,3-dimethyl-2- | | DMSO-d₆) δ 8.27 (d, | following Example 1 using |
| | oxoindolin-1-yl)-2-oxo- | | J = 2.8 Hz, 1H), 8.16 (d, | 5-cyclopropyl-4,7-difluoro- |
| | 5-phenylpyridin-1(2H)- | | J = 2.6 Hz, 1H), 7.70- | 3,3-dimethylindolin-2-one |
| | yl)butanoic acid | | 7.58 (m, 2H), 7.46 (t, | (Intermediate 3) as the |
| | | | J = 7.4 Hz, 2H), 7.34 (t, | indolinone (i) reagent and |
| | | | J = 7.4 Hz, 1H), 6.78 | tert-butyl 4-(3-iodo-2-oxo-5- |
| | | | (d, J = 11.8 Hz, 1H), | phenylpyridin-1(2H)- |
| | | | 4.15 (d, J = 6.6 Hz, | yl)butanoate as the lactam |
| | | | 1H), 3.99 (d, J = 13.2 | (ii) reagent, using 3-iodo-5- |
| | | | Hz, 1H), 2.27 (t, J = | phenylpyridin-2(1H)-one as |
| | | | 7.4 Hz, 2H), 1.97 (t, | the step 1 reagent. |
| | | | J = 14.8 Hz, 3H), 1.50 | |
| | | | (d, J = 22.8 Hz, 6H), | |
| | | | 0.95 (d, J = 9.2 Hz, | |
| | | | 2H), 0.72 (d, J = 5.6 | |
| | | | Hz, 2H). | |
| 14 | 4-(5-cyclopropyl-3-(5- | 457.10 | ¹H NMR (400 MHz, | Compound 14 was prepared |
| | cyclopropyl-4,7- | | DMSO-d₆) δ 7.61 (d, | following Example 1 using |
| | difluoro-3,3-dimethyl-2- | | J = 2.6 Hz, 1H), 7.48 (d, | 5-cyclopropyl-4,7-difluoro- |
| | oxoindolin-1-yl)-2- | | J = 2.6 Hz, 1H), 6.74 | 3,3-dimethylindolin-2-one |
| | oxopyridin-1(2H)- | | (d, J = 11.8 Hz, 1H), | (Intermediate 3) as the |
| | yl)butanoic acid | | 4.00 (d J = 12.8 Hz, | indolinone (i) reagent and |
| | | | 1H), 3.83 (d, J = 12.6 | tert-butyl 4-(5-cyclopropyl- |
| | | | Hz, 1H), 2.21 (t, J = | 3-iodo-2-oxopyridin-1(2H)- |
| | | | 7.6 Hz, 2H), 2.01 (t, | yl)butanoate as the lactam |
| | | | J = 8.61 Hz, 1H), 1.91- | (ii) reagent, using 5- |
| | | | 1.73 (m, 3H), 1.49 (s, | cyclopropyl-3-iodopyridin- |
| | | | 3H), 1.43 (s, 3H), 0.94 | 2(1H)-one as the step 1 |
| | | | (d, J = 8.8 Hz, 2H), | reagent. |
| | | | 0.89-0.80 (m, 2H), | |
| | | | 0.75-0.65 (m, 2H), | |
| | | | 0.62-0.51 (m, 2H). | |
| 15 | 4-(4-(5-cyclopropyl-4,7- | 467.10 | ¹H NMR (400 MHz, | Compound 15 was prepared |
| | difluoro-3,3-dimethyl-2- | | DMSO-d₆) δ 9.03 (s, | following Example 1 using |
| | oxoindolin-1-yl)-3- | | 1H), 7.74 (d, J = 8.6 | 5-cyclopropyl-4,7-difluoro- |
| | oxoisoquinolin-2(3H)- | | Hz, 1H), 7.41 (t, J = | 3,3-dimethylindolin-2-one |
| | yl)butanoic acid | | 7.8 Hz, 1H), 7.15 (d, | (Intermediate 3) as the |
| | | | J = 8.9 Hz, 1H), 7.07- | indolinone (i) reagent and |
| | | | 6.97 (m, 1H), 6.74- | tert-butyl 4-(4-iodo-3- |
| | | | 6.63 (m, 1H), 4.39- | oxoisoquinolin-2(3H)- |
| | | | 4.26 (m, 1H), 4.25- | yl)butanoate as the lactam |
| | | | 4.11 (m, 1H), 2.14 (s, | (ii) reagent, using 4- |
| | | | 1H), 2.07-1.91 (m, | iodoisoquinolin-3(2H)-one |
| | | | 3H), 1.59-1.57 (m, | as the step 1 reagent. |
| | | | 4H), 1.49 (s, 3H), 1.36 | |
| | | | (s, 1H), 0.97-0.90 (m, | |
| | | | 2H), 0.75-0.66 (m, 2H). | |
| 17 | 4-(3-(4,7-difluoro-3,3- | 446.2 | ¹H NMR (400 MHz, | Compound 17 was prepared |
| | dimethyl-2-oxo-5- | | Methanol-d₄) δ 7.83 | following Example 8 using |
| | (trifluoromethyl)indolin- | | (d, J = 4.4 Hz, 1H), | 4,7-difluoro-3,3-dimethyl-5- |
| | 1-yl)-2-oxopyrazin- | | 7.51-7.47 (m, 2H), | (trifluoromethyl)indolin-2- |
| | 1(2H)-yl)butanoic acid | | 4.25-4.06 (m, 2H), | one (Intermediate 9) as the |
| | | | 2.40 (t, J = 7.2 Hz, | indolinone (i) reagent and |
| | | | 2H), 2.10 (p, J = 7.2 | tert-butyl 4-(3-iodo-2- |
| | | | Hz, 2H), 1.63 (d, J = | oxopyrazin-1(2H)- |
| | | | 6.4 Hz, 6H). | yl)butanoate as the lactam |
| | | | | (ii) reagent. |
| 18 | 4-(3-(5-cyclopropyl-4- | 389.2 | — | 18 (rac-18), 18A* and |
| | fluoro-3,3-dimethyl-2- | | | 18B* were prepared |
| | oxoindolin-1-yl)-2- | | | following Example 10 using |
| | oxopyrrolidin-1- | | | 5-cyclopropyl-4-fluoro-3,3- |
| | yl)butanoic acid | | | dimethylindolin-2-one |
| 18A* | (R)-4-(3-(5-cyclopropyl- | 389.2 | ¹H NMR (300 MHz, | (Intermediate 14) as the |
| | 4-fluoro-3,3-dimethyl-2- | | Methanol-d₄) δ 6.96- | indolinone (i) reagent and |
| | oxoindolin-1-yl)-2- | | 6.84 (m, 1H), 6.62 (d, | tert-butyl 4-(3-bromo-2- |
| | oxopyrrolidin-1- | | J = 8.2 Hz, 1H), 5.15 | oxopyrrolidin-1- |
| | yl)butanoic acid | | (d, J = 9.7 Hz, 1H), | yl)butanoate as the lactam |
| | | | 3.69-3.56 (m, 2H), | (ii) reagent, using 3- |
| | | | 3.56-3.37 (m, 2H), | bromopyrrolidin-2-one as |
| | | | 2.55-2.27 (m, 4H), | the step 1 reagent to prepare |
| | | | 2.10-1.82 (m, 3H), | intermediate tert-butyl 4-(3- |
| | | | 1.53-1.45 (m, 6H), | (5-cyclopropyl-4-fluoro-3,3- |
| | | | 1.03-0.90 (m, 2H), | dimethyl-2-oxoindolin-1- |
| | | | 0.72-0.61 (m, 2H). | yl)-2- |
| 18B* | (S)-4-(3-(5-cyclopropyl- | 389.2 | ¹H NMR (300 MHz, | oxopyrrolidin-1- |
| | 4-fluoro-3,3-dimethyl-2- | | Methanol-d₄) δ 6.96- | yl)butanoate (18-OtBu). 18- |
| | oxoindolin-1-yl)-2- | | 6.84 (m, 1H), 6.62 (d, | OtBu was then subjected to |
| | oxopyrrolidin-1- | | J = 8.1 Hz, 1H), 5.15 | chiral separation conditions |
| | yl)butanoic acid | | (d, J = 9.7 Hz, 1H), | (CHIRAL ART Cellulose- |
| | | | 3.71-3.54 (m, 2H), | SC, 2*25 cm, 5 μm; Mobile |
| | | | 3.43 (s, 2H), 2.51- | Phase A: Hexanes (0.1% |
| | | | 2.39 (m, 2H), 2.39- | TFA), Mobile Phase B: |
| | | | 2.29 (m, 2H), 2.11- | EtOH:DCM = 1:1; Flow |
| | | | 1.97 (m, 1H), 1.97- | rate: 20 mL/min; Gradient: |
| | | | 1.82 (m, 2H), 1.53- | 40% B to 40% B in 9 min; |
| | | | 1.45 (m, 6H), 1.02- | Wave Length: 220/254 nm) |
| | | | 0.92 (m, 2H), 0.73- | to provide as the first eluting |
| | | | 0.61 (m, 2H). | peak tert-butyl (R)-4-(3-(5- |
| | | | | cyclopropyl-4-fluoro-3,3- |
| | | | | dimethyl-2-oxoindolin-1- |
| | | | | yl)-2-oxopyrrolidin-1- |
| | | | | yl)butanoate (18A*-OtBu; |
| | | | | Chiral RT (min): 6.55) and |
| | | | | as the second eluting peak |
| | | | | tert-butyl (S)-4-(3-(5- |
| | | | | cyclopropyl-4-fluoro-3,3- |
| | | | | dimethyl-2-oxoindolin-1- |
| | | | | yl)-2-oxopyrrolidin-1- |
| | | | | yl)butanoate (18B*-OtBu; |
| | | | | Chiral RT (min): 8.34). |
| | | | | 18A*-OtBu and 18B*- |
| | | | | OtBu were hydrolyzed as in |
| | | | | Example 8 to provide 18A* |
| | | | | and 18B*. |
| | | | | *Stereochemistry of 18A* |
| | | | | and 18B* were rationally |
| | | | | assigned based on hWB |
| | | | | inhibitory activity of 18A* |
| | | | | and known stereochemistry |
| | | | | of 16A. |
| 21 | 4-(3-(5-cyclopropyl-4- | 400.2 | ¹H NMR (400 MHz, | Compound 21 was prepared |
| | fluoro-3,3-dimethyl-2- | | Methanol-d₄) δ 7.78 | following Example 8 using |
| | oxoindolin-1-yl)-2- | | (d, J = 4.4 Hz, 1H), | 5-cyclopropyl-4-fluoro-3,3- |
| | oxopyrazin-1(2H)- | | 7.46 (d, J = 4.4 Hz, | dimethylindolin-2-one |
| | yl)butanoic acid | | 1H), 6.96-6.82 (m, | (Intermediate 14) as the |
| | | | 1H), 6.63 (d, J = 8.4 | indolinone (i) reagent and |
| | | | (m, 2H), 2.64-2.33 | tert-butyl 4-(3-iodo-2- |
| | | | (m, 2H), 2.21-1.99 | oxopyrazin-1(2H)- |
| | | | (m, 3H), 1.59 (s, 6H), | yl)butanoate as the lactam |
| | | | 1.09-0.94 (m, 2H), | (ii) reagent. |
| | | | 0.81-0.59 (m, 2H). | |
| 23 | 4-(3-(5-chloro-4,7- | 401.1 | — | 23 (rac-23), 23A* and |
| | difluoro-3,3-dimethyl-2- | | | 23B* were prepared |
| | oxoindolin-1-yl)-2- | | | following Example 8 using |
| | oxopyrrolidin-1- | | | 5-chloro-4,7-difluoro-3,3- |
| | yl)butanoic acid | | | dimethylindolin-2-one |
| 23A* | (R)-4-(3-(5-chloro-4,7- | 401.1 | ¹H NMR (400 MHz, | (Intermediate 23) as the |
| | difluoro-3,3-dimethyl-2- | | Methanol-d₄) δ 7.41- | indolinone (i) reagent and |
| | oxoindolin-1-yl)-2- | | 7.26 (m, 1H), 5.51- | tert-butyl 4-(3-bromo-2- |
| | oxopyrrolidin-1- | | 5.02 (m, 1H), 3.68- | oxopyrrolidin-1- |
| | yl)butanoic acid | | 3.52 (m, 2H), 3.52- | yl)butanoate as the lactam |
| | | | 3.38 (m, 2H), 2.61- | (ii) reagent, using 3- |
| | | | 2.32 (m, 3H), 2.22 (s, | bromopyrrolidin-2-one as |
| | | | 1H), 1.99-1.84 (m, | the step 1 reagent to prepare |
| | | | 2H), 1.58-1.41 (m, 6H). | intermediate tert-butyl 4-(3- |
| 23B* | (S)-4-(3-(5-chloro-4,7- | 401.1 | ¹H NMR (400 MHz, | (5-chloro-4,7-difluoro-3,3- |
| | difluoro-3,3-dimethyl-2- | | Methanol-d₄) δ 7.39- | dimethyl-2-oxoindolin-1- |
| | oxoindolin-1-yl)-2- | | 7.28 (m, 1H), 5.50- | yl)-2-oxopyrrolidin-1- |
| | oxopyrrolidin-1- | | 5.02 (m, 1H), 3.69- | yl)butanoate (23-OtBu). 23- |
| | yl)butanoic acid | | 3.51 (m, 2H), 3.51- | OtBu was subjected to |
| | | | 3.38 (m, 2H), 2.60- | chiral separation conditions |
| | | | 2.34 (m, 3H), 2.22 (d, | (CHIRALPAK IG, 2*25 cm, |
| | | | J = 3.6 Hz, 1H), 2.01- | 5 μm; Mobile Phase A: |
| | | | 1.86 (m, 2H), 1.65- | Hexanes (0.5% 2M NH₃- |
| | | | 1.44 (m, 6H). | MeOH), Mobile Phase B: |
| | | | | EtOH:DCM = 1:1; Flow rate: |
| | | | | 20 mL/min; Gradient: 20% |
| | | | | B to 20% B in 11 min; |
| | | | | Wave Length: 220/254 nm) |
| | | | | to provide as the first eluting |
| | | | | peak tert-butyl (R)-4-(3-(5- |
| | | | | chloro-4,7-difluoro-3,3- |
| | | | | dimethyl-2-oxoindolin-1- |
| | | | | yl)-2-oxopyrrolidin-1- |
| | | | | yl)butanoate (23A*-OtBu, |
| | | | | Chiral RT (min): 7.63), and |
| | | | | as the second eluting peak |
| | | | | tert-butyl (S)-4-(3-(5-chloro- |
| | | | | 4,7-difluoro-3,3-dimethyl-2- |
| | | | | oxoindolin-1-yl)-2- |
| | | | | oxopyrrolidin-1- |
| | | | | yl)butanoate |
| | | | | (23B*-OtBu, Chiral RT |
| | | | | (min): 9.8). 23A*-OtBu and |
| | | | | 23B*-OtBu were |
| | | | | hydrolyzed as in Example 8 |
| | | | | to provide 23A* and 23B*. |
| | | | | *Stereochemistry of 23A* |
| | | | | and 23B* were rationally |
| | | | | assigned based on hWB |
| | | | | inhibitory activity of 23A* |
| | | | | and known stereochemistry |
| | | | | of 16A. |
| 25 | 4-(4-(5-cyclopropyl-4,7- | 435.3 | — | 25 (rac-25), 25A* and |
| | difluoro-3,3-dimethyl-2- | | | 25B* were prepared |
| | oxoindolin-1-yl)-2,2- | | | following Example 8 using |
| | dimethyl-5- | | | 5-cyclopropyl-4,7-difluoro- |
| | oxopyrrolidin-1- | | | 3,3-dimethylindolin-2-one |
| | yl)butanoic acid | | | (Intermediate 3) as the |
| 25A* | (R)-4-(4-(5-cyclopropyl- | 435.3 | ¹H NMR (400 MHz, | indolinone (i) reagent and |
| | 4,7-difluoro-3,3- | | Methanol-d₄) δ 6.67- | tert-butyl 4-(4-chloro-2,2- |
| | dimethyl-2-oxoindolin- | | 6.60 (m, 1H), 5.67 (d, | dimethyl-5-oxopyrrolidin-1- |
| | 1-yl)-2,2-dimethyl-5- | | J = 8.4 Hz, 1H), 3.34 | yl)butanoate as the lactam |
| | oxopyrrolidin-1- | | (s, 1H), 3.32 (s, 1H), | (ii) reagent, using 3-bromo- |
| | yl)butanoic acid | | 2.67 (d, J = 8.0 Hz, | 5,5-dimethylpyrrolidin-2- |
| | | | 1H), 2.36-2.29 (m, | one as the step 1 reagent to |
| | | | 2H), 2.11-1.90 (m, | prepare intermediate tert- |
| | | | 4H), 1.53 (s, 3H), 1.49 | butyl 4-(4-(5-cyclopropyl- |
| | | | (s, 3H), 1.44 (s, 3H), | 4,7-difluoro-3,3-dimethyl-2- |
| | | | 1.38 (s, 3H), 1.06- | oxoindolin-1-yl)-2,2- |
| | | | 0.96 (m, 2H), 0.73- | dimethyl-5-oxopyrrolidin-1- |
| | | | 0.68 (m, 2H). | yl)butanoate (25-OtBu). 25- |
| 25B* | (S)-4-(4-(5-cyclopropyl- | 435.3 | ¹H NMR (400 MHz, | OtBu was subjected to |
| | 4,7-difluoro-3,3- | | Methanol-d₄) δ 6.66 | chiral separation conditions |
| | dimethyl-2-oxoindolin- | | (d, J = 10.8 Hz, 1H), | (CHIRALPAK IG, 2*25 cm, |
| | 1-yl)-2,2-dimethyl-5- | | 5.67 (d, J = 8.4 Hz, | 5 μm; Mobile Phase A: |
| | oxopyrrolidin-1- | | 1H), 3.34 (s, 1H), 3.32 | Hexanes (0.5% 2M NH₃- |
| | yl)butanoic acid | | (s, 1H), 2.67 (d, J =8.0 | MeOH), Mobile Phase B: |
| | | | Hz, 1H), 2.37-2.32 | EtOH:DCM = 1:1; Flow |
| | | | (m, 2H), 2.09-1.86 | rate: 20 mL/min; Gradient: |
| | | | (m, 4H), 1.53 (s, 3H), | 25% B to 25% B in 7 min; |
| | | | 1.49 (s, 3H), 1.44 (s, | Wave Length: 220 nm/254 |
| | | | 3H), 1.38 (s, 3H), 1.05- | nm) to provide as the first |
| | | | 0.97 (m, 2H), 0.76- | eluting peak tert-butyl (R)- |
| | | | 0.69 (m, 2H). | 4-(4-(5-cyclopropyl-4,7- |
| | | | | difluoro-3,3-dimethyl-2- |
| | | | | oxoindolin-1-yl)-2,2- |
| | | | | dimethyl-5-oxopyrrolidin-1- |
| | | | | yl)butanoate (25A*-OtBu; |
| | | | | Chiral RT (min): 4.97), and |
| | | | | as the second eluting peak |
| | | | | tert-butyl (S)-4-(4-(5- |
| | | | | cyclopropyl-4,7-difluoro- |
| | | | | 3,3-dimethyl-2-oxoindolin- |
| | | | | 1-yl)-2,2-dimethyl-5- |
| | | | | oxopyrrolidin-1- |
| | | | | yl)butanoate (25B*-OtBu; |
| | | | | Chiral RT (min): 6.07). |
| | | | | 25A*-OtBu and 25B*- |
| | | | | OtBu were hydrolyzed as in |
| | | | | Example 8 to provide 25A* |
| | | | | and 25B*. |
| | | | | *Stereochemistry of 25A* |
| | | | | and 25B* were arbitrarily |
| | | | | assigned. |

### Assay Methods

Human Whole Blood (hWB) NLRP3 Assay

The objective of this assay to is demonstrate if a test compound is able to interfere with human NLRP3 function in a whole blood system.

Human whole blood is drawn from healthy volunteers after obtaining written informed consent. Heparin lithium coated tubes are used to collect blood from volunteers. Blood samples are distributed on 96 well plates using 90 μl per well.

Priming procedure: Priming is performed by adding 5 μl of LPS (026:B6; Sigma L-2654) at a final concentration of 1 μg/ml for 4.5 hours in a humidified incubator with 37° C., 5% CO₂. Thirty minutes prior to NLRP3 activation, 5 μl of a 20× compound solution or vehicle (2% dimethylsulfoxide (DMSO)) is added to each well and plates were incubated on a shaker (450 rpm) in a humidified incubator with 37° C., 5% CO₂.

Alternative priming procedure: Thirty minutes prior to LPS priming, 5 μl of a 20× compound solution or vehicle (2% dimethylsulfoxide (DMSO)) is added to each well and plates were incubated on a shaker (450 rpm) in a humidified incubator with 37° C., 5% CO₂. Priming is performed by adding 5 μl of LPS (026:B6; Sigma L-2654) at a final concentration of 1 μg/ml for 5 hours in a humidified incubator with 37° C., 5% CO₂.

Activation is then performed by adding 3.3 μl of a 31×ATP solution per well. At the end of the 30 minutes stimulation, the plates are centrifuged (800 g, 10 min, room temperature) and the plasma from each well is frozen at −80° C. IL-10 levels in the supernatant were analyzed using a mesoscale discovery assay (MSD K151TUK) according to the manufacturers' instructions.

There is no detectable difference in assay IC₅₀ results using the priming procedure versus the alternative priming procedure.

Data provided in Table C, below. hWB IL-10 Assay: A represents an IC₅₀ value <0.3 μM; B represents an IC₅₀ value ≥0.3 μM and <0.5 μM; C represents an IC₅₀ value ≥0.5 μM and <1.0 μM; D represents an IC₅₀ value ≥1.0 μM and <10 μM; E represents an IC₅₀ value ≥10 μM. Dashed (--) lines indicate no data available.

TABLE C
Activity Data

| Compound # | hWB IL-1β IC₅₀ (μM) |
|---|---|
| 1 | D |
| 2 | D |
| 3 | D |
| 4A* | E |
| 4B* | — |
| 5 | D |
| 6 | D |
| 7 | D |
| 8 | B |
| 9 | D |
| 10 | B |
| 11 | C |
| 12 | D |
| 13 | E |
| 14 | D |
| 15 | E |
| 16A | A |
| 16B | D |
| 17 | C |
| 18A* | D |
| 18B* | E |
| 19A* | A |
| 19B* | — |
| 20A* | — |
| 20B* | — |
| 20C* | C |
| 20D* | C |
| 21 | D |
| 22A* | A |
| 22B* | — |
| 23A* | D |
| 23B* | — |
| 24A* | B |
| 24B* | — |
| 24C* | E |
| 24D* | D |
| 25A* | E |
| 25B* | E |
| 26A* | B |
| 26B* | — |
| 26C* | — |
| 26D* | E |

Compounds of Formula (I), where R² and R⁷ are joined to form a heterocyclic or a heteroaryl lactam ring, as defined herein, comprise features that aid in achieving inhibitory activity against NLRP3, and include a non-hydrogen R¹ group (e.g., cyclopropyl or —CF₃), di-substitution at the R⁵/R⁵ position (e.g., di-methyl), and an —R⁴—Z moiety (e.g., when R⁴ is a substituted or unsubstituted —C₃alkylene- and Z is —CO₂H).

When R² and R⁷ are joined to form a saturated heterocyclic lactam ring, stereochemistry at the carbon to which R² is attached may be an important feature. For example, as shown in below Table D, Compound 18A*, rationally assigned as having R-stereochemistry, is >147-fold more active than 18B*, rationally assigned as having S-stereochemistry.

TABLE D
Hereocyclic lactams

| | hWB IL- |
|---|---|
| | 1β |
| Compound | IC₅₀ (μM) |
| 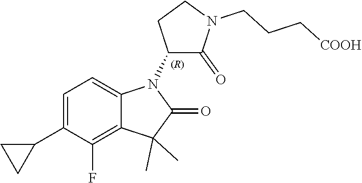 | 3.4 (“D” activity) |
| Compound 18A* | |
| 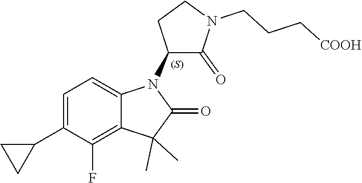 | >500 (“E” activity) |
| Compound 18B* | |

When R² and R⁷ are Joined to form a heteroaryl lactam ring, inclusion of an additional ring N atom may also be an important feature. For example, as shown below in Table E, inclusion of an additional N ring atom ortho (Compound 11), meta (Compound 8), or para (Compound 10) to the lactam ring N atom results in a 3-5-fold improvement in activity compared to a compound with the lactam ring N as the sole N ring atom (Compound 3).

TABLE E
Hereoaryl lactams

| | hWB |
|---|---|
| Compound | IL-1β |
| | IC₅₀ (μM) |
| 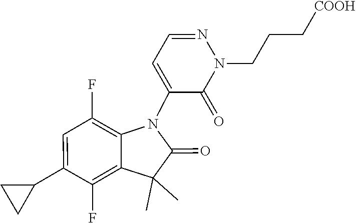 [SMILES: CC1(C)C(=O)N(c2ccnn(CCCC(=O)O)c2=O)c2c(F)cc(C3CC3)c(F)c21] | 0.61 (“C” activity) |
| Compound 11 (ortho N) | |
| 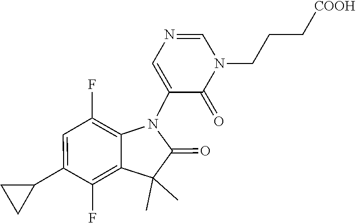 [SMILES: CC1(C)C(=O)N(c2cncn(CCCC(=O)O)c2=O)c2c(F)cc(C3CC3)c(F)c21] | 0.47 (“B” activity) |
| Compound 8 (meta N) | |
| 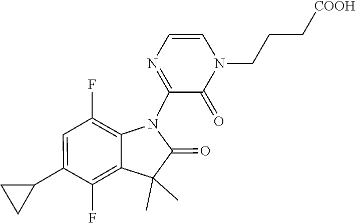 [SMILES: CC1(C)C(=O)N(c2nccn(CCCC(=O)O)c2=O)c2c(F)cc(C3CC3)c(F)c21] | 0.39 (“B” activity) |
| Compound 10 (para N) | |
| 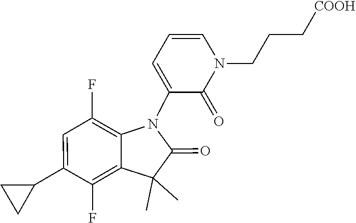 [SMILES: CC1(C)C(=O)N(c2cccn(CCCC(=O)O)c2=O)c2c(F)cc(C3CC3)c(F)c21] | 1.9 (“D” activity) |
| Compound 3 | |

### Other Embodiments

Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims or embodiments is introduced into another claim or embodiment. For example, any claim that is dependent on another claim can be modified to include one or more limitations found in any other claim that is dependent on the same base claim. Where the present disclosure recites elements presented as lists, e.g., in Markush group format, each subgroup of the elements is also disclosed, and any element(s) can be removed from the group. It should be further understood that when any variable (e.g., an R group) is present more than one time in a given Markush structure (e.g., 2 or more times), and that variable may be selected from a given list of two or more elements, unless otherwise stated or understood within the context of the present disclosure, that variable (e.g., the R group) at each repeated occurrence (e.g., 2 or more times) is independent of each other, being independently selected from that given list.

It should also be understood that, in general, where the present disclosure, or aspects of the present disclosure, is/are referred to as comprising particular elements and/or features, certain embodiments of the present disclosure or aspects of the present disclosure also consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are understood to be included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the present disclosure, the present disclosure shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

The foregoing has been described of non-limiting embodiments of the present disclosure. Those of ordinary skill in the art will appreciate that various changes and modifications to this description may be made without departing from the spirit or scope of the present disclosure, as defined in the following claims.

What is claimed is:

1. A compound of Formula (I): 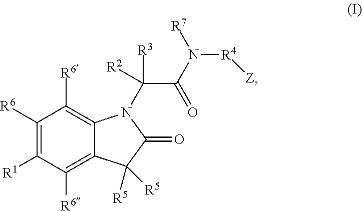
or a pharmaceutically acceptable salt thereof, wherein:
R¹ is halo, C₁-C₆ alkyl, C₁-C₆ haloalkyl, or C₃-C₆ cycloalkyl;
R⁷ and R² are joined to form a 5- to 10-membered monocyclic or bicyclic heterocyclyl or 5- to 10-membered monocyclic or bicyclic heteroaryl, wherein the heterocyclyl or heteroaryl are optionally substituted with one or more R^{7a}, wherein each R^{7a} is independently halo, —CN, —OH, C₁-C₆ alkyl, C₁-C₆ haloalkyl, C₁-C₆ alkoxy, C₁-C₆ haloalkoxy, C₆ aryl, 5- to 6-membered heteroaryl, C₃-C₅ cycloalkyl, or 4 to 5-membered heterocyclyl, wherein the alkyl, haloalkyl, alkoxy, haloalkoxy, aryl, heteroaryl, cycloalkyl, and heterocyclyl are optionally substituted with one or more R^{7b}, further wherein R^{7b} is halo, —CN, —OH, C₁-C₆ alkyl, C₁-C₆ haloalkyl, C₁-C₆ alkoxy, or C₁-C₆ haloalkoxy;
R³ is absent, H, C₁-C₆ alkyl, or C₁-C₆ haloalkyl;
R⁴ is —C₁-C₆ alkylene- optionally substituted with one or more R^{4a}, wherein each R^{4a} is independently halo, —CN, —OH, C₁-C₆ alkyl, C₁-C₆ haloalkyl, C₁-C₆ alkoxy, C₁-C₆ haloalkoxy, C₃₋₄ cycloalkyl, or 4- to 5-membered heterocyclyl, or two R^{4a} are joined to form a C₃-C₅ cycloalkyl or 4-6 membered heterocyclyl ring, wherein the alkyl, haloalkyl, alkoxy, haloalkoxy, cycloalkyl, and heterocyclyl are optionally substituted with one or more R^{4b}, further wherein R^{4b} is halo, —CN, —OH, C₁-C₆ alkyl, C₁-C₆ haloalkyl, C₁-C₆ alkoxy, or C₁-C₆ haloalkoxy;
Z is a —CO₂H or —CO₂PG, wherein PG is C₁-C₆ alkyl, C₆ aryl, or (C₆ aryl)(C₁-C₆ alkyl), further wherein alkyl and aryl are optionally substituted with one or more halo, C₁-C₆ alkyl, C₁-C₆ alkoxy, or C₁-C₆ haloalkoxy;
each R⁵ is independently halo, C₁-C₆ alkyl, or C₁-C₆ haloalkyl; and
each R⁶, R^{6′}, or R^{6″} is independently H, halo, —OH, C₁-C₆ alkoxy, C₁-C₆ haloalkoxy, C₁-C₆ alkyl, or C₁-C₆ haloalkyl.

2. The compound of claim 1, or a pharmaceutically acceptable salt thereof, wherein:
R¹ is halo, C₁-C₆ haloalkyl, or C₃-C₄ cycloalkyl;
R⁷ and R² are joined to form a 5- to 10-membered monocyclic or bicyclic heteroaryl, wherein the heteroaryl is optionally substituted with one or more R^{7a}, wherein each R^{7a} is independently C₁-C₆ alkyl, C₁-C₆ haloalkyl, C₆ aryl, or C₃-C₅ cycloalkyl;
R³ is absent;
R⁴ is —C₁-C₆ alkylene-;
Z is a —CO₂H;
each R⁵ is independently C₁-C₆ alkyl; and
each R⁶, R^{6′}, or R^{6″} is independently H, halo, or —OH.

3. The compound of any one of the preceding claims, or a pharmaceutically acceptable salt thereof, wherein R¹ is Br, Cl, —CF₃, or cyclopropyl.

4. The compound of any one of the preceding claims, or a pharmaceutically acceptable salt thereof, wherein R³ is absent.

5. The compound of any one of the preceding claims, or a pharmaceutically acceptable salt thereof, wherein each instance of R is the same group.

6. The compound of claim 5, or a pharmaceutically acceptable salt thereof, wherein each R⁵ is methyl.

7. The compound of any one of the preceding claims, or a pharmaceutically acceptable salt thereof, wherein R⁶ is H.

8. The compound of any one of the preceding claims, or a pharmaceutically acceptable salt thereof, wherein R^{6′} is F or —OH.

9. The compound of any one of the preceding claims, or a pharmaceutically acceptable salt thereof, wherein R^{6″} is H or F.

10. The compound of any one of the preceding claims, or a pharmaceutically acceptable salt thereof, wherein R⁷ and R² are joined to form a 6-membered monocyclic heteroaryl optionally substituted with one or more R^{7a}.

11. The compound of any one of claims 1-9, or a pharmaceutically acceptable salt thereof, wherein R⁷ and R² are joined to form a 10-membered bicyclic heteroaryl.

12. The compound of any one of the preceding claims, or a pharmaceutically acceptable salt thereof, wherein R^{7a} is methyl, —CF₃, C₆ aryl, or cyclopropyl.

13. The compound of any one of the preceding claims, or a pharmaceutically acceptable salt thereof, wherein R⁴ is —C₃ alkylene-.

14. The compound of any one of the preceding claims, or a pharmaceutically acceptable salt thereof, wherein Z is CO₂H.

15. The compound of any one of the preceding claims, wherein the compound is of Formula (I-A): 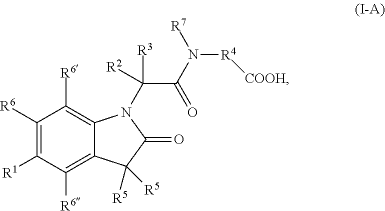
or a pharmaceutically acceptable salt thereof.

16. The compound of claim 15, wherein the compound is of Formula (I-A-a): 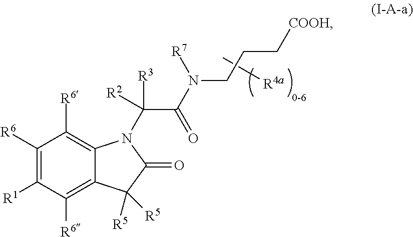
or a pharmaceutically acceptable salt thereof.

17. The compound of claim 16, wherein the compound is of Formula (I-B′), (I-C′), (I-D′), (I-E′), (I-F′), or (I-G′): 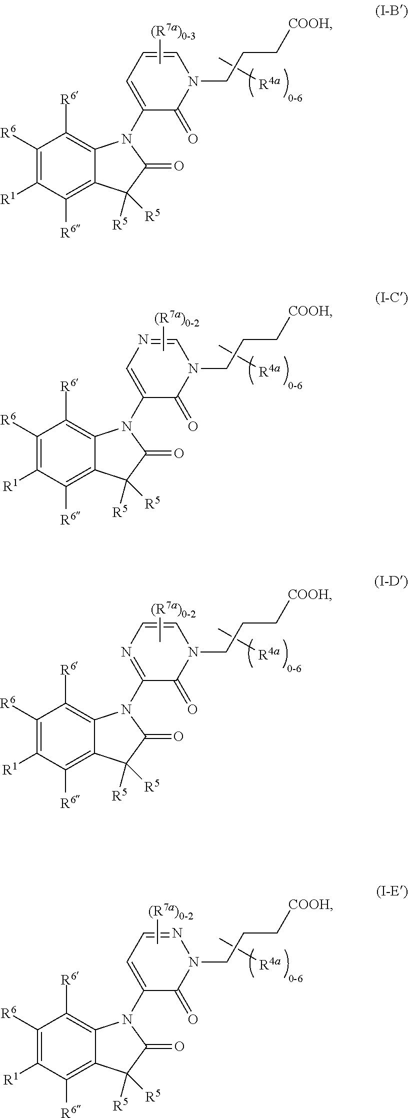 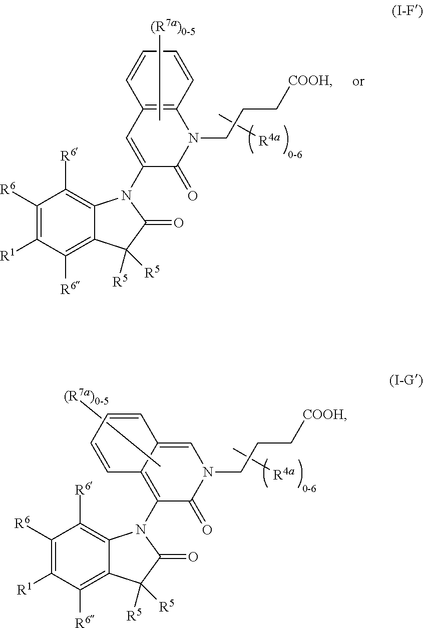
or a pharmaceutically acceptable salt thereof.

18. The compound of claim 16, wherein the compound is of Formula (I-J′): 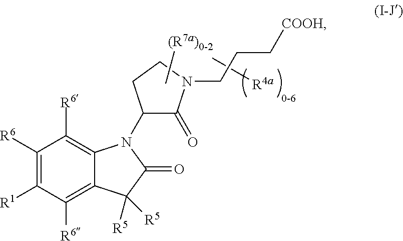
or a pharmaceutically acceptable salt thereof.

19. The compound of claim 18, wherein the compound is of Formula (I-J′-a): 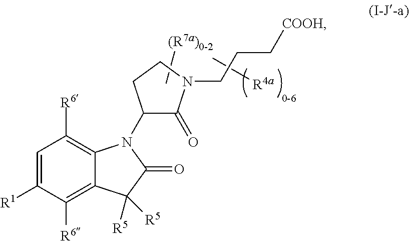
or a pharmaceutically acceptable salt thereof.

20. The compound of claim 19, wherein the compound is of Formula (I-J′-a-R): 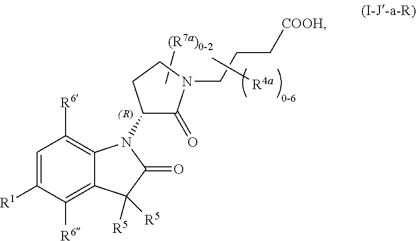
or a pharmaceutically acceptable salt thereof.

21. The compound of any one of claims 16-20, or a pharmaceutically acceptable salt thereof, wherein R^{4a} is absent.

22. The compound of any one of claims 16-21, or a pharmaceutically acceptable salt thereof, wherein R^{7a} is absent.

23. The compound of any one of claims 16-22, or a or a pharmaceutically acceptable salt thereof, wherein R¹ is —CF₃ or cyclopropyl.

24. The compound of any one of claims 16-23, or a or a pharmaceutically acceptable salt thereof, wherein R^{6′} is halo and R^{6″} is H, or each of R^{6′} and R^{6″} is independently halo.

25. The compound of any one of the preceding claims, wherein the compound is selected from the compounds described in Table 1 or Tables 2A-2B, or a pharmaceutically acceptable salt thereof.

26. A pharmaceutical composition comprising the compound of any one of claims 1-25, or a pharmaceutically acceptable salt thereof, and one or more pharmaceutically acceptable carriers.

27. A method of treating or preventing a disease or disorder, the method comprising administering to the subject a compound of any one of claims 1-25, or a pharmaceutically acceptable salt thereof, or a pharmaceutical composition of claim 26.

28. A process for preparing a compound of Formula (I), or a pharmaceutically acceptable salt thereof, the method comprising coupling a substituted indolinone (i), or salt thereof, with a lactam of formula (ii), or salt thereof: 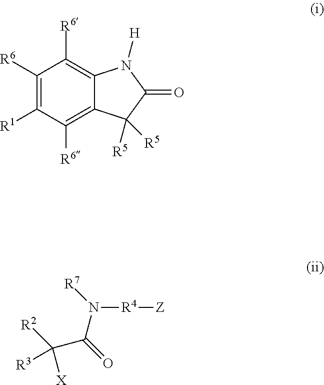
wherein R¹, R², R³, R⁴, R⁵, R⁶, R^{6′}, and R^{6″} are as defined in claim 1, X is a leaving group, and Z is —CO₂PG, wherein PG is as defined in claim 1, to provide a compound of Formula (I), or salt thereof, wherein Z is —CO₂PG; optionally followed by deprotection to provide a compound of Formula (I), or salt thereof, wherein Z is —CO₂H.
